(19) United States
(12) Patent Application Publication
(10) Pub. No.: US 2024/0293392 A1
(43) Pub. Date: Sep. 5, 2024

(54) INHIBITOR COMPOUNDS

(71) Applicant: NeoPhore Limited, Altrincham Cheshire (GB)

(72) Inventors: Julian Blagg, Cambridgeshire (GB); Martin Drysdale, Glasgow (GB); David Clark, Saffron Walden (GB); Paul Winship, Saffron Walden (GB)

(21) Appl. No.: 18/570,244

(22) PCT Filed: Jul. 18, 2022
(86) PCT No.: PCT/GB2022/051846

§ 371 Date: Dec. 14, 2023

(30) Foreign Application Priority Data
Jul. 19, 2021 (GB) 2110373.4

(51) Int. Cl. A61K 31/4725; A61K 31/4035; A61K 31/4375; A61K 31/496; A61K 31/501; C07D 209/44; C07D 217/06; C07D 401/12; C07D 405/14; C07D 471/04

(52) U.S. Cl. CPC A61K 31/4725; A61K 31/4035; A61K 31/4375; A61K 31/496; A61K 31/501; C07D 209/44; C07D 217/06; C07D 401/12; C07D 405/14; C07D 471/04

(57) ABSTRACT

The present invention relates to compounds of Formula (I) that target the MLH1 or MLH1 and PMS2 proteins that are components of the DNA Mismatch Repair (MMR) process: Formula (I) wherein R6, Y1, Y2, A1, A2, A3, A4, R11, R12, R13 and R14 are each as defined herein. The present invention also relates to processes for the preparation of these compounds, to pharmaceutical compositions comprising them, and to their use in the treatment of proliferative disorders, such as cancer, as well as other diseases or conditions in which MLH1 or MLH1 and PMS2 activity is implicated.

### INTRODUCTION

The present invention relates to certain compounds that function as selective inhibitors of MLH1 or MLH1 and PMS2 protein activity. The compounds of the present invention may be used to treat disease or conditions mediated, at least in part, by inappropriate MLH1 and PMS2 activity, for example, cancer. The invention furthermore relates to the use of the compounds as pharmaceuticals, processes for making them and pharmaceutical compositions comprising them.

### BACKGROUND OF THE INVENTION

Cancer is caused by altered cellular proliferation. Precisely what causes a cell to become malignant and proliferate in an uncontrolled and unregulated manner has been the focus of intense research over recent decades. This research has led to the identification of molecular targets associated with key pathways that enable such malignancies.

Mismatch repair (MMR) is a highly conserved DNA repair pathway that plays a major role during DNA replication, repair and recombination, as well as during meiosis in eukaryotes and immunoglobulin maturation/diversification in mammals. MMR promotes genome stability in all organisms by correcting DNA base mismatches and insertion/deletion (indel) loops that can occasionally arise during normal DNA replication process. Base pair mismatches occur when incorrect nucleotides are inserted into the newly synthesized DNA strand and escape the proofreading function of DNA polymerases. Indel loops commonly arise in the context of microsatellites—highly polymorphic short repetitive DNA sequences distributed throughout both prokaryotic and eukaryotic genomes. Typically, at microsatellites, the template and primer strands are prone to slippage (dissociation and reannealing) during replication, which can generate loop structures and a discordant number of repeat units between the template and newly synthesized strand.

DNA mismatch repair is a bidirectional excision and re-synthesis system that initiates at a defined strand scission 3′- or 5′- to a mismatch; the excision tract extends just past the mismatch. MMR can be divided into four steps: 1) mismatch recognition by MSH proteins; 2) recruitment of MLH proteins that connect the mismatch recognition signal to where the distant DNA strand scission begins; 3) excision of the errant DNA strand, and 4) re-synthesis of the excision gap using the remaining DNA strand as a template [1]. MMR is a highly conserved biological pathway. In humans, mismatch recognition by hMutSa (MSH2-MSH6) or hMutSβ (MSH2-MSH3) initiates the MMR pathway. Binding of hMutSα or hMutSβ to the mismatch site results in the recruitment of MutLα (MLH1-PMS2) to form a ternary complex whose protein-protein and protein-DNA interactions are modulated by ATP/ADP cofactors. Proliferating cell nuclear antigen (PCNA) may play a role in the recruitment of MMR proteins to the vicinity of the replication fork [1]. PCNA may also activate a latent endonuclease activity in eukaryotic MutLα proteins. After DNA incision, exonuclease 1 (EXO1) is recruited which excises the newly synthesized DNA strand and the DNA excision gap is re-synthesized by DNA polymerase 6 (Pol 6). When DNA re-synthesis is complete, the remaining nick is ligated by DNA ligase to restore the integrity of the duplex [2]. Consistent with this function, MMR is an important tumor suppressor pathway that is lost in up to 40% of sporadic cancers. Moreover, individuals with germline mutations in MMR genes develop cancer predisposition conditions.

Lynch Syndrome (LS, formerly designated as hereditary non-polyposis colorectal cancer) is the most common cause of hereditary colorectal cancer (CRC), accounting for 2-5% of all cases. LS is also characterized by an increased risk of malignancies at certain extracolonic sites such as the endometrium, ovary, stomach and small bowel, among others [3]. LS has an autosomal dominant inheritance pattern and is caused by germline mutations in MMR genes MLH1, MSH2, MSH6 or PMS2. Gene expression from the one wild-type allele is sufficient for adequate MMR activity until a second hit inactivates the wild-type allele leading to MMR deficiency.

Constitutional mismatch repair deficiency (CMMRD) syndrome is a distinct childhood cancer predisposition syndrome that results from biallelic germline mutations in one of the four MMR genes, MLH1, MSH2, MSH6 or PMS2. Patients may have either homozygous biallelic alterations or heterozygous alterations of MMR genes.

MMR-deficient cancers are commonly and typically characterized by the accumulation of DNA mutations at higher rates than normal cells and other tumours; for example, CMMRD tumours commonly have an ultra-hypermutated phenotype (>250 substitution mutations/Mb) [4]. MMR deficiency also results in gains or losses in the repeat length of microsatellites, referred to as microsatellite instability (MSI). Cancers that possess more than 40% microsatellite variations (positive for two or more of five microsatellite markers routinely tested) are described as high frequency MSI (MSI-H). Tumours that have no MSI are microsatellite stable (MSS) and those that possess less than 40% microsatellite variations (one out of the five markers showing microsatellite instability) are low frequency MSI (MSI-L) [5]. MSI analysis is a widely used diagnostic biomarker of MMR-deficient tumours and MSI status is linked with a high prevalence of frameshift (FS) mutations that can occur because of insertion/deletion within coding microsatellites. In addition to altering downstream functions of the protein, the FS creates a new amino acid sequence that serves as a substrate for antigen processing and presentation [6], stimulating the activation of CD8+ T cells (class I) and the “helper” function of CD4+ T cells (class II).

Cancers with a greater number of neoantigens are more prone to immune surveillance and have an increased likelihood of responding to immunotherapy [7]; higher neoantigen load is associated with overall lymphocytic infiltration, TILs, memory T cells, and survival in colorectal cancer [8, 9]. This feature supports a rationale for immunotherapy-based treatment strategies [6]. Consistent with this notion, immune checkpoint inhibitors now offer a significant therapeutic advance in the treatment of MMR-deficient cancers. Inhibitors of PD-1; for example, pembrolizumab (Keytruda) and nivolumab (Opdivo), have been approved by the Food and Drug Administration (FDA) for patients with MMR-D or MSI-H metastatic CRC based upon the significant survival benefit they provide. The CTLA-4 inhibitor ipilimumab (Yervoy), has been approved for use in combination with nivolumab for the treatment of MMR-D or MSI-H CRC patients who were previously treated with chemotherapy. Importantly, the FDA has approved the use of pembrolizumab in MMR-D/MSI-H cancers regardless of histological tumour type [10].

It is now accepted that clinical responses to immune checkpoint inhibitors require the existence of tumour neoantigens and infiltration of T cells that recognize such neoantigens. Higher neoantigen load is associated with response to CTLA-4 and PD-1 blockade in patients with melanoma and non-small-cell lung cancer [11, 12, 13]. The number of neoantigens is linked to TMB, and several large studies have confirmed that high TMB correlates with enhanced checkpoint inhibitor responses and improved overall survival in certain tumour types, such as urothelial carcinoma [14], non-small cell lung cancer [15-18] and small cell lung cancer [19].

Germano et al. recently proposed that MMR inactivation through silencing of MLH1 increases TMB and leads to “dynamic mutational profiles”, resulting in persistent renewal of neoantigens both in vitro and in vivo. This triggers immune surveillance and leads to the control of tumour growth, particularly in combination with immune checkpoint inhibition, in mouse models [20]. Similar results are observed upon silencing of MSH2 [21].

Guan et al. and Lu et al. report that MLH1 deficiency leads to cytosolic DNA release, activation of the cGAS-STING pathway and IFN-3 production. Guan et al. demonstrate that MLH1 loss leads to DNA hyperexcision, RPA exhaustion, chromosomal instability and accumulation of cytosolic DNA. Lu et al. report that the sensing of cytosolic DNA by the cGAS STING pathway contributes to the clinical benefit of immunotherapy in patients harboring MMR deficient tumours. Together these reports suggest that abrogation of MMR activity may elicit beneficial immune activation through activation of the cGAS-STING pathway.

There is therefore a robust biological and clinical rationale highlighting the need for inhibitors that target the MLH1 or MLH1 and PMS2 proteins, key components of DNA MMR to reawaken an anti-tumour immune response.

Thus, the present invention provides methods for the treatment of cancer by binding to and modulating the function of the DNA MMR components MLH1 or MLH1 and PMS2 using small molecules as single agents and in combination with immunotherapy agents, other DNA damage response pathway modulators and/or standard-of-care chemotherapeutic agents.

Outside of the cancer field, triplet repeat disorders comprise over 30 human neurodegenerative and neuromuscular inherited diseases such as Huntington's disease (HD), myotonic dystrophy type 1 (DM1), fragile X syndrome type A (FRAXA), Friedreich's ataxia (FRDA), and spinocerebellar ataxias (SCAs). Such disorders are characterized by the expansion of simple repeats in genomic DNA. These unstable repeats are commonly found at different regions of several genes and their expansion can cause disease by a variety of both loss- and gain-of-function pathways, for instance through interfering with the expression or properties of the gene products, or by affecting splicing or antisense regulation. Several mechanisms including errors during DNA replication, meiotic recombination, transcription, DNA repair, and chromatin remodeling have been proposed to contribute to repeat instability, which can occur at various stages of the cell cycle. There is evidence that a functional MMR pathway is required for maintaining the stability of microsatellite sequences: for example, Msh2−/− transgenic mice bearing a copy of the human HD exon 1 (containing the CAG repeats) showed reduced expansion of the introduced (CAG)n repeats when compared with Msh2+/+HD exon 1 mice counterparts [22].

Thus, there is a further need for compounds that target MLH1 or MLH1 and PMS2 components of the DNA MMR process for treating triplet repeat disorders. The present invention was devised with the foregoing in mind.

### REFERENCES

1. Martin-Lopez, J. V. and R. Fishel, The mechanism of mismatch repair and the functional analysis of mismatch repair defects in Lynch syndrome. Fam Cancer, 2013. 12(2): p. 159-68.
2. Liu, D., G. Keijzers, and L. J. Rasmussen, DNA mismatch repair and its many roles in eukaryotic cells. Mutat Res, 2017. 773: p. 174-187.
3. Lynch, H. T., et al., Review of the Lynch syndrome: history, molecular genetics, screening, differential diagnosis, and medicolegal ramifications. Clin Genet, 2009. 76(1): p. 1-18.
4. Shlien, A., et al., Combined hereditary and somatic mutations of replication error repair genes result in rapid onset of ultra-hypermutated cancers. Nat Genet, 2015. 47(3): p. 257-62
5. Sehgal, R., et al., Lynch syndrome: an updated review. Genes (Basel), 2014. 5(3): p. 497-507
6. Willis, J. A., et al., Immune Activation in Mismatch Repair-Deficient Carcinogenesis: More Than Just Mutational Rate. Clin Cancer Res, 2019.
7. Gubin, M. M. and R. D. Schreiber, CANCER. The odds of immunotherapy success. Science, 2015. 350(6257): p. 158-9.
8. Kloor, M. and M. von Knebel Doeberitz, The Immune Biology of Microsatellite-Unstable Cancer. Trends Cancer, 2016. 2(3): p. 121-133.
9. Giannakis, M., et al., Genomic Correlates of Immune-Cell Infiltrates in Colorectal Carcinoma. Cell Rep, 2016. 17(4): p. 1206.
10. Lemery, S., P. Keegan, and R. Pazdur, First FDA Approval Agnostic of Cancer Site—When a Biomarker Defines the Indication. N Engl J Med, 2017. 377(15): p. 1409-1412.
11. Le, D. T., et al., PD-1 Blockade in Tumors with Mismatch-Repair Deficiency. N Engl J Med, 2015. 372(26): p. 2509-20.
12. Rizvi, N. A., et al., Cancer immunology. Mutational landscape determines sensitivity to PD-1 blockade in non-small cell lung cancer. Science, 2015. 348(6230): p. 124-8.
13. Van Allen, E. M., et al., Genomic correlates of response to CTLA-4 blockade in metastatic melanoma. Science, 2015. 350(6257): p. 207-211.
14. Rosenberg, J. E., et al., Atezolizumab in patients with locally advanced and metastatic urothelial carcinoma who have progressed following treatment with platinum-based chemotherapy: a single-arm, multicentre, phase 2 trial. Lancet, 2016. 387(10031): p. 1909-20.
15. Hellmann, M. D., et al., Genomic Features of Response to Combination Immunotherapy in Patients with Advanced Non-Small-Cell Lung Cancer. Cancer Cell, 2018. 33(5): p. 843-852 e4.
16. Rizvi, H., et al., Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol, 2018. 36(7): p. 633-641.
17. Carbone, D. P., et al., First-Line Nivolumab in Stage IV or Recurrent Non-Small-Cell Lung Cancer. N Engl J Med, 2017. 376(25): p. 2415-2426.
18. Hellmann, M. D., et al., Nivolumab plus Ipilimumab in Lung Cancer with a High Tumor Mutational Burden. N Engl J Med, 2018. 378(22): p. 2093-2104.
19. Hellmann, M. D., et al., Tumor Mutational Burden and Efficacy of Nivolumab Monotherapy and in Combination with Ipilimumab in Small-Cell Lung Cancer. Cancer Cell, 2018. 33(5): p. 853-861 e4.
20. Germano, G., et al., Inactivation of DNA repair triggers neoantigen generation and impairs tumour growth. Nature, 2017. 552(7683): p. 116-120.
21. Mandal, R., et al., Genetic diversity of tumors with mismatch repair deficiency influences anti-PD-1 immunotherapy response. Science, 2019. 364(6439): p. 485-491.
22. Lu, C., et al. DNA sensing in mismatch repair-deficient tumor cells is essential for anti-tumor immunity. Cancer Cell. 2021, 39 (1), 96-108.
23. Guan J., et al., MLH1 deficiency-triggered DNA hyperexcision by exonuclease 1 activates the cGAS-STING pathway. Cancer Cell. 2021, 39 (1), 109-121,
24. Manley, K., et al., Msh2 deficiency prevents in vivo somatic instability of the CAG repeat in Huntington disease transgenic mice. Nat Genet, 1999. 23(4): p. 471-3.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting MLH1 activity or MLH1 and PMS2 activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which MLH1 activity or MLH1 and PMS2 activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a triplet disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound, or a pharmaceutically acceptable salt, hydrate or solvate thereof, or a pharmaceutical composition as defined herein for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, or a pharmaceutical composition as defined herein for use in the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a compound, or a pharmaceutically acceptable salt, hydrate or solvate thereof, as defined herein for use in the inhibition of MLH1 and/or PMS2 activity (e.g. MLH1 activity or PMS2 activity or MLH1 and PMS2 activity).

According to a further aspect of the present invention, there is provided a compound, or a pharmaceutically acceptable salt, hydrate or solvate thereof, as defined herein for use in the treatment of a disease or disorder in which MLH1 activity or MLH1 and PMS2 activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided the use of a compound, or a pharmaceutically acceptable salt, hydrate or solvate thereof, as defined herein in the manufacture of a medicament for the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a use of a compound, or a pharmaceutically acceptable salt, hydrate or solvate thereof, as defined herein in the manufacture of a medicament for the inhibition of MLH1 activity or MLH1 and PMS2 activity.

According to a further aspect of the present invention, there is provided a use of a compound, or a pharmaceutically acceptable salt, hydrate or solvate thereof, as defined herein in the manufacture of a medicament for the treatment of a disease or disorder in which MLH1 activity or MLH1 and PMS2 activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

In the above-outlined aspects of the invention, the proliferative disorder is suitably cancer, and the cancer is suitably a human cancer. In particular, the compounds of the present invention will be useful for the treatment of any cancer in which mis-match repair inhibition and/or cGAS/STING pathway activation is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

### Probe Compounds

The present invention also resides in the discovery of a new series of probe compounds that effectively bind to the ATP-binding site of the GHKL-family proteins MLH1 or MLH1 and PMS2. These probe compounds can be used in assays and methods for assessing the binding affinity of test compounds to the ATP-binding site of MLH1 or MLH1 and PMS2. In addition, these probe compounds can be used to determine the location and/or quantity of MLH1 or MLH1 and PMS2 in a biological sample.

Thus, in another aspect, the present invention provides a probe compound of formula I, or a salt thereof, as defined herein, wherein one of R¹² or R¹³ is a group L-Q or Lₓ-X as defined herein.

In another aspect, the present invention provides a method of synthesising a probe compound of formula I, or a salt thereof, as defined herein.

In another aspect, the present invention provides the use of a probe compound of formula I, or a salt thereof, in a displacement assay to determine the binding affinity of a test molecule to the ATP-binding site of a target protein. In an embodiment, the target protein is selected from MLH1 or MLH1 and PMS2. In a particular embodiment, the target protein is MLH1.

In another aspect, the present invention provides a probe compound of formula I, or a salt thereof, for use in a displacement assay to determine the binding affinity of a test molecule for the ATP-binding site of a target protein. In an embodiment, the target protein is selected from MLH1 or MLH1 and PMS2. In a particular embodiment, the target protein is MLH1.

In another aspect, the present invention provides an assay for determining the binding affinity of a test molecule for the ATP-binding site of a target protein, the assay comprising:
(i) incubating a test molecule with the target protein in the presence of a probe compound of formula I, or a salt thereof, as defined herein; and
(ii) determining whether a probe compound of the formula I is displaced from the ATP-binding site of the target protein.
   In an embodiment, the target protein is selected from MLH1 or MLH1 and PMS2. In a particular embodiment, the target protein is MLH1.

In another aspect, the present invention provides a method for determining the binding affinity of a test molecule for the ATP-binding site of a target protein, the assay comprising:
(i) incubating a test molecule with the target protein in the presence of a probe compound of formula I, or a salt thereof, as defined herein; and
(ii) determining whether a probe compound of the formula I is displaced from the ATP-binding site of the target protein.

In another aspect, the present invention provides an assay for determining the location and/or quantity of a target protein present within a biological sample, the assay comprising:
(i) contacting the biological sample with a probe compound of formula I, or a salt thereof, as defined herein; and
(ii) determining location and/or quantity of the compound of formula I present within the sample by detecting the location and/or intensity of the detection moieties of the compounds of formula I present within the biological sample.

In another aspect, the present invention provides an assay for determining the location and/or quantity of a target protein present within a biological sample, the assay comprising:
(i) contacting the biological sample with a probe compound of formula I, or a salt thereof, wherein one of R¹² or R¹³ is a group Lₓ-X as defined herein; and
(ii) contacting the biological sample with a detection moiety Q, or a compound of the formula Q-L₂-Y as defined herein, that is capable of reacting with the functional group X present on the compound of formula I to form a compound of formula I wherein one of R¹² or R¹³ is a group L-Q as defined herein in situ within the biological sample;
(iii) determining location and/or quantity of the compound of formula I present within the sample by detecting the location and/or intensity of the detection moieties Q of the probe compounds of formula I present within the biological sample.

In another aspect, the present invention provides a method for determining the location and/or quantity of a target protein present within a biological sample, the method comprising:
(i) contacting the biological sample with a probe compound of formula I, or a salt thereof, as defined herein; and
(ii) determining location and/or quantity of the compound of formula I present within the sample by detecting the location and/or intensity of the detection moieties Q of the probe compounds of formula I present within the biological sample.

In another aspect, the present invention provides a method for determining the location and/or quantity of a target protein present within a biological sample, the method comprising:
(i) contacting the biological sample with a probe compound of formula I, or a salt thereof, wherein one of R¹² or R¹³ is a group Lₓ-X as defined herein; and
(ii) contacting the biological sample with a detection moiety Q, or a compound of the formula Q-L₂-Y as defined herein, that is capable of reacting with the functional group X present on the compound of formula I to form a probe compound of formula I wherein one of R¹² or R¹³ is a group L-Q as defined herein in situ within the biological sample;
(iii) determining location and/or quantity of the compound of formula I present within the sample by detecting the location and/or intensity of the detection moieties Q of the compounds of formula I present within the biological sample.

In an embodiment, the target protein is selected from MLH1 or MLH1 and PMS2. In a particular embodiment, the target protein is MLH1, i.e. the probe is selective for MLH1.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from: a benzene ring fused to a 5- or 6-membered ring containing 1, 2 or 3 ring heteroatoms; a pyridine ring fused to a 5- or 6-membered ring containing 1, 2 or 3 ring heteroatoms; a pyrimidine ring fused to a 5- or 6-membered ring containing 1 or 2 ring heteroatoms; a pyrrole ring fused to a 5- or 6-membered ring containing 1, 2 or 3 ring heteroatoms; a pyrazole ring fused to a 5- or 6-membered ring containing 1 or 2 ring heteroatoms; a pyrazine ring fused to a 5- or 6-membered ring containing 1 or 2 ring heteroatoms; an imidazole ring fused to a 5- or 6-membered ring containing 1 or 2 ring heteroatoms; an oxazole ring fused to a 5- or 6-membered ring containing 1 or 2 ring heteroatoms; an isoxazole ring fused to a 5- or 6-membered ring containing 1 or 2 ring heteroatoms; a thiazole ring fused to a 5- or 6-membered ring containing 1 or 2 ring heteroatoms; an isothiazole ring fused to a 5- or 6-membered ring containing 1 or 2 ring heteroatoms; a thiophene ring fused to a 5- or 6-membered ring containing 1, 2 or 3 ring heteroatoms; a furan ring fused to a 5- or 6-membered ring containing 1, 2 or 3 ring heteroatoms; a cyclohexyl ring fused to a 5- or 6-membered heteroaromatic ring containing 1, 2 or 3 ring heteroatoms; and a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to a compound, or a pharmaceutically acceptable salt, hydrate or solvate thereof, having the structural Formula (I), shown below:

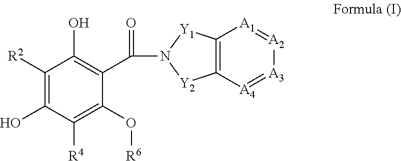

wherein
R² is hydrogen or fluoro;
R⁴ is hydrogen or fluoro;
R⁶ is (1-6C)alkyl, (3-8C)cycloalkyl, or a 4- to 7-membered heterocyclyl ring comprising one heteroatom selected from N, O or S,
   or a group having a structure according to formula (A) shown below:

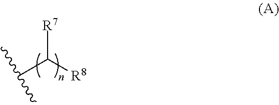

wherein
   R⁷ is hydrogen or (1-3C)alkyl;
   n is 1 or 2;
   R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂— or —C(═O)—;
Y₂ is —CH₂—, —C(═O)—, —CHR_{y2a}, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
A₄ is selected from N, CH or CR¹⁴;
   with the proviso that only one or two of A₁, A₂, A₃ or A₄ can be N;
R¹¹ is cyano, halo, (1-2C)alkyl, (1-2C)alkoxy, wherein any (1-2C)alkyl or (1-2C)alkoxy is optionally substituted by one or more halo or (1-2C)alkoxy;
   or —(CHR_{z})ₐ—Z¹¹,
      wherein R_{z} is hydrogen or methyl;
      wherein a is 0 or 1; and
      Z¹¹ is —OR¹⁵, —NR¹⁶R¹⁷, —C(O)NR¹⁶R¹⁷ or —NR¹⁸C(O)R¹⁹;
         wherein R¹⁵ is (1-4C)alkyl, (3-7C)cycloalkyl, phenyl, a carbon-linked 4 to 8-membered heterocyclyl, a 5 or 6 membered heteroaryl, —(CHR_{y})_{b}—(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-7C)cycloalkyl, phenyl, a carbon-linked 4 to 8-membered heterocyclyl, a 5 or 6 membered heteroaryl, —(CHR_{w})_{c}-(3-7C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1
         or R¹⁶ and R¹⁷ are linked, such that, together with the nitrogen atom to which they are attached, they form a 4-8 membered heterocyclic ring;
         R¹⁸ is hydrogen or (1-2C)alkyl;
         R¹⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₓ)_{d}-(3-7C)cycloalkyl, —(CHRₓ)_{d}-phenyl, —(CHRₓ)_{d}-[4 to 8-membered heterocyclyl] or —(CHRₓ)_{d}-[5 or 6 membered heteroaryl];
         wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
R¹² is selected from one of the following options:
   (i) when R¹¹ is a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹² is —(CHRᵥ)_{d}—Z¹²,
      wherein Rᵥ is hydrogen or methyl;
      wherein d is 0 or 1; and
      Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen or methyl and e is 0 or 1;
         R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or (1-2C)alkyl;
         R²⁴ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
   wherein each of R²⁰, R²¹, R²², R²³ or R²⁴, or any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
         R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or (1-2C)alkyl;
         R²⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein Rⱼ is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or (1-2C)alkyl;
         R³⁴ is (1-6C)alkyl, (2-6C)alkynyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[4 to 8-membered heterocyclyl] or —(CHRₕ)_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group

-L¹-X¹-Q¹
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —N(R¹⁴)—C(O)—NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, (2-4C)alkenyl, (2-4C)alkynyl, or (3-6C)cycloalkyl;
   wherein when Q¹ is (1-4C)alkyl, (2-4C)alkenyl, (2-4C)alkynyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy;
and wherein one of R¹² or R¹³ is optionally a group:

-L-Q

or

-Lₓ-X
wherein L is a linker;
Lₓ is a linker;
X is a functional group; and
Q is a detection moiety.

In one aspect, the present invention relates to a compound, or a pharmaceutically acceptable salt, hydrate or solvate thereof, having the structural Formula (I), shown below:

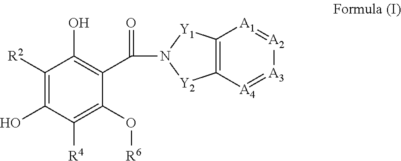

wherein
R² is hydrogen or fluoro;
R⁴ is hydrogen or fluoro;
R⁶ is (1-6C)alkyl, (3-8C)cycloalkyl, or a 4- to 7-membered heterocyclyl ring comprising one heteroatom selected from N, O or S,
   or a group having a structure according to formula (A) shown below:

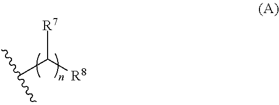

wherein
   R⁷ is hydrogen or (1-3C)alkyl;
   n is 1 or 2;
   R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂— or —C(═O)—;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
A₄ is selected from N, CH or CR¹⁴;
   with the proviso that only one or two of A₁, A₂, A₃ or A₄ can be N;
R¹¹ is cyano, halo, (1-2C)alkyl, (1-2C)alkoxy, wherein any (1-2C)alkyl or (1-2C)alkoxy is optionally substituted by one or more halo or (1-2C)alkoxy;
   or —(CHR_{z})ₐ—Z¹¹,
      wherein R_{z} is hydrogen or methyl;
      wherein a is 0 or 1; and
      Z¹¹ is —OR¹⁵, —NR¹⁶R¹⁷, —C(O)NR¹⁶R¹⁷ or —NR¹⁸C(O)R¹⁹;
         wherein R¹⁵ is (1-4C)alkyl, (3-7C)cycloalkyl, phenyl, a carbon-linked 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-7C)cycloalkyl, phenyl, a carbon-linked 4 to 8-membered heterocyclyl, a 5 or 6 membered heteroaryl, —(CHR_{w})_{c}-(3-7C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1
         or R¹⁶ and R¹⁷ are linked, such that, together with the nitrogen atom to which they are attached, they form a 4-8 membered heterocyclic ring;
         R¹⁸ is hydrogen or (1-2C)alkyl;
         R¹⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₓ)_{d}-(3-7C)cycloalkyl, —(CHRₓ)_{d}-phenyl, —(CHRₓ)_{d}-[4 to 8-membered heterocyclyl] or —(CHRₓ)_{d}-[5 or 6 membered heteroaryl];
         wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
R¹² is selected from one of the following options:
   (iii) when R¹¹ is a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   (iv) when R¹¹ is not a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹² is —(CHRᵥ)_{d}—Z¹²,
      wherein Rᵥ is hydrogen or methyl;
      wherein d is 0 or 1; and
      Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen or methyl and e is 0 or 1;
         R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or (1-2C)alkyl;
         R²⁴ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
   wherein each of R²⁰, R²¹, R²², R²³ or R²⁴, or any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
         R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or (1-2C)alkyl;
         R²⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein Rⱼ is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or (1-2C)alkyl;
         R³⁴ is (1-6C)alkyl, (2-6C)alkynyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[4 to 8-membered heterocyclyl] or —(CHRₕ)_{q}-[5 or 6 membered heteroaryl], wherein Rₕ is hydrogen or methyl and q is 0 or 1;
   wherein R³⁰, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group

-L¹-X¹-Q¹
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —N(R¹⁴)—C(O)—NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, (2-4C)alkenyl, (2-4C)alkynyl, or (3-6C)cycloalkyl
and wherein one of R¹² or R¹³ is optionally a group:

-L-Q

or

-Lₓ-X
wherein L is a linker;
Lₓ is a linker;
X is a functional group; and
Q is a detection moiety.

In a further aspect, the present invention provides a probe compound of formula I defined herein, wherein R², R⁴, R⁶, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

-L-Q

or

-Lₓ-X
wherein L is a linker;
Lₓ is a linker;
X is a functional group; and
Q is a detection moiety.

Particular compounds of the invention include, for example, compounds of the Formula (I), or pharmaceutically acceptable salts, hydrates and/or solvates thereof, wherein, unless otherwise stated, each of R², R⁴, R⁶, Y₁, Y₂, A₁, A₂, A₃, A₄, L, Lₓ, X, Q and any associated substituent groups has any of the meanings defined hereinbefore or in any of paragraphs (1) to (125) hereinafter:
(1) R² is hydrogen;
(2) R² is fluoro;
(3) R⁴ is hydrogen;
(4) R⁴ is fluoro;
(5) R⁶ is (1-6C)alkyl, (3-6C)cycloalkyl, or a 4- to 6-membered heterocyclyl ring comprising one heteroatom selected from N, O or S,
   or a group having a structure according to formula (A) shown below:

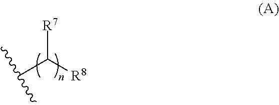

wherein
   R⁷ is hydrogen or (1-3C)alkyl;
   n is 1 or 2;
   R⁸ is (3-7C)cycloalkyl, phenyl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
(6) R⁶ is (1-6C)alkyl, (3-6C)cycloalkyl, or a 4- to 6-membered heterocyclyl ring comprising one heteroatom selected from N, O or S,
   or a group having a structure according to formula (A) shown below:

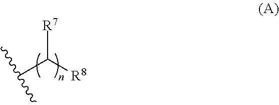

wherein
   R⁷ is hydrogen or (1-3C)alkyl;
   n is 1 or 2;
   R⁸ is (3-8C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
(7) R⁶ is (1-6C)alkyl or a group having a structure according to formula (A) shown below:

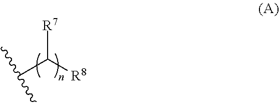

wherein
   R⁷ is hydrogen or (1-3C)alkyl;
   n is 1 or 2;
   R⁸ is (3-6C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy;
(8) R⁶ is (1-4C)alkyl or a group having a structure according to formula (A) shown below:

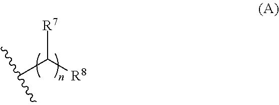

wherein
   R⁷ is hydrogen or (1-2C)alkyl;
   n is 1 or 2;
   R⁸ is (3-6C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy;
(9) R⁶ is (1-4C)alkyl or a group having a structure according to formula (A) shown below:

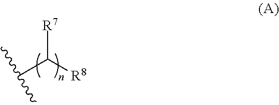

wherein
   R⁷ is hydrogen or methyl;
   n is 1;
   R⁸ is (4-6C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy;
(10) R⁶ is (1-3C)alkyl or a group having a structure according to formula (A) shown below:

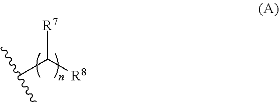

wherein
   R⁷ is hydrogen or methyl;
   n is 1;
   R⁸ is cyclohexyl, phenyl, 6-membered heterocyclyl, 6-membered heteroaryl, each of which is optionally substituted with one or more R⁹; where each R⁹ is independently selected from the group consisting of halogen, methyl, methoxy, trifluoromethyl, or trifluoromethoxy;
(11) R⁶ is a group having a structure according to formula (A) shown below:

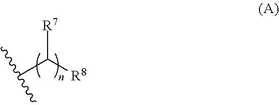

wherein
   R⁷ is hydrogen or methyl;
   n is 1;
   R⁸ is cyclohexyl, phenyl, 6-membered heterocyclyl, 6-membered heteroaryl, each of which is optionally substituted with one or more R⁹; where each R⁹ is independently selected from the group consisting of halogen, methyl, methoxy, trifluoromethyl, or trifluoromethoxy;
(12) R⁶ is a group having a structure according to formula (A) shown below:

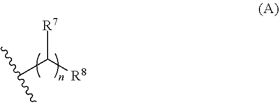

wherein
   R⁷ is hydrogen or methyl;
   n is 1;
   R⁸ is cyclohexyl, phenyl, 6-membered heterocyclyl, pyridyl, each of which is optionally substituted with one or more R⁹; where each R⁹ is independently selected from the group consisting of halogen, methyl, methoxy, trifluoromethyl, or trifluoromethoxy;
(13) R⁶ is a group having a structure according to formula (A) shown below:

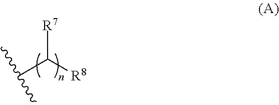

wherein
   R⁷ is hydrogen or methyl;
   n is 1;
   R⁸ is cyclohexyl, phenyl, pyridyl, each of which is optionally substituted with one or more R⁹; where each R⁹ is independently selected from the group consisting of fluoro, chloro, methyl, or methoxy;
(14) R⁶ is a group having a structure according to formula (A) shown below:

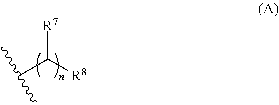

wherein
   R⁷ is hydrogen or methyl;
   n is 1;
   R⁸ is phenyl optionally substituted with one or more R⁹; where each R⁹ is independently selected from the group consisting of fluoro, chloro, methyl, or methoxy;
(15) Y₁ is —CH₂—;
(16) Y₁ is —C(═O)—;
(17) Y₂ is —CH₂—, —CHR_{y2a}—, —CH₂—CH₂—, —CH₂—CHR_{y2b}—, or —CHR_{y2a}—CH₂—; where R_{y2a} is selected from halo, cyano, methyl, CF₃ or hydroxymethyl, and R_{y2b} is selected from halo, cyano, hydroxy, methyl, methoxy, CF₃, —OCF₃ or hydroxymethyl;
(18) Y₂ is —CH₂—, —CHR_{y2a}—, —CH₂—CH₂—, —CH₂—CHR_{y2b}—, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl;
(19) Y₂ is —CH₂—, —CH₂—CH₂—, or —CH₂—CHR_{y2b}—; where R_{y2b} is selected from halo, cyano, hydroxy, methyl, methoxy, CF₃, —OCF₃ or hydroxymethyl;
(20) Y₂ is —CH₂— or —CH₂—CH₂—;
(21) Y₂ is —CH₂—;
(22) Y₂ is —CH₂—CH₂—;
(23) A₁ is selected from CH or CR¹¹;
(24) A₁ is CH;
(25) A₁ is CR¹¹;
(26) A₂ is selected from CH or CR¹²;
(27) A₂ is N;
(28) A₂ is CH;
(29) A₂ is CR¹²;
(30) A₃ is selected from CH or CR¹³
(31) A₃ is N;
(32) A₃ is CH;
(33) A₃ is CR¹³;
(34) A₄ is selected from CH or CR¹⁴;
(35) A₄ is N;
(36) A₄ is CH;
(37) A₄ is CR¹⁴;
(38) A₁ is selected from N, CH or CR¹¹;
   A₂ is selected from N, CH or CR¹²;
   A₃ is selected from N, CH or CR¹³;
   A₄ is selected from N, CH or CR¹⁴;
   with the proviso that only one of A₁, A₂, A₃ or A₄ can be N;
(39) A₁ is selected from CH or CR¹;
   A₂ is selected from N, CH or CR¹²;
   A₃ is selected from N, CH or CR¹³;
   A₄ is selected from N, CH or CR¹⁴;
   with the proviso that only one of A₂, A₃ or A₄ can be N;
(40) A₁ is CR¹¹;
   A₂ is selected from N, CH or CR¹²;
   A₃ is selected from N, CH or CR¹³;
   A₄ is selected from N, CH or CR¹⁴;
   with the proviso that only one of A₂, A₃ or A₄ can be N;
(41) A₁ is CR¹¹;
   A₂ is selected from N or CH;
   A₃ is selected from N, CH or CR¹³;
   A₄ is selected from N or CH;
   with the proviso that only one of A₂, A₃ or A₄ can be N;
(42) A₁ is CR¹¹;
   A₂ is CH;
   A₃ is CH or CR¹³;
   A₄ is CH;
(43) A₁ is CR¹¹;
   A₂ is CH;
   A₃ is CH;
   A₄ is CH;
(44) A₁ is CH;
   A₂ is CR¹²;
   A₃ is CH;
   A₄ is CH;
(45) A₁ is CH;
   A₂ is CH;
   A₃ is CR¹³;
   A₄ is CH;
(46) R¹¹ is cyano, halo or (1-2C)alkyl optionally substituted by one or more halo or (1-2C)alkoxy;
   or —(CHR_{z})ₐ—Z¹¹,
      wherein R_{z} is hydrogen or methyl;
      wherein a is 0 or 1; and
      Z¹¹ is —OR¹⁵, —NR¹⁶R¹⁷, —C(O)NR¹⁶R¹⁷ or —NR¹⁸C(O)R¹⁹;
         wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
         or R¹⁶ and R¹⁷ are linked, such that, together with the nitrogen atom to which they are attached, they form a 4-6 membered heterocyclic ring;
         R¹⁸ is hydrogen or (1-2C)alkyl;
         R¹⁹ is (1-6C)alkyl, (3-6C)cycloalkyl, phenyl, a carbon-linked 4 to 6-membered heterocyclyl, a 5 or 6 membered heteroaryl, —(CHRₓ)_{d}-(3-6C)cycloalkyl, —(CHRₓ)_{d}-phenyl, —(CHRₓ)_{d}-[4 to 6-membered heterocyclyl] or —(CHRₓ)_{d}-[5 or 6 membered heteroaryl];
         wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (69) to (73) or (121) to (125) hereinafter;
(47) R¹¹ is cyano, fluoro, chloro or (1-2C)alkyl optionally substituted by one or more fluoro, chloro or (1-2C)alkoxy;
   or —(CHR_{z})ₐ—Z¹¹,
      wherein R_{z} is hydrogen or methyl;
      wherein a is 0 or 1; and
      Z¹¹ is —OR¹⁵, —NR¹⁶R¹⁷, —C(O)NR¹⁶R¹⁷ or —NR¹$C(O)R¹⁹;
         wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl,
         —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
         or R¹⁶ and R¹⁷ are linked, such that, together with the nitrogen atom to which they are attached, they form a 4-6 membered heterocyclic ring;
         R¹⁸ is hydrogen or (1-2C)alkyl;
         R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
         wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (69) to (73) or (121) to (125) hereinafter;
(48) R¹¹ is cyano or —(CHR_{z})ₐ—Z¹¹,
   wherein R_{z} is hydrogen or methyl;
   wherein a is 0 or 1; and
   Z¹¹ is —OR¹⁵, —NR¹⁶R¹⁷ or —NR¹⁸C(O)R¹⁹;
      wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
      wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (69) to (73) or (121) to (125) hereinafter;
(49) R¹¹ is —(CHR_{z})ₐ—Z¹¹,
   wherein R_{z} is hydrogen;
   wherein a is 0 or 1; and
   Z¹¹ is —OR¹⁵, —NR¹⁶R¹⁷ or —NR¹⁸C(O)R¹⁹;
      wherein R¹⁵ is (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, or a 5 or 6 membered heteroaryl;
      wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a}:
   wherein R^{a} is as defined hereinbefore or in any one of paragraphs (69) to (73) or (121) to (125) hereinafter;
(50) R¹¹ is —(CHR_{z})ₐ—Z¹¹,
   wherein R_{z} is hydrogen or methyl;
   wherein a is 0 or 1; and
   Z¹¹ is —OR¹⁵, —NR¹⁶R¹⁷ or —NR¹⁸C(O)R¹⁹;
      wherein R¹⁵ is (3-6C)cycloalkyl, a carbon-linked 4 to 6-membered heterocyclyl, 5 or 6 membered heteroaryl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1;
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, or a 5 or 6 membered heteroaryl;
      wherein Rₓ is hydrogen or methyl and d is 0 or 1;
   wherein each of R¹⁵, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (69) to (73) or (121) to (125) hereinafter;
(51) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷:
      wherein R¹⁵ is (3-6C)cycloalkyl, a carbon-linked 4 to 6-membered heterocyclyl, 5 or 6 membered heteroaryl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from (3-6C)cycloalkyl, phenyl, a carbon-linked 4 to 6-membered heterocyclyl, a 5 or 6 membered heteroaryl;
   wherein each of R¹⁵ or R¹⁷ is optionally substituted with one or more R^{a};
   wherein R^{a} is as defined hereinbefore or in any one of paragraphs (69) to (73) or (121) to (125) hereinafter;
(52) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from hydrogen, (1-6C)alkyl, (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1;
   wherein each of R¹⁵ or R¹⁷ is optionally substituted with one or more R^{a};
   wherein R^{a} is as defined hereinbefore or in any one of paragraphs (69) to (73) or (121) to (125) hereinafter;
(53) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1;
   wherein each of R¹⁵ or R¹⁷ is optionally substituted with one or more R^{a};
   wherein R^{a} is as defined hereinbefore or in any one of paragraphs (69) to (73) or (121) to (125) hereinafter;
(54) R¹¹ is —(CHR_{z})ₐ—Z¹¹,
   wherein R_{z} is hydrogen;
   wherein a is 0 or 1; and
   Z¹¹ is —NR¹⁶R¹⁷;
      wherein R¹⁶ is hydrogen and R¹⁷ is selected from (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1;
   wherein R¹⁷ is optionally substituted with one or more R^{a};
   wherein R^{a} is as defined hereinbefore or in any one of paragraphs (69) to (73) or (121) to (125) hereinafter;
(55) R¹¹ is —(CHR_{z})ₐ—Z¹¹,
   wherein R_{z} is hydrogen;
   wherein a is 0 or 1; and
   Z¹¹ is —NHR¹⁷;
      wherein R¹⁷ is selected from;
      (i) a group of the formula (A), (B), (C) or (D):

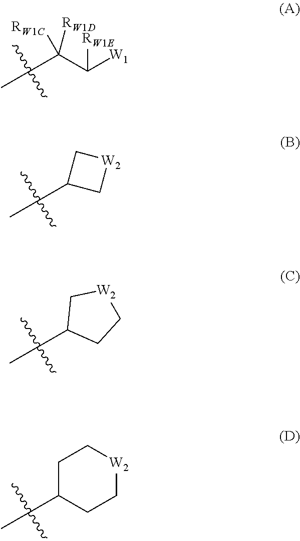

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen or methyl, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a}
W₂ is selected from O, NR_{w2}, S or S(O)₂ where R_{w2} is Selected from hydrogen, (1-4C)alkyl or (2-4C)alkanoyl;
and each of formulae (B), (C) and (D) is optionally substituted by one or more R^{a};
wherein R^{a} is as defined hereinbefore or in any one of paragraphs (69) to (73) or (121) to (125) hereinafter;
(ii) a bridged version of formulae (C) and (D) above, optionally selected from one of formulae (E) to (J) below:

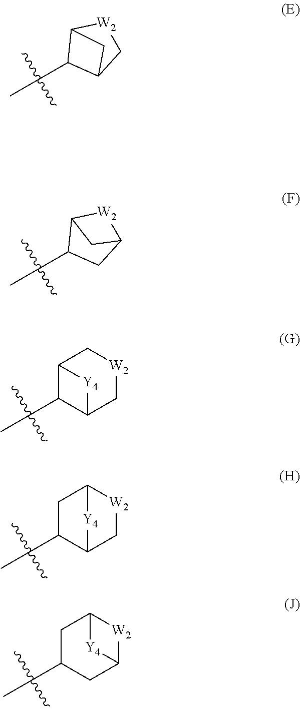

wherein Y₄ is selected from —CH₂—, —CH₂CH₂—,
and each of formulae (E), (F), (G), (H) and (J) is optionally substituted by one or more R^{a};
wherein R^{a} is as defined hereinbefore or in any one of paragraphs (69) to (73) or (121) to (125) hereinafter;
(iii) a group of the formula (K), (L) or (M) below:

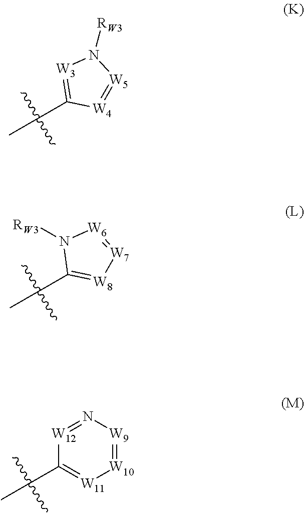

wherein:
R_{W3} is selected from hydrogen or (1-2C)alkyl; W₃, W₄ and W₅ are selected from N or CR_{w4} where R_{w4} is hydrogen, halo, (1-2C)alkyl or (1-2C)alkoxy;
one of W₆ and W₇ is N and the other is CR_{w5} and W₈ is N or CR_{w5}, where R_{w5} is hydrogen, halo, (1-2C)alkyl or (1-2C)alkoxy;
W₉, W₁₀, W₁₁ and W₁₂ are selected from N or CR_{w6} where R_{w6} is hydrogen, halo, (1-2C)alkyl or (1-2C)alkoxy; with the proviso that only one or two of W₉, W₁₀, W₁₁, and W₁₂ are N.
(56) R¹¹ is —(CHR_{z})ₐ—Z¹¹,
   wherein R_{z} is hydrogen;
   wherein a is 0 or 1; and
   Z¹¹ is —NHR¹⁷:
      wherein R¹⁷ is selected from;
      (i) a group of the formula (A), (B), (C) or (D):

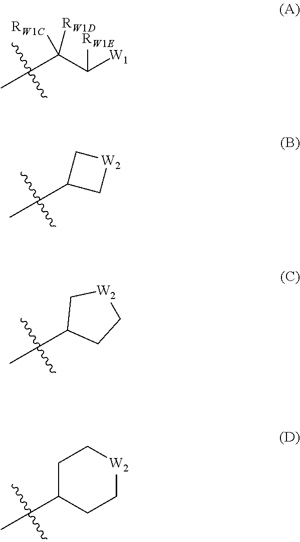

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined hereinbefore or in any one of paragraphs (69) to (73) or (121) to (125) hereinafter;
(ii) a bridged version of formulae (C) and (D) above, optionally selected from one of formulae (E) to (J) below:

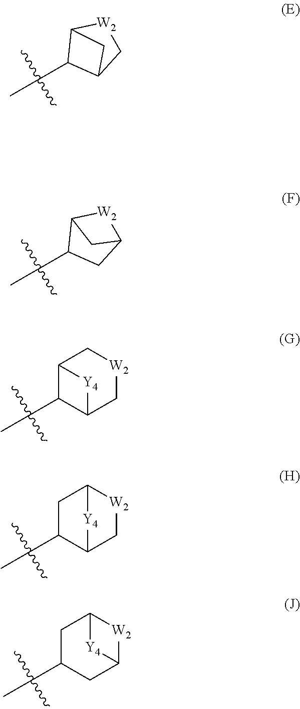

wherein Y₄ is selected from —CH₂—, —CH₂CH₂—,
and each of formulae (E), (F), (G), (H) and (J) is optionally substituted by one or more R^{a};
wherein R^{a} is as defined hereinbefore or in any one of paragraphs (69) to (73) hereinafter or (121) to (125);
(iii) a group of the formula (K), (L) or (M) below:

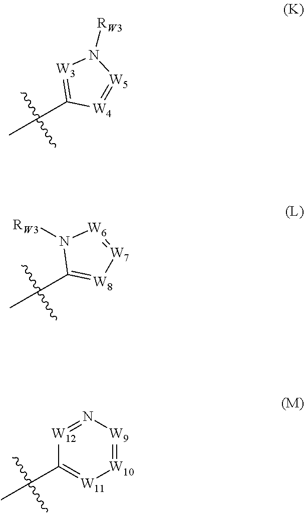

wherein:
R_{W3} is selected from hydrogen or (1-2C)alkyl;
W₃, W₄ and W₅ are selected from N or CH;
one of W₆ and W₇ is N and the other is CH and W₈ is N or CH;
W₉, W₁₀, W₁₁, and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N.
(57) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²,
         wherein Rᵥ is hydrogen or methyl;
         wherein d is 0 or 1; and
         Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen or methyl and e is 0 or 1;
         R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})ᵣ-[⁴ to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or (1-2C)alkyl;
         R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
      wherein each of R²⁰, R²¹, R²², R²³ or R²⁴, or any ring formed when R²¹ and R²² are linked, is optionally substituted with one or more R^{a};
      wherein R^{a} is as defined hereinbefore or in any one of paragraphs (69) to (73) or (121) to (125) hereinafter;
(58) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein Rᵥ is hydrogen or methyl;
         wherein d is 0 or 1; and
         Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen and e is 0 or 1;
         R²¹ and R²² are each independently selected from hydrogen, (1-6C)alkyl, (3-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})ᵣ-[⁴ to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or methyl;
         R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
      wherein each of R²⁰, R²¹, R²², R²³ or R²⁴, or any ring formed when R²¹ and R²² are linked, is optionally substituted with one or more R^{a}:
      wherein R^{a} is as defined hereinbefore or in any one of paragraphs (69) to (73) or (121) to (125) hereinafter;
(59) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein Rᵥ is hydrogen;
         wherein d is 0 or 1; and
         Z¹² is —OR²⁰, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, or a 5 or 6 membered heteroaryl;
         R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
         or R²¹ and R²² are linked, such that, together with the nitrogen atom to which they are attached, they form a 4-8 membered heterocyclic ring;
         R²³ is hydrogen;
         R²⁴ is (1-6C)alkyl, (3-7C)cycloalkyl, phenyl, a carbon-linked 4 to 8-membered heterocyclyl, or a 5 or 6 membered heteroaryl;
      wherein each of R²⁰, R²¹, R²² or R²⁴, or any ring formed when R²¹ and R²² are linked, is optionally substituted with one or more R^{a};
      wherein R^{a} is as defined hereinbefore or in any one of paragraphs (69) to (73) or (121) to (125) hereinafter;
(60) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein Rᵥ is hydrogen;
         wherein d is 0 or 1; and
         Z¹² is —OR²⁰, —NR²¹R²² or —C(O)NR²¹R²²;
         wherein R²⁰ is (1-4C)alkyl;
         R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
         or R²¹ and R²² are linked, such that, together with the nitrogen atom to which they are attached, they form a 4-8 membered heterocyclic ring;
      wherein each of R²⁰, R²¹ or R²², or any ring formed when R²¹ and R²² are linked, is optionally substituted with one or more R^{a};
      wherein R^{a} is as defined hereinbefore or in any one of paragraphs (69) to (73) or (121) to (125) hereinafter;
(61) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy;
(62) R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
         R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or methyl;
         R²⁹ is (1-6C)alkyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R²⁵, R²⁶, R²⁷ or R²⁹ or any ring formed when R²⁶ and R²⁷ are linked, is optionally substituted with one or more R^{a};
   wherein R^{a} is as defined hereinbefore or in any one of paragraphs (69) to (73) or (121) to (125) hereinafter;
(63) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, —NR²⁶R²⁷, —C(O)NR²⁶R²⁷ or —NR²⁸C(O)R²⁹;
         wherein R²⁵ is (1-4C)alkyl, (3-7C)cycloalkyl, a carbon-linked 4 to 8-membered heterocyclyl, —(CHRₙ)ᵢ-(3-7C)cycloalkyl, or —(CHRₙ)ᵢ-[4 to 8-membered heterocyclyl], wherein Rₙ is hydrogen or methyl and i is 0 or 1;
         R²⁶ and R²⁷ are each independently selected from hydrogen, (1-6C)alkyl, (3-7C)cycloalkyl, a carbon-linked 4 to 8-membered heterocyclyl, —(CHRₘ)ⱼ-(3-7C)cycloalkyl, or —(CHRₘ)ⱼ-[4 to 8-membered heterocyclyl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or methyl;
         R²⁹ is (1-6C)alkyl, (3-7C)cycloalkyl, a carbon-linked 4 to 8-membered heterocyclyl, —(CHRₗ)ₖ-(3-7C)cycloalkyl, or —(CHRₗ)ₖ-[4 to 8-membered heterocyclyl], wherein Rₗ is hydrogen or methyl and k is 0 or 1;
   wherein each of R²⁵, R²⁶, R²⁷ or R²⁹ or any ring formed when R²⁶ and R²⁷ are linked, is optionally substituted with one or more R^{a};
   wherein R^{a} is as defined hereinbefore or in any one of paragraphs (69) to (73) or (121) to (125) hereinafter;
(64) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, —NR²⁶R²⁷, or —C(O)NR²⁶R²⁷;
         wherein R²⁵ is (1-4C)alkyl, a carbon-linked 4 to 8-membered heterocyclyl, or —(CHRₙ)ᵢ-[4 to 8-membered heterocyclyl], wherein Rₙ is hydrogen or methyl and i is 0 or 1;
         R²⁶ and R²⁷ are each independently selected from hydrogen, (1-6C)alkyl, a carbon-linked 4 to 8-membered heterocyclyl, or —(CHRₘ)ⱼ-[4 to 8-membered heterocyclyl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
   wherein each of R²⁵, R²⁶, R²⁷ or any ring formed when R²⁶ and R²⁷ are linked, is optionally substituted with one or more R^{a}:
   wherein R^{a} is as defined hereinbefore or in any one of paragraphs (69) to (73) or (121) to (125) hereinafter;
(65) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein Rⱼ is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or methyl;
         R³⁴ is (1-6C)alkyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[4 to 8-membered heterocyclyl] or —(CHRₕ)_{q}-[5 or 6 membered heteroaryl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (69) to (73) or (121) to (125) hereinafter;
(66) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ-Z¹⁴,
      wherein Rₖ is hydrogen;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[4 to 8-membered heterocyclyl], wherein Rⱼ is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or methyl;
         R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[4 to 8-membered heterocyclyl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (69) to (73) or (121) to (125) hereinafter;
(67) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, a carbon-linked 4 to 8-membered heterocyclyl, or —(CHRⱼ)ₒ-[4 to 8-membered heterocyclyl], wherein Rⱼ is hydrogen or methyl and o is 0 or 1;
         R³¹ and R³² are each independently selected from hydrogen, (1-6C)alkyl, a carbon-linked 4 to 8-membered heterocyclyl, or —(CHRᵢ)ₚ-[4 to 8-membered heterocyclyl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen;
         R³⁴ is (1-6C)alkyl, a carbon-linked 4 to 8-membered heterocyclyl, or —(CHRₕ)_{q}-[4 to 8-membered heterocyclyl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (69) to (73) or (121) to (125) hereinafter;
(68) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy;
(69) each R^{a} is independently selected from the group consisting of oxo, halogen, cyano, hydroxy, (1-4C)alkyl, or a group

-L¹-X¹-Q
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, or (3-6C)cycloalkyl;
(70) each R^{a} is independently selected from the group consisting of oxo, halogen, cyano, hydroxy, (1-2C)alkyl, or a group

-L¹-X¹-Q
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-2C)alkyl, or (3-6C)cycloalkyl;
(71) each R^{a} is independently selected from the group consisting of oxo, halogen, cyano, hydroxy, (1-2C)alkyl, or a group

-L¹-X¹-Q
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-2C)alkyl;
(72) each R^{a} is independently selected from the group consisting of oxo, halogen, cyano, hydroxy, (1-2C)alkyl, or a group

-L¹-X¹-Q
wherein:
   L¹ is absent;
   X¹ is absent or is selected from the group consisting of —O—, —C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-2C)alkyl;
(73) each R^{a} is independently selected from the group consisting of oxo, halogen, cyano, hydroxy, (1-2C)alkyl, or a group

-L¹-X¹-Q¹
wherein:
   L¹ is absent;
   X¹ is absent or is selected from the group consisting of —O—, —C(O)—, —C(O)—N(R¹⁴)—, or —NR¹⁴—, where R¹⁴ is hydrogen or (1-2C)alkyl;
   Q¹ is selected from the group consisting of hydrogen or (1-2C)alkyl;
(121) each R^{a} is independently selected from the group consisting of oxo, halogen, cyano, hydroxy, (1-4C)alkyl, or a group

-L¹-X¹-Q
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, or (3-6C)cycloalkyl;
   wherein when Q¹ is (1-4C)alkyl or (3-6C)cycloalkyl, Q¹ is optionally substituted with one or more Q^{a}, where each Q^{a} is independently selected from the group consisting of (1-3C)alkoxy, (1-3C)haloalkyl, or (1-3C)haloalkoxy.
(122) each R^{a} is independently selected from the group consisting of oxo, halogen, cyano, hydroxy, (1-2C)alkyl, or a group

-L¹-X¹-Q
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-3C)alkyl, or (3-6C)cycloalkyl;
   wherein when Q¹ is (1-3C)alkyl or (3-6C)cycloalkyl, Q¹ is optionally substituted with one or more Q^{a}, where each Q^{a} is independently selected from the group consisting of (1-2C)alkoxy, (1-2C)haloalkyl, or (1-2C)haloalkoxy.
(123) each R^{a} is independently selected from the group consisting of oxo, halogen, cyano, hydroxy, (1-2C)alkyl, or a group

-L¹-X¹-Q¹
wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-3C)alkyl;
   wherein when Q¹ is (1-3C)alkyl, Q¹ is optionally substituted with one or more Q^{a}, where each Q^{a} is independently selected from the group consisting of (1-3C)alkoxy.
(124) each R^{a} is independently selected from the group consisting of oxo, halogen, cyano, hydroxy, (1-2C)alkyl, or a group

-L¹-X¹-Q
wherein:
   L¹ is absent;
   X¹ is absent or is selected from the group consisting of —O—, —C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-3C)alkyl;
   wherein when Q¹ is (1-3C)alkyl, Q¹ is optionally substituted with one or more Q^{a}, where each Q^{a} is independently selected from the group consisting of (1-2C)alkoxy.
(125) each R^{a} is independently selected from the group consisting of oxo, halogen, cyano, hydroxy, (1-2C)alkyl, or a group

-L¹-X¹-Q¹
wherein:
   L¹ is absent;
   X¹ is absent or is selected from the group consisting of —O—, —C(O)—, —C(O)—N(R¹⁴)—, or —NR¹⁴—, where R¹⁴ is hydrogen or (1-2C)alkyl;
   Q¹ is selected from the group consisting of hydrogen or (1-3C)alkyl;
wherein when Q¹ is (1-3C)alkyl, Q¹ is substituted with one or more Q^{a}, where each Q^{a} is methoxy.

### Probe Compounds

(74) L is a (2-20C)alkylene linker, wherein the alkylene chain optionally further comprises one or more —O—, —C(O)—, —C(O)NR₁₀—, —NR₁₀C(O)—, —C(O)O—, —OC(O)—, piperazine or triazole ring linkages positioned either within the alkylene chain and/or at one of its termini, wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(75) L is a (3-20C)alkylene linker, wherein the alkylene chain optionally further comprises one or more —O—, —C(O)—, —C(O)NR₁₀—, —NR₁₀C(O)—, —C(O)O—, —OC(O)—, piperazine or triazole ring linkages positioned either within the alkylene chain and/or at one of its termini, wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(76) L is a (2-20C)alkylene linker, wherein the alkylene chain further comprises one to eight —O— linkages and/or one to four —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(77) L is a (3-20C)alkylene linker, wherein the alkylene chain further comprises one to eight —O— linkages and/or one to four —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(78) L is a (2-18C)alkylene linker, wherein the alkylene chain optionally further comprises one to five —O— linkages; and/or one to four —C(O)—, —C(O)NR₁₀—, —NR₁₀C(O)—, —C(O)O—, —OC(O)— linkages or one piperazine or triazole ring linkage positioned either within the alkylene chain and/or at one of its termini,
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(79) L is a (3-12C)alkylene linker, wherein the alkylene chain optionally further comprises one to five —O— linkages; and/or one to four —C(O)—, —C(O)NR₁₀—, —NR₁₀C(O)—, —C(O)O—, —OC(O)— or one piperazine or triazole ring linkages positioned either within the alkylene chain and/or at one of its termini,
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(80) L is a (2-18C)alkylene linker, wherein the alkylene chain further comprises one to five —O— linkages, and/or one to three —C(O)—, —C(O)NR₁₀— or —NR₁₀C(O)— linkages or one piperazine or triazole ring linkage positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(81) L is a (3-12C)alkylene linker, wherein the alkylene chain further comprises one to five —O— linkages and/or one to three —C(O)—, —C(O)NR₁₀— or —NR₁₀C(O)— linkages or one piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(82) L is a (2-18C)alkylene linker, wherein the alkylene chain further comprises one to five —O— linkages and/or one to three —C(O)—, —C(O)NR₁₀— or —NR₁₀C(O)— linkages or one piperazine or triazole ring linkage positioned either within the alkylene chain and/or at one of its termini and wherein R₁₀ is hydrogen or (1-2C)alkyl.
(83) L is a (3-12C)alkylene linker, wherein the alkylene chain further comprises one to five —O— linkages and/or one to three —C(O)—, —C(O)NR₁₀— or —NR₁₀C(O)— linkages or one piperazine or triazole ring linkage positioned either within the alkylene chain and/or at one of its termini and wherein R₁₀ is hydrogen or (1-2C)alkyl.
(84) L is a (2-18C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(85) L is a (3-12C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(86) L is a (2-18C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(87) L is a (3-12C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(88) L is a (2-17C)alkylene linker, wherein the alkylene chain optionally further comprises one to four —O— linkages and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(89) L is a (3-10C)alkylene linker, wherein the alkylene chain optionally further comprises one to four —O— linkages and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(90) L is a (2-17C)alkylene linker, wherein the alkylene chain further comprises one to four —O— linkages; and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(91) L is a (3-10C)alkylene linker, wherein the alkylene chain further comprises one to four —O— linkages; and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(92) L is a (2-17C)alkylene linker, wherein the alkylene chain further comprises one to four —O— linkages and/or one to three —C(O)—, —C(O)NR₁₀—, —NR₁₀C(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(93) L is a (3-10C)alkylene linker, wherein the alkylene chain further comprises one to four —O— linkages and/or one to three —C(O)—, —C(O)NR₁₀—, —NR₁₀C(O)—, piperazine or triazole ring linkages positioned either within the alkylene chain and/or at one of its termini; wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(94) L is a (2-17C)alkylene linker, wherein the alkylene chain further comprises one to four —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(95) L is a (3-10C)alkylene linker, wherein the alkylene chain further comprises one to four —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(96) L is a (2-17C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(97) L is a (3-10C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(98) L is a (2-17C)alkylene linker, wherein the alkylene chain further comprises one —O— linkage and/or one —C(O)—, —C(O)NR₁₀— or —NR₁₀C(O)— linkage or one triazole ring linkage positioned either within the alkylene chain and/or at one of its termini, and wherein R₁₀ is hydrogen or (1-2C)alkyl.
(99) L is a (3-10C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(100) L is a linker having the formula:

-Lₚ₁-Xₚ₁-Lₚ₂-Xₚ₂-Lₚ₃-
wherein
Lₚ₁ is absent or a (1-8C)alkylene;
Xₚ₁ is absent or selected from —O—, —C(O)—, —C(O)NRₚ₁—, —NRₚ₁C(O)—, —C(O)O—, —OC(O)— or a piperazine or triazole ring;
Lₚ₂ is a (1-10C)alkylene or a group of the formula —[CH₂CH₂—O]ₐ—[CH₂]b- or —[CH₂]b-[O—CH₂CH₂]ₐ—, wherein a is 1 to 10 and b is 2 to 4;
Xₚ₂ is absent or selected from —O—, —C(O)—, —C(O)NRₚ₁—, —NRₚ₁C(O)—, —C(O)O—, —OC(O)— or a piperazine or triazole ring;
Lp₃ is absent or a (1-8C)alkylene;
wherein Rₚ₁ is hydrogen or (1-2C)alkyl; and
with the proviso that at least one of Xₚ₁ or Xₚ₂ is present.
(101) L is a linker having the formula:

-Lₚ₁-Xₚ₁-Lₚ₂-Xₚ₂-Lₚ₃-
wherein
Lₚ₁ is absent or a (1-6C)alkylene;
Xₚ₁ is absent or selected from —O—, —C(O)—, —C(O)NRₚ₁—, —NRₚ₁C(O)—, —C(O)O—, —OC(O)— or piperazine or a triazole ring;
Lₚ₂ is a (1-10C)alkylene or a group of the formula —[CH₂CH₂-0]ₐ —[CH₂]_{b}— or —[CH₂]_{b}—[O—CH₂CH₂]ₐ—, wherein a is 2 to 10 and b is 2 to 4;
Xₚ₂ is absent or selected from —O—, —C(O)—, —C(O)NRₚ₁—, —NRₚ₁C(O)—, —C(O)O—, —OC(O)— or a piperazine or triazole ring;
Lp₃ is absent or a (1-6C)alkylene;
wherein Rₚ₁ is hydrogen or methyl; and
with the proviso that at least one of Xₚ₁ or Xₚ₂ is present.
(102) L is a linker having the formula:

-Lₚ₁-Xₚ₁-Lₚ₂-Xₚ₂-Lₚ₃-
wherein
Lₚ₁ is absent or a (2-6C)alkylene;
Xₚ₁ is absent or selected from —O—, —C(O)—, —C(O)NRₚ₁—, —NRₚ₁C(O)— or a piperazine or triazole ring;
Lₚ₂ is a (2-10C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
Xₚ₂ is absent or selected from —O—, —C(O)—, —C(O)NRₚ₁—, —NRₚ₁C(O)— or a piperazine or triazole ring;
Lp₃ is absent or a (1-6C)alkylene;
wherein Rₚ₁ is hydrogen or methyl; and
with the proviso that at least one of Xₚ₁ or Xₚ₂ is present.
(103) L is a linker having the formula:

-Lₚ₁-Xₚ₁-Lₚ₂-Xₚ₂-Lₚ₃-
wherein
Lₚ₁ is absent or a (2-6C)alkylene;
Xₚ₁ is absent or selected from —O—, —C(O)—, —C(O)NRₚ₁—, —NRₚ₁C(O)— or a piperazine or triazole ring;
Lₚ₂ is a (2-6C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
Xₚ₂ is absent or selected from —O—, —C(O)—, —C(O)NRₚ₁—, —NRₚ₁C(O)— or a piperazine or triazole ring;
Lp₃ is absent or a (2-6C)alkylene;
wherein Rₚ₁ is hydrogen or methyl; and
with the proviso that at least one of Xₚ₁ or Xₚ₁ is present.
(104) L is a linker having the formula:

-Lₚ₁-Xₚ₁-Lₚ₂-Xₚ₂-Lₚ₃-
wherein
Lₚ₁ is absent;
Xₚ₁ is absent;
Lₚ₂ is absent or a (2-6C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
Xₚ₂ is selected from —O—, —C(O)—, —C(O)NRₚ₁—, —NRₚ₁C(O)— or a triazole ring;
Lₚ₃ is absent or a (2-6C)alkylene;
wherein Rₚ₁ is hydrogen or methyl; and
with the proviso that at least one of Xₚ₁ or Xₚ₂ is present.
(105) L is a linker having the formula:

-Lₚ₁-Xₚ₁-Lₚ₂-Xₚ₂-Lₚ₃-
wherein
Lₚ₁ is a (2-6C)alkylene;
Xₚ₁ is selected from —O—, —C(O)NRₚ₁—, —NRₚ₁C(O)— or a piperazine or triazole ring;
Lₚ₁ is a (2-6C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
Xₚ₂ is selected from —O—, —C(O)NRₚ₁—, —NRₚ₁C(O)— or a triazole ring;
Lp₃ is a (2-6C)alkylene;
wherein Rₚ₁ is hydrogen or methyl; and
with the proviso that at least one of Xₚ₁ or Xₚ₂ is present.
(106) Q is a detection moiety selected from the group consisting of a fluorophore, an oligonucleotide, a biomolecule, a molecular sensor, a protein, or a peptide.
(107) Q is a fluorophore.
(108) Q is a fluorophore selected from the group consisting of AlexaFluor dyes, Cyanine dyes, fluorescein, BODIPY or BODIPY derivatives (e.g. BODIPY TMR), TAMRA, Oregon Green dyes, FITC, Ru(bpy)3, Rhodamine dyes, Acridine orange, and Texas Red.
(109) Q is a fluorophore selected from the group consisting of AlexaFluor-647, AlexaFluor-633, AlexaFluor-594, AlexaFluor-488, Cyanine-5B, Cyanine-3B, Fluorescein, BODIPY or BODIPY derivatives (e.g. BODIPY TMR), TAMRA, Oregon Green 488, Oregon Green 514, FITC, Ru(bpy)3, Rhodamine dyes, Acridine orange, and Texas Red.
(110) X is a functional group that is capable of reacting with a functional group present on a detection moiety so as to covalently bind the detection moiety to the compound of formula II.
(111) X is a functional group that is capable of reacting with a functional group present on a detection moiety so as to covalently bind the detection moiety to the compound of formula II, wherein the detection moiety selected from the group consisting of a fluorophore, an oligonucleotide, a biomolecule, a molecular sensor, a protein, or a peptide.
(112) X is a functional group X that is capable of reacting with a functional group present on a fluorophore so as to covalently bind the fluorophore to the compound of formula II.
(113) X is a functional group that is capable of reacting with a functional group present on a fluorophore so as to covalently bind the fluorophore to the compound of formula II, and wherein the fluorophore is as defined in paragraph (44) or (45) above.
(114) X is a functional group selected from halo, N₃, or ethynyl

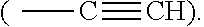

(115) X is ethynyl

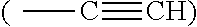

(116) Lₓ is a linker group L as defined in any one of paragraphs (10) to (41) above.
(117) Lₓ is a (1-8C)alkylene linker, wherein the alkylene chain optionally further comprises one or more —O—, —C(O)NR₁₀—, —NR₁₀C(O)—, —C(O)O—, —OC(O)— or triazole ring linkages positioned either within the alkylene chain and/or at one of its termini, wherein R₁₀ is hydrogen or (1-2C)alkyl.
(118) Lₓ is a (1-8C)alkylene linker, wherein the alkylene chain optionally further comprises one —O—, —C(O)NR₁₀—, —NR₁₀C(O)—, —C(O)O—, —OC(O)— or triazole ring linkage positioned either within the alkylene chain and/or at one of its termini, wherein R₁₀ is hydrogen or (1-2C)alkyl.
(119) Lₓ is a (1-5C)alkylene linker.
(120) Lₓ is a (1-3C)alkylene linker.

Suitably, R² is as defined in numbered paragraph (1) above.

Suitably, R⁴ is as defined in paragraph (3) above.

Suitably, R⁶ is as defined in any one of numbered paragraphs (5) to (14) above. More suitably, R⁶ is as defined in any one of numbered paragraphs (10) to (14) above. Most suitably, R⁶ is as defined numbered paragraphs (12), (13) or (14) above.

Suitably, Y₁ is as defined in numbered paragraphs (15) or (16). Most suitably, Y₁ is as defined in numbered paragraph (15).

Suitably, Y₂ is as defined in any one of numbered paragraphs (17) to (22). More suitably, Y₂ is as defined in numbered paragraphs (18), (19), (21) or (22). Most suitably, Y₂ is as defined in numbered paragraphs (21) or (22).

Suitably, A₁ is as defined in any one of numbered paragraphs (23) to (25). Most suitably, A₁ is as defined in numbered paragraph (23).

Suitably, A₂ is as defined in any one of numbered paragraphs (26) to (29). Most suitably, A₂ is as defined in numbered paragraph (26).

Suitably, A₃ is as defined in any one of numbered paragraphs (30) to (33). Most suitably, A₃ is as defined in numbered paragraph (30).

Suitably, A₄ is as defined in any one of numbered paragraphs (34) to (37). Most suitably, A₄ is as defined in numbered paragraphs (34).

Suitably, A₁, A₂, A₃ and A₄ are as defined in any one of numbered paragraphs (38) to (45). More suitably, A₁, A₂, A₃ and A₄ are as defined in any one of numbered paragraphs (40) to (45). Most suitably, A₁, A₂, A₃ and A₄ are as defined in numbered paragraph (42).

Suitably, is as defined in any one of numbered paragraphs (26) to (29). Most suitably, A₂ is as defined in numbered paragraph (26).

Suitably, is as defined in any one of numbered paragraphs (30) to (33). Most suitably, A₃ is as defined in numbered paragraph (30).

Suitably, is as defined in any one of numbered paragraphs (34) to (37). Most suitably, A₄ is as defined in numbered paragraphs (34).

Suitably, R¹¹ is as defined in any one of numbered paragraphs (46) to (56) above. More suitably, R¹¹ is as defined in any one of numbered paragraphs (50) to (56) above. Most suitably, R¹¹ is as defined numbered paragraph (53), (54), (55) or (56) above.

Suitably, R¹² is as defined in any one of numbered paragraphs (57) to (61) above. More suitably, R¹² is as defined in any one of numbered paragraphs (59) to (61) above. Most suitably, R¹² is as defined numbered paragraphs (60) or (61) above.

Suitably, R¹³ is as defined in any one of numbered paragraphs (62) to (64) above. More suitably, R¹³ is as defined in numbered paragraph (63) or (64) above. Most suitably, R¹³ is as defined numbered paragraph (64) above.

Suitably, R¹⁴ is as defined in any one of numbered paragraphs (65) to (68) above. More suitably, R¹⁴ is as defined in numbered paragraph (66), (67) or (68) above. Most suitably, R¹⁴ is as defined numbered paragraph (68) above.

Suitably, R^{a} is as defined in any one of numbered paragraphs (69) to (73) above. More suitably, R^{a} is as defined in any one of numbered paragraphs (70) to (73) above. Most suitably, R^{a} is as defined numbered paragraphs (71), (72) or (73) above.

Suitably, R^{a} is as defined in any one of numbered paragraphs (121) to (125) above. More suitably, R^{a} is as defined in any one of numbered paragraphs (122) to (125) above. Most suitably, R^{a} is as defined numbered paragraphs (123), (124) or (125) above.

Suitably, L is as defined in any one of numbered paragraphs (74) to (105) above. More suitably, L is as defined in any one of numbered paragraphs (101) to (105) above. Most suitably, L is as defined numbered paragraphs (101), (103) or (105) above.

Suitably, Q is as defined in any one of numbered paragraphs (106) to (109) above. More suitably, Q is as defined in any one of numbered paragraphs (107) to (109) above. Most suitably, Q is as defined numbered paragraph (107) above.

Suitably, X is as defined in any one of numbered paragraphs (110) to (115) above. More suitably, X is as defined in any one of numbered paragraphs (113) to (115) above. Most suitably, X is as defined numbered paragraph (114) above.

Suitably, Lₓ is as defined in any one of numbered paragraphs (116) to (120) above. More suitably, Lₓ is as defined in any one of numbered paragraphs (118) to (120) above. Most suitably, Lₓ is as defined numbered paragraph (118) above.

In a particular group of compounds of the invention, compounds have a structure according to formula I-I (which is a sub-definition of formula I), or a pharmaceutically acceptable salt, hydrate and/or solvate thereof:

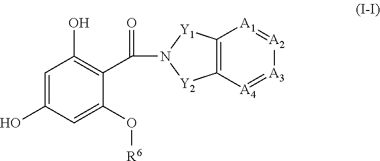

wherein R⁶, Y₁, Y₂, A₁, A₂, A₃, A₄, and any associated subgroups, are as defined herein or in any of the numbered paragraphs appearing hereinbefore.

In an embodiment of the compounds of formula I-I, or a pharmaceutically acceptable salt, hydrate and/or solvate thereof:
R⁶ is as defined in any one of numbered paragraphs (5) to (14) above;
Y₁ is —CH₂— or —C(═O)—;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
A₄ is selected from N, CH or CR¹⁴;
with the proviso that only one or two of A₁, A₂, A₃ or A₄ can be N;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above;
R¹⁴ is as defined in any one of numbered paragraphs (65) to (68) above.

In an embodiment of the compounds of formula I-I, or a pharmaceutically acceptable salt, hydrate and/or solvate thereof: R⁶ is as defined in any one of numbered paragraphs (5) to (14) above; Y₁ is —CH₂— or —C(═O)—; Y₂ is as defined in any one of numbered paragraphs (17) to (22) above;
A₁, A₂, A₃ and A₄ are as defined in any one of paragraphs (39) to (45) above;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above;
R¹⁴ is as defined in any one of numbered paragraphs (65) to (68) above.

In an embodiment of the compounds of formula I-I, or a pharmaceutically acceptable salt, hydrate and/or solvate thereof:
R⁶ is as defined in any one of numbered paragraphs (12) to (14) above;
Y₁ is as defined in numbered paragraph (15) above;
Y₂ is as defined in any one of numbered paragraphs (18) or (19) above;
A₁, A₂, A₃ and A₄ are as defined in any one of paragraph (39) to (45) above;
R¹¹ is as defined in any one of numbered paragraphs (50) to (56) above;
R¹² is as defined in any one of numbered paragraphs (59) to (61) above;
R¹³ is as defined in any one of numbered paragraphs (63) to (64) above;
R¹⁴ is as defined in any one of numbered paragraphs (66) to (68) above.

In an embodiment of the compounds of formula I-I, or a pharmaceutically acceptable salt, hydrate and/or solvate thereof:
R⁶ is as defined in numbered paragraph (12) above;
Y₁ is as defined in numbered paragraph (15) above;
Y₂ is as defined numbered paragraph (18) above;
A₁, A₂, A₃ and A₄ are as defined in paragraph (39) above;
R¹¹ is as defined in numbered paragraph (49) above;
R¹² is as defined in numbered paragraph (59) above;
R¹³ is as defined in numbered paragraph (63) above;
R¹⁴ is as defined in numbered paragraph (66) above.

In an embodiment of the compounds of formula I-I, or a pharmaceutically acceptable salt, hydrate and/or solvate thereof:
R⁶ is as defined in numbered paragraph (13) above;
Y₁ is as defined in numbered paragraph (15) above;
Y₂ is as defined numbered paragraph (19) above;
A₁, A₂, A₃ and A₄ are as defined in paragraph (40) above;
R¹¹ is as defined in numbered paragraph (50) above;
R¹² is as defined in numbered paragraph (60) above;
R¹³ is as defined in numbered paragraph (63) above;
R¹⁴ is as defined in numbered paragraph (67) above.

In an embodiment of the compounds of formula I-I, or a pharmaceutically acceptable salt, hydrate and/or solvate thereof:
R⁶ is as defined in numbered paragraph (13) above;
Y₁ is as defined in numbered paragraph (15) above;
Y₂ is as defined numbered paragraph (20) above;
A₁, A₂, A₃ and A₄ are as defined in paragraph (41) above;
R¹¹ is as defined in numbered paragraph (55) above;
R¹² is as defined in numbered paragraph (60) above;
R¹³ is as defined in numbered paragraph (63) above;
R¹⁴ is as defined in numbered paragraph (66) above.

In an embodiment of the compounds of formula I-I, or a pharmaceutically acceptable salt, hydrate and/or solvate thereof:
R⁶ is as defined in numbered paragraph (14) above;
Y₁ is as defined in numbered paragraph (15) above;
Y₂ is as defined numbered paragraph (20) above;
A₁, A₂, A₃ and A₄ are as defined in paragraph (41) above;
R¹¹ is as defined in numbered paragraph (55) above;
R¹² is as defined in numbered paragraph (60) above;
R¹³ is as defined in numbered paragraph (63) above;
R¹⁴ is as defined in numbered paragraph (66) above.

In an embodiment of the compounds of formula I-I, or a pharmaceutically acceptable salt, hydrate and/or solvate thereof:
R⁶ is as defined in numbered paragraph (14) above;
Y₁ is as defined in numbered paragraph (15) above;
Y₂ is as defined numbered paragraph (21) above;
A₁, A₂, A₃ and A₄ are as defined in paragraph (42) above;
R¹¹ is as defined in numbered paragraph (56) above;
R¹³ is as defined in numbered paragraph (64) above.

In an embodiment of the compounds of formula I-I, or a pharmaceutically acceptable salt, hydrate and/or solvate thereof, R⁶, Y₁, Y₂, A₁, A₂, A₃, A₄, R¹¹, R¹², R¹³ and R¹⁴ are as defined above, with the proviso that one of R¹² or R¹³ is a group:

-L-Q

or

-Lₓ-X
wherein L is a linker:
Lₓ is a linker;
X is a functional group; and
Q is a detection moiety.

In an embodiment of the compounds of formula I-I, or a pharmaceutically acceptable salt, hydrate and/or solvate thereof, R⁶, Y₁, Y₂, A₁, A₂, A₃, A₄, R¹¹, R¹², R¹³ and R¹⁴ are as defined above, with the proviso that one of R¹² or R¹³ is a group:

-L-Q
wherein L is a linker as defined in any one of numbered paragraphs (74) to (105) above; and
Q is a detection moiety as defined in any one of numbered paragraphs (106) to (109) above.

In an embodiment of the compounds of formula I-I, or a pharmaceutically acceptable salt, hydrate and/or solvate thereof, R⁶, Y₁, Y₂, A₁, A₂, A₃, A₄, R¹¹, R¹², R¹³ and R¹⁴ are as defined above, with the proviso that one of R¹² or R¹³ is a group:

-L-Q
wherein L is a linker as defined in any one of numbered paragraphs (101), (103) or (105) above; and
Q is a detection moiety as defined in numbered paragraph (107) above.

In a particular group of compounds of the invention, compounds have a structure according to formula I-II (which is a sub-definition of formula I), or a pharmaceutically acceptable salt, hydrate and/or solvate thereof:

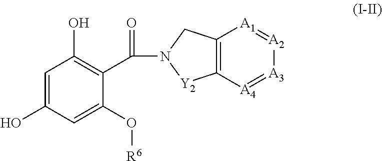

wherein R⁶, Y₂, A₁, A₂, A₃, A₄, and any associated subgroups, are as defined herein or in any of the numbered paragraphs appearing hereinbefore.

In an embodiment of the compounds of formula I-II, or a pharmaceutically acceptable salt, hydrate and/or solvate thereof:
R⁶ is as defined in any one of numbered paragraphs (5) to (14) above;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
A₄ is selected from N, CH or CR¹⁴;
with the proviso that only one or two of A₁, A₂, A₃ or A₄ can be N;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above;
R¹⁴ is as defined in any one of numbered paragraphs (65) to (68) above.

In an embodiment of the compounds of formula I-II, or a pharmaceutically acceptable salt, hydrate and/or solvate thereof:
R⁶ is as defined in any one of numbered paragraphs (5) to (14) above;
Y₂ is as defined in any one of numbered paragraphs (17) to (22) above;
A₁, A₂, A₃ and A₄ are as defined in any one of paragraphs (39) to (45) above;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above;
R¹⁴ is as defined in any one of numbered paragraphs (65) to (68) above.

In an embodiment of the compounds of formula I-II, or a pharmaceutically acceptable salt, hydrate and/or solvate thereof:
R⁶ is as defined in any one of numbered paragraphs (12) to (14) above;
Y₂ is as defined in any one of numbered paragraphs (18) or (19) above;
A₁, A₂, A₃ and A₄ are as defined in any one of paragraph (39) to (45) above;
R¹¹ is as defined in any one of numbered paragraphs (50) to (56) above;
R¹² is as defined in any one of numbered paragraphs (59) to (61) above;
R¹³ is as defined in any one of numbered paragraphs (63) to (64) above;
R¹⁴ is as defined in any one of numbered paragraphs (66) to (68) above.

In an embodiment of the compounds of formula I-II, or a pharmaceutically acceptable salt, hydrate and/or solvate thereof:
R⁶ is as defined in numbered paragraph (12) above;
Y₂ is as defined numbered paragraph (18) above;
A₁, A₂, A₃ and A₄ are as defined in paragraph (39) above;
R¹¹ is as defined in numbered paragraph (49) above;
R¹² is as defined in numbered paragraph (59) above;
R¹³ is as defined in numbered paragraph (63) above;
R¹⁴ is as defined in numbered paragraph (66) above.

In an embodiment of the compounds of formula I-II, or a pharmaceutically acceptable salt, hydrate and/or solvate thereof:
R⁶ is as defined in numbered paragraph (13) above;
Y₂ is as defined numbered paragraph (19) above;
A₁, A₂, A₃ and A₄ are as defined in paragraph (40) above;
R¹¹ is as defined in numbered paragraph (50) above;
R¹² is as defined in numbered paragraph (60) above;
R¹³ is as defined in numbered paragraph (63) above;
R¹⁴ is as defined in numbered paragraph (67) above.

In an embodiment of the compounds of formula I-II, or a pharmaceutically acceptable salt, hydrate and/or solvate thereof:
R⁶ is as defined in numbered paragraph (13) above;
Y₂ is as defined numbered paragraph (20) above;
A₁, A₂, A₃ and A₄ are as defined in paragraph (41) above;
R¹¹ is as defined in numbered paragraph (55) above;
R¹² is as defined in numbered paragraph (60) above;
R¹³ is as defined in numbered paragraph (63) above;
R¹⁴ is as defined in numbered paragraph (66) above.

In an embodiment of the compounds of formula I-II, or a pharmaceutically acceptable salt, hydrate and/or solvate thereof:
R⁶ is as defined in numbered paragraph (14) above;
Y₂ is as defined numbered paragraph (20) above;
A₁, A₂, A₃ and A₄ are as defined in paragraph (41) above;
R¹¹ is as defined in numbered paragraph (55) above;
R¹² is as defined in numbered paragraph (60) above;
R¹³ is as defined in numbered paragraph (63) above;
R¹⁴ is as defined in numbered paragraph (66) above.

In an embodiment of the compounds of formula I-II, or a pharmaceutically acceptable salt, hydrate and/or solvate thereof:
R⁶ is as defined in numbered paragraph (14) above;
Y₂ is as defined numbered paragraph (21) above;
A₁, A₂, A₃ and A₄ are as defined in paragraph (42) above;
R¹¹ is as defined in numbered paragraph (56) above;
R¹³ is as defined in numbered paragraph (64) above.

In an embodiment of the compounds of formula I-II, or a pharmaceutically acceptable salt, hydrate and/or solvate thereof, R⁶, Y₂, A₁, A₂, A₃, A₄, R¹¹, R¹², R¹³ and R¹⁴ are as defined above, with the proviso that one of R¹² or R¹³ is a group:

-L-Q

or

-Lₓ-X
wherein L is a linker;
Lₓ is a linker;
X is a functional group; and
Q is a detection moiety.

In an embodiment of the compounds of formula I-II, or a pharmaceutically acceptable salt, hydrate and/or solvate thereof, R⁶, Y₂, A₁, A₂, A₃, A₄, R¹¹, R¹², R¹³ and R¹⁴ are as defined above, with the proviso that one of R¹² or R¹³ is a group:

-L-Q
wherein L is a linker as defined in any one of numbered paragraphs (74) to (105) above; and
Q is a detection moiety as defined in any one of numbered paragraphs (106) to (109) above.

In an embodiment of the compounds of formula I-II, or a pharmaceutically acceptable salt, hydrate and/or solvate thereof, R⁶, Y₂, A₁, A₂, A₃, A₄, R¹¹, R¹², R¹³ and R¹⁴ are as defined above, with the proviso that one of R¹² or R¹³ is a group:

-L-Q
wherein L is a linker as defined in any one of numbered paragraphs (101), (103) or (105) above; and
Q is a detection moiety as defined in numbered paragraph (107) above.

In a particular group of compounds of the invention, compounds have a structure according to formula I-III (which is a sub-definition of formula I), or a pharmaceutically acceptable salt, hydrate and/or solvate thereof:

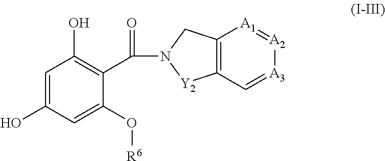

wherein R⁶, Y₂, A₁, A₂, A₃, A₄, and any associated subgroups, are as defined herein or in any of the numbered paragraphs appearing hereinbefore.

In an embodiment of the compounds of formula I-III, or a pharmaceutically acceptable salt, hydrate and/or solvate thereof:
R⁶ is as defined in any one of numbered paragraphs (5) to (14) above;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
with the proviso that only one of A₁, A₂ and A₃ can be N;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above.

In an embodiment of the compounds of formula I-III, or a pharmaceutically acceptable salt, hydrate and/or solvate thereof:
R⁶ is as defined in any one of numbered paragraphs (5) to (14) above;
Y₂ is as defined in any one of numbered paragraphs (17) to (22) above;
A₁, A₂ and A₃ are as defined in any one of paragraphs (39) to (45) above;
R¹¹ is as defined in any one of numbered paragraphs (46) to (56) above;
R¹² is as defined in any one of numbered paragraphs (57) to (61) above;
R¹³ is as defined in any one of numbered paragraphs (62) to (64) above.

In an embodiment of the compounds of formula I-III, or a pharmaceutically acceptable salt, hydrate and/or solvate thereof:
R⁶ is as defined in any one of numbered paragraphs (12) to (14) above;
Y₂ is as defined in any one of numbered paragraphs (18) or (19) above;
A₁, A₂ and A₃ are as defined in any one of paragraph (39) to (45) above;
R¹¹ is as defined in any one of numbered paragraphs (50) to (56) above;
R¹² is as defined in any one of numbered paragraphs (59) to (61) above;
R¹³ is as defined in any one of numbered paragraphs (63) to (64) above.

In an embodiment of the compounds of formula I-III, or a pharmaceutically acceptable salt, hydrate and/or solvate thereof:
R⁶ is as defined in numbered paragraph (12) above;
Y₂ is as defined numbered paragraph (18) above;
A₁, A₂ and A₃ are as defined in paragraph (39) above;
R¹¹ is as defined in numbered paragraph (49) above;
R¹² is as defined in numbered paragraph (59) above;
R¹³ is as defined in numbered paragraph (63) above.

In an embodiment of the compounds of formula I-III, or a pharmaceutically acceptable salt, hydrate and/or solvate thereof:
R⁶ is as defined in numbered paragraph (13) above;
Y₂ is as defined numbered paragraph (19) above;
A₁, A₂ and A₃ are as defined in paragraph (40) above;
R¹¹ is as defined in numbered paragraph (50) above;
R¹² is as defined in numbered paragraph (60) above;
R¹³ is as defined in numbered paragraph (63) above.

In an embodiment of the compounds of formula I-III, or a pharmaceutically acceptable salt, hydrate and/or solvate thereof:
R⁶ is as defined in numbered paragraph (13) above;
Y₂ is as defined numbered paragraph (20) above;
A₁, A₂ and A₃ are as defined in paragraph (41) above;
R¹¹ is as defined in numbered paragraph (55) above;
R¹² is as defined in numbered paragraph (60) above;
R¹³ is as defined in numbered paragraph (63) above.

In an embodiment of the compounds of formula I-III, or a pharmaceutically acceptable salt, hydrate and/or solvate thereof:
R⁶ is as defined in numbered paragraph (14) above;
Y₂ is as defined numbered paragraph (20) above;
A₁, A₂ and A₃ are as defined in paragraph (41) above;
R¹¹ is as defined in numbered paragraph (55) above;
R¹² is as defined in numbered paragraph (60) above;
R¹³ is as defined in numbered paragraph (63) above.

In an embodiment of the compounds of formula I-III, or a pharmaceutically acceptable salt, hydrate and/or solvate thereof:
R⁶ is as defined in numbered paragraph (14) above;
Y₂ is as defined numbered paragraph (21) above;
A₁, A₂ and A₃ are as defined in paragraph (42) above;
R¹¹ is as defined in numbered paragraph (56) above;
R¹³ is as defined in numbered paragraph (64) above.

In an embodiment of the compounds of formula I-III, or a pharmaceutically acceptable salt, hydrate and/or solvate thereof, R⁶, Y₂, A₁, A₂, A₃, R¹¹, R¹² and R¹³ are as defined above, with the proviso that one of R¹² or R¹³ is a group:

-L-Q

or

-Lₓ-X
wherein L is a linker;
Lₓ is a linker;
X is a functional group; and
Q is a detection moiety.

In an embodiment of the compounds of formula I-III, or a pharmaceutically acceptable salt, hydrate and/or solvate thereof, R⁶, Y₂, A₁, A₂, A₃, R¹¹, R¹² and R¹³ are as defined above, with the proviso that one of R¹² or R¹³ is a group:

-L-Q
wherein L is a linker as defined in any one of numbered paragraphs (74) to (105) above; and
Q is a detection moiety as defined in any one of numbered paragraphs (106) to (109) above.

In an embodiment of the compounds of formula I-III, or a pharmaceutically acceptable salt, hydrate and/or solvate thereof, R⁶, Y₂, A₁, A₂, A₃, R¹¹, R¹² and R¹³ are as defined above, with the proviso that one of R¹² or R¹³ is a group:

-L-Q
wherein L is a linker as defined in any one of numbered paragraphs (101), (103) or (105) above; and
Q is a detection moiety as defined in numbered paragraph (107) above.

In a particular group of compounds of formulae I, I-I, I-II or I-III defined herein, R⁶ is as defined in paragraph (12) above.

In a particular group of compounds of formulae I, I-I, I-II or I-III defined herein, R⁶ is as defined in paragraph (13) above.

In a particular group of compounds of formulae I, I-I, I-II or I-III defined herein, R⁶ is as defined in paragraph (14) above.

In a particular group of compounds of formulae I, I-I, I-II or I-III defined herein, Y₂ is as defined in paragraph (18) above.

In a particular group of compounds of formulae I, I-I, I-II or I-III defined herein, Y₂ is as defined in paragraph (19) above.

In a particular group of compounds of formulae I, I-I, I-II or I-III defined herein, Y₂ is as defined in paragraph (20) above.

In a particular group of compounds of formulae I, I-I, I-II or I-III defined herein, Y₂ is as defined in paragraph (21) above.

In a particular group of compounds of formulae I, I-I, I-II or I-III defined herein, Y₂ is as defined in paragraph (22) above.

In a particular group of compounds of formulae I, I-I, I-II or I-III defined herein, R¹¹ is as defined in paragraph (50) above.

In a particular group of compounds of formulae I, I-I, I-II or I-III defined herein, R¹¹ is as defined in paragraph (51) above.

In a particular group of compounds of formulae I, I-I, I-II or I-III defined herein, R¹¹ is as defined in paragraph (52) above.

In a particular group of compounds of formulae I, I-I, I-II or I-III defined herein, R¹¹ is as defined in paragraph (53) above.

In a particular group of compounds of formulae I, I-I, I-II or I-III defined herein, R¹¹ is as defined in paragraph (54) above.

In a particular group of compounds of formulae I, I-I, I-II or I-III defined herein, R¹¹ is as defined in paragraph (55) above.

In a particular group of compounds of formulae I, I-I, I-II or I-III defined herein, R¹¹ is as defined in paragraph (56) above.

In a particular group of compounds of formulae I, I-I, I-II or I-III defined herein, R¹² is as defined in paragraph (59) above.

In a particular group of compounds of formulae I, I-I, I-II or I-III defined herein, R¹² is as defined in paragraph (60) above.

In a particular group of compounds of formulae I, I-I, I-II or I-III defined herein, R¹² is as defined in paragraph (61) above.

In a particular group of compounds of formulae I, I-I, I-II or I-III defined herein, R¹³ is as defined in paragraph (63) above.

In a particular group of compounds of formulae I, I-I, I-II or I-III defined herein, R¹² is as defined in paragraph (64) above.

In a particular group of compounds of formulae I, I-I, I-II or I-III defined herein, R¹³ is as defined in paragraph (66) above.

In a particular group of compounds of formulae I, I-I, I-II or I-III defined herein, R¹³ is as defined in paragraph (67) above.

In a particular group of compounds of formulae I, I-I, I-II or I-III defined herein, R¹³ is as defined in paragraph (68) above.

In a particular group of compounds of formulae I, I-I, I-II or I-III defined herein, A₄ is N or CH, especially CH.

Particular compounds of the present invention include any of the compounds exemplified in the present application, or a pharmaceutically acceptable salt or solvate thereof, and, in particular, any of the following:
(R)-(2-(Benzyloxy)-4,6-dihydroxyphenyl)(8-((tetrahydrofuran-3-yl)amino)-3,4-dihydro-isoquinolin-2(1H)-yl)methanone;
(S)-(2-(Benzyloxy)-4,6-dihydroxyphenyl)(8-((tetrahydrofuran-3-yl)amino)-3,4-dihydro-isoquinolin-2(1H)-yl)methanone;
(2-(Benzyloxy)-4,6-dihydroxyphenyl)(5-(piperazin-1-ylmethyl)isoindolin-2-yl)methanone;
(2-(Benzyloxy)-4,6-dihydroxyphenyl)(5-((methylamino)methyl)isoindolin-2-yl)methanone; 3-((2-(2-(Benzyloxy)-4,6-dihydroxybenzoyl)isoindolin-4-yl)oxy)cyclobutane-1-carbonitrile;
(2-(Benzyloxy)-4,6-dihydroxyphenyl)(4-((tetrahydrofuran-3-yl)amino)isoindolin-2-yl)methanone;
(2-(Benzyloxy)-4,6-dihydroxyphenyl)(4-(oxetan-3-ylamino)isoindolin-2-yl)methanone;
(2-(Benzyloxy)-4,6-dihydroxyphenyl)(4-(3-hydroxypiperidin-1-yl)isoindolin-2-yl)methanone;
(2-(Benzyloxy)-4,6-dihydroxyphenyl)(4-((4-methylpiperazin-1-yl)methyl)isoindolin-2-yl)methanone;
(2-(Benzyloxy)-4,6-dihydroxyphenyl)(4-(morpholinomethyl)isoindolin-2-yl)methanone;
(2-(Benzyloxy)-4,6-dihydroxyphenyl)(5-((dimethylamino)methyl)isoindolin-2-yl)methanone;
(2-(Benzyloxy)-4,6-dihydroxyphenyl)(5-(morpholinomethyl)isoindolin-2-yl)methanone;
(2-(Benzyloxy)-4,6-dihydroxyphenyl)(1-(hydroxymethyl)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((tetrahydro-2H-pyran-4-yl)amino)isoindolin-2-yl)methanone;
(2,4-Dihydroxy-6-methoxyphenyl)(4-((tetrahydrofuran-3-yl)amino)isoindolin-2-yl)methanone;
(2-Ethoxy-4,6-dihydroxyphenyl)(4-((tetrahydrofuran-3-yl)amino)isoindolin-2-yl)methanone; 4-((2-(2-(Benzyloxy)-4,6-dihydroxybenzoyl)isoindolin-4-yl)amino)-1-methylpyrrolidin-2-one;
(2-(Benzyloxy)-4,6-dihydroxyphenyl)(4-(pyrimidin-5-ylamino)isoindolin-2-yl)methanone;
(2-(Benzyloxy)-4,6-dihydroxyphenyl)(4-((oxazol-4-ylmethyl)amino)isoindolin-2-yl)methanone;
(2-(Benzyloxy)-4,6-dihydroxyphenyl)(4-((1-methyl-1H-pyrazol-4-yl)amino)isoindolin-2-yl)methanone;
(S)-(2-(Benzyloxy)-4,6-dihydroxyphenyl)(4-((tetrahydrofuran-3-yl)amino)isoindolin-2-yl)methanone;
(R)-(2-(Benzyloxy)-4,6-dihydroxyphenyl)(4-((tetrahydrofuran-3-yl)amino)isoindolin-2-yl)methanone;
4-((2-(2-(benzyloxy)-4,6-dihydroxybenzoyl)isoindolin-4-yl)amino)-5-methylpyridin-2(1H)-one;
(2-(Cyclopropylmethoxy)-4,6-dihydroxyphenyl)(4-((tetrahydrofuran-3-yl)amino)isoindolin-2-yl)methanone;
(2-(cyclohexylmethoxy)-4,6-dihydroxyphenyl)(4-((tetrahydrofuran-3-yl)amino)isoindolin-2-yl)methanone;
(2-(Benzyloxy)-4,6-dihydroxyphenyl)(5-((tetrahydrofuran-3-yl)amino)-3,4-dihydroisoquinolin-2(1H)-yl)methanone;
(2-(Cyclohexylmethoxy)-4,6-dihydroxyphenyl)(8-((tetrahydrofuran-3-yl)amino)-3,4-dihydroisoquinolin-2(1H)-yl)methanone;
(2-(Benzyloxy)-4,6-dihydroxyphenyl)(4-(methylamino)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((5,5-difluorotetrahydro-2H-pyran-3-yl)amino)isoindolin-2-yl)methanone;
(2-(Benzyloxy)-4,6-dihydroxyphenyl)(4-(((tetrahydrofuran-2-yl)methyl)amino)isoindolin-2-yl)methanone;
(4-((1H-Pyrazol-4-yl)amino)isoindolin-2-yl)(2-(benzyloxy)-4,6-dihydroxyphenyl)methanone;
(2-(Benzyloxy)-4,6-dihydroxyphenyl)(4-((2-methyl-2H-1,2,3-triazol-4-yl)amino)isoindolin-2-yl)methanone; 1-(3-((2-(2-(Benzyloxy)-4,6-dihydroxybenzoyl)isoindolin-4-yl)amino)pyrrolidin-1-yl)ethan-1-one;
(2-((4-Fluorobenzyl)oxy)-4,6-dihydroxyphenyl)(4-((tetrahydrofuran-3-yl)amino)isoindolin-2-yl)methanone;
(2-((3,4-Difluorobenzyl)oxy)-4,6-dihydroxyphenyl)(4-((tetrahydrofuran-3-yl)amino)isoindolin-2-yl)methanone;
(2,4-Dihydroxy-6-((4-methoxybenzyl)oxy)phenyl)(4-((tetrahydrofuran-3-yl)amino)isoindolin-2-yl)methanone;
(2,4-Dihydroxy-6-(pyridin-2-ylmethoxy)phenyl)(4-((tetrahydrofuran-3-yl)amino)isoindolin-2-yl)methanone;
(2-(Benzyloxy)-4,6-dihydroxyphenyl)(8-(oxetan-3-ylamino)-3,4-dihydroisoquinolin-2(1H)-yl)methanone;
4-((2-(2-(Benzyloxy)-4,6-dihydroxybenzoyl)-1,2,3,4-tetrahydroisoquinolin-8-yl)amino)-1-methylpyrrolidin-2-one;
(2-(Benzyloxy)-4,6-dihydroxyphenyl)(8-(methylamino)-3,4-dihydroisoquinolin-2(1H)-yl)methanone;
(2-(Benzyloxy)-4,6-dihydroxyphenyl)(8-((2-methoxyethyl)amino)-3,4-dihydroisoquinolin-2(1H)-yl)methanone;
(2-(Benzyloxy)-4,6-dihydroxyphenyl)(4-((1-(oxetan-3-yl)ethyl)amino)isoindolin-2-yl)methanone;
(2-Benzyloxy-4,6-dihydroxy-phenyl)-[6-[(dimethylamino)methyl]-3,4-dihydro-1H-isoquinolin-2-yl]methanone;
(2-Benzyloxy-4,6-dihydroxy-phenyl)-[7-[(dimethylamino)methyl]-3,4-dihydro-1H-isoquinolin-2-yl]methanone;
(2-Benzyloxy-4,6-dihydroxy-phenyl)-(6-methoxy-3,4-dihydro-1H-isoquinolin-2-yl)methanone;
(2-Benzyloxy-4,6-dihydroxy-phenyl)-(7-methoxy-3,4-dihydro-1H-isoquinolin-2-yl)methanone;
(2-Benzyloxy-4,6-dihydroxy-phenyl)-(4-hydroxy-3,4-dihydro-1H-isoquinolin-2-yl)methanone;
(2-Benzyloxy-4,6-dihydroxy-phenyl)-(4-hydroxy-3,4-dihydro-1H-isoquinolin-2-yl)methanone;
(2-(Benzyloxy)-4,6-dihydroxyphenyl)(4-((1-methyl-2-oxabicyclo[2.1.1]hexan-4-yl)amino)isoindolin-2-yl)methanone;
(4-((2-Oxabicyclo[2.2.1]heptan-4-yl)amino)isoindolin-2-yl)(2-(benzyloxy)-4,6-dihydroxyphenyl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
(S)-(2,4-Dihydroxy-6-(pyridin-2-ylmethoxy)phenyl)(8-((tetrahydrofuran-3-yl)amino)-3,4-dihydroisoquinolin-2(1H)-yl)methanone;
(R)-(2,4-dihydroxy-6-(pyridin-2-ylmethoxy)phenyl)(8-((tetrahydrofuran-3-yl)amino)-3,4-dihydroisoquinolin-2(1H)-yl)methanone;
(2,4-Dihydroxy-6-(pyridin-2-ylmethoxy)phenyl)(8-(oxetan-3-ylamino)-3,4-dihydroisoquinolin-2(1H)-yl)methanone;
(S)-4-((2-(2,4-Dihydroxy-6-(pyridin-2-ylmethoxy)benzoyl)-1,2,3,4-tetrahydroisoquinolin-8-yl)amino)-1-methylpyrrolidin-2-one;
(R)-4-((2-(2,4-dihydroxy-6-(pyridin-2-ylmethoxy)benzoyl)-1,2,3,4-tetrahydroisoquinolin-8-yl)amino)-1-methylpyrrolidin-2-one;
(R)-(2-(Benzyloxy)-4,6-dihydroxyphenyl)(6-(2-methoxyethoxy)-8-((tetrahydrofuran-3-yl)amino)-3,4-dihydroisoquinolin-2(1H)-yl)methanone;
4-((2-(2-(Benzyloxy)-4,6-dihydroxybenzoyl)-1,2,3,4-tetrahydroisoquinolin-8-yl)amino)-1-methylpyrrolidin-2-one;
4-((2-(2-(Benzyloxy)-4,6-dihydroxybenzoyl)-1,2,3,4-tetrahydroisoquinolin-8-yl)amino)-1-methylpyrrolidin-2-one;
(S)-(2-(Benzyloxy)-4,6-dihydroxyphenyl)(4-((tetrahydrofuran-3-yl)amino)-7,8-dihydro-1,6-naphthyridin-6(5H)-yl)methanone;
(S)-(2-(Benzyloxy)-4,6-dihydroxyphenyl)(6-(2-methoxyethoxy)-8-((tetrahydrofuran-3-yl)amino)-3,4-dihydroisoquinolin-2(1H)-yl)methanone;
(S)-1-(3-((2-(2-(Benzyloxy)-4,6-dihydroxybenzoyl)-1,2,3,4-tetrahydroisoquinolin-8-yl)amino)pyrrolidin-1-yl)ethan-1-one;
(R)-1-(3-((2-(2-(Benzyloxy)-4,6-dihydroxybenzoyl)-1,2,3,4-tetrahydroisoquinolin-8-yl)amino)pyrrolidin-1-yl)ethan-1-one;
(S)-1-(3-((2-(2-(Benzyloxy)-4,6-dihydroxybenzoyl)-1,2,3,4-tetrahydroisoquinolin-8-yl)amino)piperidin-1-yl)ethan-1-one;
(R)-1-(3-((2-(2-(benzyloxy)-4,6-dihydroxybenzoyl)-1,2,3,4-tetrahydroisoquinolin-8-yl)amino)piperidin-1-yl)ethan-1-one;
1-(4-((2-(2-(Benzyloxy)-4,6-dihydroxybenzoyl)-1,2,3,4-tetrahydroisoquinolin-8-yl)amino)piperidin-1-yl)ethan-1-one;
5-((2-(2-(Benzyloxy)-4,6-dihydroxybenzoyl)-1,2,3,4-tetrahydroisoquinolin-8-yl)amino)-1-methylpiperidin-2-one;
(2-(Benzyloxy)-4,6-dihydroxyphenyl)(8-(pyrimidin-5-ylamino)-3,4-dihydroisoquinolin-2(1H)-yl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
3-((2-(2-(Benzyloxy)-4,6-dihydroxybenzoyl)-1,2,3,4-tetrahydroisoquinolin-8-yl)amino)benzamide;
4-((2-(2-(Benzyloxy)-4,6-dihydroxybenzoyl)-1,2,3,4-tetrahydroisoquinolin-8-yl)amino)benzamide;
(2-(Benzyloxy)-4,6-dihydroxyphenyl)(8-(cyclopentylamino)-3,4-dihydroisoquinolin-2(1H)-yl)methanone;
(2-(Benzyloxy)-4,6-dihydroxyphenyl)(8-((1-methylpiperidin-4-yl)amino)-3,4-dihydroisoquinolin-2(1H)-yl)methanone;
(2-(Benzyloxy)-4,6-dihydroxyphenyl)(8-((1-methylpyrrolidin-3-yl)amino)-3,4-dihydroisoquinolin-2(1H)-yl)methanone;
(2-(Benzyloxy)-4,6-dihydroxyphenyl) (4-((methylamino)methyl) isoindolin-2-yl)methanone;
(2-(Benzyloxy)-4,6-dihydroxyphenyl)(4-(((tetrahydrofuran-3-yl)amino)methyl)isoindolin-2-yl)methanone;
(2-(Benzyloxy)-4,6-dihydroxyphenyl)(4-((oxetan-3-ylamino)methyl)isoindolin-2-yl)methanone;
(2-(Benzyloxy)-4,6-dihydroxyphenyl)(4-(((3-methyloxetan-3-yl)amino)methyl)isoindolin-2-yl)methanone;
(4-(Aminomethyl)isoindolin-2-yl)(2-(benzyloxy)-4,6-dihydroxyphenyl)methanone hydrochloride;
(2-(Benzyloxy)-4,6-dihydroxyphenyl)(7-((oxetan-3-ylamino)methyl)-3,4-dihydroisoquinolin-2(1H)-yl)methanone;
(2-(Benzyloxy)-4,6-dihydroxyphenyl)(7-(((tetrahydrofuran-3-yl)amino)methyl)-3,4-dihydroisoquinolin-2(1H)-yl)methanone;
2-(2-(Benzyloxy)-4,6-dihydroxybenzoyl)-N-methylisoindoline-5-carboxamide;
2-(2-(Benzyloxy)-4,6-dihydroxybenzoyl)-N,N-dimethylisoindoline-5-carboxamide;
(2-(Benzyloxy)-4,6-dihydroxyphenyl)(4-((tetrahydrofuran-3-yl)oxy)isoindolin-2-yl)methanone;
(2-(Benzyloxy)-4,6-dihydroxyphenyl)(4-(pyrimidin-5-ylmethoxy)isoindolin-2-yl)methanone;
(4-(Azetidin-3-ylmethoxy)isoindolin-2-yl)(2-(benzyloxy)-4,6-dihydroxyphenyl) methanone;
2-(2-(Benzyloxy)-4,6-dihydroxybenzoyl)isoindoline-4-carbonitrile;
1-(2-(2-(Benzyloxy)-4,6-dihydroxybenzoyl)isoindolin-4-yl)azetidine-3-carbonitrile;
(4-Aminoisoindolin-2-yl)(2-(benzyloxy)-4,6-dihydroxyphenyl)methanone;
N-(2-(2-(benzyloxy)-4,6-dihydroxybenzoyl)isoindolin-4-yl)acetamide;
N-((2-(2-(Benzyloxy)-4,6-dihydroxybenzoyl)isoindolin-4-yl)methyl)acetamide;
(2,4-Dihydroxy-6-(pyridin-3-ylmethoxy)phenyl)(4-((tetrahydrofuran-3-yl)amino)isoindolin-2-yl)methanone;
(2,4-Dihydroxy-6-(pyridin-4-ylmethoxy)phenyl)(4-((tetrahydrofuran-3-yl)amino)isoindolin-2-yl)methanone;
(2,4-Dihydroxy-6-((tetrahydro-2H-pyran-4-yl)methoxy)phenyl)(4-((tetrahydrofuran-3-yl)amino)isoindolin-2-yl)methanone;
(2,4-Dihydroxy-6-(pyridazin-3-ylmethoxy)phenyl)(4-((tetrahydrofuran-3-yl)amino)isoindolin-2-yl)methanone;
8-Amino-3,4-dihydroisoquinolin-2(1H)-yl(2-(benzyloxy)-4,6-dihydroxyphenyl)methanone;
N-(2-(2-(benzyloxy)-4,6-dihydroxybenzoyl)-1,2,3,4-tetrahydroisoquinolin-8-yl)acetamide;
N-(2-(2-(Benzyloxy)-4,6-dihydroxybenzoyl)-1, 2, 3, 4-tetrahydroisoquinolin-8-yl) tetrahydrofuran-3-carboxamide;
t-Butyl (5-(4-((2-(2-(benzyloxy)-4,6-dihydroxybenzoyl)isoindolin-5-yl)methyl) piperazin-1-yl)pentyl)carbamate;
(5-((4-(5-Aminopentyl)piperazin-1-yl)methyl)isoindolin-2-yl)(2-(benzyloxy)-4,6 dihydroxyphenyl)methanone hydrochloride;
(2-(Benzyloxy)-4,6-dihydroxyphenyl)(4-(((tetrahydrofuran-3-yl)methyl)amino)isoindolin-2-yl)methanone;
3-((2-(2-(Benzyloxy)-4,6-dihydroxybenzoyl)-1,2,3,4-tetrahydroisoquinolin-8-yl)amino)-N,N-dimethylpyrrolidine-1-carboxamide;
4-((2-(2-(Benzyloxy)-4,6-dihydroxybenzoyl)-1,2,3,4-tetrahydroisoquinolin-8-yl)amino)-1-methylpiperidin-2-one;
(S)-(2-(Benzyloxy)-4,6-dihydroxyphenyl)(4-((tetrahydrofuran-3-yl)amino)-7,8-dihydropyrido[4,3-d]pyrimidin-6(5H)-yl)methanone;
(2-(Benzyloxy)-4,6-dihydroxyphenyl)(7-fluoro-8-((tetrahydrofuran-3-yl)amino)-3,4-dihydroisoquinolin-2(1H)-yl)methanone;
(2-(Benzyloxy)-4,6-dihydroxyphenyl)(8-((1-methyl-1H-pyrazol-4-yl)amino)-3,4-dihydroisoquinolin-2(1H)-yl)methanone;
(2-(Benzyloxy)-4,6-dihydroxyphenyl)(8-(pyridin-3-ylamino)-3,4-dihydroisoquinolin-2(1H)-yl)methanone;
(2-(Benzyloxy)-4,6-dihydroxyphenyl)(8-(pyrazin-2-ylamino)-3,4-dihydroisoquinolin-2(1H)-yl)methanone;
(2-(Benzyloxy)-4,6-dihydroxyphenyl)(8-(pyridazin-4-ylamino)-3,4-dihydroisoquinolin-2(1H)-yl)methanone;
(S)-(2-(Benzyloxy)-4,6-dihydroxyphenyl)(6-((4-methylpiperazin-1-yl)methyl)-8-((tetrahydrofuran-3-yl)amino)-3,4-dihydroisoquinolin-2(1H)-yl)methanone;
(R)-(2-(Benzyloxy)-4,6-dihydroxyphenyl)(6-((4-methylpiperazin-1-yl)methyl)-8-((tetrahydrofuran-3-yl)amino)-3,4-dihydroisoquinolin-2(1H)-yl)methanone;
(2-(Benzyloxy)-4,6-dihydroxyphenyl)(8-((4-methylpyrimidin-5-yl)amino)-3,4-dihydroisoquinolin-2(1H)-yl)methanone;
(S)-(2,4-Dihydroxy-6-((3-methylbenzyl)oxy)phenyl)(8-((tetrahydrofuran-3-yl)amino)-3,4-dihydroisoquinolin-2(1H)-yl)methanone;
(S)-(2-(Benzyloxy)-4,6-dihydroxyphenyl)(6-((dimethylamino)methyl)-8-((tetrahydrofuran-3-yl)amino)-3,4-dihydroisoquinolin-2(1H)-yl)methanone;
(R)-(2-(Benzyloxy)-4,6-dihydroxyphenyl)(6-((dimethylamino)methyl)-8-((tetrahydrofuran-3-yl)amino)-3,4-dihydroisoquinolin-2(1H)-yl)methanone;
(S)-(2-(2-(Benzyloxy)-4,6-dihydroxybenzoyl)-8-((tetrahydrofuran-3-yl)amino)-1,2,3,4-tetrahydroisoquinolin-6-yl)(4-methylpiperazin-1-yl)methanone;
(2-(Benzyloxy)-4,6-dihydroxyphenyl)(6-(2-methoxyethoxy)-8-(methylamino)-3,4-dihydroisoquinolin-2(1H)-yl)methanone;
(R)-2-(2-(Benzyloxy)-4,6-dihydroxybenzoyl)-N,N-dimethyl-8-((tetrahydrofuran-3-yl)amino)-1,2,3,4-tetrahydroisoquinoline-6-carboxamide;
(S)-2-(2-(Benzyloxy)-4,6-dihydroxybenzoyl)-N,N-dimethyl-8-((tetrahydrofuran-3-yl)amino)-1,2,3,4-tetrahydroisoquinoline-6-carboxamide;
(R)-(2-(Benzyloxy)-4,6-dihydroxyphenyl)(6-(2-(dimethylamino)ethoxy)-8-((tetrahydrofuran-3-yl)amino)-3,4-dihydroisoquinolin-2(1H)-yl)methanone;
(S)-(2-(Benzyloxy)-4,6-dihydroxyphenyl)(6-(2-(dimethylamino)ethoxy)-8-((tetrahydrofuran-3-yl)amino)-3,4-dihydroisoquinolin-2(1H)-yl)methanone;
4-((2-(2-(Benzyloxy)-4,6-dihydroxybenzoyl)-1,2,3,4-tetrahydro-isoquinolin-8-yl)amino)-1-methylpyridin-2(1H)-one;
5-((2-(2-(Benzyloxy)-4,6-dihydroxybenzoyl)-1,2,3,4-tetrahydroisoquinolin-8-yl)amino)pyridin-2(1H)-one;
5-((2-(2-(Benzyloxy)-4,6-dihydroxybenzoyl)-1,2,3,4-tetrahydroisoquinolin-8-yl)amino)-1-methylpyridin-2(1H)-one;
(R)-4-((2-(2-(Benzyloxy)-4,6-dihydroxybenzoyl)isoindolin-4-yl)amino)-1-methylpyrrolidin-2-one;
(S)-4-((2-(2-(Benzyloxy)-4,6-dihydroxybenzoyl)isoindolin-4-yl)amino)-1-methylpyrrolidin-2-one;
(R)-(2-(Benzyloxy)-4,6-dihydroxyphenyl)(6-((1-methylpiperidin-4-yl)oxy)-8-((tetrahydrofuran-3-yl)amino)-3,4-dihydroisoquinolin-2(1H)-yl)methanone;
(S)-(2-(Benzyloxy)-4,6-dihydroxyphenyl)(6-((1-methylpiperidin-4-yl)oxy)-8-((tetrahydrofuran-3-yl)amino)-3,4-dihydroisoquinolin-2(1H)-yl)methanone;
(S)-(2-(Cyclohexylmethoxy)-4,6-dihydroxyphenyl)(8-((tetrahydrofuran-3-yl)amino)-3,4-dihydroisoquinolin-2(1H)-yl)methanone;
(R)-(2-(Cyclopropylmethoxy)-4,6-dihydroxyphenyl)(6-(2-methoxyethoxy)-8-((tetrahydrofuran-3-yl)amino)-3,4-dihydroisoquinolin-2(1H)-yl)methanone;
(R)-(2,4-Dihydroxy-6-(pyridin-2-ylmethoxy)phenyl)(6-(2-methoxyethoxy)-8-((tetrahydrofuran-3-yl)amino)-3,4-dihydroisoquinolin-2(1H)-yl)methanone;
(2-(Benzyloxy)-4,6-dihydroxyphenyl)(8-(methyl(tetrahydrofuran-3-yl)amino)-3,4-dihydroisoquinolin-2(1H)-yl)methanone; 2-(Benzyloxy)-4,6-dihydroxyphenyl-(5-((oxetan-3-ylamino)methyl)isoindolin-2-yl)methanone;
(2-(Benzyloxy)-4,6-dihydroxyphenyl)(5-(((tetrahydrofuran-3-yl)amino)methyl)isoindolin-2-yl)methanone;
1-(3-((2-(2-(Benzyloxy)-4,6-dihydroxybenzoyl)-1,2,3,4-tetrahydroisoquinolin-8-yl)amino)azetidin-1-yl)ethan-1-one;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((4-methoxytetrahydrofuran-3-yl)amino)-3,4-dihydroisoquinolin-2(1H)-yl)methanone;
1-(3-((2-(2-(Benzyloxy)-4,6-bis(methoxymethoxy)benzoyl)-1,2,3,4-tetrahydroisoquinolin-8-yl)(methyl)amino)azetidin-1-yl)ethan-1-one;
(S)-1-(3-((2-(2-(Benzyloxy)-4,6-dihydroxybenzoyl)-1,2,3,4-tetrahydroisoquinolin-8-yl) (methyl)amino) pyrrolidin-1-yl)-4-methoxybutan-1-one;
(S)-1-(3-((2-(2-(Benzyloxy)-4,6-dihydroxybenzoyl)-1,2,3,4-tetrahydroisoquinolin-8-yl) amino) pyrrolidin-1-yl)-4-methoxybutan-1-one;
5-((5-(4-((2-(2-(Benzyloxy)-4,6-dihydroxybenzoyl)isoindolin-5-yl)methyl)piperazin-1-yl)pentyl)carbamoyl)-2-(6-(dimethylamino)-3-(dimethyliminio)-3H-xanthen-9-yl)benzoate;
t-Butyl(5-(2-(2-(benzyloxy)-4,6-dihydroxybenzoyl)isoindoline-5-carboxamido)pentyl)carbamate;
N-(5-Aminopentyl)-2-(2-(benzyloxy)-4,6-dihydroxybenzoyl)isoindoline-5-carboxamide hydrochloride;
5-((5-(2-(2-(Benzyloxy)-4,6-dihydroxybenzoyl)isoindoline-5-carboxamido)pentyl) carbamoyl)-2-(6-(dimethylamino)-3-(dimethyliminio)-3H-xanthen-9-yl)benzoate;
1-(6-((5-(2-(2-(Benzyloxy)-4,6-dihydroxybenzoyl)isoindoline-5-carboxamido)pentyl)amino)-6-oxohexyl)-3,3-dimethyl-2-((1E,3E)-5-((E)-1,3,3-trimethylindolin-2-ylidene)penta-1,3-dien-1-yl)-3H-indol-1-ium formate;
N-(5-(4-((2-(2-(Benzyloxy)-4,6-dihydroxybenzoyl)isoindolin-5-yl)methyl)piperazin-1-yl)pentyl)-3-(5,5-difluoro-7-(1H-pyrrol-2-yl)-5H-4λ⁴,5λ⁴-dipyrrolo[1,2-c:2′,1′-f][1,3,2]diazaborinin-3-yl)propenamide; and
2-(2-(Benzyloxy)-4,6-dihydroxybenzoyl)-N-(5-(3-(5,5-difluoro-7-(1H-pyrrol-2-yl)-5H-4λ⁴,5λ⁴-dipyrrolo[1,2-c:2′,1′-f][1,3,2]diazaborinin-3-yl)propanamido)pentyl)isoindoline-5-carboxamide.

The various functional groups and substituents making up the compounds of the Formula (I), or sub-formulae (I-I) to (I-XII), are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z- isomers).

It is to be understood that the present invention encompasses all optical, diastereoisomers and geometric isomers and mixtures thereof that possess activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the Formula (I), or sub-formulae (1-1) to (I-XII), may exist in solvated as well as unsolvated forms such as, for example, hydrated forms. It is to be understood that the invention encompasses all such solvated forms that possess activity.

It is also to be understood that certain compounds of the Formula (I), or sub-formulae (1-1) to (I-XII), may exhibit polymorphism, and that the invention encompasses all such forms that possess activity.

Compounds of the Formula (I), or sub-formulae (I-I) to (I-XII), may exist in a number of different tautomeric forms and references to compounds of the Formula (I), or sub-formulae (I-I) to (I-XII), include all such forms. For the avoidance of doubt, where a compound can exist in one of several tautomeric forms, and only one is specifically described or shown, all others are nevertheless embraced by Formula (I), or sub-formulae (I-I) to (I-XII). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

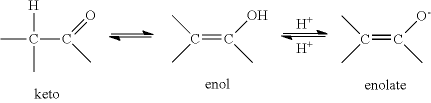

Compounds of the Formula (I), or sub-formulae (I-I) to (I-XII), containing an amine function may also form N-oxides. A reference herein to a compound of the Formula (I), or sub-formulae (1-1) to (I-XII), that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae (I-I) to (I-XII), may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae (1-1) to (I-XII), and in-vivo cleavable amide derivatives that may be formed at a carboxy group or an amino group in a compound of the Formula (I), or sub-formulae (I-I) to (I-XII).

Accordingly, the present invention includes those compounds of the Formula (I), or sub-formulae (1-1) to (I-XII), as defined hereinbefore, when made available by organic synthesis and when made available within the human or animal body by way of cleavage of a pro-drug thereof. Accordingly, the present invention includes those compounds of the Formula (I), or sub-formulae (1-1) to (I-XII), that are produced by organic synthetic means and also such compounds that are produced in the human or animal body by way of metabolism of a precursor compound, that is a compound of the Formula (I), or sub-formulae (I-I) to (I-XII), may be a synthetically-produced compound or a metabolically-produced compound.

A suitable pharmaceutically acceptable pro-drug of a compound of the Formula (I), or sub-formulae (I-I) to (I-XII), is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191
(1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae (I-I) to (I-XII), that possesses a carboxy group is, for example, an in vivo cleavable ester thereof. An in vivo cleavable ester of a compound of the Formula I, or sub-formulae (I-I) to (I-XII), containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae (I-I) to (I-XII), that possesses a hydroxy group is, for example, an in vivo cleavable ester or ether thereof. An in vivo cleavable ester or ether of a compound of the Formula (I), or sub-formulae (I-I) to (I-XII), containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I), or sub-formulae (I-I) to (I-XII), that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I), or sub-formulae (I-I) to (I-XII), that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae (I-I) to (I-XII), may be exerted in part by one or more metabolites that are formed within the human or animal body after administration of a compound of the Formula (I), or sub-formulae (I-I) to (I-XII). As stated hereinbefore, the in vivo effects of a compound of the Formula (I), or sub-formulae (I-I) to (I-XII), may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae (1-1) to (1-Ill), will vary depending on the nature of any substituent groups or subgroups associated therewith. Suitable processes for their preparation are described further in the accompanying Examples.

Once a compound of Formula (I), or sub-formulae (I-I) to (1-Ill), has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups R², R⁴, R⁶, R¹¹, R¹², R¹³ or R¹⁴ may be further reacted to change the nature of the group and provide an alternative compound of Formula (I).

The resultant compounds of Formula (I), or sub-formulae (I-I) to (1-Ill), can be isolated and purified using techniques well known in the art.

The compounds of Formula (I) may be synthesised by the synthetic routes shown in the Examples section below.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the MLH1 in vitro assay described in the Example section and, in some cases, in the MLH1 and PMS2 in vitro assays described in the Example section.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of MLH1 activity or MLH1 and PMS2 activity.

The compounds of Formula (I), or a pharmaceutically acceptable salt thereof, therefore have potential therapeutic uses in a variety of disease states in which the inhibition of MLH1 activity or MLH1 and PMS2 activity is beneficial.

The present invention therefore provides a method of treating a disease or disorder in which the inhibition MLH1 activity or MLH1 and PMS2 activity is beneficial in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of inhibiting MLH1 activity or MLH1 and PMS2 activity, in vitro or in vivo, said method comprising contacting a cell with an effective amount of a compound or a pharmaceutically acceptable salt, hydrate or solvate thereof as defined herein.

The present invention provides a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use as a medicament.

The present invention provides a compound or a pharmaceutically acceptable salt, hydrate or solvate thereof as defined herein, or a pharmaceutical composition as defined herein, for use in the treatment of a proliferative disorder.

The present invention provides a compound, or a pharmaceutically acceptable salt, hydrate or solvate thereof, or a pharmaceutical composition as defined herein for use in the treatment of cancer. In a particular embodiment, the cancer is human cancer. In a particular embodiment, the cancer is human cancer, in particular oestrogen positive cancers, such as breast cancer, or androgen receptor positive cancers, such as prostate cancer.

The present invention provides a compound, or a pharmaceutically acceptable salt, hydrate or solvate thereof, as defined herein for use in the inhibition of MLH1 activity or MLH1 and PMS2 activity.

The present invention provides a compound, or a pharmaceutically acceptable salt, hydrate or solvate thereof, as defined herein for use in the treatment of a disease or disorder in which the inhibition of MLH1 activity or MLH1 and PMS2 activity is beneficial.

The present invention provides a use of a compound, or a pharmaceutically acceptable salt, hydrate or solvate thereof, as defined herein in the manufacture of a medicament for the treatment of a proliferative disorder.

The present invention provides a use of a compound, or a pharmaceutically acceptable salt, hydrate or solvate thereof, as defined herein in the manufacture of a medicament for the treatment of cancer.

The present invention provides a use of a compound, or a pharmaceutically acceptable salt, hydrate or solvate thereof, as defined herein in the manufacture of a medicament for the inhibition of MLH1 activity or MLH1 and PMS2 activity.

The present invention provides a use of a compound, or a pharmaceutically acceptable salt, hydrate or solvate thereof, as defined herein in the manufacture of a medicament for the treatment of a disease or disorder in which the inhibition of MLH1 activity or MLH1 and PMS2 activity is beneficial.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In the aspects of the invention outlined herein, the proliferative disorder is suitably cancer, and the cancer is suitably a human cancer. In particular, the compounds of the present invention will be useful for the treatment of any cancer in which mis-match repair inhibition and/or cGAS/STING pathway activation is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dubé Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

The compounds of the present invention may also be used to treat triplet diseases.

Thus, a further aspect of the present invention provides a method of treating a triplet disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided the use of a compound, or a pharmaceutically acceptable salt, hydrate or solvate thereof, as defined herein in the manufacture of a medicament for the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery or radiotherapy or chemotherapy or a targeted agent. Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034).
(vi) vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan; (viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, A2a receptor antagonists, PI3 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

Combination Therapy with Immune Modulating Treatments

### Immune Checkpoint Inhibitors

Immune checkpoint proteins present on immune cells and/or cancer cells [e.g. CTLA4 (also known as cytotoxic T-lymphocyte-associated protein 4 and CD152), LAG3 (also known as lymphocyte-activation gene 3 and CD223), PD1 (also known as programmed cell death protein 1 and CD279), PD-L1 (also known as programmed death-ligand 1 and CD274), TIM-3 (also known as T-cell immunoglobulin mucin-3) and TIGIT (also known as T-cell Immunoreceptor with Ig and ITIM domains) are molecular targets that have been found to play an important role in regulating anti-tumour immune responses. Inhibitors of these immune checkpoint proteins (e.g. CTLA4, LAG3, PD1, PD-L1, TIM-3 and/or TIGIT inhibitors) promote an anti-tumour immune response that can be utilised to effectively treat certain forms of cancer.

### Immune Stimulators

Monoclonal antibodies, bispecific antibodies, recombinant ligands and small molecule therapeutics that bind to stimulatory receptors on immune cells can facilitate an effective anti-tumour response. Such receptors may be involved in cell-to-cell contact for example contact between tumour cell and immune cell or between two types of immunce cells, other receptors may bind to soluble factors that stimulate an immune response. In one such embodiment antibodies, bispecifics, recombindant proteins or small molecule therapeutics can activate stimulatory receptors, including, but not limited to, 4-1 BB, OX40, cGAS-STING, CD27, CD40, and DR3 that enhance anti-tumour immunity.

Modulators of antigen processing may facilitate the presentation of neoantigenic peptides on the cell surface to enhance an effective anti-tumour response. In one such embodiment inhibitors of the endoplasmic reticulum aminopeptidases ERAP1 and ERAP2 may stimulate anti-tumour immunity.

In one aspect, the present invention relates to a combination comprising a compound as defined herein, or a pharmaceutically acceptable salt thereof, and an immune checkpoint inhibitor or immune stimulator as defined herein, or a pharmaceutically acceptable salt thereof, for use in the treatment of a proliferative disorder.

In another aspect, the present invention relates to a use of a combination comprising a compound as defined herein, or a pharmaceutically acceptable salt thereof, and an immune checkpoint inhibitor or immune stimulator as defined herein, or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and an immune checkpoint inhibitor or immune stimulator as defined herein, or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administeration with an immune checkpoint inhibitor, or immune stimulator, or a pharmaceutically acceptable salt thereof.

In another aspect, the present invention relates to an immune checkpoint inhibitor or immune stimulator, or a pharmaceutically acceptable salt thereof, for use in the treatment of a proliferative disorder, wherein the immune checkpoint inhibitor is for simultaneous, separate or sequential administeration with a compound as defined herein, or a pharmaceutically acceptable salt thereof, as defined herein.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administeration with an immune checkpoint inhibitor or immune stimulator, or a pharmaceutically acceptable salt thereof.

In another aspect, the present invention relates to a use of an immune checkpoint inhibitor or immune stimulator, or a pharmaceutically acceptable salt thereof, in the manufacture of a medicament for treating a proliferative disorder, wherein the medicament is for simultaneous, separate or sequential administeration with a compound as defined herein, or a pharmaceutically acceptable salt thereof.

In another aspect, the present invention relates to a method of treating a proliferative disorder comprising adminstering to a subject in need thereof a therapeutically effective amount of a compound as defined herein, or a pharmaceutically acceptable salt thereof, as defined herein and an immune checkpoint inhibitor or immune stimulator as defined herein, or a pharmaceutically acceptable salt thereof, either sequentially, separately or simultaneously

Any immune checkpoint inhibitor or immune stimulator may be used in the combination therapy defined herein.

In one embodiment, the immune stimulator is selected from a 4-1 BB stimulator, a OX40 stimulator, a CD27 stimulator, a CD40 stimulator, and a DR3 stimulator. In another embodiment the immune checkpoint inhibitor is selected from a PD1-inhibitor, a PD-L1 inhibitor, a LAG3 inhibitor, CTLA-4 inhibitor, a TIM-3 inhibitor and/or a TIGIT inhibitor. In a particular embodiment, the immune checkpoint inhibitor is a PD1 or PD-L1 inhibitor.

PD-1 is a cell surface receptor protein present on immune cells such as T cells. PD-1 plays an important role in down-regulating the immune system and promoting self-tolerance by suppressing T cell activation. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD1 binds two ligands, PD-L1 and PD-L2. PD-L1 is of particular interest as it is highly expressed in several cancers and hence the role of PD1 in cancer immune evasion is well established. Monoclonal antibodies targeting PD-1 that boost the immune system are approved or are being developed for the treatment of cancer. Many tumour cells express PD-L1, an immunosuppressive PD-1 ligand; inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda) and nivolumab (Opdivo). These drugs have been shown to be effective in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (Imfinzi). These drugs have also been shown to be helpful in treating different types of cancer, including bladder cancer, non-small cell lung cancer, and Merkel cell skin cancer (Merkel cell carcinoma). They are also being studied for use against other types of cancer.

Examples of LAG3 inhibitors include BMS-986016/Relatlimab, TSR-033, REGN3767, MGD013 (bispecific DART binding PD-1 and LAG-3), GSK2831781 and LAG525.

Examples of CTLA-4 inhibitors include MDX-010/Ipilimumab, AGEN1884, and CP-675,206/Tremelimumab.

Examples of TIM-3 inhibitors include MBG453 (Novartis), TSR-022 (Tesaro), and LY3321367 (Lilly).

Examples of TIGIT inhibitors include Tiragolumab (MTIG7192A; RG6058; Genentech/Roche), AB154 (Arcus Bioscience), MK-7684 (Merck), BMS-986207 (Bristol-Myers Squibb), ASP8374 (Astellas Pharma; Potenza Therapeutics).

In one embodiment, the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab, durvalumab, MBG453, TSR-022, LY3321367, Tiragolumab (MTIG7192A; RG6058), AB154, MK-7684, BMS-986207, and/or ASP8374 or a pharmaceutically acceptable salt or solvate thereof.

Combination Therapy with DNA Damage Response Modulators

The compounds of the present invention are particularly suited to use in combination with agents that act as DNA damage response modulators, e.g. PARP inhibitors, ATM inhibitors and ATR inhibitors.

In one aspect, the present invention relates to a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, for use in the treatment of a proliferative disorder.

In another aspect, the present invention relates to a use of a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administeration with a DNA damage response modulator (e.g. a PARP inhibitor, an ATM inhibitor and/or an ATR inhibitor), or a pharmaceutically acceptable salt thereof.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administeration with a DNA damage response modulator (e.g. a PARP inhibitor, an ATM inhibitor and/or an ATR inhibitor), or a pharmaceutically acceptable salt thereof.

In another aspect, the present invention relates to a method of treating a proliferative disorder comprising adminstering to a subject in need thereof a therapeutically effective amount of a compound as defined herein, or a pharmaceutically acceptable salt thereof, as defined herein and a DNA damage response modulator (e.g. a PARP inhibitor, an ATM inhibitor and/or an ATR inhibitor), or a pharmaceutically acceptable salt thereof, either sequentially, separately or simultaneously

Any DNA damage response modulator (e.g. a PARP inhibitor, an ATM inhibitor and/or an ATR inhibitor) may be used in the combination therapy defined herein.

Combination Therapy with DNA Damage Response Modulators

The compounds of the present invention are particularly suited to use in combination with agents that act as DNA damage response modulators, e.g. PARP inhibitors, ATM inhibitors and ATR inhibitors.

In one aspect, the present invention relates to a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, for use in the treatment of a proliferative disorder.

In another aspect, the present invention relates to a use of a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administeration with a DNA damage response modulator (e.g. a PARP inhibitor, an ATM inhibitor and/or an ATR inhibitor), or a pharmaceutically acceptable salt thereof.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administeration with a DNA damage response modulator (e.g. a PARP inhibitor, an ATM inhibitor and/or an ATR inhibitor), or a pharmaceutically acceptable salt thereof.

In another aspect, the present invention relates to a method of treating a proliferative disorder comprising adminstering to a subject in need thereof a therapeutically effective amount of a compound as defined herein, or a pharmaceutically acceptable salt thereof, as defined herein and a DNA damage response modulator (e.g. a PARP inhibitor, an ATM inhibitor and/or an ATR inhibitor), or a pharmaceutically acceptable salt thereof, either sequentially, separately or simultaneously

Any DNA damage response modulator (e.g. a PARP inhibitor, an ATM inhibitor and/or an ATR inhibitor) may be used in the combination therapy defined herein.

### Probe Compounds of the Invention

In another aspect, the present invention provides a probe compound of formula I, I-I, I-II, 1-Ill or a salt thereof, as defined herein, wherein one of R¹² or R¹³ is a group -L-Q or -Lₓ-X as defined herein.

The probe compounds of the present invention are, depending on the nature of the compound of formula I, selective for either MLH1 or MLH1 and PMS2.

The linker group L may be any suitable linker moiety that connects the detection moiety Q to the remainder of the probe compound of formula I, I-I, I-II or I-III defined herein.

Suitably, the linker group L is 3 to 30 atoms in length, more suitably, 4 to 20 atoms in length and even more suitably 5 to 18 atoms in length. In a particular group of probe compounds of formula I or II defined herein, L is 5 to 12 atoms in length.

Suitably, L is as defined in any one paragraphs (74) to (105) above. More suitably as defined in either of paragraphs (101), (103) or (105) above. In a particular group of probe compounds of formula I or II, L is as defined in paragraph (103) or (105) above.

The detection moiety Q can be any moiety that can enable the probe compound of formula I to be detected and quantified. As described further below, the probe compounds of formula I defined herein are designed to be used in displacement assays, whereby the ability of a test compound to displace the probe compound of formula I from the ATP-binding site of target protein (e.g. MLH1 or PMS2) can be used to determine the binding affinity of that test compound for the ATP-binding site of the target protein. Thus, the detection moiety Q can be any moiety that can be readily detected and quantified. In certain circumstances, the detection moiety Q enables any probe compounds of formula I that have been displaced (i.e. is “unbound”) from the ATP-binding site of the target protein to be detected and quantified. In certain embodiments of the invention, this may be achieved by collecting any displaced or “unbound” compound of formula I from the test sample and assaying the sample to determine how much unbound probe compound is present. This will in turn give an indication of how much of the probe compound present in the sample has been displaced by the test compound.

It will therefore be appreciated that the nature of detection moiety Q is not critical as long as it can be used to enable the amount of the probe compound of formula I present in a sample to be determined. A person skilled in the art will be able to select a suitable detection moiety Q and a suitable methodology for detecting and quantifying the amount of the compound of formula I in a sample, in particular to detect the amount of the probe compound of formula I that has been displaced from the ATP binding site of a target protein by a test compound.

Suitably, the detection moiety Q is selected from the group consisting of a fluorophore, an oligonucleotide, a biomolecule, a molecular sensor, a protein, or a peptide.

In embodiments where the detection moiety Q is an oligonucleotide, a biomolecule, a molecular sensor, a protein, or a peptide, then any suitable technique known in the art for detecting and quantifying the amount of the oligonucleotide, biomolecule, molecular sensor, protein, or peptide present may be utilised. For example, a fluorescently labelled secondary probe may be used that is capable of specifically binding to the detection moiety Q of the probe compound of formula I and, once any excess of the secondary probe has been removed, the amount of binding of the secondary probe to detection moiety Q of the compound for formula I can be detected and quantified, thereby enabling the amount of the probe compound of formula I to be determined.

For example, if Q is an oligonucleotide, then a secondary probe having a suitable detectable label, e.g. a fluorophore or radio-label, and a complimentary oligonucleotide sequence capable of hybridising to Q can be used to detect and quantify the amount of the probe compound of formula I present in sample (and suitably the amount of the probe compound displaced from the ATP-binding site of the target protein by a test compound). Similarly, if Q is a protein or peptide, then a secondary probe may be an antibody capable of selectively binding to that protein or peptide and a suitable detectable label, e.g. a fluorophore or radio-label.

More suitably, the detection moiety Q is a fluorophore. In such cases, the compound for formula I can be used in a fluorescence polarisation assay. In a particular group of probe compounds of formula I, the detection moiety Q is a fluorophore selected from the group consisting of AlexaFluor dyes, Cyanine dyes, fluorescein, BODIPY or BODIPY derivatives (e.g. BODIPY TMR), TAMRA, Oregon Green dyes, FITC, Ru(bpy)3, Rhodamine dyes, Acridine orange, and Texas Red. In a further group of probe compounds of formula I, the detection moiety Q is a fluorophore selected from the group consisting of AlexaFluor-647, AlexaFluor-633, AlexaFluor-594, AlexaFluor-488, Cyanine-5B, Cyanine-3B, Fluorescein, BODIPY TMR, TAMRA, Oregon Green 488, Oregon Green 514, FITC, Ru(bpy)3, Rhodamine dyes, Acridine orange, and Texas Red.

Suitably, Q is as defined in any one of numbered paragraphs (106) to (109) above. More suitably, Q is as defined in any one of numbered paragraphs (107) to (109) above. Most suitably, Q is as defined numbered paragraph (107) above.

It will be appreciated that X may be any suitable functional group that is capable of reacting with a functional group present on a detection moiety Q, or a compound of the formula Q-L₂-Y as defined herein, to covalently bind the detection moiety to the compound of formula II and thereby form a compound of formula I as defined herein.

A person skilled in the art will be familiar with appropriate functional groups that can be used to form covalent linkages with a suitable detection moiety. For example, X may be selected from halo, N₃ or ethynyl (—C≡CH).

In a particular embodiment, X is N₃ or ethynyl.

Suitably, X is a functional group that is capable of reacting with a functional group present on the detection moiety to form a bond or triazole linkage between the detection moiety and the compound of formula I, II. In a particular embodiment, the linkage formed is a triazole linkage (which can be formed by CLICK chemistry).

Suitably, X is as defined in any one of numbered paragraphs (110) to (115) above. More suitably, X is as defined in any one of numbered paragraphs (113) to (115) above. Most suitably, X is as defined numbered paragraph (114) above.

In certain embodiments of the invention, a compound of formula I, I-I, I-II or I-III in which one of R¹² or R¹³ is a group -L-Q is formed by reacting a compound of formula I in which one of R¹² or R¹³ is a group -Lₓ-X with a group of the formula:

Q-L_{y}-Y

wherein:
Q is a detection moiety as defined hereinbefore:
L_{y} is absent or a linker group which, when covalently bound to the group Lₓ of the compound of formula II forms a linker L of the compound of formula I, I-I, I-II or I-III;
Y is a functional group capable of reacting with the functional group X present on the compound of formula II to covalently link Q-L₂ to Lₓ of the compound of formula I, I-I, I-II or I-III and form a group -L-Q.

In an embodiment, L_{y} is a (1-8C)alkylene linker, wherein the alkylene chain optionally further comprises one or more —O—, —C(O)NR_{q1}—, —NR_{q1}C(O)—, —C(O)O—, —OC(O)— or triazole ring linkages positioned either within the alkylene chain and/or at one of its termini, wherein R_{q1} is hydrogen or (1-2C)alkyl.

In another embodiment, L_{y} is a (1-8C)alkylene linker, wherein the alkylene chain optionally further comprises one —O—, —C(O)NR_{q1}—, —NR_{q1}C(O)—, —C(O)O—, —OC(O)— or triazole ring linkage positioned either within the alkylene chain and/or at one of its termini, wherein R_{q1} is hydrogen or (1-2C)alkyl.

In a further embodiment, L_{y} is a (1-5C)alkylene linker.

In another embodiment, L_{y} is a (1-3C)alkylene linker.

It will be appreciated that Y may be any suitable functional group that is capable of reacting with a functional group X present on a probe compound of formula I to covalently bind the group Q-L_{y} to the compound of formula II and thereby form a compound of formula I as defined herein.

A person skilled in the art will be familiar with appropriate functional groups that can be used to form covalent linkages with a functional group X on the compound of formula I, I-I, I-II or I-III. For example, Y may be selected from halo, N₃ or ethynyl.

In a particular embodiment, Y is N₃ when X is ethynyl or Y is ethynyl when X is N₃.

Suitably, Y is a functional group that is capable of reacting with a functional group X present on the probe compound of formula I, I-I, I-II or I-III to form a bond or triazole linkage. In a particular embodiment, the linkage formed is a triazole linkage (which can be formed by CLICK chemistry).

The linker group Lₓ may be any suitable linker moiety that connects the functional group X to the remainder of the probe compound of formula I, I-I, I-II or I-III defined herein. Suitably, Lₓ is as defined in any one of numbered paragraphs (116) to (120) above. More suitably, Lₓ is as defined in any one of numbered paragraphs (118) to (120) above. Most suitably, Lₓ is as defined numbered paragraph (118) above.

In a particular group of probe compounds of the present invention, the probe compound is a compound of formula I, wherein R², R⁴, R⁶, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

-L-Q

wherein L is a linker; and Q is a detection moiety.

Suitably, in the probe compounds of formula I, L is a linker as defined in any one of numbered paragraphs (74) to (105) above; and Q is a detection moiety as defined in any one of numbered paragraphs (106) to (109) above. More suitably, L is a linker as defined in any one of numbered paragraphs (101), (103) or (105) above; and Q is a detection moiety as defined in numbered paragraph (107) above.

In another group of probe compounds of the present invention, the compound is a compound of the formula I-I shown above, or a pharmaceutically acceptable salt, hydrate and/or solvate thereof, wherein R⁶, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

-L-Q

wherein L is a linker and Q is a detection moiety.

Suitably, in the probe compounds of formula I-I, L is a linker as defined in any one of numbered paragraphs (74) to (105) above; and Q is a detection moiety as defined in any one of numbered paragraphs (106) to (109) above. More suitably, L is a linker as defined in any one of numbered paragraphs (101), (103) or (105) above; and Q is a detection moiety as defined in numbered paragraph (107) above.

In another group of probe compounds of the present invention, the compound is a compound of the formula I-II shown above, or a pharmaceutically acceptable salt, hydrate and/or solvate thereof, wherein R⁶, Y₂, A₁, A₂, A₃, A₄, R¹¹, R¹², R¹³ and R¹⁴ each have any one of the definitions provided hereinbefore, with the proviso that one of R¹² or R¹³ is a group:

-L-Q

wherein L is a linker and Q is a detection moiety.

Suitably, in the probe compounds of formula I-II, L is a linker as defined in any one of numbered paragraphs (74) to (105) above; and Q is a detection moiety as defined in any one of numbered paragraphs (106) to (109) above. More suitably, L is a linker as defined in any one of numbered paragraphs (101), (103) or (105) above; and Q is a detection moiety as defined in numbered paragraph (107) above.

In another group of probe compounds of the present invention, the compound is a compound of the formula I-III shown above, or a pharmaceutically acceptable salt, hydrate and/or solvate thereof, wherein R⁶, Y₂, A₁, A₂, A₃, R¹¹, R¹² and R¹³ each have any one of the definitions provided hereinbefore, with the proviso that one of R¹² or R¹³ is a group:

-L-Q

wherein L is a linker and Q is a detection moiety.

Suitably, in the probe compounds of formula I-III, L is a linker as defined in any one of numbered paragraphs (74) to (105) above; and Q is a detection moiety as defined in any one of numbered paragraphs (106) to (109) above. More suitably, L is a linker as defined in any one of numbered paragraphs (101), (103) or (105) above; and Q is a detection moiety as defined in numbered paragraph (107) above.

In a particular group of probe compounds of formulae I, I-I, I-II or I-III defined herein, R⁶ is as defined in paragraph (12) above.

In a particular group of probe compounds of formulae I, I-I, I-II or I-III defined herein, R⁶ is as defined in paragraph (13) above.

In a particular group of probe compounds of formulae I, I-I, I-II or I-III defined herein, R⁶ is as defined in paragraph (14) above.

In a particular group of probe compounds of formulae I, I-I, I-II or I-III defined herein, Y₂ is as defined in paragraph (18) above.

In a particular group of probe compounds of formulae I, I-I, I-II or I-III defined herein, Y₂ is as defined in paragraph (19) above.

In a particular group of probe compounds of formulae I, I-I, I-II or I-III defined herein, Y₂ is as defined in paragraph (20) above.

In a particular group of probe compounds of formulae I, I-I, I-II or I-III defined herein, Y₂ is as defined in paragraph (21) above.

In a particular group of probe compounds of formulae I, I-I, I-II or I-III defined herein, Y₂ is as defined in paragraph (22) above.

In a particular group of probe compounds of formulae I, I-I, I-II or I-III defined herein, R¹¹ is as defined in paragraph (50) above.

In a particular group of probe compounds of formulae I, I-I, I-II or I-III defined herein, R¹¹ is as defined in paragraph (51) above.

In a particular group of probe compounds of formulae I, I-I, I-II or I-III defined herein, R¹¹ is as defined in paragraph (52) above.

In a particular group of probe compounds of formulae I, I-I, I-II or I-III defined herein, R¹¹ is as defined in paragraph (53) above.

In a particular group of probe compounds of formulae I, I-I, I-II or I-III defined herein, R¹¹ is as defined in paragraph (54) above.

In a particular group of probe compounds of formulae I, I-I, I-II or I-III defined herein, R¹¹ is as defined in paragraph (55) above.

In a particular group of probe compounds of formulae I, I-I, I-II or I-III defined herein, R¹¹ is as defined in paragraph (56) above.

In a particular group of probe compounds of formulae I, I-I, I-II or I-III defined herein, R¹² is as defined in paragraph (59) above.

In a particular group of probe compounds of formulae I, I-I, I-II or I-III defined herein, R¹² is as defined in paragraph (60) above.

In a particular group of probe compounds of formulae I, I-I, I-II or I-III defined herein, R¹² is as defined in paragraph (61) above.

In a particular group of probe compounds of formulae I, I-I, I-II or I-III defined herein, R¹³ is as defined in paragraph (63) above.

In a particular group of probe compounds of formulae I, I-I, I-II or I-III defined herein, R¹² is as defined in paragraph (64) above.

In a particular group of probe compounds of formulae I, I-I, I-II or I-III defined herein, R¹³ is as defined in paragraph (66) above.

In a particular group of probe compounds of formulae I, I-I, I-II or I-III defined herein, R¹³ is as defined in paragraph (67) above.

In a particular group of probe compounds of formulae I, I-I, I-II or I-III defined herein, R¹³ is as defined in paragraph (68) above.

In a particular group of probe compounds of formulae I, I-I, I-II or I-III defined herein, A₄ is N or CH, especially CH.

Particular probe compounds of the present invention include any of the compounds exemplified in the present application, or a pharmaceutically acceptable salt or solvate thereof, and, in particular, any of the following:
5-((5-(2-(2-(Benzyloxy)-4,6-dihydroxybenzoyl)isoindoline-5-carboxamido)pentyl) carbamoyl)-2-(6-(dimethylamino)-3-(dimethyliminio)-3H-xanthen-9-yl)benzoate;
1-(6-((5-(2-(2-(Benzyloxy)-4,6-dihydroxybenzoyl)isoindoline-5-carboxamido)pentyl)amino)-6-oxohexyl)-3,3-dimethyl-2-((1E,3E)-5-((E)-1,3,3-trimethylindolin-2-ylidene)penta-1,3-dien-1-yl)-3H-indol-1-ium formate;
N-(5-(4-((2-(2-(Benzyloxy)-4,6-dihydroxybenzoyl)isoindolin-5-yl)methyl)piperazin-1-yl)pentyl)-3-(5,5-difluoro-7-(1H-pyrrol-2-yl)-5H-4λ⁴,5λ⁴-dipyrrolo[1,2-c:2′,1′-f][1,3,2]diazaborinin-3-yl)propenamide; and
2-(2-(Benzyloxy)-4,6-dihydroxybenzoyl)-N-(5-(3-(5,5-difluoro-7-(1H-pyrrol-2-yl)-5H-4λ⁴,5λ⁴-dipyrrolo[1,2-c:2′,1′-f][1,3,2]diazaborinin-3-yl)propanamido)pentyl)isoindoline-5-carboxamide. 5-((5-(4-((2-(2-(Benzyloxy)-4,6-dihydroxybenzoyl)isoindolin-5-yl)methyl)piperazin-1-yl)pentyl)carbamoyl)-2-(6-(dimethylamino)-3-(dimethyliminio)-3H-xanthen-9-yl)benzoate
t-Butyl (5-(2-(2-(benzyloxy)-4,6-dihydroxybenzoyl)isoindoline-5-carboxamido)pentyl)carbamate
N-(5-Aminopentyl)-2-(2-(benzyloxy)-4,6-dihydroxybenzoyl)isoindoline-5-carboxamide hydrochloride

A suitable salt of a probe compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable salt of a compound of the invention which is sufficiently acidic is an alkali metal salt, for example a sodium or potassium salt, an alkaline earth metal salt, for example a calcium or magnesium salt, an ammonium salt or a salt with an organic base.

The present invention also encompasses probe compounds of the invention as defined herein which comprise one or more isotopic substitutions. For example, H may be in any isotopic form, including ¹H, ²H (D) and ³H (T); C may be in any isotopic form, including ¹²C, ¹³C and ¹⁴C; and O may be in any isotopic form, including ¹⁶O and ¹⁸O; and the like.

It is also to be understood that certain probe compounds of the formula (I) may exist in solvated as well as unsolvated forms such as, for example, hydrated forms.

It is also to be understood that certain probe compounds of the formula (I) may exhibit polymorphism, and that the invention encompasses all such polymorphic forms.

Certain probe compounds of formula (I) may also exist in a number of different tautomeric forms and references to compounds of the formula (I) include all such forms.

### Synthesis of the Probe Compounds

In another aspect, the present invention provides a method of synthesising a probe compound of formula I, I-I, I-II or I-III, or a salt thereof, as defined herein.

The probe compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green; 4^{th} Edition; 2006 (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

In one aspect, the present invention provides a process for synthesising a compound of formula I defined herein, the process comprising reacting a compound of formula I, I-I, I-II or I-III in which one of R¹² or R¹³ is a group -Lₓ-X as defined herein, with a compound of the formula Q-L₂-Y as defined herein to form a compound of formula I, I-I, I-II or I-III; and optionally thereafter:
(i) removing any protecting groups present;
(ii) forming a salt of the compound of formula I; and/or
(iii) isolating and purifying the compound of formula I, I-I, I-II or I-III.

### Applications of the Probe Compounds

In one aspect, the present invention provides the use of a probe compound of formula I, I-I, I-II or I-III, or a salt thereof, as defined herein in a displacement assay to determine the binding affinity of a test molecule for the ATP-binding site of a target protein.

In another aspect, the present invention provides a probe compound of formula I, I-I, I-II or I-III, or a salt thereof, for use in a displacement assay to determine the binding affinity of a test molecule for the ATP-binding site of a target protein.

In an embodiment, the target protein is selected from MLH1 or MLH1 and PMS2. In a particular embodiment, the target protein is MLH1 and PMS2. In a specific embodiment, the target protein is only MLH1.

Displacement assays are well known in the art. In embodiments of the invention in which Q is a fluorophore, the displacement assay is a fluorescence polarisation assay. Fluorescence polarisation assays are well known in the art.

The test compound may be any biologic or small molecule compound that is to be screened for binding to the ATP-binding site of the target protein (e.g. MLH1 or PMS2).

In another aspect, the present invention provides an assay for determining the binding affinity of a test molecule for the ATP-binding site of a target protein, the assay comprising:
(i) incubating a test molecule with the target protein in the presence of a probe compound of formula I, I-I, I-II or I-III, or a salt thereof, as defined herein; and
(ii) determining whether any probe compound is displaced from the ATP-binding site of the target protein.

In an embodiment, the target protein is selected from MLH1 or MLH1 and PMS2. In a particular embodiment, the target protein is MLH1 and PMS2. In a specific embodiment, the target protein is only MLH1.

Suitably, the assay is a displacement assay. Displacement assays are well known in the art. In embodiments of the invention in which Q is a fluorophore, the displacement assay is a fluorescence polarisation assay. Fluorescence polarisation assays are well known in the art.

The step of determining whether any probe compound is displaced from the ATP-binding site of the target protein suitably comprises detecting whether the test compound results in an increase in the proportion of probe compound of formula I, I-I, I-II or I-III that is unbound to the ATP-binding site. This can be achieved by either detecting whether there is an increase in the amount of unbound probe compound (relative to a control) or, if the amount of bound probe compound can be readily detected (e.g. by fluorescence polarisation) then detecting whether there is a decrease in the amount of bound probe compound (again relative to a control).

The determination of the proportion of the probe compound that is bound to the ATP-binding site of the target protein relative to the amount that is unbound can be achieved by techniques known in the art. The particular techniques utilised will depend on the nature of the detection moiety Q in the compound of formula I, I-I, I-II or I-III. Suitably, Q is a fluorophore and the assay is a fluorescence polarisation assay. In such assays, the difference in the fluorescence caused by the displacement of probe compound from the ATP-binding site of the target protein can be detected and used to quantify the amount of any displaced probe compound—and thereby determine the binding affinity of the test compound for the ATP binding site of the target protein.

In another aspect, the present invention provides a method for determining the binding affinity of a test molecule for the ATP-binding site of a target protein, the assay comprising:
(i) incubating a test molecule with the target protein in the presence of a probe compound of formula I, I-I, I-II or I-III, or a salt thereof, as defined herein; and
(ii) determining whether any probe compound is displaced from the ATP-binding site of the target protein.

In an embodiment, the target protein is selected from MLH1 or MLH1 and PMS2. In a particular embodiment, the target protein is MLH1 and PMS2. In a specific embodiment, the target protein is only MLH1.

Suitably, the method is a displacement assay. Displacement assays are well known in the art. In embodiments of the invention in which Q is a fluorophore, the displacement assay is a fluorescence polarisation assay. Fluorescence polarisation assays are well known in the art.

The step of determining whether any probe compound is displaced from the ATP-binding site of the target protein can be conducted in the manner described above.

In another aspect, the present invention provides an assay for determining the location and/or quantity of a target protein present within a biological sample, the assay comprising:
(i) contacting the biological sample with a probe compound of formula I, I-I, I-II or I-III, or a salt thereof, as defined herein; and
(ii) determining location and/or quantity of the compound of formula I, I-I, I-II or I-III present within the sample by detecting the location and/or intensity of the detection moieties of the compounds of formula I present within the biological sample.

In another aspect, the present invention provides an assay for determining the location and/or quantity of a target protein present within a biological sample, the assay comprising:
(i) contacting the biological sample with a probe compound of formula I, I-I, I-II or I-III in which one of R¹² or R¹³ is a group -Lₓ-X as defined herein, or a salt thereof, as defined herein; and
(ii) contacting the biological sample with a detection moiety Q, or a compound of the formula Q-L₂-Y as defined herein, that is capable of reacting with the functional group X present on the compound of formula I, I-I, I-II or I-III to form a compound of formula I, I-I, I-II or I-III in which one of R¹² or R¹³ is a group -L-Q as defined herein in situ within the biological sample;
(iii) determining location and/or quantity of the compound of formula I present within the sample by detecting the location and/or intensity of the detection moieties of the compounds of formula I, I-I, I-II or I-III present within the biological sample.

In another aspect, the present invention provides a method for determining the location and/or quantity of a target protein present within a biological sample, the method comprising:
(i) contacting the biological sample with a probe compound of formula I, I-I, I-II or I-III, or a salt thereof, as defined herein; and
(ii) determining location and/or quantity of the compound of formula I, I-I, I-II or I-III present within the sample by detecting the location and/or intensity of the detection moieties of the compounds of formula I, I-I, I-II or I-III present within the biological sample.

In another aspect, the present invention provides a method for determining the location and/or quantity of a target protein present within a biological sample, the method comprising:
(i) contacting the biological sample with a detection moiety Q, or a compound of the formula Q-L₂-Y as defined herein, that is capable of reacting with the functional group X present on the compound of formula I, I-I, I-II or I-III in which one of R¹² or R¹³ is a group -Lₓ-X as defined herein, to form a compound of formula I, I-I, I-II or I-III in which one of R¹² or R¹³ is a group -L-Q as defined herein in situ within the biological sample;
(ii) determining location and/or quantity of the compound of formula I, I-I, I-II or I-III present within the sample by detecting the location and/or intensity of the detection moieties of the compounds of formula I present within the biological sample.

In an embodiment, the target protein is selected from MLH1 or MLH1 and PMS2. In a particular embodiment, the target protein is MLH1 and PMS2. In a specific embodiment, the target protein is only MLH1.

Suitably the step of contacting the biological sample with a probe compound of formula I, I-I, I-II or I-III (step (i) in the above assays and methods), involves incubating the biological sample with the probe compound under conditions that enable it to bind to the ATP-binding site of a target protein (e.g. MLH1 or MLH1 and PMS2).

The determination of the distribution and quantity of the compound of formula I within the biological sample can be achieved by techniques known in the art. The particular techniques utilised will depend on the nature of the detection moiety Q in the compound of formula I.

Suitably, Q is a fluorophore and the determination of location and quantity of the compound of formula I within the sample can be achieved by fluorescence microscopy/imaging. Appropriate calibration of the fluorescence intensity can be used to determine the quantity of the compound of formula I at various locations within the sample.

The assays and methods of the invention defined herein may optionally further comprise one or more washing steps conducted after step (i) to remove excess probe compound of formula I, I-I, I-II or I-III from the biological sample.

### EXAMPLES

Abbreviations
Boc for tert-butyloxycarbonyl
DAST for diethylaminosulfur trifluoride
DBU for 1,8-diazabicyclo(5.4.0)undec-7-ene
DCC for dicyclohexylcarbodiimide
DCE for 1,1-dichloroethane
DCM for dichloromethane
DEA for diethanolamine
DEAD for diethyl azodicarboxylate
DIAD for diisopropyl azodicarboxylate
DIBAL for Diisobutylaluminium hydride
DIPEA for N,N-diisopropylethylamine, Hünig's base
DMA for N,N-dimethylacetamide
DMAP for 4-(dimethylamino) pyridine
DMF for N,N-dimethylformamide
DMSO for dimethylsulfoxide.
EDC for 1-Ethyl-3-(3-dimethylaminopropyl)carbodiimide
EtOAc for ethyl acetate
h for hours
HATU for N-[(dimethylamino)-1H-1,2,3-triazolo-[4,5-b]pyridin-1-ylmethylene]-N-methylmethanaminium hexafluorophosphate N-oxide
HBTU for (1H-benzotriazol-1-yloxy)(dimethylamino)-N,N-dimethylmethaniminium hexafluorophosphate
HOBT for N-hydroxybenzotriazole
HPLC for High Pressure Liquid Chromatography.
LAH for lithium aluminium hydride
IPA for isopropyl alcohol
LCMS for Liquid Chromatography-Mass Spectrometry
LDA for Lithium diisopropylamide
LiHMDS for Lithium bis(trimethylsilyl)amide
mCPBA for meta-chloroperoxybenzoic acid
MI for Molecular Ion
Min for minutes
MgSO₄ anhydrous magnesium sulfate
MW for microwave
NBS for N-bromosuccinamide
NCS for N-chlorosuccinamide
NFOBS for N-fluoro-o-benzenedisulfonimide
NFSI for N-fluorobenzenesulfonimide
NHS for N-hydroxysuccinimide
NIS for N-iodosuccinamide
NMM for N-methylmorpholine
NMP for 1-methyl-2-pyrrolidinone
NMR for Nuclear Magnetic Resonance.
PdCl₂(PPh₃)₂ for Bis(triphenylphosphine)palladium chloride
Pd(dppf)₂Cl₂ for [1,1′-bis(diphenylphosphino)ferrocene]dichloropalladium(II)
Pd(dppf)₂Cl₂.DCM for [1,1′-bis(diphenylphosphino)ferrocene]dichloropalladium(II) complex with DCM
(Pd(dba)₂) for bis(dibenzylideneacetone)palladium
Rbf for round bottomed flask
RT for Retention Time.
SCX-2 for a silica-based sorbent with a chemically bonded propylsulfonic acid functional group
SFC for supercritical fluid chromatography
STAB for Sodium triacetoxyborohydride
TBAF for tetra-n-butylammonium fluoride
TBDMS for tert-butyldimethylsilyl
TFAA for trifluoroacetic anhydride
TFA for trifluoroacetic acid
THF for tetrahydrofuran
TPP for tripotassium phosphate
Ts for toluenesulfonyl
XPhos-Pd-G1 for 2-Dicyclohexylphosphino-2′,4′,6′-triisopropyl-1,1′-biphenyl)[2-(2-aminoethyl)phenyl)]palladium(II) chloride
XPhos-Pd-G2 for Chloro(2-dicyclohexylphosphino-2′,4′,6′-triisopropyl-1,1′-biphenyl)[2-(2′-amino-1,1′-biphenyl)]palladium(II)

### Analytical Methods

Commercially available starting materials, reagents and dry solvents were used as supplied. Flash column chromatography or glass column chromatography was performed using Merck silica gel 230-400 mesh size. Column chromatography was also performed using glass column chromatography or Flash chromatography. Flash chromatography was performed on combi-flash RF Teledyne Isco machine. Preparative TLC was performed on Merck plates. ¹H NMR spectra were recorded on a Bruker Avance-400 or Bruker Avance III 400 MHz. Samples were prepared as solutions in a suitable deuterated solvent and referenced to the appropriate internal non-deuterated solvent peak or tetramethylsilane; chemical shifts were recorded in ppm (δ) downfield of tetramethylsilane. Flash chromatography was also performed on combi-flash RF Teledyne Isco machine. Preparative TLC was performed on Merck plates.

### Liquid Chromatography-Mass Spectrometry Methods

### Method A

Waters Acquity UPLC with binary solvent manager, PDA detector and Acquity QDA performance mass detector, column: X-Bridge BEH C18, 50×2.1 mm, 2.5 micron, column temperature: 35° C., auto sampler temperature: 5′C, mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method B

Waters Acquity with PDA detector and SQ Detector, column: X-Bridge BEH C18, 50×2.1 mm, 2.5 micron, column temperature: 35° C., auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min (97% A, 3% B) flow: 0.5 mL/min; end of run at t=4 min (97% A, 3% B), flow rate: 0.5 mL/min, analysis time 4 min. Mass detection parameter: ionization mode was cycled through positive and negative mode with cone voltage 10 V and 30 V and 3.25 kV capillary voltage, temperature of source and probe were 120° C. and 400° C. respectively.

### Method C

Waters Acquity UPLC with binary solvent manager, PDA detector and Acquity QDA performance mass detector, column: YMC Tri-art C18, 50×2 mm, 1.9 micron, column temperature: 350C, auto sampler temperature: 5′C, mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min
(0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method D

Waters Acquity UPLC with quaternary solvent manager, with PDA detector and SQ detector, column: X-Bridge BEH C18, 50*2.1 mm,2.5 micron, column temperature: 350C, auto sampler temperature: 5° C., mobile phase A: 0.1% (v/v) Formic acid in water (pH=2.70), mobile phase B: 0.1% (v/v) formic acid in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ESI capillary probe, ionization mode cycled through positive and negative modes with cone voltage 10 V and 30V and 0.8 kV capillary voltage, temperature of source and probe were 120° C. and 400° C. respectively.

### Method E

Waters Acquity UPLC with binary solvent manager, PDA detector and Acquity QDA performance mass detector, column: Welch Xtimate C18, 50×2.1 mm, 1.8 micron, column temperature: 35° C., auto sampler temperature: 5′C, mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method F

Waters Acquity UPLC with PDA detector and SQ Detector, column: Welch-Xtimate,C18 4.6*50 mm,5 micron, column temperature: 350C, auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min
(97% A, 3% B) flow: 0.5 mL/min; end of run at t=4 min (97% A, 3% B), flow rate: 0.5 mL/min, analysis time 4 min. Mass detection parameter: ionization mode was cycled through positive and negative mode with cone voltage 10 V and 30 V and 3.25 kV capillary voltage, temperature of source and probe were 120° C. and 400° C. respectively.

### Method G

Waters Acquity UPLC with binary solvent manager, PDA detector and Acquity QDA performance mass detector, column: X-Bridge C18 2.1*50 mm3.5 micron, column temperature: 350C, auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min (97% A, 3% B) flow: 0.5 mL/min; end of run at t=4 min (97% A, 3% B), flow rate: 0.5 mL/min, analysis time 4 min. Mass detector parameter: ionization mode was cycled through positive and negative modes with cone voltage 10 V and 30 V and 0.8 kV capillary voltage, temperature of source and probe were 120° C. and 600° C. respectively.

### Method H

Waters Acquity with PDA detector and SQ Detector, column: X-bridge C18, 50×4.6 mm, 3.5 micron, column temperature: 25° C., mobile phase A: 5 mM ammonium bicarbonate in ddH₂O (pH=7.35), mobile phase B: MeOH, mobile phase gradient details: t=0 min (92% A, 8% B); t=0.75 min (92% A, 8% B); gradient to t=3 min (30% A, 70% B gradient to t=3.75 min (5% A, 95% B); gradient to t=4.20 min (0% A, 100% B); t=5.20 min (0% A, 100% B); gradient to t=5.21 min (92% A, 8% B); end of run at t=7 min (92% A, 8% B), Flow rate: 1 mL/min, analysis time 7 min. Mass detector parameter: ionization mode was cycled through positive and negative mode with cone voltage 10 V and 30 V and 3.25 kV capillary voltage, temperature of source and probe were 120° C. and 600° C. respectively.

### Method I

Acquity UPLC (binary pump/PDA detector) coupled to Waters ZQ Mass Spectrometer. Column: Acquity UPLC BEH Shield RP18 1.7 μm 100×2.1 mm. (Plus guard cartridge), maintained at 40° C. Mobile phase: MeCN in water (with 10 mM ammonium bicarbonate), from 5% to 95% within 6 min; Flow rate: 0.5 ml/min; Wavelength: 210-400 nm DAD.

### Method-J

LC/MS was performed on Acquity UPLC (binary pump/PDA detector) coupled to Waters ZQ Mass Spectrometer. Column: Acquity UPLC HSS C18 1.8 μm 100×2.1 mm. (Plus guard cartridge), maintained at 40° C. Mobile phase: MeCN (0.1% formic acid) in water (0.1% formic acid), from 5% to 95% within 6 min; Flow rate: 0.5 ml/min; Wavelength: 210-400 nm DAD.

### Analytical HPLC Methods

### Method A

Agilent 1100 Series instrument with PDA detector, Sunfire (C18 150 mm×4.6 mm) 3.5 m column, temperature: 250C; auto sampler temperature: 250C, Mobile Phase A: 0.05% Trifluoro acetic acid in Milli Q water (pH=2.1), Mobile Phase B: Acetonitrile (100%). Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min; Run Time: 17 min.

### Method B

Agilent 1260 Series instrument with PDA detector, Sunfire C18 (150 mm×4.6 mm) 3.5 m column, temperature: 250C; auto sampler temperature: 250C, Mobile Phase A: 0.05% Trifluoro acetic acid in Milli Q water (pH=2.1), Mobile Phase B: Acetonitrile (100%). Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min, Run Time: 17 min.

### Method C

Waters Alliance e2695 instrument with 2998 PDA detector, Atlantis C18 (150 mm×4.6 mm) 5 m column, temperature: 25° C.; Auto sampler temperature: 25° C., Mobile Phase A: 0.1% ammonium hydroxide solution in HPLC water. Mobile Phase B: Acetonitrile (100%). Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.0 mL/min; Run Time: 17 min.

### NMR

¹H Nuclear magnetic resonance (NMR) spectroscopy was carried out using a Bruker Avance-400 instrument operating at 400 MHz using the stated solvent at room temperature unless otherwise stated. Samples were prepared as solutions in a suitable deuterated solvent and referenced to the appropriate internal non-deuterated solvent peak or tetramethylsilane. Chemical shifts were recorded in ppm (δ) downfield of tetramethylsilane. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; m, multiplet; br, broad.

### Purification Methods

### Preparative HPLC Purification

### Preparatory HPLC Method A

Shimadzu Prep-20-AD with binary pump with UV/Visible wave-length detector, Column: C18, 250×20 mm, 5 micron, column temperature: room temp., mobile phase A: 0.1% formic acid in water, mobile phase B: Acetonitrile: Methanol:2-Propanol(65:25:10); mobile phase gradient details: t=0 min (55% A, 45% B); t=17 min (55% A, 45% B); gradient to t=17.01 min (2% A, 98% B); t=19 min (2% A, 98% B); gradient to t=19.01 min (55% A, 45% B); end of run at t=21 min (55% A, 45% B), flow rate: 27 mL/min, analysis time 21 min.

### Preparatory HPLC Method B

Shimadzu Prep-20-AD with binary pump with UV/Visible wave-length detector, Column: C18, 250*20 mm, 5 micron, column temperature: room temp., mobile phase A: 0.1% formic acid in water, mobile phase B: Acetonitrile; mobile phase gradient details: t=0 min (72% A, 28% B); t=19 min (72% A, 28% B); gradient to t=19.01 min (2% A, 98% B); t=21 min (2% A, 98% B); gradient to t=21.01 min (72% A, 28% B); end of run at t=24 min (72% A, 28% B), flow rate: 20 mL/min, analysis time 24 min.

### Preparatory HPLC Method C

Sample purification was performed on Agilent Series 1260 Infinity-II purification system with PDA detector, Column: Waters Sunfire C18, 150*4.6 mm, 3.5 micron, Column temperature: room temp., auto sampler temperature: 15° C., mobile phase A: 0.05% (v/v) tri-fluoroacetic acid in Milli-Q water (pH˜2.5), mobile phase B: 100% acetonitrile; mobile phase gradient details: t=0 min (90% A, 10% B; gradient to t=7 min (10% A, 90% B); gradient to t=9 min (0% A, 100% B); t=14 min (0% A, 100% B); gradient to t=14.01 min (90% A, 10% B); end of run at t=17 min (90% A, 10% B) unless otherwise stated; flow rate: 1 mL/min, analysis time 17 min.

### Preparatory HPLC Method D

Sample purification was performed on Agilent Technologies 1260 Infinity purification system and Agilent 6120 series Single Quadrupole Mass Spectrometer; column: Waters X-bridge Phenyl 100×19 mm id 10 μm Mobile phase: MeOH in water (0.1% (NH₄)₂CO₃), Flow rate: 20 ml/min; Wavelength: 210-400 nm DAD. Sample injected in DMSO, 23 min non-linear gradient from 5% to 100% MeCN, centered on a specific focused gradient.

### Preparatory HPLC Method E

Chromatographic separation and isolation were conducted with Waters 2545 quaternary system with waters 2489 UV detector. The column used was YMC-ODS AQ C18-s,250×20 mm, 5 micron and the compounds were eluted with mobile phase A: 0.1% formic acid in water, mobile phase B: 20% Mobile phase A in Acetonitrile with a gradient of T=0 min (50% A, 50% B); gradient to T=23.0 min (50% A, 50% B); T=23.01 min (2% A, 98% B); gradient to T=26.00 min (2% A, 98% B); T=26.01 min (50% A, 50% B); gradient to T=29.00 min (50% A, 50% B); with Flow rate=16 mL/min; analysis time 29 min.

### Preparatory HPLC Method F

Chromatographic separation and isolation were conducted with Shimadzu LC20AP with UV detector. The column used was Repack C18, 5 micron and the compounds were eluted with mobile phase A: water, mobile phase B: Acetonitrile with a gradient of T=0 min (60% A, 40% B); gradient to T=17.0 min (40% A, 60% B); T=17.01 min (2% A, 98% B); gradient to T=19.00 min (2% A, 98% B); T=19.01 min (60% A, 40% B); gradient to T=22.00 min (60% A, 40% B); with Flow rate=25 mL/min; analysis time 22 min.

### Preparatory HPLC Method G

Chromatographic separation and isolation were conducted with Biotage flash chromatography system with UV detector. The column used was YMC C18 120 gm, 50 micron and the compounds were eluted with, mobile phase A: 0.1% Formic acid in Milli Q water, mobile phase B: Acetonitrile with gradient of T=0 min (100% A, 0% B); gradient T=5.0 min (100% A, 0% B); gradient to T=40.0 min (60% A, 40% B); T=40.01 min (0% A, 100% B); gradient to T=45.00 min (0% A, 100% B); T=45.01 min (100% A, 0% B); gradient to T=50.00 min (100% A, 0% B); with Flow rate=80 mL/min; analysis time 50 min.

### Preparatory HPLC Method H

Chromatographic separation and isolation were conducted with Shimadzu LC20AP with UV detector. The column used was Sunfire prep C18 OBD,250×19 mm, 5 micron and the compounds were eluted with, mobile phase A: 0.1% Formic acid in water, mobile phase B: Acetonitrile with a gradient of T=0 min (88% A, 12% B); gradient to T=20.0 min (82% A, 18% B); T=20.01 min (2% A, 98% B); gradient to T=23.00 min (2% A, 98% B); T=23.01 min (88% A, 12% B); gradient to T=27.00 min (88% A, 12% B); with Flow rate=22 mL/min; analysis time 27 min.

### Preparatory HPLC Method I

Chromatographic separation and isolation were conducted with Biotage flash chromatography system with UV detector. The column used was YMC C18 120 gm, 50 micron and the compounds were eluted with, mobile phase A: 0.1% Formic acid in Milli Q water, mobile phase B: Acetonitrile with a gradient of T=0 min (100% A, 0% B); gradient T=5.0 min (95% A, 5% B);gradient to T=30.0 min (60% A, 40% B); T=30.01 min (0% A, 100% B); gradient to T=35.00 min (0% A, 100% B); T=35.01 min (100% A, 0% B); gradient to T=40.00 min (100% A, 0% B); with Flow rate=80 mL/min; analysis time 40 min.

### Preparatory HPLC Method J

Chromatographic separation and isolation were conducted with Waters 2545 quaternary system with waters 2489 UV detector. The column used was X-bridge prep C18 OBD 250×19 mm, 5 micron and the compounds were eluted with, mobile phase A: 0.1% Formic acid in water, mobile phase B: 20% Mobile phase A in Acetonitrile with a gradient of T=0 min (95% A, 5% B); gradient T=2.0 min (80% A, 20% B); gradient to T=22.0 min (76% A, 24% B); T=22.01 min (2% A, 98% B); gradient to T=24.00 min (2% A, 98% B); T=24.01 min (70% A, 30% B); gradient to T=28.00 min (95% A, 5% B); with Flow rate=23 mL/min; analysis time 28 min.

### Preparatory HPLC Method K

Chromatographic separation and isolation were conducted with Waters 2545 quaternary system with waters 2489 UV detector. The column used was X-bridge prep C18 OBD 250×19 mm, 5 micron and the compounds were eluted with, mobile phase A: 0.1% Formic acid in water, mobile phase B: Acetonitrile with gradient of T=0 min (92% A, 8% B); gradient to T=15.0 min (68% A, 32% B); T=15.01 min (2% A, 98% B); gradient to T=17.00 min (2% A, 98% B); T=17.01 min (68% A, 32% B); gradient to T=20.00 min (68% A, 32% B); with Flow rate=20 m L/min; analysis time 20 min.

### Preparatory HPLC Method L

Chromatographic separation and isolation were conducted with Waters 2545 quaternary system with waters 2489 UV detector. The column used was Sunfire prep C18 OBD 150×19 mm, 5 micron and the compounds were eluted with, mobile phase A: 0.1% Formic acid in water, mobile phase B: 20% Mobile phase A+5% Tetrahydrofuran in Acetonitrile with a gradient of T=0 min (70% A, 30% B); gradient T=2.0 min (57% A, 43% B); gradient to T=21.0 min (57% A, 43% B); T=21.01 min (2% A, 98% B); gradient to T=23.00 min (2% A, 98% B); T=23.01 min (70% A, 30% B); gradient to T=25.00 min (70% A, 30% B); with Flow rate=21 mL/min; analysis time 25 min.

### Preparatory HPLC Method M

Chromatographic separation and isolation were conducted with Biotage flash chromatography system with UV detector. The column used was YMC C18 120 gm, 50 micron and the compounds were eluted with, mobile phase A: 0.1% Formic acid in water, mobile phase B: Acetonitrile with gradient of T=0 min (100% A, 0% B); gradient to T=35.0 min (65% A, 35% B); T=35.01 min (2% A, 98% B); gradient to T=38.00 min (2% A, 98% B); T=38.01 min (100% A, 0% B); gradient to T=40.00 min (100% A, 0% B); with Flow rate=80 mL/min; analysis time 40 min.

### Chiral Prep HPLC Methods

The enantiomeric separation of compounds was achieved by Chiral Prep HPLC purification methods. Below is a list of Chiral Prep HPLC purification methods and conditions used to resolve enantiomers or to determine enantiomeric purity (ee).

| Chiral Prep | |
|---|---|
| HPLC | |
| methods | Conditions for chiral resolution or ee determination |
| Method A | Waters binary gradient with 600-controller pump with Waters 2487-UV/Visible |
| | wave-length detector, Column: YMC-CHIRAL ART CELLULOSE-SC, 250*20 |
| | mm, 5 micron, column temperature: room temp. Mobile phase A: n-Heptane, |
| | mobile phase B: 2-propanol with isocratic flow A:B~70:30, flow rate: 17 |
| | mL/min, analysis time 55 min. |
| Method B | Agilent 1200 series infinity-II purification system with UV detector. Column: |
| | Chiral Pak IG SFC 21 × 250 mm, 5 μm. Compounds eluted with: Mobile |
| | Phase A: 0.1% diethyl amine in n-heptane, Mobile Phase B: Isopropyl |
| | alcohol: ethanol: methyl t-butyl ether (70:20:10) with isocratic flow (83% A, |
| | 17% B); for 55 min. Flow rate: 18 mL/min, analysis time 55 min. |
| Method C | Agilent 1200 series Infinity-II purification system with UV detector. Column: |
| | YMC chiral-Art cellulose-SC, 20 × 250 mm, 5 μm. Compounds eluted with: |
| | Mobile Phase A: n-Heptane; Mobile Phase B: Isopropyl alcohol: |
| | Methanol:(70:30) +1% Acetonitrile with isocratic flow (80% A, 20% B) for |
| | 45 min. Flow rate: 20 mL/min, analysis time 45 min. |
| Method D | Waters 2545 purification system with UV detector. Column: Chiral Pak AD, |
| | 30 × 250 mm, 10 μm. Compounds eluted with: Mobile Phase A: 0.1% |
| | Diethylamine in n-heptane, Mobile Phase B: Isopropylalcohol:Methanol |
| | (70:30) with isocratic flow: T = 0 min (78% A, 22% B); gradient to T = 50 min |
| | (78% A, 22% B); Flow rate = 35 ml/min; analysis time 50 min. |
| Method E | Waters SFC investigator with 2998 PDA detector. Column: Chiral Pak IG SFC |
| | 21 × 250 mm, 5 μm. Compounds eluted with: Mobile Phase A: Liquid CO₂, |
| | Mobile Phase B: 0.1% Diethylamine in methanol with isocratic flow (50% A, |
| | 50% B) for 30 min. Flow rate: 10 mL/min, analysis time 30 min. |
| Method F | Waters SFC purification system with UV detector. Column: Chiral Pak IG SFC |
| | 21 × 250 mm, 5 μm. Compounds eluted with: Mobile Phase A: Liquid CO₂, |
| | Mobile Phase B: Methanol. Isocratic method (75% A, 25% B) with flow rate of |
| | 90 ml/min; analysis time 9 min. |

For Example 110 and Example 111: NanoBRET® 590 SE, NanoBRET® 590-O4-SE, and NanoBRET® 590-C4-SE were obtained from Promega Corp. Madison, WI. Mass spectra were recorded on a Thermo Fisher Vanquish™ (LC-MS) and purity (≥95%) determined by reverse-phase high pressure liquid chromatography (RP-HPLC) using a Kinetex 5 μm EVO C18 100 Å LC Column 30×2.1 mm column or a Phenomenex Synergi 2.5 μm Max-RP 100 Å LC column. Compounds were purified on a Waters LC Prep 150 using a Waters XBridge Prep C18 OBD 30×250 mm column. Method 1: Initial—90% aqueous (0.1% TFA in H₂O), 10% acetonitrile to 0% aqueous, 100% acetonitrile, 30 min linear gradient.

### Synthesis

Several methods for the chemical synthesis of compounds of the present application are described herein. These and/or other well-known methods may be modified and/or adapted in various ways to facilitate the synthesis of additional compounds within the scope of the present application and claims. Such alternative Methods and modifications should be understood as being within the spirit and scope of this application and claims. Accordingly, Methods set forth in the following descriptions, Schemes and Examples are intended for illustrative purposes and are not to be construed as limiting the scope of the disclosure.

In one approach (Scheme 1), compounds of formula [I] may be prepared by the reaction of a substituted aromatic carboxylic acid of formula [II] with oxalyl chloride or thionyl chloride in a solvent such as DCM, with an amine of general formula [III] in the presence of a tertiary amine base such as Et₃N, DIPEA or NMM. The reaction is suitably conducted at RT. After reaction work up, typically by liquid-liquid extraction, the reaction product is purified by flash column chromatography, reverse phase preparative HPLC or re-crystallisation (Method A). Compounds of compounds of formula [I] may also be prepared by the reaction of a substituted aromatic carboxylic acid of formula [II] with an amine of general formula [III] and a suitable coupling agent such as HOBT/EDC, HBTU or HATU in a polar aprotic solvent such as DMA or DMF in the presence of a tertiary amine base such as Et₃N, DIPEA or NMM. After reaction work up, typically by liquid-liquid extraction, the reaction product is purified by flash column chromatography, reverse phase preparative HPLC or re-crystallisation (Method B). The bis-tosyl protected intermediate (from Method A or B) is then subject to deprotection with a suitable base such as K₂CO₃ or KOH in a suitable protic solvent such as MeOH or EtOH. The reaction is suitably conducted at high temperature. After reaction work up, typically by liquid-liquid extraction, the reaction product is purified by flash column chromatography, reverse phase preparative HPLC or re-crystallisation, to yield compounds of general the Formula [1]

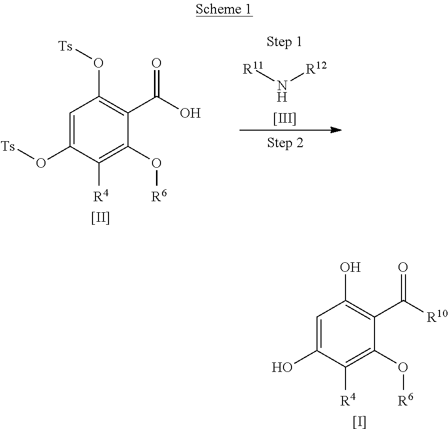

### Example 1: (R)-(2-(Benzyloxy)-4,6-dihydroxyphenyl)(8-((tetrahydrofuran-3-yl)amino)-3,4-dihydro-isoquinolin-2(1H)-yl)methanone

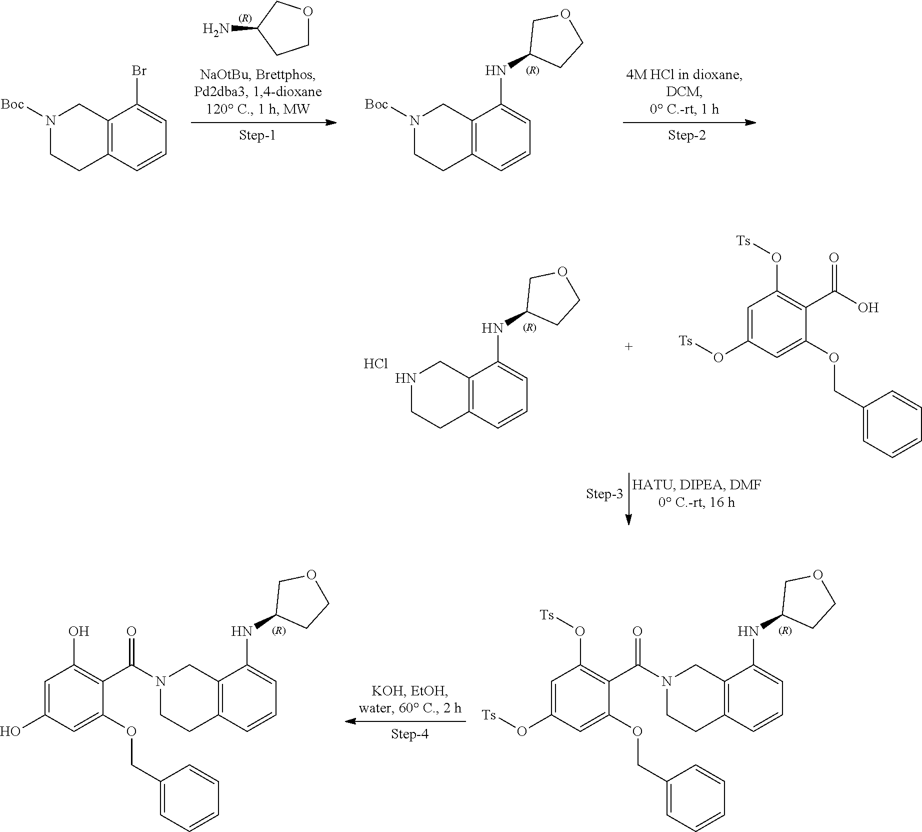

### Step-1: t-Butyl (R)-8-((tetrahydrofuran-3-yl)amino)-3,4-dihydroisoquinoline-2(1H)-carboxylate

A stirred solution of t-butyl 8-bromo-3,4-dihydroisoquinoline-2(1H)-carboxylate (0.70 g, 2.24 mmol, 1 eq) (CAS: 893566-75-1), (R)-tetrahydrofuran-3-amine (0.234 g, 2.68 mmol, 1.5 eq) (CAS: 111769-26-7), and NaOtBu (0.537 g, 5.60 mmol, 2.5 eq) in 1,4-dioxane (7 ml) in a 35 mL microwave sealed tube was degassed using N₂ gas for 15-20 min. Brettphos (0.007 g, 0.013 mmol, 0.006 eq) and Pd₂(dba)₃ (0.006 g, 0.006 mmol, 0.003 eq) were added to reaction mixture at room temperature. The resulting reaction mixture was heated to 120° C. under microwave irradiation for 1 h. The reaction mixture was poured into water (100 mL) and extracted in ethyl acetate (3×100 mL). The combined organic layer was dried over Na₂SO₄, filtered and concentrated under vacuum. The obtained crude material was purified by flash chromatography (product eluted in 15% EtOAc in hexane) yielding the title compound as a yellow solid (0.430 g, Yield: 56.1%). ^{t}H NMR (DMSO-d6, 400 MHz): δ ppm 1.42 (s, 9H), 1.74 (m, 1H), 1.84-1.85 (m, 1H), 2.14-2.19 (m, 1H), 2.66-2.68 (m, 2H), 3.33-3.50 (m, 2H), 3.56-3.59 (m, 1H), 3.69-3.74 (m, 1H), 3.80-3.85 (m, 1H), 3.90-3.93 (m, 1H), 3.99-4.05 (m, 1H), 4.27 (s, 2H), 4.84 (bs, 1H), 6.45 (d, J=8 Hz, 2H), 6.99 (t, J=8 Hz, 15.6 Hz, 1H). LCMS (Method A): 2.127 min, MS: ES+263.10 (M−56).

### Step-2: (R)—N-(Tetrahydrofuran-3-yl)-1,2,3,4-tetrahydroisoquinolin-8-amine hydrochloride (Intermediate 40)

A stirred solution of t-butyl (R)-8-((tetrahydrofuran-3-yl)amino)-3,4-dihydroisoquinoline-2(1H)-carboxylate (0.4 g, 1.25 mmol, 1.0 eq) in DCM (4 mL) was cooled to 0° C. and 4M HCl in dioxane (2 mL) was added dropwise. The reaction mixture was stirred at room temperature for 1 h. The reaction mixture was concentrated under vacuum and crude material was triturated using diethyl ether (2×50 mL) followed by n-pentane (3×50 mL). The obtained solid material was dried under high vacuum yielding the title compound as a brown solid (0.36 g, quantitative). ¹H NMR (DMSO-d6, 400 MHz): δ ppm 1.84-1.91 (m, 1H), 2.13-2.22 (m, 1H), 2.92-2.95 (m, 2H), 3.25-3.38 (m, 2H), 3.62-3.74 (m, 2H), 3.82-3.92 (m, 2H), 3.99-4.03 (m, 3H), 6.58-6.64 (m, 2H), 7.03-7.12 (m, 1H), 9.83 (bs, 2H). LCMS (Method A): 0.882 min, MS: ES+219.14 (M+1).

### Step-3: (R)-5-(Benzyloxy)-4-(8-((tetrahydrofuran-3-yl)amino)-1,2,3,4-tetrahydroisoquinoline-2-carbonyl)-1,3-phenylene bis(4-methylbenzenesulfonate

A stirred solution of 2-(benzyloxy)-4,6-bis(tosyloxy)benzoic acid (Intermediate 61) (0.50 g, 0.88 mmol, 1 eq) in DMF (5 mL) at 0° C. was treated with HATU (0.501 g, 1.32 mmol, 1.5 eq), DIPEA (0.23 g, 1.78 mmol, 2 eq) and the mixture stirred (10 min). (R)—N-(tetrahydrofuran-3-yl)-1,2,3,4-tetrahydroisoquinolin-8-amine hydrochloride (Intermediate 40) (0.228 g, 1.04 mmol, 1.2 eq) was added to reaction mixture at 0° C. The resulting reaction mixture was stirred at room temperature for 16 h, poured into water (100 mL) and extracted using ethyl acetate (3×120 mL). The combined organic layer dried over Na₂SO₄, filtered, and concentrated under vacuum. Crude material was purified by flash chromatography (product eluted in 25% EtOAc in hexane) yielding the title compound as an off-white solid (0.380 g, Yield: 59.2%). ¹H NMR (DMSO-d6, 400 MHz). LCMS (Method A): 2.652 min, 2.768 min, MS: ES+769.2 (M+1).

### Step-4: (R)-(2-(Benzyloxy)-4,6-dihydroxyphenyl)(8-((tetrahydrofuran-3-yl)amino)-3,4-dihydroisoquinolin-2(1H)-yl)methanone

A stirred solution of (R)-5-(benzyloxy)-4-(8-((tetrahydrofuran-3-yl)amino)-1,2,3,4-tetrahydroisoquinoline-2-carbonyl)-1,3-phenylene bis(4-methylbenzenesulfonate (0.350 g, 0.45 mmol, 1 eq.) in EtOH:H₂O (1:1) at room temperature was treated with KOH (1.021 g, 18.19 mmol, 40 eq.). The reaction mixture was heated to 60° C. and stirred for 2 h. The reaction mixture was poured into water (100 mL), acidified using 1N HCl solution (pH˜6) and extracted in ethyl acetate (3×120 mL). The combined organic layer was dried over Na₂SO₄, filtered, and concentrated under vacuum. The crude material was purified by flash chromatography (product was eluted at 3.5% MeOH in DCM) yielding the title compound as an off-white solid (0.08 g, Yield: 33%). High temperature ¹H NMR (DMSO-d6, 400 MHz, 346K): δ 1.85-1.87 (m, 1H), 2.17-2.22 (m, 1H), 2.67-2.69 (m, 2H), 3.46-3.57 (m, 2H), 3.59-3.71 (m, 1H), 3.73-3.76 (m, 1H), 3.84-3.91 (m, 1H), 3.95-4.01 (m, 1H), 4.04-4.10 (m, 1H), 4.40-4.60 (m, 2H), 4.97 (s, 2H), 6.01 (s, 1H), 6.43-6.50 (m, 2H), 6.99 (t, J=8.4 Hz, 1H), 7.25-7.26 (m, 5H), 9.19-9.22 (m, 2H). LCMS (Method A): 1.813 min, MS ES+461.12 (M+1).

### Example 2: (S)-(2-(Benzyloxy)-4,6-dihydroxyphenyl)(8-((tetrahydrofuran-3-yl)amino)-3,4-dihydro-isoquinolin-2(1H)-yl)methanone

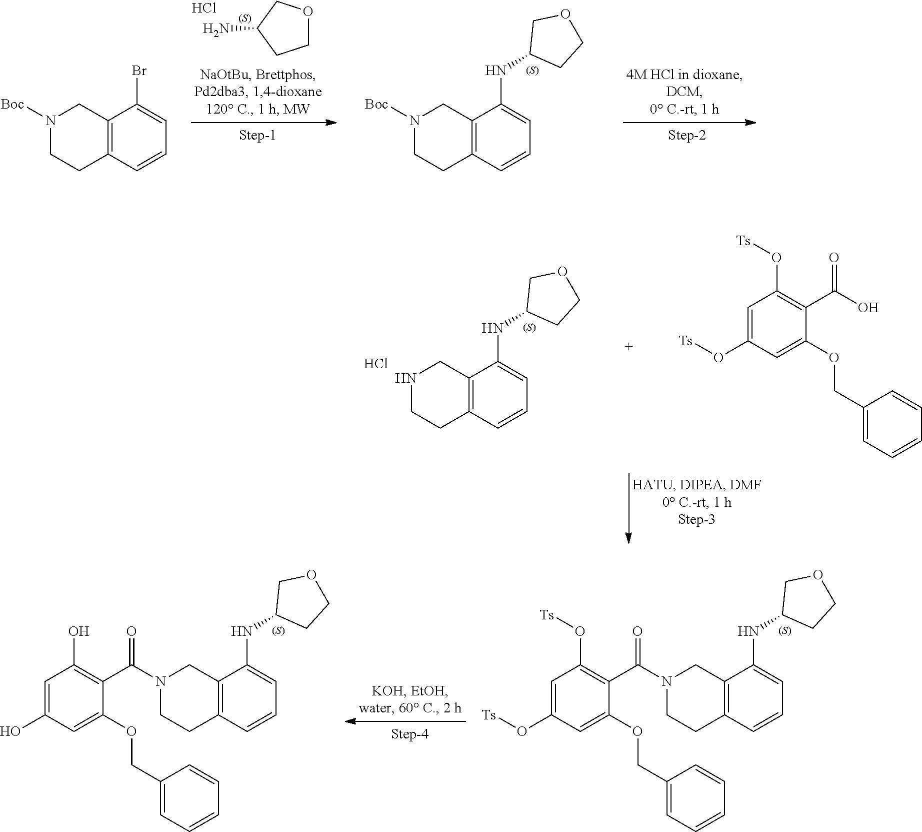

### Step-1: t-Butyl (S)-8-((tetrahydrofuran-3-yl)amino)-3,4-dihydroisoquinoline-2(1H)-carboxylate

A stirred solution of t-butyl 8-bromo-3,4-dihydroisoquinoline-2(1H)-carboxylate (0.6 g, 1.92 mmol, 1.0 eq) (CAS: 893566-75-1), (S)-tetrahydrofuran-3-amine hydrochloride (0.35 g, 2.88 mmol, 1.5 eq) (CAS: 204512-95-8), NaOtBu (0.46 g, 4.8 mmol,2.5 eq) in 1, 4-dioxane (8 mL) at room temperature was treated with Brettphos (0.06 g, 0.11 mmol, 0.06 eq) at room temperature under nitrogen atmosphere. The reaction mixture was degassed using N₂ (g) for 15 minutes. Pd₂ (dba)₃ (0.07 g, 0.07 mmol, 0.04 eq) was added and the reaction mixture stirred at 120° C. under microwave irradiation for 1 h. The reaction mixture was poured into water (30 mL) and extracted with ethyl acetate (3×50 mL). The combined organic layer was dried with Na₂SO₄, filtered, and concentrated under vacuum. The crude material was purified by flash chromatography (product eluted with 5% EtOAc in hexane) yielding the title compound (0.36 g, Yield: 58.8%). ¹H NMR (DMSO-d6, 400 MHz): δ ppm 1.43 (s, 9H), 1.84-1.91 (m, 1H), 2.13-2.22 (m, 1H), 2.66-2.69 (m, 2H), 3.37-3.50 (m, 2H), 3.56-3.59 (m, 1H), 3.69-3.74 (m, 1H), 3.80-3.85 (m, 1H), 3.90-3.93 (m, 1H), 3.99-4.05 (m, 1H), 4.27 (s, br, 2H), 4.84 (s, br, 1H), 6.45 (d, J=8 Hz, 2H), 6.99 (t, J=8 Hz, 15.6 Hz, 1H). LCMS (Method A): 2.122 min, MS: ES+263.10 (M−56).

### Step-2: (S)—N-(Tetrahydrofuran-3-yl)-1,2,3,4-tetrahydroisoquinolin-8-amine hydrochloride (Intermediate 39)

A stirred solution of t-butyl (S)-8-((tetrahydrofuran-3-yl)amino)-3,4-dihydroisoquinoline-2(1H)-carboxylate (0.31 g, 0.97 mmol, 1.0 eq) in DCM (5 mL) was cooled to 0° C. 4M HCl in dioxane (3 mL) was added dropwise to reaction mixture at 0° C. The reaction mixture was stirred at room temperature for 1 h, concentrated under vacuum and the crude solid material was triturated using diethyl ether (3×10 mL) and further dried over high vacuum yielding the title compound (0.22 g, Yield: 98.76%, 1.00 mmol). ¹H NMR (DMSO-d6, 400 MHz): 1.81-1.90 (m, 1H), 2.13-2.22 (m, 1H), 2.92-2.93 (m, 2H), 3.26-3.27 (m, 2H), 3.58-3.60 (m, 2H), 3.70-3.74 (m, 1H), 3.80-3.89 (m, 1H), 3.90-4.01 (m, 3H), 6.51-6.75 (m, 2H), 7.03-7.10 (m, 2H), 9.47-9.67 (bs, 2H). LCMS (Method B): 2.11 min, MS: ES+219.3 (M+1).

### Step-3: (S)-5-(Benzyloxy)-4-(8-((tetrahydrofuran-3-yl) amino)-1,2,3,4-tetrahydroisoquinoline-2-carbonyl)-1,3-phenylene bis(4-methylbenzenesulfonate)

A stirred solution of 2-(benzyloxy)-4,6-bis(tosyloxy)benzoic acid (Intermediate 61) (0.42 g, 0.739 mmol, 1.0 eq) in DMF (10 mL) at 0° C. was treated with DIPEA (0.19 g, 1.47 mmol, 2.0 eq), HATU (0.42 g, 1.10 mmol, 1.5 eq) and stirred for 5 mins. (S)—N-(Tetrahydrofuran-3-yl)-1,2,3,4-tetrahydroisoquinolin-8-amine hydrochloride (Intermediate 39) (0.209 g, 0.96 mmol, 1.3 eq) was added and the resulting reaction mixture was stirred at room temperature for 1 h. The reaction mixture was poured in to water (100 mL) and extracted with ethyl acetate (3×100 mL). The combined organic layer was dried by Na₂SO₄, filtered, and concentrated under vacuum. The crude material was purified by flash chromatography (product was eluted at 15% EtOAc in hexane) yielding the title compound (0.6 g, Yield: quantitative, 0.78 mmol). LCMS (Method A): 2.659 min, 2.733 min. MS: ES+769.06 (M+1).

### Step-4: (S)-(2-(Benzyloxy)-4,6-dihydroxyphenyl)(8-((tetrahydrofuran-3-yl)amino)-3,4-dihydroisoquinolin-2(1H)-yl)methanone

A stirred solution of (S)-5-(benzyloxy)-4-(8-((tetrahydrofuran-3-yl)amino)-1,2,3,4-tetrahydroisoquinoline-2-carbonyl)-1,3-phenylene bis(4-methylbenzenesulfonate) (0.5 g, 0.65 mmol, 1.0 eq) in EtOH:H₂O (7:3) (15 mL) at room temperature was treated with KOH (1.46 g, 26.01 mmol, 40 eq). The reaction mixture was heated to 60° and stirred for 2 h. The reaction mixture was cooled to 0° C., acidified using saturated KHSO₄ solution and extracted using ethyl acetate (3×100 mL). The combined organic layer was dried with Na₂SO₄, filtered, and concentrated under vacuum. The crude material was purified by flash chromatography followed by Prep.TLC (5% MeOH in DCM) yielding the title compound as an off-white solid (0.035 g, Yield: 11.7%, 0.075 mmol). High temperature ¹H NMR (DMSO-d6, 400 MHz, 346K): δ 1.87 (s, 1H), 2.17-2.22 (m, 1H), 2.69-2.84 (m, 2H), 3.46-3.53 (m, 2H), 3.58-3.71 (m, 1H), 3.71-3.76 (m, 1H), 3.83-3.91 (m, 1H), 3.93-3.95 (m, 1H), 3.95-4.03 (m, 1H), 4.42-4.67 (m, br, 2H), 4.97-5.00 (m, 2H), 5.98-6.01 (m, 2H), 6.43-6.50 (m, 2H), 6.99 (t, J=8.0 Hz, 1H), 7.25-7.37 (m, 5H), 9.20 (s, 2H).

LCMS (Method A): 1.807 min, MS ES+461.17 (M+1).

Examples in Table 1 were prepared according to procedures described for Examples 1 and 2 using the stated amine and 2-(benzyloxy)-4,6-bis(tosyloxy)benzoic acid (Intermediate 61), unless otherwise stated, followed by protecting group removal.

TABLE 1

| Ex. | | | |
|---|---|---|---|
| No. | Name | Structure | Data |
| 3 | (2-(Benzyloxy)-4,6- dihydroxyphenyl)(5- (piperazin-1- ylmethyl)isoindolin- 2-yl)methanone | 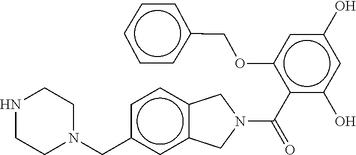 | Prepared from (9H-fluoren-9-yl)methyl 4- (isoindolin-5-ylmethyl)piperazine-1- carboxylate (Intermediate 1). ¹H NMR (DMSO-d6, D₂O exchange, 400 MHz): δ 1.96 (s, br, 4H), 4.41-4.57 (m, 4H), 4.70- 4.79 (m, br, 6H), 4.87-4.89 (m, br, 2H), 6.28 (s, br, 2H), 7.22-7.44 (m, 8H), 8.4- 8.44 (m, 2H), 8.59 (s, br 1H). LCMS (Method A): 0.904 min, MS: ES+ 460.3 (M + 1). |
| 4 | (2-(Benzyloxy)-4,6- dihydroxyphenyl)(5- ((methylamino) methyl)isoindolin-2- yl)methanone | 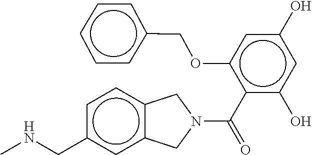 | Prepared from 1-(isoindolin-5-yl)-N- methyl methanamine (CAS 1267150-69- 5). ¹H NMR (DMSO, D₂O exchange, 400 MHz): δ 2.41 and 2.42 (2 singlets, 3H), 3.92-3.96 (m, 2H), 4.46 (s, br, 2H), 4.71 (s, br, 2H), 5.02 (s, 2H), 5.99 and 6.01 (2 singlets, 2H), 7.23-7.40 (m, 8H), 8.33 (s, 1H). LCMS (Method A): 1.093 min, MS: ES+ 405.22 (M + 1). |
| 5 | 3-((2-(2- (Benzyloxy)-4,6- dihydroxybenzoyl) isoindolin-4- yl)oxy)cyclobutane- 1-carbonitrile | 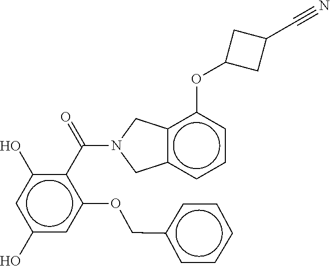 | Prepared from 3-(isoindolin-4- yloxy)cyclobutane-1-carbonitrile (Intermediate 2). ¹H NMR (DMSO-d6, 400 MHz): δ 2.74-2.82 (m, 3H), 3.45- 3.49 (m, 2H), 4.35-4.47 (m, 2H), 4.62- 4.73 (m, 2H), 4.96-5.07 (m, 3H), 6.00 (s, 2H), 6.70-6.75 (m, 1H), 6.84 (d, J = 7.2 Hz, 1H), 6.96 (d, J = 7.6 Hz, 1H), 7.21- 7.29 (m, 5H), 9.56 (s, 2H). LCMS (Method A): 2.048 min, 100%, 210 nm, MS: ES+ 457.08 (M + 1). |
| 6 | (2-(Benzyloxy)-4,6- dihydroxyphenyl)(4- ((tetrahydrofuran- 3- yl)amino)isoindolin- 2-yl)methanone | 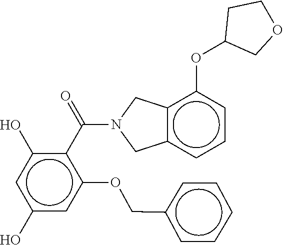 | Prepared from N-(tetrahydrofuran-3- yl)isoindolin-4-amine hydrochloride (Intermediate 3). ¹H NMR (DMSO-d6, 400 MHz): δ 1.76-1.86 (m, 1H), 2.08- 2.02 (m, 1H), 3.53-3.59 (m, 1H), 3.68- 3.79 (m, 1H), 3.85-3.87 (m, 1H), 3.92- 3.93 (m, 1H), 4.00-4.06 (m, 1H), 4.41 (d, J = 14.4 Hz, 2H), 4.66 (d, J = 30 Hz, 2H), 5.03 (s, 2H), 5.34-5.46 (m, 1H), 6.01 (s, 2H), 6.42-6.49 (m, 1H), 6.59-6.61 (m, 1H), 7.04-7.12 (m, 1H), 7.23-30 (m, 5H), 9.47-9.56 (m, 2H). LCMS (Method E): 1.779 min, MS ES+ 447.20 (M + 1). |
| 7 | (2-(Benzyloxy)-4,6- dihydroxyphenyl)(4- (oxetan-3- ylamino)isoindolin- 2-yl)methanone | 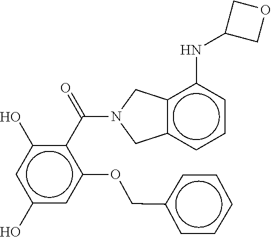 | Prepared from N-(oxetan-3-yl)isoindolin- 4-amine (Intermediate 4). ¹H NMR (DMSO-d6, 400 MHz): δ ppm 4.42 (s, 2H), 4.49-52 (m, 2H), 4.55-4.61 (m, 1H), 4.67 (d, J = 18.4 Hz, 2H), 4.82 (d, J = 26.8 Hz, 2H), 5.03-5.04 (m, 2H), 5.95-6.19 (m, 4H), 6.51-6.64 (m, 1H), 7.02-7.09 (m, 1H), 7.23-7.31 (m, 4H), 9.49-9.57 (m, 2H). LCMS (Method E): 1.791 min, MS: ES+ 433.2 (M + 1). |
| 8 | (2-(Benzyloxy)-4,6- dihydroxyphenyl)(4- (3- hydroxypiperidin-1- yl)isoindolin-2- yl)methanone | 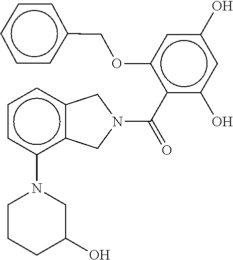 | Prepared from 1-(isoindolin-4-yl)piperidin- 3-ol hydrochloride (Intermediate 5). ¹H NMR (DMSO-d6, D₂O exchange, 400 MHz): δ 1.27-1.41 (m, 2H), 1.61 (m, 1H), 1.84-1.92 (m, 2H), 2.91 (m, 1H), 3.28 (m, 1H), 3.5-3.54 (m, 1H), 3.67 (bs, 1H), 4.48 (s, br, 2H), 4.62 (s, br, 2H), 5.04 (s, 2H), 6.04-6.05 (2 singlets, 2H), 6.84- 6.86 (m, 2H), 7.24-7.26 (m, 6H). LCMS (Method E): 1.740 min, MS: ES+ 461.3 (M + 1). |
| 9 | (2-(Benzyloxy)-4,6- dihydroxyphenyl)(4- ((4- methylpiperazin-1- yl)methyl) isoindolin- 2-yl)methanone | 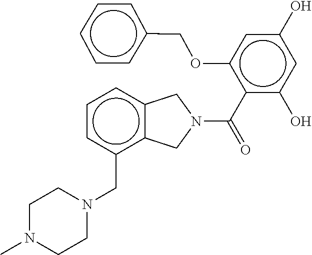 | Prepared from 4-((4-methylpiperazin-1- yl)methyl)isoindoline hydrochloride (Intermediate 6). ¹H NMR (DMSO-d6, D₂O exchange, 400 MHz): δ 2.02 and 2.10 (2 singlets, 3H), 2.20-2.29 (m, 4H), 3.25 (s, br 2H), 3.41 (s, br, 2H), 4.54-4.51 (m, 2H), 4.69-4.73 (m, 2H), 5.00 (m, 2H), 5.91-5.96 (m, 2H), 7.08-7.25 (m, 8H). LCMS (Method A): 1.255 min, MS ES+ 474.32 (M + 1). |
| 10 | (2-(Benzyloxy)-4,6- dihydroxyphenyl)(4- (morpholinomethyl) isoindolin-2- yl)methanone | 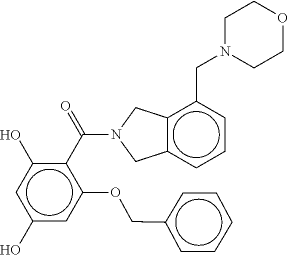 | Prepared from 4-(isoindolin-4- ylmethyl)morpholine hydrochloride (Intermediate 7). ¹H NMR (DMSO-d6, 400 MHz): δ 2.17-2.19 (m, 2H), 2.34- 2.35 (m, 2H), 3.31 3.32 (m, 1H), 3.33- 3.34 (m, 2H), 3.47 (s, 1H), 3.56 (s, 2H), 4.50 (s, 1H), 4.58 (s, 1H), 4.76 (d, J = 20.8 Hz, 2H), 5.03-5.04 (m, 2H), 6.00-6.02 (m, 2H), 7.17-7.28 (m, 8H), 9.50 9.58 (m, 2H). LCMS (Method A): 1.173 min, MS: ES+ 461.2 (M + 1). |
| 11 | (2-(Benzyloxy)-4,6- dihydroxyphenyl)(5- ((dimethylamino) methyl)isoindolin-2- yl)methanone | 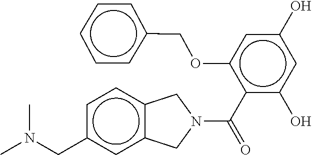 | Prepared from 1-(isoindolin-5-yl)-N,N- dimethylmethanamine (CAS 137453-31- 7). ¹H NMR (DMSO-d6, 400 MHz): δ 2.14 and 2.15 (2 singlets, 6H), 3.38-3.41 (m, 2H), 4.46 (s, 2H), 4.71 (s, 2H), 5.02 (s, 2H), 6.01 (m, 2H), 7.19-7.30 (m, 8H), 9.67 (bs, 2H). LCMS (Method A): 1.117 min, MS ES+ 419.22 (M + 1). |
| 12 | (2-(Benzyloxy)-4,6- dihydroxyphenyl)(5- (morpholinomethyl) isoindolin-2- yl)methanone | 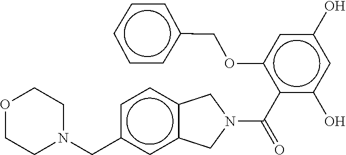 | Prepared from 4-(isoindolin-5-ylmethyl) morpholine (CAS 905905-37-5). ¹H NMR (DMSO-d6, 400 MHz): δ 2.32-2.34 (m, 4H), 3.42-3.45 (m, 2H), 3.54-3.55 (m, 4H), 4.46 (s, 2H), 4.71 (s, 2H), 5.02 (s, 2H), 6.00 (s, 2H), 7.19-7.31 (m, 8H), 9.49- 9.56 (m, 2H). LCMS (Method A): 1.118 min, MS ES+ 461.27 (M + 1). |
| 13 | (2-(Benzyloxy)-4,6- dihydroxyphenyl)(1- (hydroxymethyl) isoindolin-2- yl)methanone | 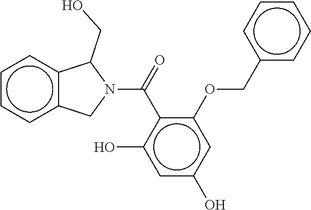 | Prepared from isoindolin-1-ylmethanol (CAS 1391249-77-6). ¹H NMR (DMSO- d6, 400 MHz): δ 3.95-3.97 (m, 1H), 4.44- 4.56 (m, 2H), 4.68-4.70 (m, 1H), 5.01 (s, 2H), 5.18-5.19 (m, 1H), 5.99-6.02 (m, 2H), 6.97-6.99 (m, 1H), 7.24-7.44 (m, 8H), 9.53-9.56 (m, 2H). LCMS (Method A): 1.559 min, MS ES+ 392.27 (M + 1). |
| 14 | (2-(Benzyloxy)-4,6- dihydroxyphenyl)(4- ((tetrahydro-2H- pyran-3- yl)amino)isoindolin- 2-yl)methanone | 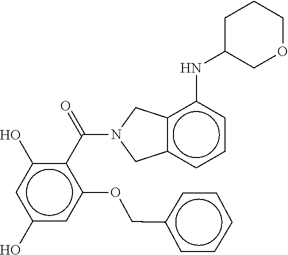 | Prepared from N-(tetrahydro-2H-pyran-3- yl)isoindolin-4-amine (Intermediate 8). ¹H NMR (DMSO-d6, 400 MHz): δ 1.50-1.57 (m, 4H), 3.01-3.26 (m, 2H), 3.72-3.87 (m, 2H), 3.86 (d, J = 22 Hz, 2H), 4.59 (d, J = 36 Hz, 2H), 4.93-5.05 (m, 3H), 6.00 (2 singlets, 2H), 6.42-6.56 (m, 2H), 7.02- 7.09 (m, 1H), 7.21-7.31 (m, 5H), 9.47- 9.56 (m, 2H). LCMS (Method A): 1.796 min, MS ES+ 461.22 (M + 1). |
| 15 | (2-(Benzyloxy)-4,6- dihydroxyphenyl)(4- ((tetrahydro-2H- pyran-4- yl)amino)isoindolin- 2-yl)methanone | 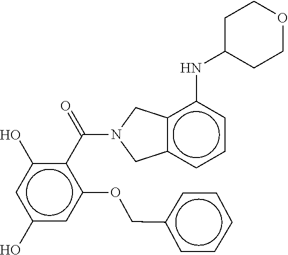 | Prepared from N-(tetrahydro-2H-pyran-4- yl)isoindolin-4-amine (Intermediate 9). ¹H NMR (DMSO-d6, 400 MHz): δ 1.42-1.52 (m, 2H), 1.78-1.89 (m, 2H), 3.37-3.53 (m, 3H), 3.82-3.90 (m, 2H), 4.41 (d, J = 16.4 Hz, 2H), 4.63 (d, J = 30 Hz, 2H), 5.03- 5.11 (m, 3H), 6.04 (m, 2H), 6.43-6.57 (m, 2H), 7.02-7.09 (m, 1H), 7.21-7.33 (m, 5H), 9.47-9.55 (m, 2H). LCMS (Method A): 1.701 min, MS: ES+ 461.1 (M + 1). |
| 16 | (2,4-Dihydroxy-6- methoxyphenyl)(4- ((tetrahydrofuran-3- yl)amino)isoindolin- 2-yl)methanone | 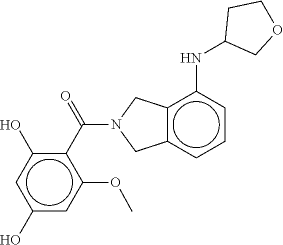 | Prepared from 2-methoxy-4,6- bis(tosyloxy)benzoic acid (Intermediate 10) and N-(tetrahydrofuran-3- yl)isoindolin-4-amine (Intermediate 3). ¹H NMR (DMSO-d6, 400 MHz): δ 1.74-1.85 (m, 1H), 2.07-2.19 (m, 1H), 3.50-3.58 (m, 1H), 3.70 (s, 3H), 3.71-3.79 (m, 1H), 3.80-4.00 (m, 2H), 3.95-4.05 (m, 1H), 4.3-4.38 (m, 2H), 4.51-4.62 (m, 2H), 5.33-5.44 (m, 1H), 5.93-6.00 (m, 2H), 6.41-6.48 (m, 1H), 6.58-6.60 (m, 1H), 7.03-7.11(m, 1H), 9.50-9.52 (m, 2H). LCMS (Method A): 1.374 min, MS: ES+ 371.27 (M + 1). |
| 17 | (2-Ethoxy-4,6- dihydroxyphenyl)(4- ((tetrahydrofuran- 3- yl)amino)isoindolin- 2-yl)methanone | 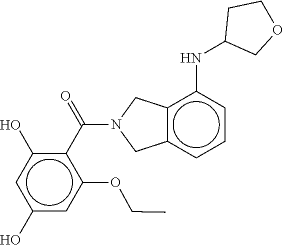 | Prepared from 2-ethoxy-4,6- bis(tosyloxy)benzoic acid (Intermediate 11) and N-(tetrahydrofuran-3- yl)isoindolin-4-amine (Intermediate 3). ¹H NMR (DMSO-d6, 400 MHz): δ 1.15-1.30 (m, 3H), 1.77-1.90 (m, 1H), 2.12-2.21 (m, 1H), 3.49-3.57 (m, 1H), 3.60-3.80 (m, 5H), 4.00-4.05 (m, 1H), 4.30-4.38 (m, 2H), 4.38-4.63 (m, 2H), 5.34, 5.44 (dd, J = 6 Hz, 5.6 Hz, 1H), 5.91-5.98 (m, 2H), 6.41-6.48 (m, 1H), 6.58-6.60 (m, 1H), 7.03-7.11(m, 1H), 9.43-9.47 (m, 2H). LCMS (Method A): 1.475 min, MS: ES+ 385.17 (M + 1). |
| 18 | 4-((2-(2- (Benzyloxy)-4,6- dihydroxybenzoyl) isoindolin-4- yl)amino)-1- methylpyrrolidin-2- one | 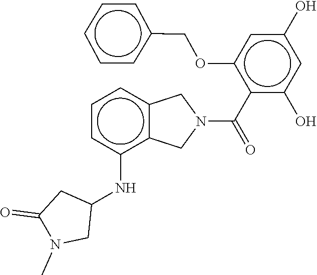 | Prepared from 4-(isoindolin-4-ylamino)-1- methylpyrrolidin-2-one hydrochloride (Intermediate 12). ¹H NMR (DMSO-d6, 400 MHz): δ 2.21-2.25 (m, 1H), 2.66- 2.75 (m, br, 4H), 3.18-3.26 (m, 1H), 3.68- 3.74 (m, 1H), 4.12-4.16 (m, 1H), 4.39- 4.43 (m, 2H), 4.59-4.68 (m, 2H), 5.02 (s, 2H), 5.32-5.47 (m, 1H), 6.03 (m, 2H), 6.42-6.64 (m, 2H), 7.07-7.10 (m, 1H), 7.23-7.31 (m, 5H), 9.46-9.56 (m, 2H). LCMS (Method A): 1.506 min, MS ES+ 474.27 (M + 1). |
| 19 | (2-(Benzyloxy)-4,6- dihydroxyphenyl)(4- (pyrimidin-5- ylamino)isoindolin- 2-yl)methanone | 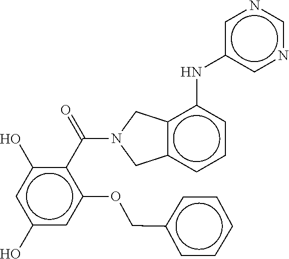 | Prepared from N-(pyrimidin-5- yl)isoindolin-4-amine hydrochloride (Intermediate 13). ¹H NMR (DMSO-d6, 400 MHz): δ 4.51 (s, br, 2H), 4.72 (d, J = 33.2 Hz, 2H), 5.03 (s, 2H), 6.01-6.02 (m, 2H), 6.90-7.01 (m, 1H), 7.15-7.23 (m, 7H), 8.01-8.21 (m, 1H), 8.50 (d, J = 22 Hz, 2H), 8.66 (d, J = 14.4 Hz, 1H), 9.5- 9.59 (m, 2H). LCMS (Method A): 1.501 min, MS: ES+ 455.1 (M + 1). |
| 20 | (2-(Benzyloxy)-4,6- dihydroxyphenyl)(4- ((oxazol-4- ylmethyl)amino) isoindolin-2- yl)methanone | 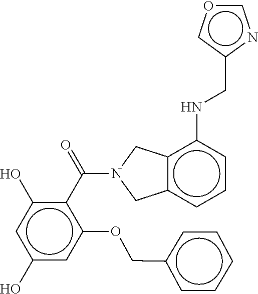 | Prepared from N-(oxazol-4- ylmethyl)isoindolin-4-amine hydrochloride (Intermediate 14). ¹H NMR (DMSO-d6, 400 MHz): δ 4.12 (d, J = 5.6 Hz, 1H), 4.22 (d, J = 6.0 Hz, 1H), 4.38 (d, J = 15.6 Hz, 2H), 4.60 (d, J = 29.9 Hz, 2H), 5.02 (s, br, 2H), 5.81-5.90 (m, 1H), 6.00-6.01 (m, 2H), 6.42-6.57 (m, 2H), 7.01 7.04 (m, 1H), 7.23-7.31 (m, 5H), 7.83-7.92 (m, 1H), 8.26-8.31 (m, 1H), 9.45-9.55 (m, 2H). LCMS (Method A): 1.648 min, MS ES+ 458.22 (M + 1). |
| 21 | (2-(Benzyloxy)-4,6- dihydroxyphenyl)(4- ((1-methyl-1H- pyrazol-4- yl)amino)isoindolin- 2-yl)methanone | 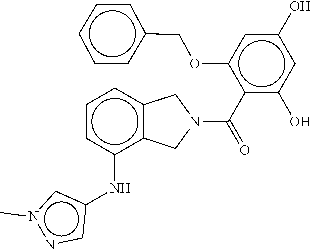 | Prepared from N-(1-methyl-1H-pyrazol-4- yl)isoindolin-4-amine hydrochloride (Intermediate 15). ¹H NMR (DMSO-d6, 400 MHz): δ 3.78 (2 singlets, 3H), 4.43 (s, 2H), 4.62-4.68 (m, 2H), 5.03 (s, 2H), 6.00- 6.01 (m, 2H), 6.52-6.69 (m, 2H), 7.04- 7.08 (m, 2H), 7.21-7.33 (m, 6H), 7.56- 7.64 (m, 1H), 9.48 (d, J = 8.4 Hz, 1H), 9.56 (d, J = 6.0 Hz, 1H), LCMS (Method A): 1.596 min, MS ES+ 457.22 (M + 1). |
| 22 | (S)-(2-(Benzyloxy)- 4,6- dihydroxyphenyl)(4- ((tetrahydrofuran- 3- yl)amino)isoindolin- 2-yl)methanone | 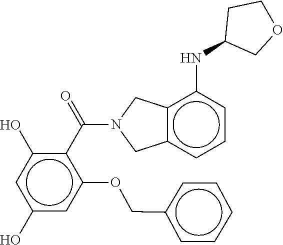 | Prepared from (S)-tetrahydrofuran-3- amine hydrochloride (Intermediate 16). ¹H NMR (DMSO-d6, 400 MHz): δ 1.75- 1.86 (m, 1H), 2.07-2.19 (m, 1H), 3.49- 3.59 (m, 1H), 3.60-3.80 (m, 2H), 3.66- 4.05 (m, 3H), 4.37-4.41 (m, 2H), 4.56- 4.65 (m, 2H), 5.02 (s, 2H), 5.34-5.43 (m, 1H), 5.99 (m, 2H), 6.42-6.48 (m, 1H), 6.59 (d, J = 7.6 Hz, 1H), 7.04-7.11 (m, 1H), 7.21-7.31 (m, 5H), 9.46-9.55 (m, 2H). LCMS (Method A): 1.691 min, MS ES+ 447.22 (M + 1). |
| 23 | (R)-(2-(Benzyloxy)- 4,6- dihydroxyphenyl)(4- ((tetrahydrofuran- 3- yl)amino)isoindolin- 2-yl)methanone | 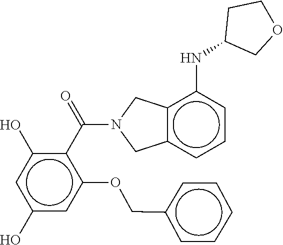 | Prepared by chiral prep. HPLC (Method- C) of (2-(benzyloxy)-4,6- dihydroxyphenyl)(4-((tetrahydro-2H- pyran-3-yl)amino)isoindolin-2- yl)methanone (Example 6). ¹H NMR (DMSO-d6, 400 MHz): δ 1.76-1.86 (m, 1H), 2.07-2.17 (m, 1H), 3.49-3.59 (m, 1H), 3.66-3.73 (m, 2H), 3.82-4.03 (m, 2H), 4.34-4.42 (m, 2H), 4.56-4.65 (m, 2H), 5.02 (s, 2H), 5.33-5.44 (m, 1H), 5.97- 5.99 (m, 2H), 6.41-6.48 (m, 2H), 6.59 (d, J = 7.2 Hz, 1H), 7.03-7.11 (m, 1H), 7.22-7.31 (m, 5H), 9.60 (bs, 2H). LCMS (Method A): 1.696 min, MS ES+ 447.20 (M + 1). |
| 24 | 4-((2-(2- (benzyloxy)-4,6- dihydroxybenzoyl) isoindolin-4- yl)amino)-5- methylpyridin- 2(1H)-one | 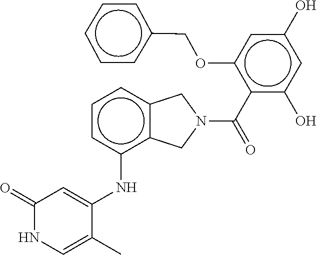 | Prepared from 4-(isoindolin-4-ylamino)-5- methylpyridin-2(1H)-one hydrochloride (Intermediate 17). High temperature ¹H NMR (DMSO-d6, 400 MHz, 349K): δ 1.92- 2.03 (m, br, 3H), 4.46 (s, br, 1H), 4.57 (d, J = 19.2 Hz, 2H), 4.80 (s, br, 1H), 5.02 (s, br 3H), 6.01 (m, br, 2H), 6.97 (d, J = 22.8 Hz, 1H), 7.08-7.10 (m, 2H), 7.23-7.41 (m, 6H), 7.51 (d, J = 8 Hz, 1H), 9.29 (bs, 2H), 10.26 (bs, 1H). LCMS (Method A): 1.346 min, MS ES+ 484.2 (M + 1). |
| 25 | (2- (Cyclopropyl- methoxy)-4,6- dihydroxyphenyl)(4- ((tetrahydrofuran- 3- yl)amino)isoindolin- 2-yl)methanone | 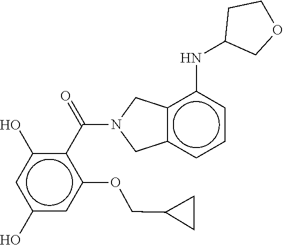 | Prepared from 2-(cyclopropylmethoxy)- 4,6-bis(tosyloxy)benzoic acid (Intermediate 18) and N- (tetrahydrofuran-3-yl)isoindolin-4-amine (Intermediate 3). High temperature ¹H NMR (DMSO-d6, 400 MHz, 340K): δ 0.20- 0.24 (m, 2H), 0.39-0.44 (m, 2H), 1.05- 1.10 (m, 1H), 1.78-1.91 (m, 1H), 2.13- 2.20 (m, 1H), 3.51-4.05 (m, 7H), 4.38- 4.44 (m, 2H), 4.59-4.67 (m, 2H), 5.13- 5.25 (m, 1H), 5.94-5.99 (m, 2H), 6.46- 6.61 (m, 2H), 7.04-7.11 (m, 1H), 9.21 (s, 2H). LCMS (Method A): 1.600 min, MS ES+ 411.22 (M + 1). |
| 26 | (2- (cyclohexyl- methoxy)-4,6- dihydroxyphenyl)(4- ((tetrahydrofuran- 3- yl)amino)isoindolin- 2-yl)methanone | 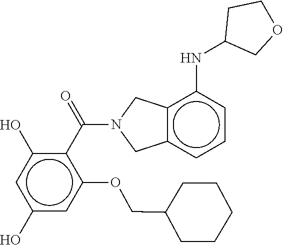 | Prepared from 2-(cyclohexylmethoxy)- 4,6-bis(tosyloxy)benzoic acid (Intermediate 19) and N- (tetrahydrofuran-3-yl)isoindolin-4-amine (Intermediate 3). High temperature ¹H NMR (DMSO-d6, 400 MHz, 345K): δ 0.88- 0.97 (m, 2H), 1.03-1.26 (m, 4H), 1.52- 1.63 (m, 6H), 1.79-1.88 (m, 1H), 2.14- 2.16 (m, 1H), 3.53-3.58 (m, 1H), 3.75- 3.78 (m, 3H), 3.85-4.06 (m, 2H), 4.35- 4.40 (m, 2H), 4.58-4.65 (m, 2H), 5.12- 5.21 (m, 1H), 5.93-5.99 (m, 2H), 6.46- 6.61 (m, 2H), 7.05-7.06 (m, 1H), 9.17 (bs, 2H). LCMS (Method A): 1.901 min, MS ES+ 453.27 (M + 1). |
| 27 | (2-(Benzyloxy)-4,6- dihydroxyphenyl)(5- ((tetrahydrofuran- 3-yl)amino)-3,4- dihydroisoquinolin- 2(1H)- yl)methanone | 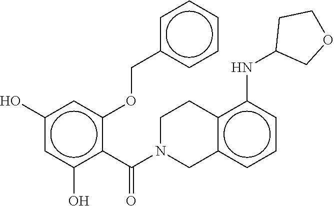 | Prepared from N-(tetrahydrofuran-3-yl)- 1,2,3,4-tetrahydroisoquinolin-5-amine hydrochloride (Intermediate 20). High temperature ¹H NMR (DMSO-d6, 400 MHz, 340K): δ 1.81-1.82 (m, 1H), 2.17- 2.21 (m, 1H), 3.54-3.55 (m, 2H), 3.71- 4.00 (m, 4H), 4.55 (bs, 3H), 4.94 (bs, 2H), 6.00 (s, 2H), 6.43 (d, J = 8 Hz, 2H), 6.99 (s, 1H), 7.22 (s, br, 5H), 9.24 (bs, 2H). LCMS (Method A): 1.690 min, MS ES+ 461.22 (M + 1). |
| 28 | (2- (Cyclohexyl- methoxy)-4,6- dihydroxyphenyl)(8- ((tetrahydrofuran- 3-yl)amino)-3,4- dihydroisoquinolin- 2(1H)- yl)methanone | 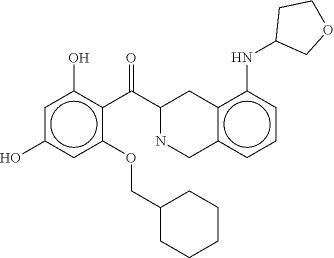 | Prepared from 2-(cyclohexylmethoxy)- 4,6-bis(tosyloxy)benzoic acid (Intermediate 19) and N- (tetrahydrofuran-3-yl)-1,2,3,4- tetrahydroisoquinolin-8-amine (Intermediate 21). ¹H NMR (DMSO-d6, 400 MHz): δ 0.85-1.27 (m, 5H), 1.46- 1.91 (m, 9H), 3.58-3.76 (m, 4H), 3.83- 4.02 (m, 3H), 4.34-4.43 (m, 1H), 4.58- 4.65 (m, 1H), 4.81 (s, br, 2H), 4.97-4.98 (m, br, 1H), 5.89-5.97 (m, 2H), 6.47 (dd, J = 7.6, 13.6 Hz, 2H), 6.99 (t, J = 8 Hz, 1H), 9.36-9.40 (m, 2H). LCMS (Method A): 2.030 min, MS ES+ 467.27 (M + 1). |
| 29 | (2-(Benzyloxy)-4,6- dihydroxyphenyl)(4- (methylamino) isoindolin-2- yl)methanone | 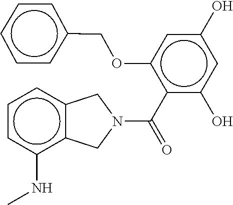 | Prepared from N-methylisoindolin-4- amine hydrochloride (CAS 1803586-30- 2). ¹H NMR (DMSO-d6, 400 MHz): δ 2.66- 2.75 (m, 3H), 4.36-4.43 (m, 2H), 4.57- 4.68 (m, 2H), 5.03 (s, 2H), 5.11-5.24 (m, 1H), 6.04 (s, 2H), 6.39-6.56 (m, 2H), 7.10 (s, 1H), 7.24-7.36 (m, 5H), 9.45-9.54 (m, 2H). LCMS (Method A): 1.704 min, MS ES+ 391.3 (M + 1). |
| 30 | (2-(Benzyloxy)-4,6- dihydroxyphenyl)(4- ((tetrahydro-2H- pyran-3- yl)amino)isoindolin- 2-yl)methanone | 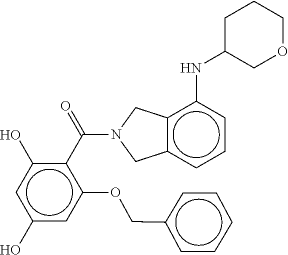 | Prepared by chiral prep. HPLC (Method- B) of (2-(benzyloxy)-4,6- dihydroxyphenyl)(4-((tetrahydro-2H- pyran-3-yl)amino)isoindolin-2- yl)methanone (Example 14); first eluting fraction. ¹H NMR (DMSO-d6, 400 MHz): δ 1.43-1.61 (m, 2H), 1.89-2.00 (m, 1H), 2.99-3.11 (m, 4H), 3.50-3.87 (m, 2H), 4.34-4.40 (m, 2H), 4.55-4.64 (m, 2H), 5.02-5.04 (m, 2H), 6.00-6.01 (m, 2H), 6.42-6.56 (m, 2H), 7.01-7.09 (m, 1H), 7.21-7.31 (m, 5H), 9.44 (bs, 2H). LCMS (Method D): 1.756 min, MS ES+ 445.2 (M + 1). |
| 31 | (2-(Benzyloxy)-4,6- dihydroxyphenyl)(4- ((tetrahydro-2H- pyran-3- yl)amino)isoindolin- 2-yl)methanone | 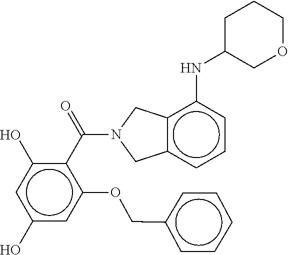 | Prepared by chiral prep. HPLC (Method- B) of (2-(benzyloxy)-4,6- dihydroxyphenyl) (4-((tetrahydro-2H- pyran-3-yl)amino)isoindolin-2- yl)methanone (Example 14); second eluting fraction. ¹H NMR (DMSO-d6, 400 MHz): δ 1.43-1.61 (m, 2H), 1.91-1.94 (m, 1H), 3.02-3.14 (m, 4H), 3.72-3.87 (m, 2H), 4.34-4.40 (m, 2H), 4.55-4.64 (m, 2H), 5.02-5.05 (m, 2H), 6.42-6.56 (m, 2H), 7.02-7.09 (m, 1H), 7.22-7.31 (m, 5H), 7.65-7.73 (m, 1H), 9.44 (bs, 2H). LCMS (Method A): 1.756 min, MS ES+ 445.2 (M + 1). |
| 32 | (2-(Benzyloxy)-4,6- dihydroxyphenyl)(4- ((5,5- difluorotetrahydro- 2H-pyran-3- yl)amino)isoindolin- 2-yl)methanone | 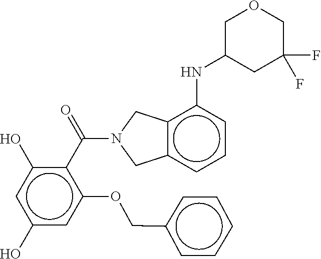 | Prepared from N-(5,5-difluorotetrahydro- 2H-pyran-3-yl)isoindolin-4-amine hydrochloride (Intermediate 22). ¹H NMR (DMSO-d6, 400 MHz): δ 2.30-2.32 (s, 3H), 3.55-3.60 (m, 1H), 3.85-3.87 (m,. 2H), 4.34-4.41 (m, 2H), 4.54-4.65 (m, 2H), 5.02 (s, 3H), 5.99-6.01 (m, 2H), 6.49- 6.62 (m, 2H), 7.07-7.11 (m, 1H), 7.23- 7.29 (m, 5H), 9.48-9.56 (m, 2H). LCMS (Method A): 1.868 min, MS ES+ 497.1 (M + 1). |
| 33 | (2-(Benzyloxy)-4,6- dihydroxyphenyl)(4- (((tetrahydrofuran- 2- yl)methyl)amino) isoindolin-2- yl)methanone | 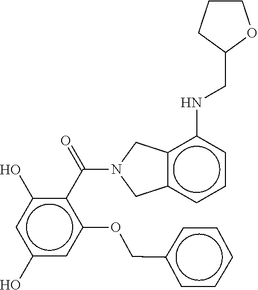 | Prepared from N-((tetrahydrofuran-2- yl)methyl)isoindolin-4-amine hydrochloride (Intermediate 23). High temperature ¹H NMR (DMSO-d6, 400 MHz, 346K): δ 1.55-1.64 (m, 2H), 1.79- 1.95 (m, 3H), 3.15-3.17 (m, 1H), 3.61- 3.79 (m, 2H), 3.95-4.03 (m, 1H), 4.36- 4.43 (m, 2H), 4.58-4.67 (m, 2H), 5.03 (s, 3H), 6.04 (s, 2H), 6.44-6.57 (m, 2H), 7.04- 7.07 (m, 1H), 7.22-7.36 (m, 5H), 9.26 (d, J = 10 Hz, 2H). LCMS (Method A): 1.804 min, MS ES+ 461.32 (M + 1). |
| 34 | (4-((1H-Pyrazol-4- yl)amino)isoindolin- 2-yl)(2-(benzyloxy)- 4,6- dihydroxyphenyl) methanone | 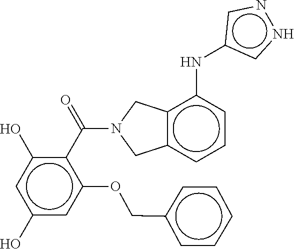 | Prepared from N-(1H-pyrazol-4- yl)isoindolin-4-amine hydrochloride (Intermediate 24). ¹H NMR (DMSO-d6, 400 MHz): δ 4.44 (d, J = 5.60 Hz, 2H), 4.61- 4.68 (m, 2H), 5.04 (d, J = 2.80 Hz, 2H), 6.00-6.02 (m, 2H), 6.52 (d, J = 7.60 Hz, 1H), 6.63-6.67 (m, 1H), 7.01-7.08 (m, 2H), 7.22-7.70 (m, 6H), 9.56 (d, J = 4.0 Hz, 1H), 12.57 (bs, 1H). LCMS (Method A): 1.490 min, MS ES+ 443.2 (M + 1). |
| 35 | (2-(Benzyloxy)-4,6- dihydroxyphenyl)(4- ((2-methyl-2H- 1,2,3-triazol-4- yl)amino)isoindolin- 2-yl)methanone | 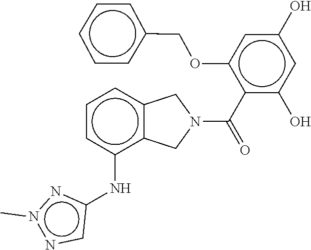 | Prepared from N-(2-methyl-2H-1,2,3- triazol-4-yl)isoindolin-4-amine hydrochloride (Intermediate 25). ¹H NMR (DMSO-d6, 400 MHz): δ 4.03 (2 singlets, 3H), 4.48 (d, J = 8.4 Hz, 2H), 4.71 (d, J = 7.6 Hz, 2H), 5.03 (s, 2H), 6.01 (s, 2H), 6.70-6.83 (dd, J = 7.2 Hz, 1H), 7.17-7.34 (m, 7H), 7.60-7.64 (m, 1H), 8.17-8.33 (m, 1H), 9.49-9.58 (m, 2H). LCMS (Method A): 1.707 min, MS ES+ 458.07 (M + 1). |
| 36 | 1-(3-((2-(2- (Benzyloxy)-4,6- dihydroxybenzoyl) isoindolin-4- yl)amino)pyrrolidin- 1-yl)ethan-1-one | 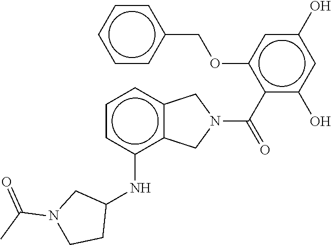 | Prepared from 1-(3-(isoindolin-4- ylamino)pyrrolidin-1-yl)ethan-1-one hydrochloride (Intermediate 26). ¹H NMR (DMSO-d6, 400 MHz): δ 1.78-1.93 (singlets, 3H), 2.32-2.49 (m, 2H), 3.40- 3.79 (m, 2H), 3.99-4.14 (m, 1H), 4.42 (d, J = 13.2 Hz, 2H), 4.60-4.68 (m, 2H), 5.02 (s, 2H), 5.35-5.51 (m, 1H), 6.04 (m, 2H), 6.50-6.55 (m, 2H), 7.05-7.13 (m, 1H), 7.24-7.31 (m, 5H), 9.46-9.56 (m, 2H). LCMS (Method D): 1.528 min, MS ES+ 488.2 (M + 1). |
| 37 | (2-((4- Fluorobenzyl)oxy)- 4,6- dihydroxyphenyl)(4- ((tetrahydrofuran- 3- yl)amino)isoindolin- 2-yl)methanone | 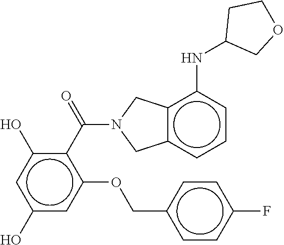 | Prepared from 2-((4-fluorobenzyl)oxy)- bis(tosyloxy)benzoic acid (Intermediate 27) and N-(tetrahydrofuran-3- yl)isoindolin-4-amine (Intermediate 3). ¹H NMR (DMSO-d6, 400 MHz): δ 1.80-1.88 (m, 1H), 2.07-2.19 (m, 1H), 3.53-3.59 (m, 1H), 3.70-3.77 (m, 2H), 3.87-3.93 (m, 2H), 4.00-4.06 (m, 1H), 4.41 (d, J = 15.2 Hz, 2H), 4.64 (d, J = 31.2 Hz, 2H), 5.01 (s, 2H), 5.32-5.44 (m, 1H), 6.05 (m, 2H), 6.42-6.48 (m, 1H), 6.60-6.61 (m, 1H), 7.03-7.07 (m, 3H), 7.34-7.37 (m, 2H), 9.47-9.56 (m, 2H). LCMS (Method A): 1.724 min, MS: ES+ 465.2 (M + 1). |
| 38 | (2-((3,4- Difluorobenzyl) oxy)-4,6- dihydroxyphenyl)(4- ((tetrahydrofuran- 3- yl)amino)isoindolin- 2-yl)methanone | 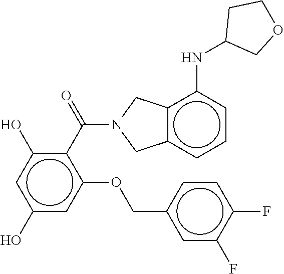 | Prepared from 2-((3,4- difluorobenzyl)oxy)-4,6- bis(tosyloxy)benzoic acid (Intermediate 28) and N-(tetrahydrofuran-3- yl)isoindolin-4-amine (Intermediate 3). ¹H NMR (DMSO-d6, 400 MHz): δ 1.78-1.88 (m, 1H), 2.11-2.18 (m, 1H), 3.45-3.59 (m, 1H), 3.67-3.78 (m, 2H), 3.85-3.96 (m, 1H), 3.98-4.05 (m, 1H), 4.30-4.40 (m, 2H), 4.60 (d, J = 21.2 Hz, 2H), 5.01- 5.10 (m, 2H), 5.30-5.45 (m, 1H), 5.98- 6.03 (m, 2H), 6.41-6.61 (m, 2H), 7.04- 7.17 (m, 2H), 7.30-7.38 (m, 2H), 9.50- 9.62 (m, 2H). LCMS (Method A): 1.744 min, MS ES+ 483.1 (M + 1). |
| 39 | (2,4-Dihydroxy-6- ((4- methoxybenzyl)oxy) phenyl)(4- ((tetrahydrofuran-3- yl)amino)isoindolin- 2-yl)methanone | 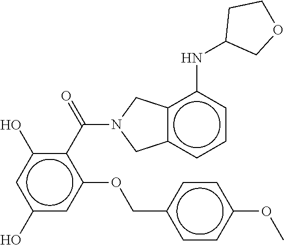 | Prepared from 2-((4-methoxybenzyl)oxy)- 4,6-bis(tosyloxy)benzoic acid (Intermediate 29) and N- (tetrahydrofuran-3-yl)isoindolin-4-amine (Intermediate 3). High temperature ¹H NMR (DMSO-d6, 400 MHz, 345K): δ ppm 1.80-1.88 (m, 1H), 2.10-2.22 (m, 2H), 3.54-3.58 (m, 1H), 3.71-3.87 (m, 4H), 3.87-4.02 (m, 1H), 4.04-4.05 (m, 1H), 4.39-4.40 (m, 2H), 4.62 (d, J = 30 Hz, 2H), 4.95 (s, 2H), 5.10-5.18 (m, 1H), 6.04 (d, J = 6.4 Hz, 2H), 6.48 (bs, 1H), 6.60 (bs, 1H), 6.80 (d, J = 8 Hz, 2H), 7.08 (bs, 1H), 7.22-7.32 (m, 2H), 9.21 (s, 2H). LCMS (Method A): 1.670 min, MS: ES+ 477.3 (M + 1). |
| 40 | (2,4-Dihydroxy-6- (pyridin-2- ylmethoxy)phenyl) (4-((tetrahydrofuran- 3- yl)amino)isoindolin- 2-yl)methanone | 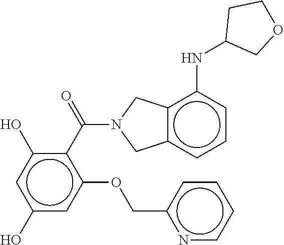 | Prepared from 2-(pyridin-2-ylmethoxy)- 4,6-bis(tosyloxy)benzoic acid (Intermediate 30) and N- (tetrahydrofuran-3-yl)isoindolin-4-amine (Intermediate 3). ¹H NMR (DMSO-d6, 400 MHz): δ 1.75-1.89 (m, 1H), 2.07- 2.33 (m, 1H), 3.48-3.60 (m, 1H), 3.64- 3.70 (m, 1H), 3.72-3.77 (m, 1H), 3.81- 3.87 (m, 1H), 3.90-3.94 (m, 1H), 3.96- 4.04 (m, 1H), 4.43 (d, J = 25.6 Hz, 2H), 4.59-4.69 (m, 2H), 5.09-5.10 (m, 2H), 5.39 (dd, J = 6 Hz, 44 Hz, 1H), 5.96-6.02 (m, 2H), 6.41-6.49 (m, 1H), 6.60-6.61 (m, 1H), 7.04-7.12 (m, 1H), 7.25-7.36 (m, 2H), 7.7 (t, J = 7.6 Hz), 8.50 (s, 1H), 9.47-9.59 (m, 2H). LCMS (Method A): 1.265 min, MS: ES+ 448.1 (M + 1). |
| 41 | (2-(Benzyloxy)-4,6- dihydroxyphenyl)(8- (oxetan-3- ylamino)-3,4- dihydroisoquinolin- 2(1H)- yl)methanone | 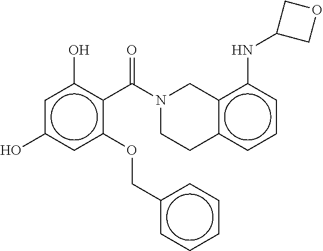 | Prepared from N-(oxetan-3-yl)-1,2,3,4- tetrahydroisoquinolin-8-amine (Intermediate 31). High temperature ¹H NMR (DMSO-d6, 400 MHz, 345K): δ 2.70 (bs, 2H), 3.52 (bs, 2H), 4.52 (s, 4H), 4.84 (s, 2H), 4.98 (s, 2H), 5.44 (bs, 1H), 6.02 (s, 2H), 6.14 (d, J = 8 Hz, 1H), 6.46 (d, J = 7.6 Hz, 1H), 6.96 (t, J = 8 Hz, 15.6 Hz, 1H), 7.26-7.27 (m, 5H), 9.21 (d, J = 11.2 Hz, 1H). LCMS (Method A): 1.713 min, MS: ES+ 447.2 (M + 1). |
| 42 | 4-((2-(2- (Benzyloxy)-4,6- dihydroxybenzoyl)- 1,2,3,4- tetrahydro- isoquinolin- 8-yl)amino)-1- methylpyrrolidin-2- one | 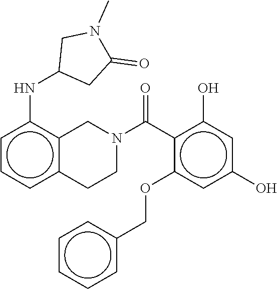 | Prepared from 1-methyl-4-((1,2,3,4- tetrahydroisoquinolin-8- yl)amino)pyrrolidin-2-one (Intermediate 32). ¹H NMR (DMSO-d6, 400 MHz): δ 2.33-2.4 (m, 1H), 2.61-2.7 (m, 3H), 2.74 (m, 3H), 3.23-3.25 (m, 1H), 3.4-3.48 (m, 2H), 3.74 (t, J = 6.8 Hz, 16.4 Hz, 1H), 4.14 (bs, 1H), 4.32-4.48 (m, 1H), 4.53-4.7 (m, 1H), 4.81-4.83 (m, 1H), 5.00 (s, 2H), 5.21-5.22 (m, 1H), 5.92-5.98 (m, 2H), 6.44 (t, J = 9.2 Hz ,18 Hz, 2H), 6.99 (t, J = 8 Hz, 15.6 Hz, 1H), 7.21-7.30 (m, 5H), 9.47 (s, 2H). LCMS (Method A): 1.622 min, MS: ES+ 488.2 (M + 1). |
| 43 | (2-(Benzyloxy)-4,6- dihydroxyphenyl)(8- (methylamino)- 3,4- dihydroisoquinolin- 2(1H)- yl)methanone | 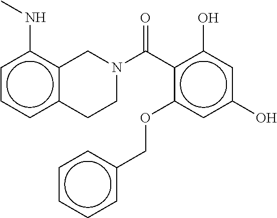 | Prepared from N-methyl-1,2,3,4- tetrahydroisoquinolin-8-amine hydrochloride (Intermediate 33). High temperature ¹H NMR (DMSO-d6, 400 MHz, 345K): δ 2.69-2.72 (m, br, 3H), 3.49 (bs, 2H), 4.36-4.38 (m, 2H), 4.97 (s, br, 3H), 6.00 (s, 2H), 6.40 (d, J = 7.6 Hz, 2H), 6.99 (t, J = 7.6 Hz, 1H), 7.25-7.26 (m, 5H), 9.21-9.24 (m, 2H). LCMS (Method A): 1.784 min, MS ES+ 405.2 (M + 1). |
| 44 | (2-(Benzyloxy)-4,6- dihydroxyphenyl)(8- ((2- methoxyethyl) amino)-3,4- dihydroisoquinolin- 2(1H)- yl)methanone | 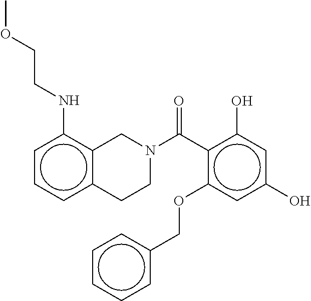 | Prepared from N-(2-methoxyethyl)- 1,2,3,4-tetrahydroisoquinolin-8-amine (Intermediate 34). High temperature ¹H NMR (DMSO-d6, 400 MHz, 345K): δ 2.67 (s, br, 2H), 3.29 (s, br, 5H), 3.53 (s, br, 4H), 4.38-4.66 (m, 2H), 4.98-5.00 (m, 2H), 5.98-6.01 (m, 2H), 6.40-6.51 (m, 2H), 6.98 (t, J = 7.6 Hz, 1H), 7.25-7.37 (m, 5H), 9.23 (d, J = 10 Hz, 2H). LCMS (Method A): 1.830 min, MS ES+ 449.17 (M + 1). |
| 45 | (2-(Benzyloxy)-4,6- dihydroxyphenyl)(4- ((1-(oxetan-3- yl)ethyl)amino) isoindolin-2- yl)methanone | 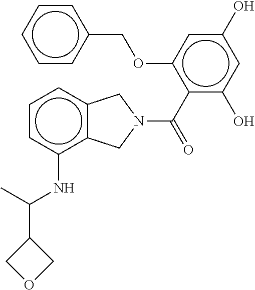 | Prepared from N-(1-(oxetan-3- yl)ethyl)isoindolin-4-amine (Intermediate 35). ¹H NMR (DMSO-d6, 400 MHz): δ 1.00-1.09 (m, 3H), 3.83-3.85 (m,1H), 4.27-4.42 (m, 4H), 4.54-4.84 (m, 5H), 5.02 (s, 2H), 6.03 (s, 2H), 6.43-6.57 (m, 2H), 7.05-7.09 (m, 1H), 7.24-7.32 (m, 5H), 9.26 (bs, 2H). LCMS (Method A): 1.729 min, MS ES+ 461.20 (M + 1). |
| 46 | (2-Benzyloxy-4,6- dihydroxy-phenyl)- [6- [(dimethylamino) methyl]-3,4- dihydro- 1H-isoquinolin-2- yl]methanone | 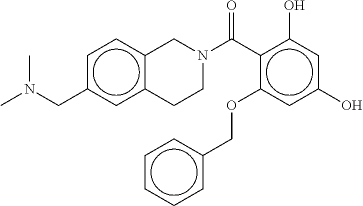 | Prepared from N,N-dimethyl-1-(1,2,3,4- tetrahydroisoquinolin-6-yl)methanamine (CAS 1107638-89-0). ¹H NMR (DMSO- d6, 400 MHz): δ ppm 2.22-2.32 (m, 6H), 2.72-2.73 (m, 2H), 3.44 (s, 1H), 2.48- 2.56 (m, 2H), 4.73 (s, 1H), 4.73-4.92 (m, 1H), 4.99 (s, 1H), 5.98 (s, 2H), 6.96-7.24 (m, 4H), 7.20-7.25 (m, 4H), 7.46 (d, J = 8 Hz, 1H), 9.47-9.49 (m, 2H). LCMS (Method A): 1.168 min, MS: ES+ 433.2 (M + 1). |
| 47 | (2-Benzyloxy-4,6- dihydroxy-phenyl)- [7- [(dimethylamino) methyl]-3,4- dihydro- 1H-isoquinolin-2- yl]methanone | 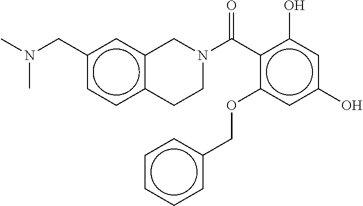 | Prepared from N,N-dimethyl-1-(1,2,3,4- tetrahydroisoquinolin-7-yl)methanamine (CAS 1362243-46-6). ¹H NMR (DMSO- d6, 400 MHz): δ ppm 2.28 (s, 2H), 2.38 (s, 6H), 2.73-2.74 (s, 2H), 3.51-3.54 (m, 2H), 3.72 (s, 2H), 4.74 (s, 1H), 4.81-4.92 (m, 1H), 5.00 (s, 1H), 5.94-5.97 (m, 2H), 7.02-7.47 (m, 8H), 9.48-9.50 (m, 2H). LCMS (Method A): 1.431 min, MS: ES+ 433.18 (M + 1). |
| 48 | (2-Benzyloxy-4,6- dihydroxy-phenyl)- (6-methoxy-3,4- dihydro-1H- isoquinolin-2- yl)methanone | 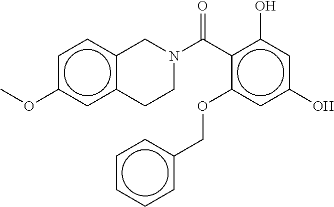 | Prepared from 6-methoxy-1,2,3,4- tetrahydroisoquinoline (CAS 42923-77-3) and. High temperature ¹H NMR (DMSO- d6, 400 MHz, 345K): δ ppm 2.04 (s, 2H), 3.54 (bs, 2H), 3.73 (s, 3H), 4.37-4.55 (m, 2H), 4.95 (s, 2H), 6.00 (s, 2H), 6.69-7.24 (m, 8H), 9.22-9.26 (s, 2H). LCMS (Method A): 1.765 min, MS: ES+ 406.17 (M + 1). |
| 49 | (2-benzyloxy-4,6- dihydroxy-phenyl)- (7-methoxy-3,4- dihydro-1H- isoquinolin-2- yl)methanone | 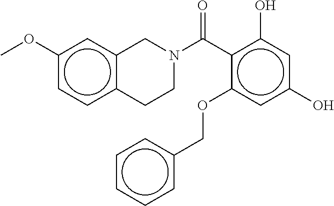 | Prepared from 7-methoxy-1,2,3,4- tetrahydroisoquinoline hydrochloride (CAS 1745-05-7). High temperature ¹H NMR (DMSO-d6, 400 MHz, 345K): δ ppm 2.69 (s, 2H), 3.45 (bs, 2H), 3.72 (s, 3H), 4.40-4.68 (m, 2H), 4.96 (s, 2H), 6.00 (s, 2H), 6.72-7.24 (m, 8H), 9.23-9.26 (s, 2H). LCMS (Method A): 1.792 min, 98.96%, 254 nm, MS: ES+ 406.22 (M + 1). |
| 50 | (2-Benzyloxy-4,6- dihydroxy-phenyl)- (4-hydroxy-3,4- dihydro-1H- isoquinolin-2- yl)methanone | 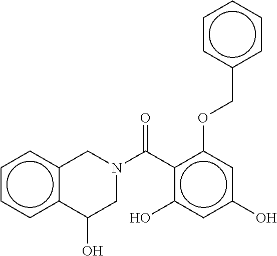 | Prepared from 1,2,3,4-tetrahydro-4- isoquinolinol (CAS 51641-23-7) followed chiral prep. HPLC (Method F) and collecting the first-eluted fraction. ¹H NMR (DMSO-d6, 400 MHz): δ ppm 2.61-2.62 (m, 4H), 3.02-3.03 (m, 1H), 4.33-4.45 (m, 2H), 4.63-4.82 (m, 2H), 4.93-5.00 (m, 2H), 5.92-6.00 (m, 2H), 5.76-5.93 (m, 2H), 6.99-7.47 (m, 9H), 9.26-s 9.30 (m, 2H). LCMS (Method A): 1.546 min, MS ES+: 392.2 (M + 1). |
| 51 | (2-Benzyloxy-4,6- dihydroxy-phenyl)- (4-hydroxy-3,4- dihydro-1H- isoquinolin-2- yl)methanone | 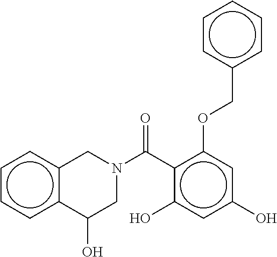 | Prepared from 1,2,3,4-tetrahydro-4- isoquinolinol (CAS 51641-23-7) followed chiral prep. HPLC (Method F) and collecting the second-eluted fraction. ¹H NMR (DMSO-d6, 400 MHz): δ ppm 2.60- 2.62 (m, 4H), 2.90-3.10 (m, 1H), 4.37- 4.42 (m, 2H), 4.91-4.92 (m, 2H), 5.66- 5.91 (m, 2H), 7.21-7.46 (m, 9H), 8.99- 9.01 (m, 2H). LCMS (Method A): 1.543 min, MS ES+: 392.2 (M + 1) |
| 52 | (2-(Benzyloxy)-4,6- dihydroxyphenyl)(4- ((1-methyl-2- oxabicyclo[2.1.1] hexan-4- yl)amino)isoindolin- 2-yl)methanone | 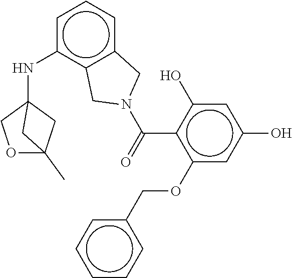 | Prepared from N-(1-methyl-2- oxabicyclo[2.1.1]hexan-4-yl)isoindolin-4- amine (Intermediate 36). ¹H NMR (DMSO-d6, 400 MHz): δ 1.34-1.38 (m, 3H), 1.75-1.88 (m, 4H), 3.58-3.66 (m, 2H), 4.37 (d, J = 20 Hz, 2H), 4.60 (d, J = 36.4 Hz, 2H), 5.02 (s, 2H), 5.93-6.04 (m, 3H), 6.47-6.60 (m, 2H), 7.02-7.08 (m, 1H), 7.23-7.35 (m, 5H), 9.46-9.55 (m, 2H). LCMS (Method A): 1.795 min, MS ES+ 473.1 (M + 1). |
| 53 | (4-((2- Oxabicyclo[2.2.1] heptan-4- yl)amino)isoindolin- 2-yl)(2-(benzyloxy)- 4,6- dihydroxyphenyl) methanone | 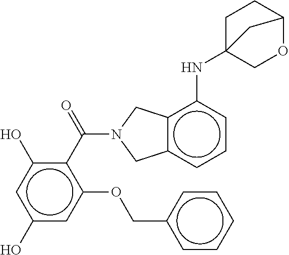 | Prepared from N-(2- oxabicyclo[2.2.1]heptan-4-yl)isoindolin-4- amine (Intermediate 37). ¹H NMR (DMSO-d6, 400 MHz): δ 1.66-1.93 (m, 6H), 3.52-3.62 (m, 1H), 3.81-3.85 (m, 1H), 4.17-4.21 (m, 1H), 4.38-4.40 (m, 2H), 4.57-4.64 (s, 2H), 5.00-5.03 (m, 2H), 5.95-6.01 (s, 2H), 6.47 (d, J = 8.0 Hz, 1H), 6.58 (d, J = 4.0 Hz, 1H), 6.64-6.68 (m, 1H), 7.01-7.06 (m, 1H), 7.23-7.36 (m, 6H), 9.46-9.55 (m, 2H). LCMS (Method A): 1.744 min, MS ES+ 473.12 (M + 1). |
| 54 | (2-(Benzyloxy)-4,6- dihydroxyphenyl)(6- methoxy-8- ((tetrahydrofuran-3- yl)amino)-3,4- dihydroisoquinolin- 2(1H)- yl)methanone | 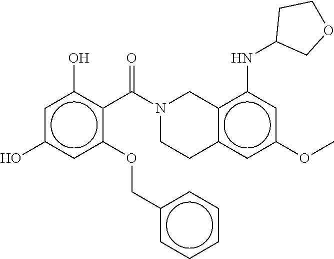 | Prepared from 6-methoxy-N- (tetrahydrofuran-3-yl)-1,2,3,4-tetrahydro- isoquinolin-8-amine (Intermediate 38). ¹H NMR (DMSO-d6, 400 MHz): δ 1.88- 1.90 (m, 1H), 2.15-2.19 (m, 1H), 2.61- 2.66 (m, 2H), 3.40-3.47 (m, 2H), 3.63- 3.75 (m, 4H), 3.81-3.99 (m, 4H), 4.30- 4.38 (m, 1H), 4.49-4.58 (m, 1H), 5.00 (m, 2H), 5.92-5.99 (m, 4H), 7.25-7.30 (m, 5H), 9.42-9.46 (m, 2H). LCMS (Method A): 1.766 min, MS ES+ 491.10 (M + 1). |
| 55 | (S)-(2,4-Dihydroxy- 6-(pyridin-2- ylmethoxy)phenyl) (8-((tetrahydrofuran- 3-yl)amino)-3,4- dihydroisoquinolin- 2(1H)- yl)methanone | 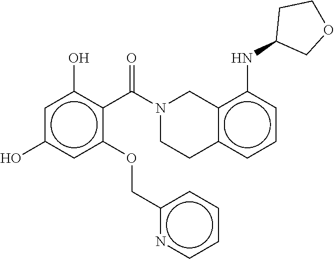 | Prepared from (S)-N-(tetrahydrofuran-3- yl)-1,2,3,4-tetrahydroisoquinolin-8-amine (Intermediate 39) and 2-(pyridin-2- ylmethoxy)-4,6-bis(tosyloxy)benzoic acid (Intermediate 30). High temperature ¹H NMR (DMSO-d6, 400 MHz, 345K): δ 1.85- 1.87 (m, 1H), 2.17-2.20 (m, 1H), 2.67- 2.71 (m, 2H), 3.49-3.52 (m, 2H), 3.58- 3.60 (m, 1H), 3.71-3.76 (m, 1H), 3.84- 3.86 (m, 1H), 3.91-3.95 (m, 1H), 4.02- 4.03 (m, 1H), 4.45-4.47 (m, 2H), 5.03 (s, 2H), 6.00 (2 singlets, 2H), 6.43-6.48 (dd, J = 8 Hz, 2H), 6.98 (t, J = 8.8 Hz, 1H), 7.25 (m, 2H), 7.64 (t, J = 7.2 Hz, 1H), 8.49 (d, J = 4.4 Hz, 1H), 9.25 (bs, 2H). LCMS (Method A): 1.339 min, MS ES+ 462.2 (M + 1). |
| 56 | (R)-(2,4-dihydroxy- 6-(pyridin-2- ylmethoxy)phenyl) (8-((tetrahydrofuran- 3-yl)amino)-3,4- dihydroisoquinolin- 2(1H)- yl)methanone | 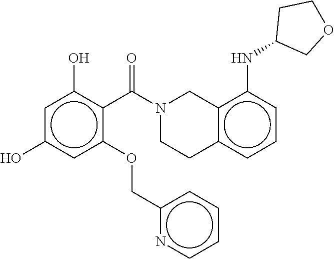 | Prepared from (R)-N-(tetrahydrofuran-3- yl)-1,2,3,4-tetrahydroisoquinolin-8-amine (Intermediate 40) and 2-(pyridin-2- ylmethoxy)-4,6-bis(tosyloxy)benzoic acid (Intermediate 30). High temperature ¹H NMR (DMSO-d6, 400 MHz, 345K): δ 1.85- 1.86 (m, 1H), 2.17-2.22 (m, 1H), 2.67- 2.71 (m, 2H), 3.45-3.59 (m, 3H), 3.71- 3.76 (m, 1H), 3.84-3.85 (m, 1H), 3.91- 3.95 (m, 1H), 4.02-4.03 (m, 1H), 4.48- 4.51 (m, 3H), 5.03 (s, 2H), 6.00 (2 singlets, 2H), 6.43-6.48 (dd, J = 8.0 Hz, 2H), 6.98 (t, J = 7.6 Hz, 1H), 7.23-7.31 (m, 2H), 7.64 (t, J = 8.0 Hz, 1H), 8.49 (d, J = 4.4 Hz, 1H), 9.25 (s, 2H). LCMS (Method A): 1.332 min, MS ES+ 462.27 (M + 1). |
| 57 | (2,4-Dihydroxy-6- (pyridin-2- ylmethoxy)phenyl) (8-(oxetan-3- ylamino)-3,4- dihydroisoquinolin- 2(1H)- yl)methanone | 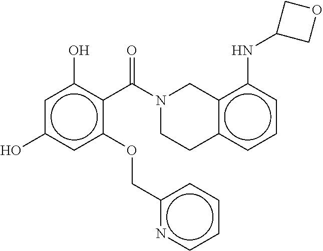 | Prepared from N-(oxetan-3-yl)-1,2,3,4- tetrahydroisoquinolin-8-amine (Intermediate 31) and 2-(pyridin-2- ylmethoxy)-4,6-bis(tosyloxy)benzoic acid (Intermediate 30). ¹H NMR (DMSO-d6, 400 MHz): δ 2.67-2.69 (m, 2H), 3.41- 3.45 (m, 2H), 4.53-4.62 (m, 2H), 4.84- 4.85 (m, 2H), 5.07 (s, 2H), 5.70 (bs, 1H), 5.97 (2 singlets, 2H), 6.11 (d, J = 8 Hz, 1H), 6.42 (d, J = 6.8 Hz, 1H), 6.93-6.97 (m, 1H), 7.25-7.31 (m, 2H), 7.63-7.64 (m, 1H), 8.50 (d, J = 4 Hz, 1H), 9.50-9.55 (m, 2H). LCMS (Method A): 1.255 min, MS ES+ 448.12 (M + 1). |
| 58 | (S)-4-((2-(2,4- Dihydroxy-6- (pyridin-2- ylmethoxy)benzoyl)- 1,2,3,4- tetrahydro- isoquinolin- 8-yl)amino)-1- methylpyrrolidin-2- one | 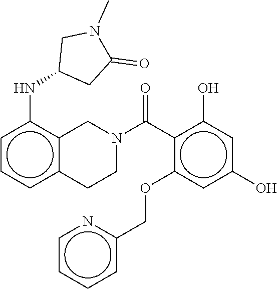 | Prepared from (S)-1-methyl-4-((1,2,3,4- tetrahydroisoquinolin-8- yl)amino)pyrrolidin-2-one (Intermediate 41) and 2-(pyridin-2-ylmethoxy)-4,6- bis(tosyloxy)benzoic acid (Intermediate 30). High temperature ¹H NMR (DMSO- d6, 400 MHz, 345K): δ 2.66-2.71 (m, 3H), 2.74 (s, 3H), 3.23-3.25 (m, 2H), 3.48- 3.49 (m, 2H), 3.72-3.76 (m, 1H), 4.15 (s, br, 1H), 4.47-4.50 (m, 2H), 5.03 (s, 2H), 5.99 (2 singlets, 2H), 6.46 (t, J = 8 Hz, 2H), 6.99 (t, J = 7.6 Hz, 1H), 7.23-7.29 (m, 2H), 7.64 (t, J = 7.6 Hz, 1H), 8.49 (d, J = 4.8 Hz, 1H), 9.26 (bs, 2H). LCMS (Method A): 1.194 min, MS ES+: 489.20 (M + 1). |
| 59 | (R)-4-((2-(2,4- dihydroxy-6- (pyridin-2- ylmethoxy)benzoyl)- 1,2,3,4- tetrahydro- isoquinolin- 8-yl)amino)-1- methylpyrrolidin-2- one | 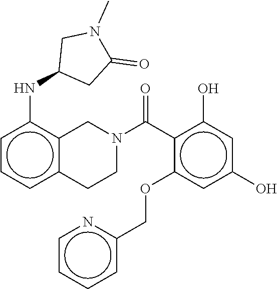 | Prepared from (R)-1-methyl-4-((1,2,3,4- tetrahydroisoquinolin-8- yl)amino)pyrrolidin-2-one (Intermediate 42) and 2-(pyridin-2-ylmethoxy)-4,6- bis(tosyloxy)benzoic acid (Intermediate 30). High temperature ¹H NMR (DMSO- d6, 400 MHz, 345K): δ 2.67-2.73 (m, 3H), 2.74 (s, 3H), 3.23-3.24 (m, 2H), 3.48- 3.49 (m, 2H), 3.72-3.76 (m, 1H), 4.15 - 4.16 (m, 1H), 4.47-4.50 (m, 2H), 5.03 (s, 2H), 5.99 (2 singlets, 2H), 6.46 (t, J = 8 Hz, 2H), 6.99 (t, J = 7.6 Hz, 1H), 7.23-7.29 (m, 2H), 7.64 (t, J = 7.6 Hz, 1H), 8.49 (d, J = 4.8 Hz, 1H), 9.26 (bs, 1H). LCMS (Method A): 1.209 min, MS ES+: 489.10 (M + 1). |
| 60 | (R)-(2-(Benzyloxy)- 4,6- dihydroxyphenyl) (6-(2- methoxyethoxy)-8- ((tetrahydrofuran-3- yl)amino)-3,4- dihydroisoquinolin- 2(1H)- yl)methanone | 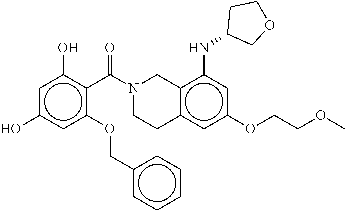 | Prepared from (R)-6-(2-methoxyethoxy)- N-(tetrahydrofuran-3-yl)-1,2,3,4- tetrahydro isoquinolin-8-amine (Intermediate 43). High temperature ¹H NMR (DMSO-d6, 400 MHz, 347K): δ 1.90- 1.93 (m, 2H), 2.15-2.25 (m, 1H), 2.62- 2.66 (m, 2H), 3.27 (s, 3H), 3.31-3.44 (m, 2H), 3.60-3.64 (m, 3H), 3.73-3.87 (m, 2H), 3.90-4.00 (m, 1H), 4.0-4.05 (m, 3H), 4.33-4.54 (m, 2H), 5.00 (s, 2H), 6.00- 6.06 (m, 3H), 7.24-7.30 (m, 4H), 9.46- 9.47 (m, 2H). LCMS (Method A): 1.689 min, MS ES+ 535.31 (M + 1). |
| 61 | 4-((2-(2- (Benzyloxy)-4,6- dihydroxybenzoyl)- 1,2,3,4- tetrahydro- isoquinolin- 8-yl)amino)-1- methylpyrrolidin-2- one | 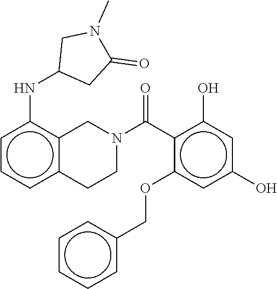 | Prepared by chiral prep. HPLC (Method- D) of (2-(benzyloxy)-4,6- dihydroxyphenyl)(4-((tetrahydro-2H- pyran-3-yl)amino)isoindolin-2- yl)methanone (Example 42); first eluting fraction. ¹H NMR (DMSO-d6, 400 MHz): δ 2.72 (s, 3H), 3.22-3.33 (m, 4H), 3.70- 3.75 (m, 1H), 4.11-4.12 (m, 1H), 4.35- 4.47 (m, 2H), 4.90-4.91 (m, 2H), 5.13- 5.14 (m, 1H), 5.75-5.83 (m, 2H), 6.41- 6.45 (m, 2H), 6.96-6.99 (m, 1H), 7.24- 7.25 (m, 5H), 9.65 (bs, 2H). (Method A): 1.616 min, MS ES+ 487.90 (M + 1). |
| 62 | 4-((2-(2- (Benzyloxy)-4,6- dihydroxybenzoyl)- 1,2,3,4- tetrahydro- isoquinolin- 8-yl)amino)-1- methylpyrrolidin-2- one | 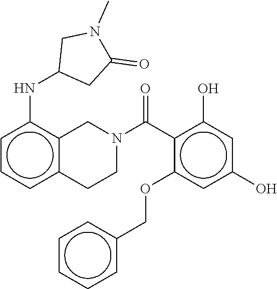 | Prepared by chiral prep. HPLC (Method- D) of (2-(benzyloxy)-4,6- dihydroxyphenyl) (4-((tetrahydro-2H- pyran-3-yl)amino)isoindolin-2- yl)methanone (Example 42); second eluting fraction. ¹H NMR (DMSO-d6, 400 MHz): δ 2.66 (s, 4H), 3.30-3.33 (bs, 6H), 4.11-4.12 (m, 1H), 4.31-4.53 (m, 2H), 4.82-4.93 (m, 2H), 5.14-5.15 (m, 1H), 5.85-5.89 (m, 2H), 6.44 (t, J = 12Hz, 2H), 6.96-7.00 (m, 1H), 7.25-7.45 (m, 5H), 7.34-7.36 (m, 1H), 9.61-9.68 (bs, 2H).LCMS (Method A): 1.609 min, MS ES+ 488.1 (M + 1). |
| 63 | (S)-(2-(Benzyloxy)- 4,6- dihydroxyphenyl)(4- ((tetrahydrofuran- 3-yl)amino)-7,8- dihydro-1,6- naphthyridin-6(5H)- yl)methanone | 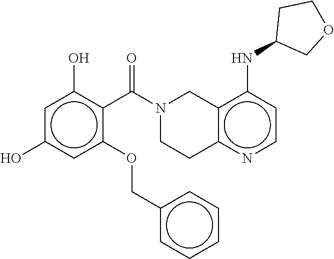 | Prepared from (S)-N-(tetrahydrofuran-3- yl)-5,6,7,8-tetrahydro-1,6-naphthyridin-4- amine (Intermediate 44). High temperature ¹H NMR (DMSO-d6, 400 MHz, 345K): δ 1.91-1.92 (m, 1H), 2.19- 2.24 (m, 1H), 2.66-2.71 (m, 2H), 3.54 (s, 2H), 3.62-3.65 (m, 1H), 3.71-3.76 (m, 1H), 3.85-3.94 (m, 2H), 4.10-4.12 (m, 1H), 4.41-4.53 (m, 2H), 4.97 (s, 2H), 5.64 (s, 1H), 6.00 (s, 2H), 6.44 (d, J = 5.6 Hz, 1H), 7.23-7.26 (m, 5H), 7.97 (d, J = 5.6 Hz, 1H), 9.21 (bs, 2H). LCMS (Method A): 1.027 min, MS ES+ 462.02 (M + 1). |
| 64 | (S)-(2-(Benzyloxy)- 4,6- dihydroxyphenyl) (6-(2- methoxyethoxy)-8- ((tetrahydrofuran-3- yl)amino)-3,4- dihydroisoquinolin- 2(1H)- yl)methanone | 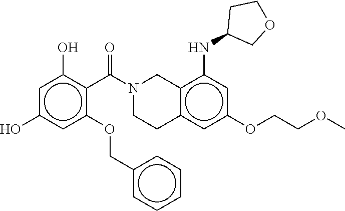 | Prepared from (S)-6-(2-methoxyethoxy)- N-(tetrahydrofuran-3-yl)-1,2,3,4- tetrahydro isoquinolin-8-amine (Intermediate 45). ¹H NMR (DMSO-d6, 400 MHz): δ 1.73-1.74 (m, 1H), 1.90- 1.98 (m, 3H), 2.15-2.19 (m, 1H), 3.28 (s, 3H), 3.60-3.62 (m, 3H), 3.61-3.65 (m, 1H), 3.83-3.85 (m, 2H), 3.90-3.99 (m, 3H), 4.33-4.54 (m, 2H), 5.00 (s, 2H), 5.98 (d, J = 7.2 Hz, 4H), 7.26-7.30 (m, 5H), 9.45 (bs, 2H). LCMS (Method A): 1.689 min, MS ES+ 535.31 (M + 1). |
| 65 | (S)-1-(3-((2-(2- (Benzyloxy)-4,6- dihydroxybenzoyl)- 1,2,3,4- tetrahydro- isoquinolin-8- yl)amino)pyrrolidin- 1-yl)ethan-1-one | 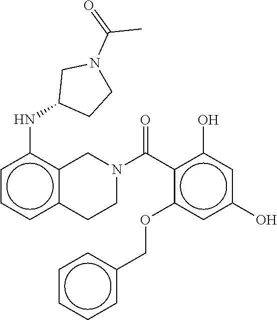 | Prepared from (S)-1-(3-((1,2,3,4- tetrahydroisoquinolin-8- yl)amino)pyrrolidin-1-yl)ethan-1-one (Intermediate 46). ¹H NMR (DMSO-d6, 400 MHz): δ 1.94 (s, 3H), 2.03-2.19 (m, 2H), 2.61-2.66 (m, 2H), 3.33-3.37 (m, 8H), 3.43-3.49 (m, 1H), 4.00-4.10 (m, 2H), 4.36-4.46 (m, 1H), 4.56-4.65 (m, 1H), 4.97-5.00 (m, 2H), 5.98 (m, 2H), 6.41 (d, J = 7.6 Hz, 1H), 6.51-6.56 (m, 1H), 7.02 (t, J = 7.2 Hz, 1H), 7.19-7.30 (m, 5H), 9.42-9.46 (m, 2H). LCMS (Method A): 1.639 min, MS ES+ 502.1 (M + 1) 523.9 (M + 23). |
| 66 | (R)-1-(3-((2-(2- (Benzyloxy)-4,6- dihydroxybenzoyl)- 1,2,3,4- tetrahydro- isoquinolin-8- yl)amino)pyrrolidin- 1-yl)ethan-1-one | 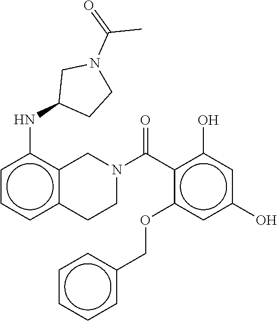 | Prepared from (R)-1-(3-((1,2,3,4- tetrahydroisoquinolin-8- yl)amino)pyrrolidin-1-yl)ethan-1-one (Intermediate 47). ¹H NMR (DMSO-d6, 400 MHz): δ 1.93 (s, 3H), 2.13-2.21 (m, 2H), 2.68-2.69 (m, 2H), 3.32-3.62 (m, 8H), 3.81-3.82 (m, 1H), 4.02-4.11 (m, 2H), 4.42-4.48 (m, 4H), 4.97 (bs, 2H), 6.00 (m, 2H), 6.44 (d, J = 7.2 Hz, 1H), 6.52- 6.58 (m, 1H), 7.00 (t, J = 7.2 Hz, 1H), 7.25- 7.26 (m,5H), 9.20-9.23 (m, 2H). LCMS (Method A): 1.642 min, MS ES+ 502 (M + 1) 523.9 (M + 23). |
| 67 | (S)-1-(3-((2-(2- (Benzyloxy)-4,6- dihydroxybenzoyl)- 1,2,3,4- tetrahydro- isoquinolin-8- yl)amino)piperidin- 1-yl)ethan-1-one | 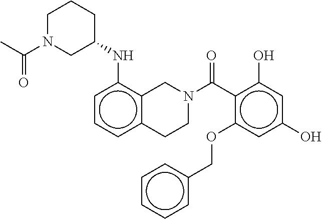 | Prepared from (S)-1-(3-((1,2,3,4- tetrahydro-isoquinolin-8- yl)amino)piperidin-1-yl)ethan-1-one (Intermediate 48). High temperature ¹H NMR (DMSO-d6, 400 MHz, 345K): δ ppm 1.58-1.71 (m, 5H), 2.27-2.32 (m, 3H), 2.50 (s, 3H), 3.47-3.48 (m, 3H), 4.41- 4.43 (m, 4H), 4.98 (s, 2H), 6.02 (s, 2H), 6.43-6.60 (m, 3H), 6.99-7.27 (m, 6H), 9.19 (bs, 2H). LCMS (Method D): 2.078 min, MS: ES+ 516.20 (M + 1). |
| 68 | (R)-1-(3-((2-(2- (benzyloxy)-4,6- dihydroxybenzoyl)- 1,2,3,4- tetrahydro- isoquinolin-8- yl)amino)piperidin- 1-yl)ethan-1-one | 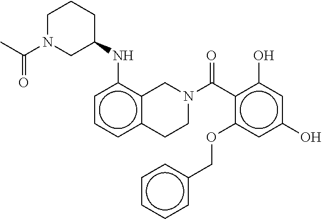 | Prepared from (R)-1-(3-((1,2,3,4- tetrahydro isoquinolin-8- yl)amino)piperidin-1-yl)ethan-1-one (Intermediate 49). High temperature ¹H NMR (DMSO-d6, 400 MHz, 345K): δ 1.4- 1.6 (m, 2H), 1.61-1.72 (m, 1H), 1.96- 1.05 (m, 5H), 2.67-2.69 (m, 3H), 3.45- 3.46 (m, 4H), 3.7-3.72 (m, 2H), 3.98- 4.00 (m, 1H), 4.41-4.42 (m, 3H), 4.98 (s, 2H), 6.01 (s, 2H), 6.42 (d, J = 7.2 Hz, 1H), 6.6 (d, J = 8 Hz, 1H), 6.7 (t, J = 7.6 Hz, 1H), 7.26-7.27 (m, 5H), 9.20-9.23 (m, 2H). LCMS (Method D): 1.986 min, MS: ES+ 517.2 (M + 1). |
| 69 | 1-(4-((2-(2- (Benzyloxy)-4,6- dihydroxybenzoyl)- 1,2,3,4- tetrahydro- isoquinolin-8- yl)amino)piperidin- 1-yl)ethan-1-one | 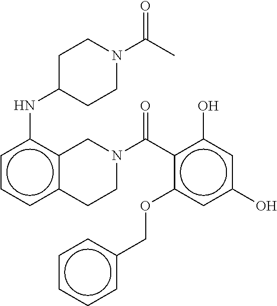 | Prepared from (1-(4-((1,2,3,4-tetrahydro- isoquinolin-8-yl)amino)piperidin-1- yl)ethan-1-one hydrochloride (Intermediate 50). High temperature ¹H NMR (DMSO-d6, 400 MHz, 345K): δ ppm 1.26-1.50 (m, 2H), 1.93-1.97 (m, 2H), 2.00 (s, 3H), 2.67-2.68 (m, 2H), 2.83- 2.84 (m, 1H), 3.49-3.53 (m, 3H), 3.72- 3.73 (m, 1H), 4.20-4.30 (m, 1H), 4.41- 4.46 (m, 3H), 4.97 (s, 2H), 6.01 (s, 2H), 6.41 (d, J = 7.2 Hz, 1H), 6.58 (d, J = 8 Hz, 1H), 6.98 (t, J = 8 Hz, 1H), 7.25-7.26 (m, 5H), 9.17-9.20 (m, 2H).LCMS (Method D): 1.687 min, MS: ES+ 516.1 (M + 1). |
| 70 | 5-((2-(2- (Benzyloxy)-4,6- dihydroxybenzoyl)- 1,2,3,4- tetrahydro- isoquinolin- 8-yl)amino)-1- methylpiperidin-2- one | 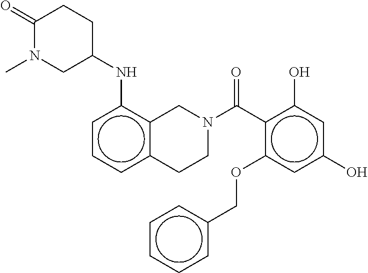 | Prepared from 1-methyl-5-((1,2,3,4- tetrahydroisoquinolin-8- yl)amino)piperidin-2-one hydrochloride (Intermediate 51). High temperature ¹H NMR (DMSO-d6, 400 MHz, 345K): δ ppm 1.81-1.82 (m, 1H), 2.00-2.04 (m, 1H), 2.31-2.35 (m, 2H), 2.66-2.69 (m, 2H), 2.81 (s, 3H), 3.24 (bs, 1H), 3.46-3.47 (m, 3H), 3.82-3.83 (m, 1H), 4.43-4.50 (m, 3H), 4.97 (s, 2H), 6.01 (t, J = 1.6 Hz, 2H), 6.44 (d, J = 7.6 Hz, 1H), 6.63 (d, J = 8.4 Hz, 1H), 6.99 (t, J = 7.6 Hz, 1H), 7.25-7.26 (m, 5H), 9.18 (bs, 2H). LCMS (Method A): 1.983 min, MS: ES+ 502.24 (M + 1). |
| 71 | (2-(Benzyloxy)-4,6- dihydroxyphenyl) (8-(pyrimidin-5- ylamino)-3,4- dihydroisoquinolin- 2(1H)- yl)methanone | 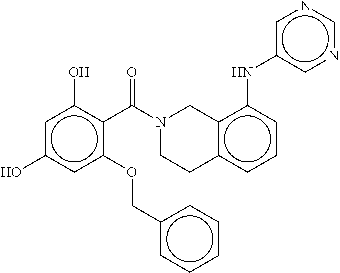 | Prepared from N-(pyrimidin-5-yl)-1,2,3,4- tetrahydroisoquinolin-8-amine (Intermediate 52). High temperature ¹H NMR (DMSO-d6, 400 MHz, 360K): δ 2.79- 2.80 (m, 2H), 3.51-3.52 (m, 2H), 4.57- 4.58 (m, 2H), 4.93 (bs, 2H), 5.98 (s, 2H), 6.92 (d, J = 7.2 Hz, 1H), 7.06 (d, J = 7.6 Hz, 1H), 7.14-7.24 (m, 6H), 7.60-7.61 (m, 1H), 8.26-8.27 (m, 2H), 9.14 (bs, 2H). LCMS (Method A): 1.553 min, MS ES+ 469.0 (M + 1). |
| 72 | (2-(Benzyloxy)-4,6- dihydroxyphenyl)(6- methoxy-8- ((tetrahydrofuran-3- yl)amino)-3,4- dihydroisoquinolin- 2(1H)- yl)methanone | 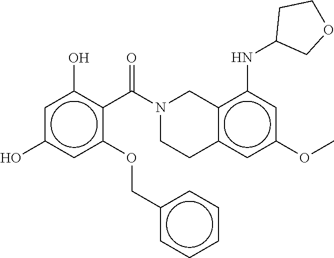 | Prepared by chiral prep. HPLC (Method- E) of (2-(benzyloxy)-4,6- dihydroxyphenyl)(4-((tetrahydro-2H- pyran-3-yl)amino)isoindolin-2- yl)methanone (Example 54); first eluting fraction. ¹H NMR (DMSO-d6, 400 MHz): δ 1.87-1.88 ((m, 1H), 2.14-2.15 (m, 1H), 2.61-2.66 (m, 1H), 3.57-3.72 (m, 5H), 3.81-3.98 (m, 4H), 4.28-4.36 (m, 1H), 4.94-4.95 (m, 2H), 5.89-5.99 (m, 4H), 7.25 (bs, 5H), 9.61 (bs, 2H). LCMS (Method A): 1.737 min, MS ES+ 491.2 (M + 1). |
| 73 | (2-(Benzyloxy)-4,6- dihydroxyphenyl) (6-methoxy-8- ((tetrahydrofuran-3- yl)amino)-3,4- dihydroisoquinolin- 2(1H)- yl)methanone | 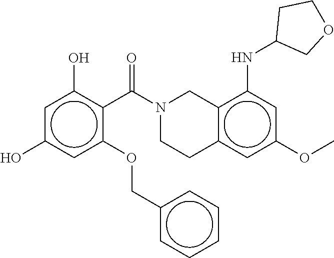 | Prepared by chiral prep. HPLC (Method- E) of (2-(benzyloxy)-4,6- dihydroxyphenyl)(4-((tetrahydro-2H- pyran-3-yl)amino)isoindolin-2- yl)methanone (Example 54); second eluting fraction. LCMS (Method A): 1.735 min, MS ES+ 491.2 (M + 1). |
| 74 | 3-((2-(2- (Benzyloxy)-4,6- dihydroxybenzoyl)- 1,2,3,4- tetrahydro- isoquinolin-8- yl)amino) benzamide | 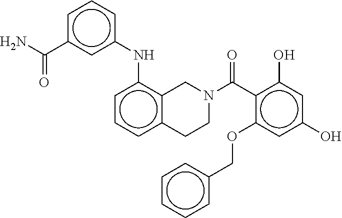 | Prepared from 3-((1,2,3,4- tetrahydroisoquinolin-8- yl)amino)benzamide hydrochloride (Intermediate 53). High temperature ¹H NMR (DMSO-d6, 400 MHz, 345K): δ ppm 2.79-2.80 (m, 2H), 3.51-3.53 (m, 2H), 4.60-4.62 (m, 2H), 4.94-4.95 (m, 4H), 5.49 (s, 2H), 5.98 (s, 2H), 6.62 (d, J = 8.4 Hz, 1H), 6.99-7.01 (m, 2H), 7.12-7.24 (m, 9H), 9.19 (bs, 2H), 9.53 (bs, 1H). LCMS (Method A): 1.479 min, MS: ES+ 510.1 (M + 1). |
| 75 | 4-((2-(2- (Benzyloxy)-4,6- dihydroxybenzoyl)- 1,2,3,4- tetrahydro- isoquinolin-8- yl)amino) benzamide | 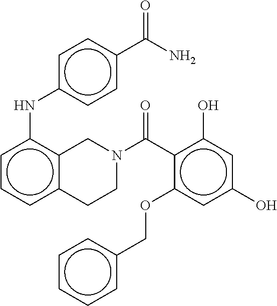 | Prepared from 4-((1,2,3,4-tetrahydro- isoquinolin-8-yl)amino)benzamide hydrochloride (Intermediate 54). High temperature ¹H NMR (DMSO-d6, 400 MHz, 345K): δ ppm 2.79-2.80 (m, 2H), 3.51-3.53 (m, 2H), 4.60-4.63 (m, 2H), 4.95-5.06 (m, 4H), 5.99 (s, 2H), 6.77 (d, J = 7.2 Hz, 1H), 7.03-7.04 (m, 2H), 7.16- 7.24 (m, 7H), 7.68 (bs, 2H), 9.19-7.28 (m, 2H). LCMS (Method A): 1.543 min, MS: ES+ 510.1 (M + 1).. |
| 76 | (2-(Benzyloxy)-4,6- dihydroxyphenyl)(8- (cyclopentylamino)- 3,4- dihydroisoquinolin- 2(1H)- yl)methanone | 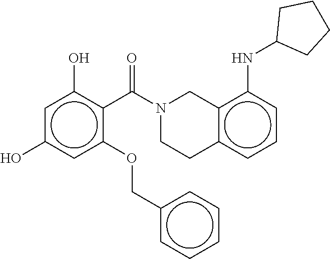 | Prepared from N-cyclopentyl-1,2,3,4- tetrahydroisoquinolin-8-amine hydrochloride (Intermediate 55). ¹H NMR (DMSO-d6, 400 MHz): δ ppm 1.50-1.54 (m, 4H), 1.69-1.70 (m, 2H), 1.92-1.94 (m, 2H), 2.60-2.72 (m, 2H), 3.42-3.50 (m, 1H), 3.64-3.72 (m, 1H), 4.36-4.41 (m, 1H), 4.56-4.65 (m, 2H), 5.00 (s, 2H), 5.93 (s, 2H), 6.34-6.49 (m, 2H), 6.95- 6.99 (m, 1H), 7.23-7.30 (m, 5H), 9.40- 9.45 (bs, 2H). LCMS (Method A): 2.142 min, MS: ES+ 459.1 (M + 1). |
| 77 | (2-(Benzyloxy)-4,6- dihydroxyphenyl)(8- ((1- methylpiperidin-4- yl)amino)-3,4- dihydroisoquinolin- 2(1H)- yl)methanone | 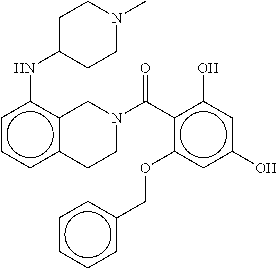 | Prepared from N-(1-methylpiperidin-4-yl)- 1,2,3,4-tetrahydroisoquinolin-8-amine hydrochloride (Intermediate 56). ¹H NMR (DMSO-d6, 400 MHz): δ ppm 1.59-1.62 (m, 2H), 1.92-1.95 (m, 2H), 2.34 (s, 3H), 2.60-2.67 (m, 3H), 2.94-2.96 (m, 2H), 3.33-3.45 (m, 2H), 4.37-4.41 (m, 1H), 4.58-4.62 (m, 2H), 5.00 (s, 2H), 5.98 (s, 2H), 6.36 (d, J = 7.6 Hz, 1H), 6.51 (d, J = 8.4 Hz, 1H), 6.95-6.99 (m, 1H), 7.20- 7.30 (m, 5H), 9.47 (bs, 2H). LCMS (Method A): 1.369 min, MS: ES+ 488.1 (M + 1). |
| 78 | (2-(Benzyloxy)-4,6- dihydroxyphenyl) (8-((1- methylpyrrolidin-3- yl)amino)-3,4- dihydroisoquinolin- 2(1H)- yl)methanone | 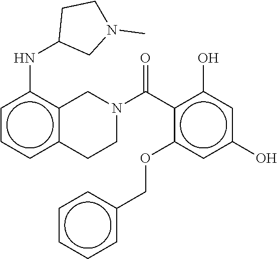 | Prepared from N-(1-methylpyrrolidin-3- yl)-1,2,3,4-tetrahydroisoquinolin-8-amine hydrochloride (Intermediate 57). ¹H NMR (DMSO-d6, 400 MHz): δ ppm 2.27 (s, 3H), 2.70-2.72 (m, 3H), 2.80-2.90 (m, 1H), 3.83-3.93 (m, 1H), 4.17-4.18 (m, 1H), 4.38-4.60 (m, 2H), 4.79 (d, J = 6 Hz, 1H), 5.00 (s, 2H), 5.98 (s, 2H), 6.37-6.43 (m, 2H), 6.96-6.99 (m, 1H), 7.25-7.29 (m, 5H), 9.58 (bs, 2H). LCMS (Method D): 1.657 min, MS: ES+ 475.2 (M + 1). |
| 112 | (2-(Benzyloxy)-4,6- dihydroxyphenyl)(4- (((tetrahydrofuran- 3- yl)methyl)amino) isoindolin-2- yl)methanone | 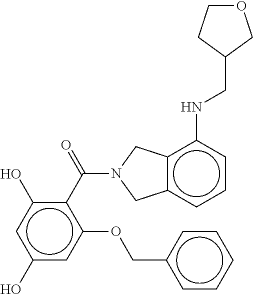 | Prepared from N-((tetrahydrofuran-3- yl)methyl)isoindolin-4-amine hydrochloride (Intermediate 74). ¹H NMR (DMSO-d6, 400 MHz): δ ppm 1.53-1.89 (m, 1H), 1.98-2.33 (m, 1H), 2.95-3.07 (m, 2H), 3.69-3.77 (m, 4H), 4.38 (d, J = 24.4 Hz, 2H), 4.60 (d, J = 41.2 Hz, 2H), 5.03 (d, J = 2.4 Hz, 2H), 5.43-5.52 (m, 1H), 6.00-6.02 (m, 2H), 6.43-6.57 (m, 2H), 7.03-7.10 (m, 1H), 7.22-7.32 (m, 5H), 9.47-9.55 (m, 2H). LCMS (Method A): 1.749 min, MS ES+: 461.07 (M + 1). Analytical HPLC (Method A): 6.484 min, 96.7%. |
| 113 | 3-((2-(2- (Benzyloxy)-4,6- dihydroxybenzoyl)- 1,2,3,4- tetrahydro- isoquinolin- 8-yl)amino)-N,N- dimethylpyrrolidine- 1-carboxamide | 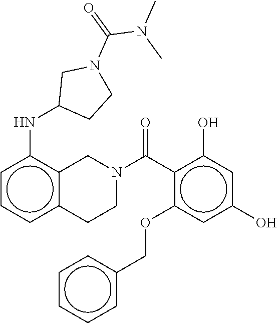 | Prepared from N,N-dimethyl-3-((1,2,3,4- tetrahydroisoquinolin-8- yl)amino)pyrrolidine-1-carboxamide hydrochloride (Intermediate 75). ¹H NMR (DMSO-d6, 400 MHz): δ ppm: 1.241 (s, 2H), 1.88-1.91 (m, 1H), 2.09-2.13 (m, 1H), 2.69 (, 1H), 2.74 (s, 6H), 3.28-3.33 (m, 2H), 3.64 (t, J = 9.6 Hz, 1H), 3.98 (m, 1H), 4.37-4.44 (m, 1H), 4.58-4.66 (m, 1H), 4.93 (d, J = 4.8 Hz, 1H), 5.01 (s, 1H), 5.99 (s, 2H), 6.41 (d, J = 7.2 Hz, 1H), 6.53 (d, J = 7.2 Hz, 1H), 7.02 (t, J= 15.6 Hz, 1H), 7.21-7.31 (m, 4H), 9.47 (s, 2H). LCMS (Method A): 1.739 min, MS ES+: 531.18 (M + 1). Analytical HPLC (Method A): 6.405 min. 100% |
| 114 | 4-((2-(2- (Benzyloxy)-4,6- dihydroxybenzoyl)- 1,2,3,4- tetrahydro- isoquinolin- 8-yl)amino)-1- methylpiperidin-2- one | 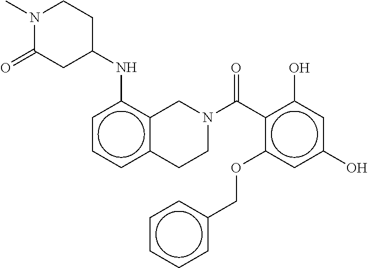 | Prepared from 1-methyl-4-((1,2,3,4- tetrahydroisoquinolin-8- yl)amino)piperidin-2-one hydrochloride (Intermediate 76). ¹H NMR (DMSO-d6, 400 MHz): δ ppm 1.26-1.29 (m, 1H), 1.75-1.76 (m, 1H), 2.10-2.11 (m, 1H), 2.61-2.68 (m, 3H), 2.74-4.78 (m, 1H), 2.84 (s, br, 3H), 3.10-3.11 (m, 2H), 3.45- 3.51 (m, 2H), 3.80-3.81 (m, 2H), 4.36- 4.47 (m, 1H), 4.58-4.68 (m, 1H), 4.84 (d, J = 5.6 Hz, 1H), 5.02 (s, 2H), 6.00 (s, 2H), 6.40 (d, J = 6.8 Hz, 1H), 6.57 (d, J = 7.6 Hz, 1H), 6.98-7.02 (m, 1H), 7.28-7.32 (m, 6H), 9.49 (d, J = 4 Hz, 2H). LCMS (Method A): 1.621 min, MS ES+: 502.13 (M + 1). Analytical HPLC (Method C): 4.976 min. 95.9% |
| 115 | (S)-(2-(Benzyloxy)- 4,6- dihydroxyphenyl)(4- ((tetrahydrofuran- 3-yl)amino)-7,8- dihydropyrido[4,3- d]pyrimidin-6(5H)- yl)methanone | 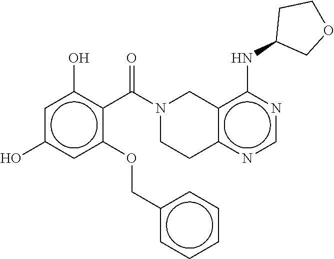 | Prepared from (S)-N-(tetrahydrofuran-3- yl)-5,6,7,8-tetrahydropyrido[4,3- d]pyrimidin-4-amine hydrochloride (Intermediate 77). ¹H NMR (DMSO-d6, 400 MHz. 353K): δ ppm 1.93-1.99 (m, 1H), 2.18-2.23 (m, 1H), 2.64 (s, 2H), 3.59- 3.60 (m, 3H), 3.72-3.77 (m, 1H), 3.86- 3.96 (m, 2H), 4.43 (s, 2H), 4.64-4.65 (m, 1H), 4.99 (s, 2H), 6.04 (d, J = 3.6 Hz, 2H), 6.66 (bs, 1H), 7.29 (s, 5H), 8.29 (s, 1H), 9.21 (d, J = 8.8 Hz, 2H). LCMS (Method A): 1.050 min, MS ES+: 463.1 (M + 1). Analytical HPLC (Method C): 3.623 min. 99.5%. |
| 116 | (2-(Benzyloxy)-4,6- dihydroxyphenyl) (7-fluoro-8- ((tetrahydrofuran-3- yl)amino)-3,4- dihydroisoquinolin- 2(1H)- yl)methanone | 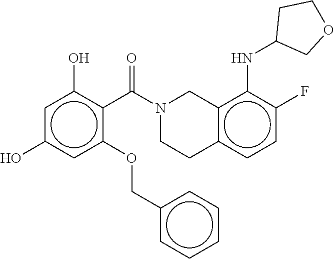 | Prepared from 7-fluoro-N- (tetrahydrofuran-3-yl)-1,2,3,4- tetrahydroisoquinolin-8-amine hydrochloride (Intermediate 78). ¹H NMR (DMSO-d6, 400 MHz, 353K): δ ppm 1.79- 1.80 (m, 2H), 2.00-2.01 (m, 2H), 2.69- 2.72 (m, 2H), 3.53-4.04 (m, 7H), 4.52- 4.54 (m, 2H), 4.97 (s, 2H), 6.03 (s, 2H), 6.64 (s, 1H), 6.90-6.95 (m, 1H), 7.26 (s, br, 5H), 9.18 (bs, 2H). LCMS (Method A): 1.784 min, MS ES+: 489.1 (M + 1). Analytical HPLC (Method A): 6.646 min. 100% |
| 117 | (2-(Benzyloxy)-4,6- dihydroxyphenyl) (8-((1-methyl-1H- pyrazol-4- yl)amino)-3,4- dihydroisoquinolin- 2(1H)- yl)methanone | 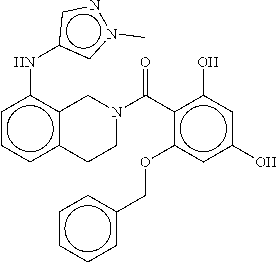 | Prepared from N-(1-methyl-1H-pyrazol-4- yl)-1,2,3,4-tetrahydroisoquinolin-8-amine hydrochloride (Intermediate 79). ¹H NMR (DMSO-d6, 400 MHz): δ ppm 2.68-2.73 (m, 2H), 3.41-3.47 (m, 2H), 3.82 (s, 3H), 4.55 (d, J = 16.8 Hz, 1H), 4.71 (d, J = 17.0 Hz, 1H), 5.02 (s, 2H), 6.01 (s, 2H), 6.42- 6.47 (m, 1H), 6.68-6.70 (m, 1H), 6.80 (s, 1H), 6.95 (t, J = 8 Hz, 2H), 7.25-7.32 (m, 5H), 9.68 (s, 2H). Formic salt. LCMS (Method A): 1.681 min, MS: ES+ 471 (M + 1). Analytical HPLC (Method A): 6.238 min. 99.5% |
| 118 | (2-(Benzyloxy)-4,6- dihydroxyphenyl) (8-(pyridin-3- ylamino)-3,4- dihydroisoquinolin- 2(1H)- yl)methanone | 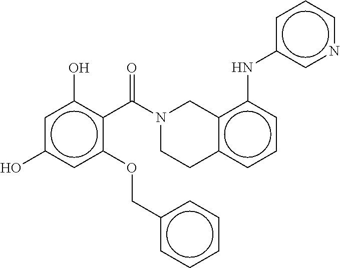 | Prepared from N-(pyridin-3-yl)-1,2,3,4- tetrahydroisoquinolin-8-amine hydrochloride (Intermediate 80). ¹H NMR (DMSO-d6, 400 MHz, 353K): δ ppm 2.69 (s, br, 2H), 3.56 (s, 2H), 4.61 (s, br, 2H), 4.96 (s, br, 2H), 6.01 (s, 2H), 6.89 (d, J = 6 Hz, 1H), 7.05 (s, 1H), 7.15 (s, br, 3H), 7.26 (s, 4H), 7.45 (s, br, 1H), 7.99 (s, 1H), 8.20 (s, 1H), 9.16 (d, J = 15.2 Hz, 2H). LCMS (Method A): 1.315 min, MS: ES+ 468.1 (M + 1). Analytical HPLC (Method: C): 5.231 min. 98.9% |
| 119 | (2-(Benzyloxy)-4,6- dihydroxyphenyl) (8-(pyrazin-2- ylamino)-3,4- dihydroisoquinolin- 2(1H)- yl)methanone | 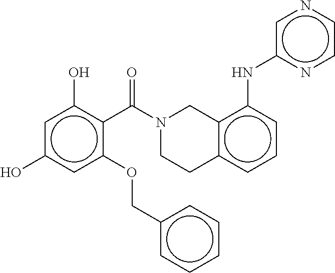 | Prepared from N-(pyrazin-2-yl)-1,2,3,4- tetrahydroisoquinolin-8-amine hydrochloride (Intermediate 81). ¹H NMR (DMSO-d6, 400 MHz, 353K): δ ppm 2.82 (s, 2H), 3.56 (s, br, 2H), 4.62 (s, br, 2H), 4.95 (s, br, 2H), 5.99 (s, 2H), 6.98 (d, J = 7.2 Hz, 1H), 7.18 (t, J = 7.6 Hz, 15.2 Hz, 1H), 7.26 (s, br, 5H), 7.86 (s, 1H), 7.96 (s, 1H), 8.13 (bs, 1H), 8.46 (s, 1H), 9.15 (d, J = 18 Hz, 2H). LCMS (Method A): 1.609 min, MS: ES+ 469.1 (M + 1). Analytical HPLC (Method: A): 5.925 min. 100% |
| 120 | (2-(benzyloxy)-4,6- dihydroxyphenyl) (8-(pyridazin-4- ylamino)-3,4- dihydroisoquinolin- 2(1H)- yl)methanone | 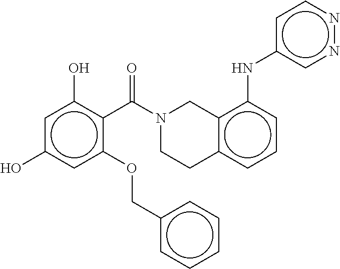 | Prepared from N-(pyridazin-4-yl)-1,2,3,4- tetrahydroisoquinolin-8-amine hydrochloride (Intermediate 82). ¹H NMR (DMSO-d6, 400 MHz): δ ppm 2.81 (s, br, 2H), 3.51-3.40 (m, 2H), 4.08-4.03 (m, 1H), 4.38-4.28 (m, 1H), 4.70-4.60 (m, 2H), 5.00 (s, 2H), 6.00 (s, 2H), 6.73-6.71 (m, 1H), 7.05-7.01 (m, 2H), 7.21-7.15 (m, 6H), 8.57-8.46 (m, 1H), 8.83-8.80 (m, 1H), 9.50-9.35 (m, 2H). LCMS (Method A): 1.282 min, MS ES+: 469.07 (M + 1). Analytical HPLC (Method A): 4.419 min. 98.7% |
| 121 | (S)-(2-(Benzyloxy)- 4,6- dihydroxyphenyl) (6-((4- methylpiperazin-1- yl)methyl)-8- ((tetrahydrofuran-3- yl)amino)-3,4- dihydroisoquinolin- 2(1H)- yl)methanone | 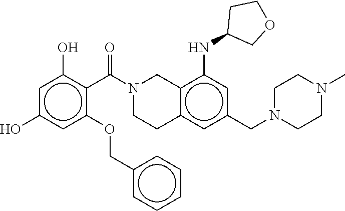 | Prepared from (S)-6-((4-methylpiperazin- 1-yl)methyl)-N-(tetrahydrofuran-3-yl)- 1,2,3,4-tetrahydroisoquinolin-8-amine hydrochloride (Intermediate 83). ¹H NMR (DMSO-d6, 400 MHz, 344K): δ ppm 1.88 (bs, 1H), 2.17 (s, 4H), 2.33-2.38 (m, 7H), 2.67 (s, 2H), 3.35-3.46 (m, 5H), 3.54- 3.60 (m, 1H), 3.72-3.77 (m, 1H), 3.84- 3.95 (m, 2H), 4.03 (s, 1H), 4.20-4.80 (m, 4H), 4.97 (s, 2H), 6.01 (s, 2H), 6.37 (s, 1H), 6.45 (s, 1H), 7.25 (s, 5H), 9.19 (s, 2H). LCMS (Method A): 1.118 min, MS ES+: 573.1 (M + 1). Analytical HPLC (Method B): 4.260 min. 99.5% |
| 122 | (R)-(2-(Benzyloxy)- 4,6- dihydroxyphenyl) (6-((4- methylpiperazin-1- yl)methyl)-8- ((tetrahydrofuran-3- yl)amino)-3,4- dihydroisoquinolin- 2(1H)- yl)methanone | 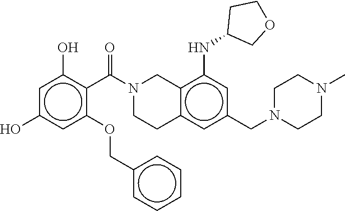 | Prepared from (R)-6-((4-methylpiperazin- 1-yl)methyl)-N-(tetrahydrofuran-3-yl)- 1,2,3,4-tetrahydroisoquinolin-8-amine hydrochloride which was, in turn, prepared using the same method as Intermediate 83 using(R)- tetrahydrofuran-3-amine (CAS:111769- 26-7). ¹H NMR (DMSO-d6, 400 MHz, 353K): δ ppm 1.89-1.92 (bs, 1H), 2.20 (s, 4H), 2.49 (s, 7H), 2.67 (s, br, 2H), 3.37 (s, 2H), 3.47 (s, br, 2H), 3.60 (s, br, 2H), 3.72- 3.77 (m, 1H), 3.84-3.86 (m, 1H), 3.92- 3.95 (m, 1H), 4.04 (s, br, 1H), 4.43 (bs, 3H), 4.97 (s, 2H), 6.02 (s, 2H), 6.38 (s, |
| | | | 1H), 6.46 (s, 1H), 7.25-7.26 (m, 5H), 9.17 |
| | | | (d, J = 16.8 Hz, 2H). LCMS (Method A): |
| | | | 1.124 min, MS ES+: 573.2 (M + 1). |
| | | | Analytical HPLC (Method B): 4.253 min |
| | | | 96.8%. |
| 123 | (2-(Benzyloxy)-4,6- dihydroxyphenyl) (8-((4- methylpyrimidin-5- yl)amino)-3,4- dihydroisoquinolin- 2(1H)- yl)methanone | 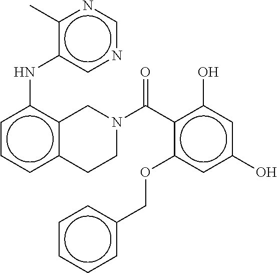 | Prepared from N-(4-methylpyrimidin-5- yl)-1,2,3,4-tetrahydroisoquinolin-8-amine hydrochloride (Intermediate 84). ¹H NMR (DMSO-d6, 400 MHz, 296K): δ ppm 1.93- 1.99 (m, 2H), 2.34 (s, 2H), 2.82 (s, 3H), 3.55-3.56 (m, 2H), 4.56-4.58 (m, 2H), 4.84-4.94 (m, 2H), 6.00 (s, 2H), 6.69 (d, J = 7.6 Hz, 1H), 6.89 (s, 1H), 7.02 (s, 1H), 7.11-7.15 (m, 2H), 7.26 (s, 6H), 7.96 (bs, 1H), 8.58 (s, 1H), 9.13-9.18 (m, 2H). LCMS (Method A): 1.527 min, MS ES+:483.18 (M + 1). Analytical HPLC (Method A): 5.327 min. 97% |
| 124 | (S)-(2,4-Dihydroxy- 6-((3- methylbenzyl)oxy) phenyl)(8- ((tetrahydrofuran-3- yl)amino)-3,4- dihydroisoquinolin- 2(1H)- yl)methanone | 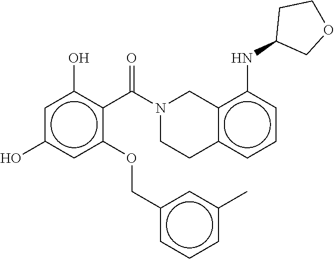 | Prepared from 2-((3-methylbenzyl)oxy)- 4,6-bis(tosyloxy)benzoic acid (Intermediate 98) and (S)-N- (tetrahydrofuran-3-yl)-1,2,3,4- tetrahydroisoquinolin-8-amine hydrochloride (Intermediate 39). ¹H NMR (DMSO-d6, 400 MHz): δ ppm 1.90-1.91 (m, 1H), 2.18-2.20 (m, 1H), 2.22 (m, 3H), 2.66-2.69 (m, 2H), 3.40-3.42 (m, 2H), 3.63-3.65 (m, 1H), 3.74-3.76 (m, 1H), 3.86-3.88 (m, 1H), 3.94-3.97 (m, 1H), 4.00-4.05 (m, 1H), 4.54-4.56 (m, 2H), 4.94-4.97 (m, 2H), 6.00 (s, 2H), 6.41- 6.49 (m, 2H), 6.98-7.17 (m, 5H), 9.45 (bs, 2H). LCMS (Method A): 1.912 min, MS ES+ 475.1 (M + 1). Analytical HPLC (Method C): 6.003 min. 97.2% |
| 125 | (S)-(2-(Benzyloxy)- 4,6- dihydroxyphenyl)(6- ((dimethylamino) methyl)-8- ((tetrahydrofuran-3- yl)amino)-3,4- dihydroisoquinolin- 2(1H)- yl)methanone | 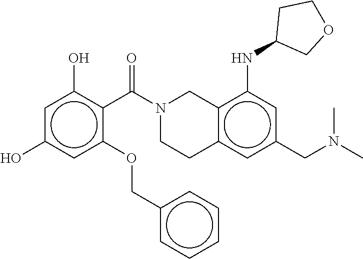 | Prepared from (S)-6- ((dimethylamino)methyl)-N- (tetrahydrofuran-3-yl)-1,2,3,4- tetrahydroisoquinolin-8-amine hydrochloride (Intermediate 85). ¹H NMR (DMSO-d6, D2O exchange, 400 MHz): δ ppm 1.90-1.92 (m, 1H), 2.15 (s, br, 6H), 2.64-2.67 (m, 2H), 3.36-3.52 (m, 6H), 3.66-4.01 (m, 4H), 4.42-4.99 (m, 2H), 4.99 (s, 2H), 5.98 and 5.99 (two singlets, 2H), 6.67-7.24 (m, 2H), 7.26-7.28 (m, 5H). LCMS (Method A): 1.186 min, MS ES+: 518.08 (M + 1). Analytical HPLC (Method A): 4.640 min. 96.7% |
| 126 | (R)-(2-(Benzyloxy)- 4,6- dihydroxyphenyl) (6- ((dimethylamino) methyl)-8- ((tetrahydrofuran-3- yl)amino)-3,4- dihydroisoquinolin- 2(1H)- yl)methanone | 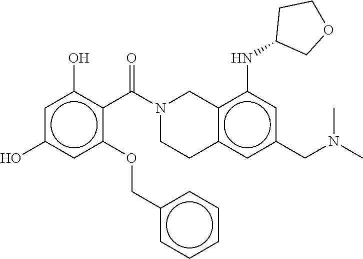 | Prepared from (R)-6- ((dimethylamino)methyl)-N- (tetrahydrofuran-3-yl)-1,2,3,4- tetrahydroisoquinolin-8-amine hydrochloride prepared from (R)- tetrahydrofuran-3-amine (CAS:111769- 26-7) using the same procedures as described for Intermediate 85. ¹H NMR (DMSO-d6, 400 MHz): δ ppm 1.91-1.93 (m, 1H), 2.18-2.29 (m, 1H), 2.36 (s, br, 6H), 2.65-2.68 (m, 3H), 3.44-3.61 (m, 2H), 3.61-3.65 (m, 2H), 3.73-3.78 (m, 1H), 3.84-3.89 (m, 1H), 3.95-4.03 (m, 2H), 4.38-4.42 (m, 1H), 4.57-4.65 (m, 1H), 5.01 (s, 2H), 5.99 (s, 2H), 6.41-6.52 (m, 2H), 7.21-7.31 (m, 5H), 9.28 (s, br, 2H). LCMS (Method A): 1.163 min, MS |
| | | | ES+: 518.13 (M + 1). Analytical HPLC |
| | | | (Method B): 4.613 min. 97.1% |
| 127 | (S)-(2-(2- (Benzyloxy)-4,6- dihydroxybenzoyl)- 8-((tetrahydrofuran- 3-yl)amino)- 1,2,3,4- tetrahydro- isoquinolin-6-yl)(4- methylpiperazin-1- yl)methanone | 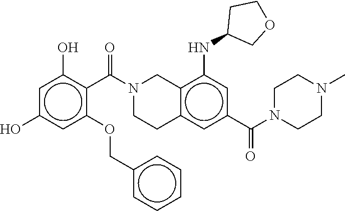 | Prepared from (S)-(4-methylpiperazin-1- yl)(8-((tetrahydrofuran-3-yl)amino)- 1,2,3,4-tetrahydroisoquinolin-6- yl)methanone hydrochloride (Intermediate 86). ¹H NMR (DMSO-d6, 400 MHz, 353K): δ ppm 1.85-1.90 (m, 1H), 2.23 (s, br, 4H), 2.33 (m, br, 4H), 2.69- 2.73 (m, 2H), 3.47-4.48 (s, br, 4H), 3.60- 3.64 (m, 2H), 3.75-3.77 (m, 2H), 3.86- 3.96 (m, 2H), 4.05-4.07 (s, 1H), 4.48- 4.50 (m, 2H), 4.99 (s, 2H), 6.04 (s, 2H), 6.45 (s, 2H), 7.29 (s, 5H), 9.18 (s, 2H). Formic acid salt. LCMS (Method A): 1.132 min, MS ES+: 587.14 (M + 1). Analytical HPLC (Method A): 4.019 min. 98.7% |
| 128 | (2-(Benzyloxy)-4,6- dihydroxyphenyl) (6-(2- methoxyethoxy)-8- (methylamino)-3,4- dihydroisoquinolin- 2(1H)- yl)methanone | 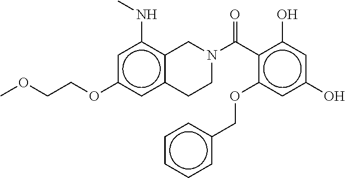 | Prepared from 6-(2-methoxyethoxy)-N- methyl-1,2,3,4-tetrahydroisoquinolin-8- amine hydrochloride (Intermediate 87). ¹H NMR (DMSO-d6, 400 MHz): δ ppm 2.64-2.71 (m, 4H), 3.30 (s, 3H), 3.62 (t, J = 4.4 Hz, 2H), 4.00-4.09 (m, 2H), 4.29- 4.50 (m, 2H), 4.87-4.89 (m, 1H), 5.01 (s, 2H), 5.25-5.24 (m, 1H), 5.94-6.03 (m, 4H), 7.24-7.30 (m, 5H), 9.56 (m, br, 2H). LCMS (Method A): 1.696 min, MS ES+: 478.8 (M + 1). Analytical HPLC (Method C): 5.078 min. 95.2% |
| 129 | (R)-2-(2- (Benzyloxy)-4,6- dihydroxybenzoyl)- N,N-dimethyl-8- ((tetrahydrofuran-3- yl)amino)-1,2,3,4- tetrahydro- isoquinoline- 6-carboxamide | 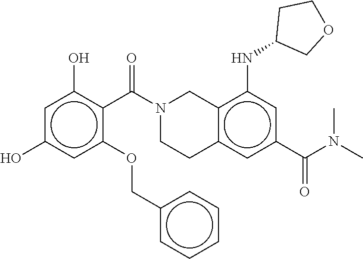 | Prepared from (R)-N,N-dimethyl-8- ((tetrahydrofuran-3-yl)amino)-1,2,3,4- tetrahydroisoquinoline-6-carboxamide hydrochloride (Intermediate 88). ¹H NMR (DMSO-d6, 400 MHz, 295K): δ ppm 1.90- 1.92 (m, 1H), 2.16-2.21 (m, 1H), 2.69- 2.71 (m, 2H), 2.92-2.94 (m, 6H), 3.64- 3.68 (m, 1H), 3.60-3.69 (m, 2H), 3.71- 3.75 (m, 2H), 4.02-4.06 (m, 1H), 4.41- 4.49 (m, 1H), 4.59-4.68 (m, 1H), 5.01 (s, 2H), 5.15-5.16 (m, 1H), 5.99 (s, 2H), 6.42 (s, 2H), 7.24-7.32 (m, 5H), 9.48-9.50 (m, 2H). LCMS (Method A): 1.475 min, MS: ES+ 532.0 (M + 1). Analytical HPLC (Method: B): 5.967 min. 97.9% |
| 130 | (S)-2-(2- (Benzyloxy)-4,6- dihydroxybenzoyl)- N,N-dimethyl-8- ((tetrahydrofuran-3- yl)amino)-1,2,3,4- tetrahydro- isoquinoline- 6-carboxamide | 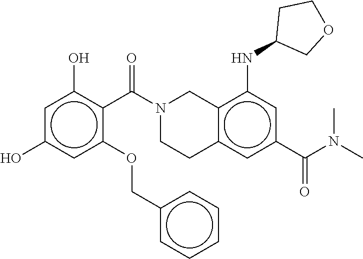 | Prepared from (S)-N,N-dimethyl-8- ((tetrahydrofuran-3-yl)amino)-1,2,3,4- tetrahydroisoquinoline-6-carboxamide hydrochloride prepared in the same manner as Intermediate 88 using (S)- tetrahydrofuran-3-amine. ¹H NMR (DMSO-d6, 400 MHz, 353K): δ ppm 1.90- 1.95 (m, 1H), 2.16-2.25 (m, 1H), 2.67- 2.72 (m, 2H), 2.94 (s, 6H), 3.44-3.49 (m, 2H), 3.62-3.63 (m, 1H), 3.72-3.77 (m, 1H), 3.85-3.87 (m, 1H), 3.91-3.95 (m, 1H), 4.04-4.06 (m, 1H), 4.45-4.49 (m, 2H), 4.82-4.84 (m, 1H), 4.98 (s, 2H), 6.02 (d, J = 2.4 Hz, 1H), 6.46 (s, 2H), 7.24-7.28 (m, 5H), 9.17-9.20 (m, 2H). LCMS (Method A): 1.473 min, MS: ES+ 531.9 (M + 1). Analytical HPLC (Method: B): 5.967 min. 99.5%. |
| 131 | (R)-(2-(Benzyloxy)- 4,6- dihydroxyphenyl) (6-(2- (dimethylamino) ethoxy)-8- ((tetrahydrofuran-3- yl)amino)-3,4- dihydroisoquinolin- 2(1H)- yl)methanone | 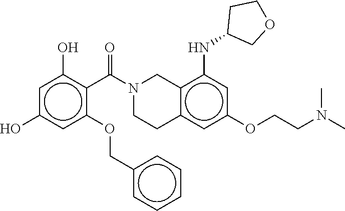 | Prepared from (R)-6-(2-(dimethylamino) ethoxy)-N-(tetrahydrofuran-3-yl)-1,2,3,4- tetrahydroisoquinolin-8-amine hydrochloride (Intermediate 89). ¹H NMR (DMSO-d6, 400 MHz): δ ppm 1.85-1.87 (m, 1H), 2.16-2.19 (m, 1H), 2.27 (s, 6H), 2.67-2.68 (m, 4H), 3.41-3.42 (m, 2H), 3.62-3.66 (m, 1H), 3.71-3.74 (m, 1H), 3.82-3.86 (m, 1H), 3.91-3.94 (m, 1H), 3.99-4.01 (m, 3H), 4.31-4.39 (m, 1H), 4.50-4.58 (m, 1H), 5.01 (s, 2H), 6.00 (d, J = 9.2 Hz, 3H), 7.12-7.49 (m, 6H), 9.46 (s, 2H). LCMS (Method A): 1.190 min, MS: ES+ 548.0 (M + 1). Analytical HPLC (Method B): 4.660 min. 98.8%. |
| 132 | (S)-(2-(Benzyloxy)- 4,6- dihydroxyphenyl) (6-(2- (dimethylamino) ethoxy)-8- ((tetrahydrofuran-3- yl)amino)-3,4- dihydroisoquinolin- 2(1H)- yl)methanone | 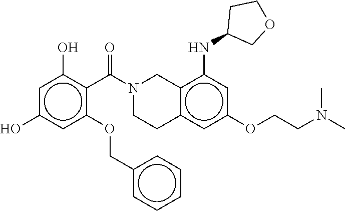 | Prepared from (S)-6-(2-(dimethylamino) ethoxy)-N-(tetrahydrofuran-3-yl)-1,2,3,4- tetrahydroisoquinolin-8-amine hydrochloride in the same manner as Intermediate 89 using (R)- tetrahydrofuran-3-amine. ¹H NMR (DMSO-d6, 400 MHz): δ ppm 1.75-1.95 (m, 1H), 1.94-1.98 (m, 1H), 2.27 (s, 6H), 2.55-2.67 (m, 4H), 3.40-3.48 (m, 2H), 3.62-3.96 (m, 4H), 3.98-4.01 (m, br, 2H), 4.34-4.98 (m, 3H), 5.01 (s, 2H), 6.00 (d, J = 9.2 Hz, 4H), 7.23-7.31 (m, 6H), 9.47 (s, 2H). LCMS (Method A): 1.201 min, MS: ES+ 548.1 (M + 1). Analytical HPLC (Method B): 4.667 min. 97.3% |
| 133 | 4-((2-(2- (Benzyloxy)-4,6- dihydroxybenzoyl)- 1,2,3,4-tetrahydro- isoquinolin-8- yl)amino)-1- methylpyridin- 2(1H)-one | 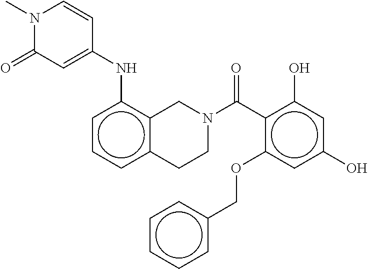 | Prepared from 1-methyl-4-((1,2,3,4- tetrahydroisoquinolin-8-yl)amino)pyridin- 2(1H)-one hydrochloride (Intermediate 90). ¹H NMR (DMSO-d6, 400 MHz, 353K): δ ppm 3.25 (s, 3H), 3.34-3.51 (m, 2H), 4.57 (m, 2H), 4.95 (s, 2H), 5.20 (s, 1H), 5.80 (s, 1H), 6.00 (s, 2H), 6.97-7.01 (m, 2H), 7.03-7.05 (m, 2H), 7.20-7.32 (m, 6H), 7.88 (m, 1H), 9.14 (m, br, 2H). LCMS (Method A): 1.456 min, MS: ES+ 497.9 (M + 1). Analytical HPLC (Method B): 5.787 min, 100% |
| 134 | 5-((2-(2- (Benzyloxy)-4,6- dihydroxybenzoyl)- 1,2,3,4- tetrahydro- isoquinolin-8- yl)amino)pyridin- 2(1H)-one | 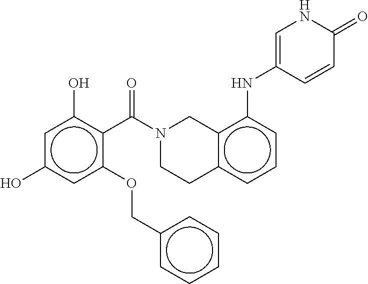 | Prepared from 5-((1,2,3,4-tetrahydroiso quinolin-8-yl)amino)pyridin-2(1H)-one hydrochloride (Intermediate 91). ¹H NMR (DMSO-d6, 400 MHz, 354K): δ ppm 2.57- 2.58 (m, 2H), 3.53 (s, br, 2H), 4.56 (s, br, 2H), 5.02 (s, 2H), 6.03 (s, 2H), 6.36-6.39 (m, 1H), 6.48-6.55 (m, 2H), 6.97 (t, J = 7.6 Hz, 1H), 7.11 (d, J = 8 Hz, 1H), 7.22- 7.36 (m, 5H), 7.47 (d, J = 8 Hz, 1H), 9.49 (d, J = 9.6 Hz, 2H). LCMS (Method A): 1.479 min, MS: ES+ 483.7 (M + 1). Analytical HPLC (Method B): 5.860 min. 96.8% |
| 135 | 5-((2-(2- (Benzyloxy)-4,6- dihydroxybenzoyl)- 1,2,3,4- tetrahydro- isoquinolin- 8-yl)amino)-1- methylpyridin- 2(1H)-one | 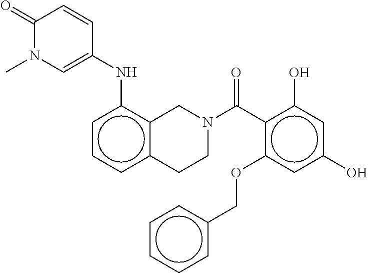 | Prepared from 1-methyl-5-((1,2,3,4- tetrahydroisoquinolin-8-yl)amino)pyridin- 2(1H)-one hydrochloride (Intermediate 92). ¹H NMR (DMSO-d6, 400 MHz, 353K): δ ppm 2.67-2.75 (m, 2H), 3.40 (s, 3H), 3.50-3.54 (m, 2H), 4.50-4.56 (m, 2H), 4.98 (s, 2H), 6.02 (s, 2H), 6.38 (d, J = 9.6 Hz, 1H), 6.54-6.60 (m, 3H), 6.98 (t, J = 7.6 Hz, 1H), 7.20-7.27 (m, 7H), 9.17- 9.20 (m, 2H). LCMS (Method A): 1.547 min, MS ES+: 497.8 (M + 1). Analytical HPLC (Method B): 6.187 min. 98.1% |
| 136 | (R)-4-((2-(2- (Benzyloxy)-4,6- dihydroxybenzoyl) isoindolin-4- yl)amino)-1- methylpyrrolidin-2- one | 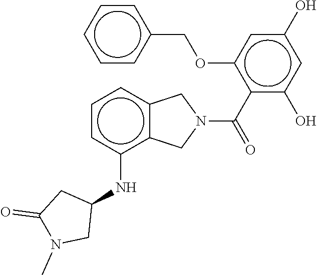 | Prepared from (R)-4-(isoindolin-4- ylamino)-1-methylpyrrolidin-2-one hydrochloride (Intermediate 93). ¹H NMR (DMSO-d6, 400 MHz, 354K): δ ppm 2.47- 2.50 (m, 1H), 2.67-2.75 (m, 3H), 3.16- 3.25 (m, 1H), 3.71-3.74 (m, 1H), 4.14- 4.15 (m, 1H), 4.43 (d, J = 16.4 Hz, 2H), 4.65 (d, J = 33.2 Hz, 2H), 5.04 (s, 2H), 5.36 (d, J = 52.4 Hz, 1H), 6.04-6.60 (m, 2H), 6.04-6.06 (m, 2H), 6.64 (bs, 1H), 7.10 (bs, 1H), 7.21-7.29 (m, 3H), 7.31-7.33 (m, 2H), 9.21 (s, 2H). LCMS (Method A): 1.505 min, MS: ES+474 (M + 1). Analytical HPLC (Method C): 4.465 min. 95% |
| 137 | (S)-4-((2-(2- (Benzyloxy)-4,6- dihydroxybenzoyl) isoindolin-4- yl)amino)-1- methylpyrrolidin-2- one | 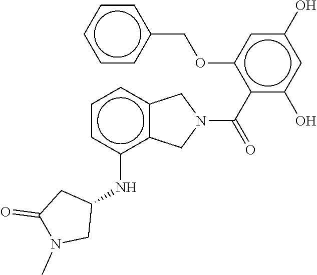 | Prepared from (S)-4-(isoindolin-4- ylamino)-1-methylpyrrolidin-2-one hydrochloride (Intermediate 94). ¹H NMR (DMSO-d6, 400 MHz, 353K): δ ppm 2.27- 2.33 (m, 1H), 2.67-2.75 (m, 4H), 3.18 (bs, 1H) 3.46-3.52 (m, 1H), 3.69-3.71 (m, 1H), 4.14-4.15 (m, 1H), 4.42 (d, J = 17.2 Hz, 2H), 4.65 (d, J = 34 Hz 2H), 5.04 (s, 2H), 6.04-6.05 (m, 2H), 6.47-6.65 (m, 2H), 7.09 (bs, 1H), 7.23-7.33 (m, 5H), 9.23 (s, 2H). LCMS (Method A): 1.505 min, MS ES+: 474.02 (M + 1). |
| 138 | (R)-(2-(Benzyloxy)- 4,6- dihydroxyphenyl) (6-((1- methylpiperidin-4- yl)oxy)-8- ((tetrahydrofuran-3- yl)amino)-3,4- dihydroisoquinolin- 2(1H)- yl)methanone | 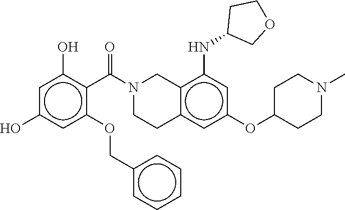 | Prepared from (R)-6-((1-methylpiperidin- 4-yl)oxy)-N-(tetrahydrofuran-3-yl)-1,2,3,4- tetrahydroisoquinolin-8-amine hydrochloride (Intermediate 95). ¹H NMR (DMSO-d6, 400 MHz): δ ppm 1.65-1.66 (m, 2H), 1.80-1.91 (m, 3H), 2.17-2.18 (m, 1H), 2.30-2.49 (m, 4H), 2.56-2.74 (m, 5H), 3.40-3.50 (m, 2H), 3.61-3.74 (m, 2H), 3.82-3.92 (m, 2H), 3.94-4.05 (m, 1H), 4.32-4.40 (m, 2H), 4.48-4.52 (m, 1H), 4.98-5.00 (m, 2H), 5.99-6.02 (m, 4H), 7.23-7.30 (m, 5H), 9.47 (s, 2H). LCMS (Method A): 1.244 min, MS ES+: 574.09 (M + 1). Analytical HPLC (Method B): 4.780 min. 100%. |
| 139 | (S)-(2-(Benzyloxy)- 4,6- dihydroxyphenyl) (6-((1- methylpiperidin-4- yl)oxy)-8- ((tetrahydrofuran-3- yl)amino)-3,4- dihydroisoquinolin- 2(1H)- yl)methanone | 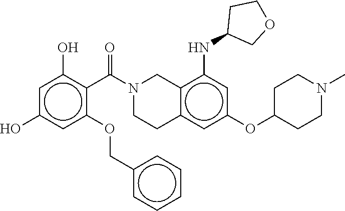 | Prepared from (S)-6-((1-methylpiperidin- 4-yl)oxy)-N-(tetrahydrofuran-3-yl)-1,2,3,4- tetrahydroisoquinolin-8-amine hydrochloride (Intermediate 96). ¹H NMR (DMSO-d6, 400 MHz): δ ppm 1.52-1.66 (m, 2H), 1.82-1.91 (m, 3H), 2.29-2.33 (m, 4H), 2.60-2.63 (m, 6H), 3.61-3.76 (m, 2H), 3.80-4.01 (m, 3H), 4.31-4.39 (m, 2H), 4.48-4.52 (m, 2H), 4.98-5.00 (m, 2H), 5.99-6.02 (m, 4H), 7.23-7.30 (m, 5H), 9.47 (s, 2H). LCMS (Method A): 1.213 min, MS: ES+ 574.1 (M + 1). Analytical HPLC (Method B): 4.747 min. 100% |
| 140 | (S)-(2- (Cyclohexyl- methoxy)-4,6- dihydroxyphenyl)(8- ((tetrahydrofuran- 3-yl)amino)-3,4- dihydroisoquinolin- 2(1H)- yl)methanone | 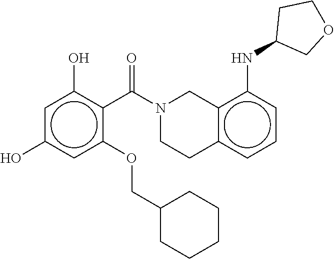 | Prepared from Intermediate 19 and (S)- N-(tetrahydrofuran-3-yl)-1,2,3,4- tetrahydroisoquinolin-8-amine hydrochloride (Intermediate 39). ¹H NMR (DMSO-d6, 400 MHz, 353K): δ ppm 0.90- 0.93 (m, 2H), 0.95-1.10 (m, 2H), 1.28 (s, 1H), 1.55-1.58 (m, 6H), 1.81-1.87 (m, 3H), 2.20-2.21 (m, 1H), 2.72 (s, 2H), 3.40-3.42 (m, 1H), 3.62 (s, br 3H), 3.73- 3.76 (m, 1H), 3.85 (s, 1H), 3.94-3.96 (m, 1H), 4.00-4.05 (m, 1H), 4.42-4.49 (m, 1H), 5.92-5.99 (m, 2H), 6.46-6.52 (m, 2H), 7.01 (t, J = 7.6 Hz, 1H), 9.06-0.12 (bs, 2H). LCMS (Method A): 2.045 min, MS ES+ 467.2 (M + 1). Analytical HPLC (Method A): 7.599 min. 97% |
| 141 | (R)-(2- (Cyclopropyl- methoxy)-4,6- dihydroxyphenyl) (6-(2- methoxyethoxy)-8- ((tetrahydrofuran-3- yl)amino)-3,4- dihydroisoquinolin- 2(1H)- yl)methanone | 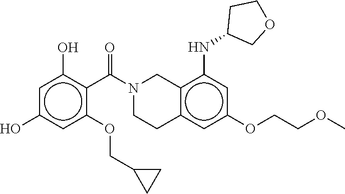 | Prepared from Intermediate 18 and (R)- 6-(2-methoxyethoxy)-N-(tetrahydrofuran- 3-yl)-1,2,3,4-tetrahydroisoquinolin-8- amine hydrochloride (Intermediate 43). ¹H NMR (DMSO-d6, 400 MHz): δ ppm 0.23 (s, br, 2H), 0.44-0.47 (m, 2H), 1.05- 1.06 (m, 1H), 1.89-1.91 (m, 1H), 2.16- 2.21 (m, 1H), 2.64 (s, br, 2H), 3.29 (s, 3H), 3.60-3.67 (m, 4H), 3.69-3.76 (m, 2H), 3.85-4.01 (m, 5H), 4.34-4.46 (m, 2H), 4.99 (d, J = 5.2 Hz, 1H), 5.89-6.01 (m, 4H), 9.31-9.41 (m, 2H). LCMS (Method A): 1.602 min, MS ES+: 499.13 (M + 1). Analytical HPLC (Method B): 6.473 min. 99% |
| 142 | (R)-(2,4-dihydroxy- 6-(pyridin-2- ylmethoxy)phenyl) (6-(2- methoxyethoxy)-8- ((tetrahydrofuran-3- yl)amino)-3,4- dihydroisoquinolin- 2(1H)- yl)methanone | 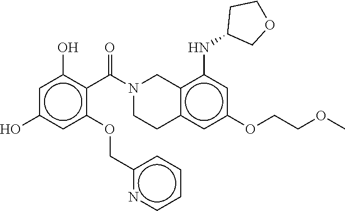 | Prepared from Intermediate 30 and (R)- 6-(2-methoxyethoxy)-N-(tetrahydrofuran- 3-yl)-1,2,3,4-tetrahydroisoquinolin-8- amine hydrochloride (Intermediate 43). ¹H NMR (DMSO-d6, 400 MHz): δ ppm 1.91-1.92 (m, 1H), 2.12-2.33 (m, 1H), 2.65-2.68 (m, 3H), 3.29 (s, 3H), 3.60- 3.66 (m, 4H), 3.73-3.74 (m, 1H), 3.91- 4.00 (m, 5H), 4.43-4.53 (m, 2H), 4.99- 5.07 (m, 2H), 5.95-5.99 (m, 4H), 7.26- 7.33 (m, 2H), 7.65-7.69 (m, 1H), 8.51 (s, 1H), 9.58 (bs, 2H). LCMS (Method A): 1.250 min, MS ES+ 536.1 (M + 1). Analytical HPLC (Method A): 4.680 min. 96.3% |
| 143 | (2-(Benzyloxy)-4,6- dihydroxyphenyl)(8- (methyl (tetrahydrofuran- 3-yl)amino)- 3,4- dihydroisoquinolin- 2(1H)- yl)methanone | 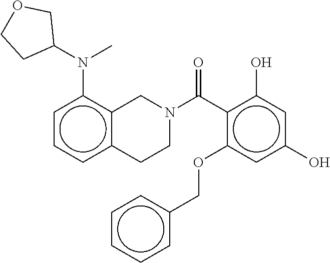 | Prepared from N-methyl-N- (tetrahydrofuran-3-yl)-1,2,3,4- tetrahydroisoquinolin-8-amine hydrochloride (Intermediate 97). ¹H NMR (DMSO-d6, 400 MHz): δ ppm 1.75-1.77 (m, 1H), 1.98-2.00 (m, 1H), 2.44-2.50 (m, 2H), 2.50-2.54 (s, br, 3H), 2.67-2.74 (m, 2H), 2.74-2.86 (m, 2H), 3.61-3.67 (m, 2H), 3.71-3.82 (m, 1H), 4.00-4.08 (m, 1H), 4.28-4.32 (m, 1H), 4.55-4.59 (m, 1H), 4.66-4.85 (m, 2H), 5.00 (bs, 1H), 5.94-5.99 (m, 2H), 6.96 (bs, 2H), 7.02- 7.07 (m, 1H), 7.15-7.22 (m, 5H), 9.38- 9.48 (m, 2H). LCMS (Method A): 1.800 min, MS: ES+ 475.28 (M + 1). Analytical HPLC (Method B): 7.17 min. 100% |

### Example 79 (2-(Benzyloxy)-4,6-dihydroxyphenyl) (4-((methylamino)methyl) isoindolin-2-yl) methanone

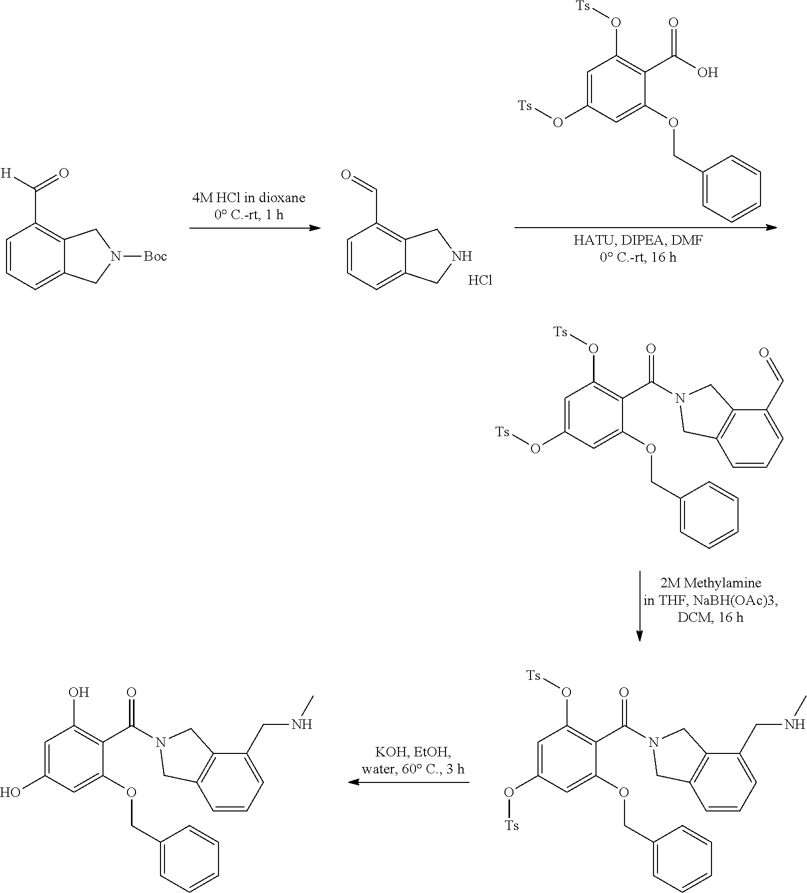

### Isoindoline-4-carbaldehyde hydrochloride

A stirred solution of t-butyl 4-formylisoindoline-2-carboxylate (Intermediate 58) (0.5 g, 2.02 mmol, 1.0 eq.) in DCM (5 mL) at 0° C. was treated with 4M HCl in dioxane (2.5 mL) and stirred for 1 h. The resulting reaction mixture was evaporated and triturated with diethyl ether (20 mL) to give the title compound as an off-white solid (0.371 g, Yield: 100%). ¹H NMR (DMSO-d6, 400 MHz): δ 4.53 (s, 2H), 4.76 (s, 2H), 7.67 (t, J=7.6 Hz, 1H), 7.75 (d, J=7.2 Hz, 1H), 7.99 (d, J=7.2 Hz, 1H), 9.86 (s, 2H), 10.0 (s, 1H). LCMS (Method B): 1.54 min, MS: ES+148.21 (M+1). 5-(Benzyloxy)-4-(4-formylisoindoline-2-carbonyl)-1,3-phenylenebis(4-methylbenzenesulfonate) (Intermediate 59)

A stirred solution of 2-(benzyloxy)-4,6-bis(tosyloxy)benzoic acid (Intermediate 61) (0.4 g, 0.70 mmol, 1.0 eq) in DMF (2 mL) at 0° C.-5° C. was treated with HATU (0.400 g, 1.05 mmol, 1.5 eq) and DIPEA (0.453 g, 3.51 mmol, 5.0 eq) and stirred for 10 mins. Isoindoline-4-carbaldehyde hydrochloride (0.155 g, 0.84 mmol 1.2 eq.) in DMF (1 mL) was added dropwise at 0-5° C. The resulting reaction mixture was stirred at rt for 3 h. The reaction mixture was poured onto ice-cold water (50 mL) and extracted with ethyl acetate (3×20 mL). The combined organic layer was dried over Na₂SO₄, filtered and concentrated under vacuum. The obtained crude material was purified by flash chromatography (neat DCM) yielding the title compound as an off-white solid (0.8 g, Yield: 81.6%) which was used directly in the next step. LCMS (Method A): 2.515 min, 2.531 min. MS: ES+698.18 (M+1).

### 5-(Benzyloxy)-4-(4-((methylamino)methyl) isoindoline-2-carbonyl)-1,3-phenylenebis(4-methylbenzenesulfonate)

A stirred solution of 5-(benzyloxy)-4-(4-formylisoindoline-2-carbonyl)-1,3-phenylene bis(4-methylbenzenesulfonate) (0.4 g, 0.573 mmol, 1.0 eq) in DCM (3 mL) at rt was treated with 2M methyl amine (1.43 mL, 5.0 eq), acetic acid (0.069 g, 1.14 mmol, 2.0 eq) and stirred for 2 h. NaBH(OAc)₃ (0.243 g, 1.14 mmol, 2.0 eq) was added to the reaction mixture at 0° C. The reaction mixture was stirred at room temperature for 16 h. Water (20 mL) was added to the reaction mixture which was then extracted with ethyl acetate (2×20 mL). The combined organic layer was dried Na₂SO₄, filtered and concentrated under vacuum. The crude material was purified by flash chromatography (8% MeOH in DCM) yielding the title compound (0.22 g. Yield: 53.8%). ¹H NMR (DMSO-d6, 400 MHz) Compound is a mixture of rotamers: δ 2.33 (s, 3H), 2.44 (s, 3H), 2.54 (s, 3H), 3.68-3.71 (m, 2H), 4.00-4.03 (m, 2H), 4.35-4.83 (m, 3H), 5.06-5.10 (m, 2H), 6.64-6.66 (m, 1H), 7.01-7.05 (m, 1H), 7.15-7.24 (m, 8H), 7.24-40 (m, 3H), 7.40-7.42 (m, 2H), 7.50-7.60 (m, 2H), 7.67-7.78 (m, 2H). LCMS (Method A): 1.852 min, 1.887 min, MS: ES+713.23 (M+1).

### (2-(Benzyloxy)-4,6-dihydroxyphenyl) (4-((methylamino)methyl) isoindolin-2-yl) methanone Example 79

A stirred solution of 5-(benzyloxy)-4-(4-((methylamino)methyl) isoindoline-2-carbonyl)-1,3-phenylene bis(4-methylbenzenesulfonate) (0.2 g, 0.28 mmol, 1.0 eq.) in EtOH:H₂O (5 mL; 2:1) at rt was treated with NaOH (0.025 g, 0.628 mmol, 3 eq). The reaction mixture was stirred at room temperature for 2 h. The resulting reaction mixture poured into water (10 mL), acidified using KHSO₄ solution (pH 2-3) and extracted with IPA:chloroform (30%) (3×30 mL). The combined organic layer was dried over Na₂SO₄, filtered, and concentrated under reduced pressure. Crude material was purified by prep HPLC (Method E) and the pure fraction lyophilized to give the title compound as an off-white solid (0.035 g. Yield: 31%). ¹H NMR (DMSO-d6, 400 MHz): δ 2.19-2.31 (m, 3H), 3.50 (bs, 2H), 3.69 (s, 1H), 4.51 (d, J=20 Hz, 2H), 4.75 (d, J=21.2 Hz, 2H), 5.02 (s, 2H), 6.02 (d, J=13.6 Hz, 2H), 7.15-7.31 (m, 8H), 9.72 (bs, 2H). LCMS (Method D): 1.153 min, MS: ES+405.17 (M+1). Analytical HPLC (Method C): 4.05 min (99.4%).

Compounds in Table 2 were prepared from Intermediate 59 by reductive amination using either Na(OAc)BH₃/AcOH in DCM or ZnCl₂/NaCNBH₃ in MeOH followed by tosyl deprotection according to the method of Example 79

TABLE 2

| Ex | | | |
|---|---|---|---|
| No | Name | Structure | Data |
| 80 | (2-(Benzyloxy)-4,6- dihydroxyphenyl)(4- (((tetrahydrofuran-3- yl)amino)methyl) isoindolin-2- yl)methanone | 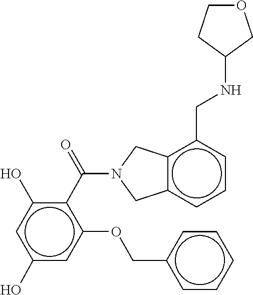 | Prepared from Intermediate 59 and tetrahydrofuran-3-amine. ¹H NMR (DMSO-d6, 400 MHz): δ 1.54 − 1.55 (m, 1H), 1.70 − 1.80 (m, 2H), 1.91 − 1.97 (m, 1H), 2.19 − 2.32 (m, 2H), 3.40 − 3.77 (m, 6H), 4.48 − 4.50 (m, 2H), 4.72 − 4.78 (m, 2H), 5.03 (s, 2H), 6.00 (s, 2H), 7.11 − 7.13 (m, 1H), 7.19 − 7.29 (m, 6H), 9.53 (bs, 2H). LCMS (Method A): 1.176 min, MS ES+ 461.22 (M + 1). |
| 81 | (2-(Benzyloxy)-4,6- dihydroxyphenyl)(4- ((oxetan-3- ylamino)methyl) isoindolin-2- yl)methanone | 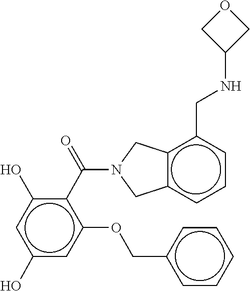 | Prepared from Intermediate 59 and 3- amino-oxetane. ¹H NMR (DMSO-d6, 400 MHz): δ 3.44 (s, 1H), 3.62 (s, 1H), 3.70 − 3.86 (m, 1H), 4.21 (t, J = 6.0 Hz, 1H), 4.30 (t, J = 6.0 Hz, 1H), 4.48 − 4.51 (m, 3H), 4.56 (t, J = 6.4 Hz, 1H), 4.72 − 4.79 (m, 2H), 5.03 (s, 2H), 6.00 (s, 2H), 7.13 − 7.30 (m, 8H), 9.54 (s, 2H). LCMS (Method A): 1.137 min, MS ES+ 447.17 (M + 1). |
| 82 | (2-(Benzyloxy)-4,6- dihydroxyphenyl)(4- (((3-methyloxetan-3- yl)amino)methyl) isoindolin-2- yl)methanone | 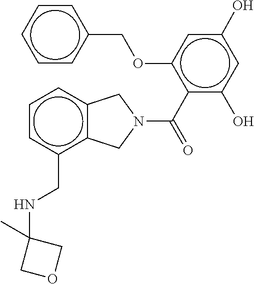 | Prepared from Intermediate 59 and 3- amino-3-methyl-oxetane. ¹H NMR (DMSO-d6, 400 MHz): δ 1.29 − 1.44 (m, 3H), 3.50 − 3.68 (m, 2H), 4.10 (d, J = 6.0 Hz, 1H), 4.20 (d, J = 5.6 Hz, 1H), 4.37 (d, J = 5.6 Hz, 1H), 4.48 − 4.53 (m, 3H), 4.72 − 4.81 (m, 2H), 5.03 (s, 2H), 6.00 (s, 2H), 7.13 − 7.29 (m, 8H), 9.48 − 9.57 (m, 2H). LCMS (Method A): 1.160 min, MS ES+ 461.2 (M + 1). |

### Example 83. (4-(Aminomethyl)isoindolin-2-yl)(2-(benzyloxy)-4,6-dihydroxyphenyl)methanone hydrochloride

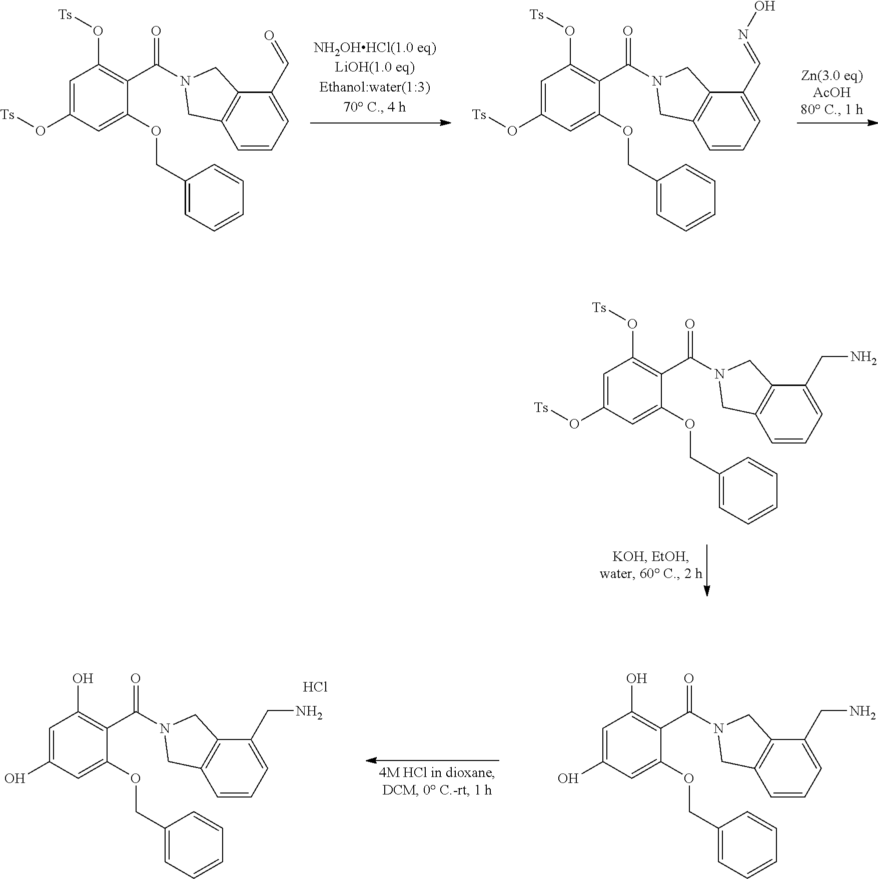 [SMILES: Cc1ccc(S(=O)(=O)Oc2cc(OCc3ccccc3)c(C(=O)N3Cc4cccc(C=O)c4C3)c(OS(=O)(=O)c3ccc(C)cc3)c2)cc1 ; Cc1ccc(S(=O)(=O)Oc2cc(OCc3ccccc3)c(C(=O)N3Cc4cccc(/C=N/O)c4C3)c(OS(=O)(=O)c3ccc(C)cc3)c2)cc1 ; Cc1ccc(S(=O)(=O)Oc2cc(OCc3ccccc3)c(C(=O)N3Cc4cccc(CN)c4C3)c(OS(=O)(=O)c3ccc(C)cc3)c2)cc1 ; NCc1cccc2c1CN(C(=O)c1c(O)cc(O)cc1OCc1ccccc1)C2 ; Cl ; NCc1cccc2c1CN(C(=O)c1c(O)cc(O)cc1OCc1ccccc1)C2]

### 4-(4-(Aminomethyl)isoindoline-2-carbonyl)-5-(benzyloxy)-1,3-phenylene bis(4-methylbenzenesulfonate)

A stirred solution of 5-(benzyloxy)-4-(4-formylisoindoline-2-carbonyl)-1,3-phenylene bis(4-methylbenzenesulfonate) (Intermediate 59) (1.0 g, 1.43 mmol, 1.0 eq) in EtOH:H₂O (1:3; 10 mL) at rt was treated with LiOH (0.034 g, 1.42 mmol, 1.0 eq) and NH—OH. HCl (0.1 g, 1.44 mmol, 1.0 eq). The reaction mixture was heated to 70° C. and stirred for 4 h, cooled, poured into ice cold water (200 mL) and extracted using ethyl acetate (3×200 mL). The combined organic layer was dried over Na₂SO₄, filtered, and concentrated under vacuum. Crude product was triturated with n-pentane (3×30 mL) and diethyl ether (30 mL) to give (E)-5-(benzyloxy)-4-(4-((hydroxyimino)methyl)isoindoline-2-carbonyl)-1,3-phenylenebis(4-methylbenzenesulfonate)compound as an off-white solid (0.95 g, Yield: 93%). LCMS (Method A): 2.390 min, 2.498 min, MS: ES+713.1 (M+1). This material (1.9 g, 26.6 mmol) was dissolved in AcOH (10 mL) and treated with Zn (0.52 g, 7.98 mmol, 3 eq) at rt. The reaction mixture was heated to 80° C. and stirred for 1 h. The reaction mixture was neutralized (pH˜7) using saturated NaHCO₃ solution and extracted with ethyl acetate (3×300 mL). The combined organic layer dried over Na₂SO₄, filtered and concentrated under vacuum. The crude product was triturated with n-pentane (3×20 mL) and diethyl ether (20 mL) yielding 4-(4-(aminomethyl)isoindoline-2-carbonyl)-5-(benzyloxy)-1,3-phenylene bis(4-methylbenzenesulfonate) as an off white solid (1.6 g, Yield: 85.9%). LCMS (Method A): 1.812 min, 1.864 min, MS: ES+699.2 (M+1).

### (4-(Aminomethyl)isoindolin-2-yl)(2-(benzyloxy)-4,6-dihydroxyphenyl)methanone hydrochloride (Example 83)

A stirred solution of 4-(4-(aminomethyl)isoindoline-2-carbonyl)-5-(benzyloxy)-1,3-phenylene bis(4-methylbenzenesulfonate) (1.6 g, 2.28 mmol, 1 eq.) in EtOH:H₂O (2:1) (18 mL) at rt was treated with KOH (5.12 g, 91.43 mmol, 40 eq.). The reaction mixture was heated at 60° C. and stirred for 2 h. The reaction mixture was acidified using saturated solution of KHSO₄ solution (pH˜7) and extracted in IPA:CHCl₃ (3×250 mL). The combined organic layer was dried over Na₂SO₄, filtered and concentrated under vacuum yielding (4-(aminomethyl)isoindolin-2-yl)(2-(benzyloxy)-4,6-dihydroxyphenyl)methanone. This material was taken up in DCM (5 mL), cooled to 0° C. and treated dropwise with 4M HCl in Dioxane (4 mL). The mixture was stirred at room temperature for 1 h, concentrated under vacuum to obtain crude material which was purified by trituration using n-pentane (3×15 mL) and diethyl ether (15 mL) followed by prep HPLC purification of 0.2 g of material (see below) yielding the title compound (0.040 g). H NMR (DMSO-d6, 400 MHz): δ ppm 3.83-3.84 (m, 1H), 4.02-4.03 (m, 2H), 4.53-4.63 (m, 2H), 4.78 (s, 1H), 4.91 (s, 1H), 5.04 (s, 2H), 6.02-6.05 (m, 2H), 7.11-7.12 (m, 1H), 7.23-7.39 (m, 7H), 7.38-7.39 (d, 1H), 8.23 (s, br, 1H), 8.29 (s, br, 2H), 9.55 (s, br 1H), 9.65 (s, br, 1H). LCMS (Method A): 1.178 min, MS: ES+391.1 (M+1). Analytical HPLC (Method C): 4.19 min, 98.3%. Prep HPLC purification was conducted using a Waters 2545 purification system with UV detector. Column YMC-ODS AQ C18 prep C18 (250×20 mm, 5 μm) compound eluted with, Mobile Phase A: 0.05% ammonium hydroxide solution in Milli Q water, Mobile Phase B: Acetonitrile with a gradient of T=0 min (85% A, 15% B) gradient to T=19 min (75% A, 25% B); T=19.01 min (2% A, 98% B) gradient to T=21 min (2% A, 98% B); T=20.01 min (85% A, 15% B) to T=23 min (85% A, 15% B); ); flow rate=17 ml/min; analysis time 23 min.

### Example 84. (2-(Benzyloxy)-4,6-dihydroxyphenyl)(7-((oxetan-3-ylamino)methyl)-3,4-dihydroisoquinolin-2(1H)-yl)methanone

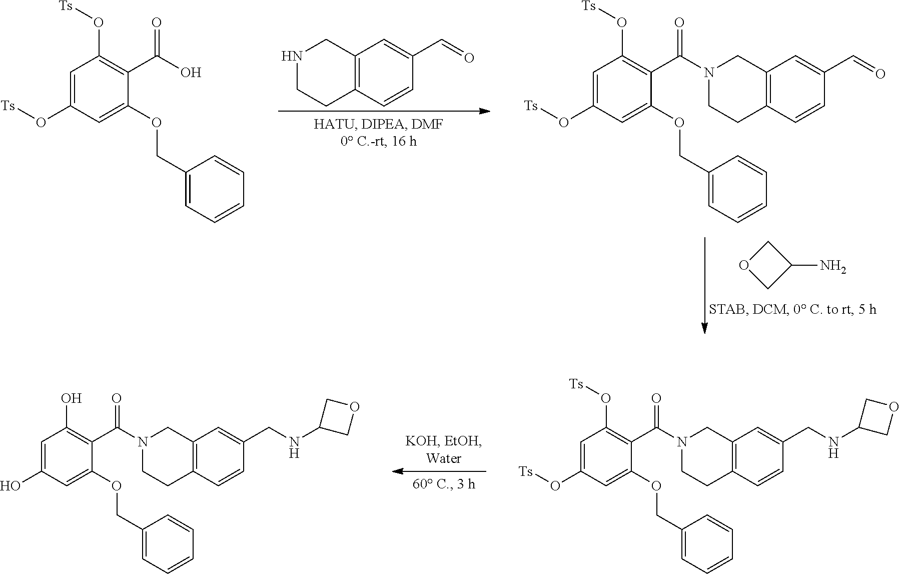

### 5-(Benzyloxy)-4-(7-formyl-1,2,3,4-tetrahydroisoquinoline-2-carbonyl)-1,3-phenylene bis(4-methylbenzenesulfonate) (Intermediate 60)

A stirred solution of 2-(benzyloxy)-4,6-bis(tosyloxy)benzoic acid (Intermediate 61) (2.5 g, 4.40 mmol, 1.0 eq.) in DMF (25 mL) at 0° C. under a nitrogen atmosphere was treated with HATU (3.34 g, 8.80 mmol, 2.0 eq.) and DIPEA (2.3 mL, 13.20 mmol, 3.0 eq.) and stirred for 15 min. 1,2,3,4-Tetrahydroisoquinoline-7-carbaldehyde hydrochloride (CAS:1205750-47-5; 0.94 g, 4.84 mmol, 1.1 eq.) was added and the reaction mixture stirred at rt for 16 h. The reaction mixture was poured into water (50 mL) and extracted using EtOAc (2×50 mL). The combined organic layer was dried over Na₂SO₄ and concentrated under vacuum to give crude material. Column chromatography (product eluted with 2% MeOH in DCM) yielded the title compound (1.8 g, Yield: 57%, 2.52 mmol,). LCMS (Method A): 2.53 min, 2.57 min, MS: ES+712 (M+1).

### 5-(Benzyloxy)-4-(7-((oxetan-3-ylamino)methyl)-1,2,3,4-tetrahydroisoquinoline-2-carbonyl)-1,3-phenylene bis(4-methylbenzenesulfonate)

A stirred solution of 5-(benzyloxy)-4-(7-formyl-1,2,3,4-tetrahydroisoquinoline-2-carbonyl)-1,3-phenylene bis(4-methylbenzenesulfonate) (Intermediate 60) (0.5 g, 0.70 mmol, 1.0 eq.) and oxetan-3-amine (CAS: 21635-88-1) (0.076 g, 1.05 mmol, 1.5 eq) in DCM (20 mL) at 0° C. under nitrogen atmosphere was treated with NaBH(OAc)₃ (0.29 g, 1.40 mmol, 2.0 eq.). The reaction mixture was stirred at room temperature for 5 h, concentrated under vacuum and the crude material diluted with water (20 mL) and extracted with EtOAc (2×25 mL). The combined organic layer was dried over Na₂SO₄ and concentrated under vacuum. The crude material was purified by column chromatography (product eluted in 3.0% MeOH in DCM) yielding the title compound as an off-white sticky solid (0.4 g, Yield: 74%, 0.52 mmol). ¹H NMR (DMSO-d6, 400 MHz): δ ppm 2.32-2.49 (m, 6H), 2.64-2.66 (m, 2H), 3.11-3.26 (m, 2H), 3.53-3.58 (m, 2H), 3.74-3.80 (m, 2H), 4.02-4.15 (m, 1H), 4.21-4.31 (m, 2H), 4.42-4.57 (m, 3H), 4.80-5.60 (m, 2H), 6.55-6.57 (m, 1H), 6.88-7.01 (m, 3H), 7.07-7.25 (m, 6H), 7.39-7.51 (m, 4H), 7.62-7.76 (m, 4H). LCMS (Method A): 1.94 min, 2.00 min, MS: ES+769.1 (M+1).

### (2-(Benzyloxy)-4,6-dihydroxyphenyl)(7-((oxetan-3-ylamino)methyl)-3,4-dihydroisoquinolin-2(1H)-yl)methanone (Example 84)

A stirred solution of 5-(benzyloxy)-4-(7-((oxetan-3-ylamino)methyl)-1,2,3,4-tetrahydroisoquinoline-2-carbonyl)-1,3-phenylene bis(4-methylbenzenesulfonate) (0.4 g, 0.52 mmol, 1.0 eq.) in EtOH:Water (8 mL, 5:3) at rt was treated with KOH (1.16 g, 20.80 mmol, 40 eq.) in water. The resulting reaction mixture was heated to 60° C. and stirred for 3 h. The reaction mixture was acidified with KHSO₄ solution and extracted using (20%) IPA:CHCl₃ (2×25 mL). The combined organic layer was dried over Na₂SO₄ and concentrated under vacuum. The crude material was purified by prep-TLC yielding the title compound (0.058 g, Yield: 24%, 0.05 mmol,). High temperature ¹H NMR (DMSO-d6, 400 MHz): δ 2.66-2.73 (s, 2H), 3.56-3.58 (s, 2H), 3.86-3.87 (m, 1H), 4.26-4.28 (m, 2H), 4.53-4.54 (m, 2H), 4.93-4.95 (m, 2H), 6.01 (s, 2H), 7.03-7.09 (m, 3H), 7.23 (s, 5H), 9.22 (d, J=15.2 Hz, 2H). LCMS (Method A): 1.171 min, MS ES+461.2 (M+1).

### Example 144: 2-(Benzyloxy)-4,6-dihydroxyphenyl-(5-((oxetan-3-ylamino)methyl)isoindolin-2-yl)methanone

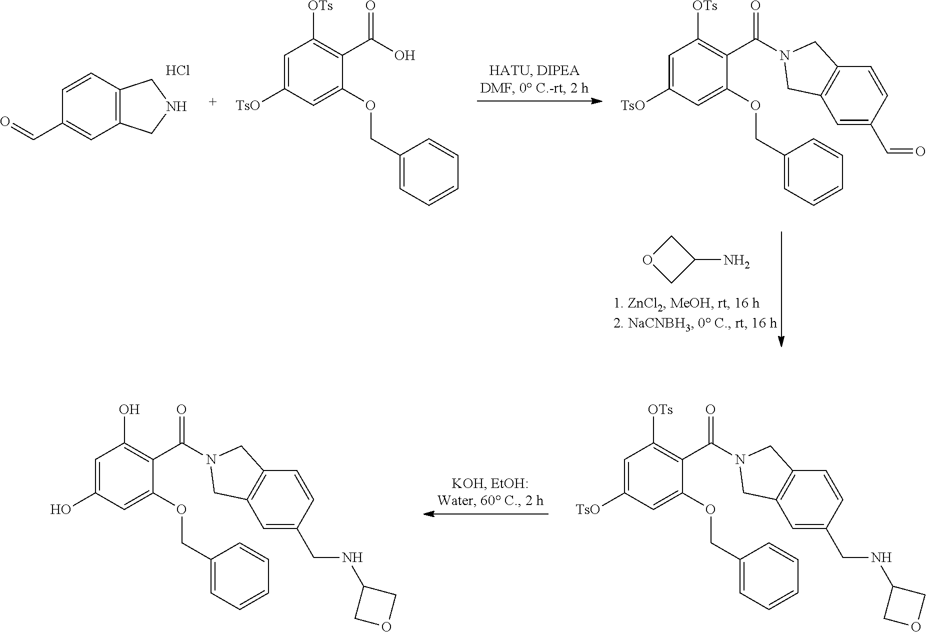

### 5-(Benzyloxy)-4-(5-formylisoindoline-2-carbonyl)-1,3-phenylene bis(4-methylbenzenesulfonate)

A stirred solution of 2-(benzyloxy)-4,6-bis(tosyloxy)benzoic acid (Intermediate 61) (4.17 g, 7.34 mmol, 1.0 eq.) in DMF (70 mL) was treated with HATU (3, 73 g, 9.80 mmol, 2.0 eq.) and DIPEA (1.9 g, 14.7 mmol, 3.0 eq.) at 0° C. and stirred for 5 mins. Isoindoline-5-carbaldehyde hydrochloride (See preparation of Intermediate 73) (0.9 g, 4.92 mmol, 1.0 eq.) was added at 0° C. and the reaction mixture stirred at room temperature for 2 h, then diluted with water (300 mL) and extracted in ethyl acetate (4×100 mL). The combined organic layer was dried over Na₂SO₄, filtered, and concentrated under reduced pressure. Crude material was purified using flash chromatography (product eluted in 2% MeOH:DCM) yielding the title compound (2.8 g, Yield: 47%). LCMS (Method A): 2.489 min, MS: ES+698 (M+1).

### 5-(Benzyloxy)-4-(5-((oxetan-3-ylamino)methyl)isoindoline-2-carbonyl)-1,3-phenylene bis(4-methylbenzenesulfonate)

A stirred solution of 5-(benzyloxy)-4-(5-formylisoindoline-2-carbonyl)-1,3-phenylene bis(4-methylbenzenesulfonate) (0.500 g, 0.716 mmol, 1.0 eq.) in MeOH (25 mL) at room temperature was treated with oxetan-3-amine (CAS #21635-88-1) (0.105 g, 1.433 mmol, 2.0 eq.) and ZnCl₂ (0.095 g, 0.716 mmol, 1.0 eq) and stirred for 16 h. The reaction mixture was cooled to 0° C., NaCNBH₃ (0.090 g, 1.433 mmol, 2.0 eq.) was added and the resulting reaction mixture stirred for 16 h at room temperature. The reaction mixture was concentrated under vacuum, water (200 mL) was added and extracted using EtOAc (3×100 mL). The combined organic layer was dried over Na₂SO₄, filtered, and concentrated under reduced pressure. Crude material (0.600 g) was used for next step directly without purification. (0.600 g, Yield: 80%, 0.795 mmol). LCMS (Method A): 1.869 min, MS: ES+755.3 (M+1).

### 2-(Benzyloxy)-4,6-dihydroxyphenyl-(5-((oxetan-3-ylamino)methyl)isoindolin-2-yl)methanone (Example 144)

A stirred solution 5-(benzyloxy)-4-(5-((oxetan-3-ylamino)methyl)isoindoline-2-carbonyl)-1,3-phenylene bis(4-methylbenzenesulfonate) (0.600 g, 0.795 mmol, 1.0 eq.) in EtOH:water (20 mL, 1:1) was treated with aqueous solution of KOH (1.8 g, 31.82 mmol, 40 eq in minimum water) at room temperature. The reaction mixture was heated to 60° C. and stirred for 2 h, allowed to cool to room temperature, poured into ice cooled water (35 mL) and neutralized with saturated solution of KHSO₄, and extracted with ethyl acetate (2×100 mL). The combined organic layer was dried over Na₂SO₄, filtered, and concentrated under reduced pressure. Crude material (0.400 g) was purified by flash chromatography (product eluted in 8% MeOH in DCM) to give the title compound (0.107 g, Yield: 30%). ¹H NMR (DMSO-d6, 400 MHz, K): ppm 3.69 (d, J=13.6 Hz, 2H), 3.82-3.88 (m, 1H), 4.25-4.31 (m, 2H), 4.45 (bs, 2H), 4.51-4.56 (m, 2H), 4.71 (bs, 2H), 5.03 (s, 2H), 6.01 (s, 2H), 7.11-7.49 (m, 9H), 9.49 (d, J=2.8 Hz, 1H), 9.56 (s, 1H). LCMS (Method A): 1.071 min, MS: ES+447.1 (M+1). Analytical HPLC (Method A): 3.939 min, 96.9%.

### Example 145: (2-(Benzyloxy)-4,6-dihydroxyphenyl)(5-(((tetrahydrofuran-3-yl)amino)methyl)isoindolin-2-yl)methanone

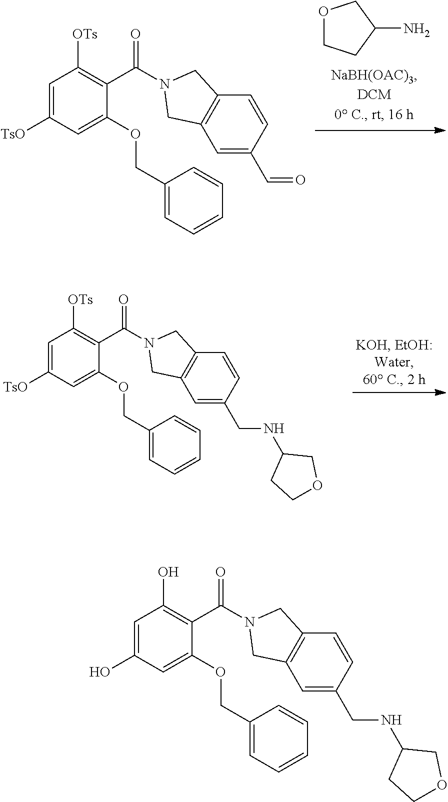 [SMILES: Cc1ccc(S(=O)(=O)Oc2cc(OCc3ccccc3)c(C(=O)N3Cc4ccc(C=O)cc4C3)c(OS(=O)(=O)c3ccc(C)cc3)c2)cc1 ; NC1CCOC1 ; Cc1ccc(S(=O)(=O)Oc2cc(OCc3ccccc3)c(C(=O)N3Cc4ccc(CNC5CCOC5)cc4C3)c(OS(=O)(=O)c3ccc(C)cc3)c2)cc1 ; O=C(c1c(O)cc(O)cc1OCc1ccccc1)N1Cc2ccc(CNC3CCOC3)cc2C1]

### 5-(Benzyloxy)-4-(5-(((tetrahydrofuran-3-yl)amino)methyl)isoindoline-2-carbonyl)-1,3-phenylene bis(4-methylbenzenesulfonate)

A stirred solution of 5-(benzyloxy)-4-(5-formylisoindoline-2-carbonyl)-1,3-phenylene bis(4-methylbenzenesulfonate) (0.350 g, 0.506 mmol, 1.0 eq.) in DCM (3.5 mL) was cooled to 0° C. Tetrahydrofuran-3-amine (CAS: 204512-94-7) (0.123 g, 0.100 mmol, 2.0 eq.) and Na(OAc)BH₃ (0.212 g, 0.100 mmol, 2.0 eq) were added to the reaction mixture at 0° C. and the mixture was stirred at room temperature for 16 h, diluted with water (50 mL) and extracted using DCM (3×30 mL). The combined organic layer was dried over Na₂SO₄, filtered, and concentrated under reduced pressure. Crude material was purified by flash chromatography (product eluted in 7% MeOH:DCM) yielding the title compound (0.180 g, Yield: 44%). LCMS (Method A): 1.902 min, 1.918 min, MS: ES+769. (M+1).

### 2-(Benzyloxy)-4,6-dihydroxyphenyl-(5-(((tetrahydrofuran-3-yl)amino)methyl)isoindolin-2-yl)methanone (Example 145)

A stirred solution 5-(benzyloxy)-4-(5-(((tetrahydrofuran-3-yl)amino)methyl)isoindoline-2-carbonyl)-1,3-phenylene bis(4-methylbenzenesulfonate) (0.320 g, 0.416 mmol, 1.0 eq.) in EtOH:water (6.4 mL, 1:1) was treated with aqueous solution KOH (0.933 g, 16.63 mmol, 40 eq. in minimum water) at room temperature. The resulting reaction mixture was heated to 60° C. and stirred for 2 h. The resulting reaction mixture was neutralized with 1 N HCl (15 mL) and extracted with ethyl acetate (3×50 mL) followed by DCM (2×50 mL). The combined organic layer was dried over Na₂SO₄, filtered, and concentrated under reduced pressure. Crude material was purified by Prep. TLC using 7% MeOH in DCM yielding the title compound (0.038 g, Yield: 20%). ¹H NMR (DMSO-d6, 400 MHz): δ ppm 1.64-1.69 (m, 1H), 1.89-1.91 (m, 1H), 3.23-3.28 (m, 2H), 3.38-3.44 (m, 2H), 3.61-3.76 (m, 5H), 4.46 (bs, 2H), 4.71 (s, 2H), 5.03 (s, 2H), 6.01 (s, 2H), 7.17-7.34 (m, 8H), 9.48-9.55 (m, 2H). LCMS (Method A): 1.121 min, MS: ES+461 (M+1). Analytical HPLC (Method A): 4.186 min. 99%.

### Example 146:1-(3-((2-(2-(Benzyloxy)-4,6-dihydroxybenzoyl)-1,2,3,4-tetrahydroisoquinolin-8-yl)amino)azetidin-1-yl)ethan-1-one

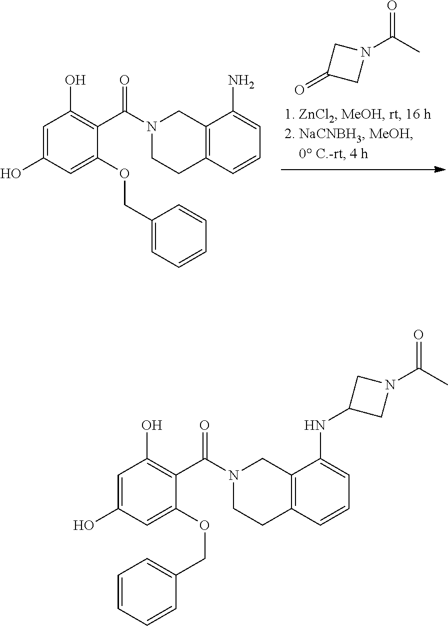

A stirred solution of 8-amino-3,4-dihydroisoquinolin-2(1H)-yl)(2-(benzyloxy)-4,6-dihydroxyphenyl)methanone (Example 100) (0.1 g, 0.25 mmol, 1 eq.) and 1-acetylazetidin-3-one (CAS: 179894-05-4) (0.057 g, 0.51 mmol, 2 eq.) in MeOH (3 mL) at room temperature was treated with ZnCl₂ (0.1 g, 0.76 mmol, 3 eq.). The reaction mixture was heated to 60° C., stirred for 16 h and cooled. NaCNBH₃ (0.08 g, 1.28 mmol, 5 eq.) was added at 0° C. and the resulting reaction mixture stirred at room temperature for 16 h, poured into ice cold water (60 mL) and extracted using ethyl acetate (3×50 mL). The combined organic layer was dried over Na₂SO₄, filtered, and concentrated under vacuum. Crude material was purified by Prep TLC using 10% MeOH in DCM as a mobile phase) yielding the title compound as a white solid (0.040 g, Yield: 16.2%). ¹H NMR (DMSO-d6, 400 MHz, 353K): δ ppm 1.79 (s, 3H), 2.72 (t, J=17.6 Hz, 2H), 3.51 (s, 2H), 3.78 (s, 2H), 3.98 (s, 2H), 4.21 (s, 2H), 4.47-4.52 (m, 3H), 5.00 (s, 3H) 5.34 (s, 1H), 6.04 (s, 2H), 6.29 (d, J=8 Hz, 1H), 6.50 (d, J=7.2 Hz, 1H), 7.01 (t, J=7.6 Hz, 1H), 7.27-7.30 (m, 5H), 9.18 (d, J=14.4 Hz, 2H). LCMS (Method A): 1.605 min, MS ES+: 488.01 (M+1). Analytical HPLC (Method A): 5.825 min. 99.7%.

### Example 147: (2-(Benzyloxy)-4,6-dihydroxyphenyl)(8-((1,1-dioxidotetrahydrothiophen-3-yl)amino)-3,4-dihydroisoquinolin-2(1H)-yl)methanone

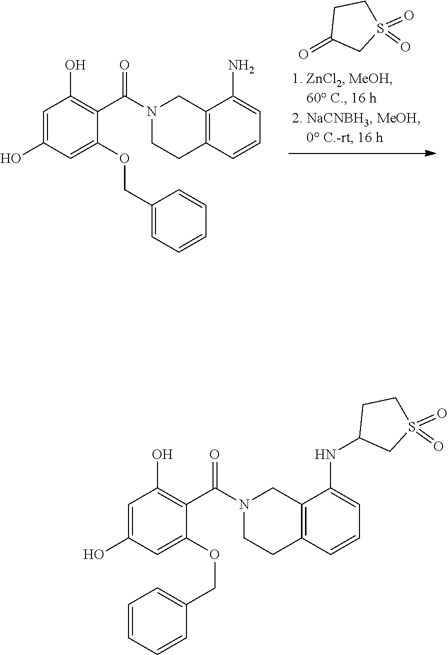 [SMILES: O=C1CCS(=O)(=O)C1 ; Nc1cccc2c1CN(C(=O)c1c(O)cc(O)cc1OCc1ccccc1)CC2 ; O=C(c1c(O)cc(O)cc1OCc1ccccc1)N1CCc2cccc(NC3CCS(=O)(=O)C3)c2C1]

A stirred solution of 8-amino-3,4-dihydroisoquinolin-2(1H)-yl-(2-(benzyloxy)-4,6-dihydroxyphenyl) methanone (Example 100) (0.1 g, 0.25 mmol, 1 eq) and dihydrothiophen-3(2H)-one 1,1-dioxide (CAS: 17115-51-4) (0.068 g, 0.51 mmol, 2 eq.) in MeOH (3 mL) at room temperature was treated with ZnCl₂ (0.1 g, 0.76 mmol, 3 eq.). The reaction mixture was heated to 60° C., stirred for 16 h and cooled. NaCNBH₃ (0.08 g, 1.28 mmol, 5 eq.) was added to reaction mixture at 0° C. and the resulting reaction mixture stirred at room temperature for 16 h. The reaction mixture was poured into ice cold water (50 mL) and extracted using ethyl acetate (3×30 mL). The combined organic layer was dried over Na₂SO₄, filtered, and concentrated under vacuum. Crude material was purified by Prep TLC using 7% MeOH in DCM as a mobile phase yielding the title compound as a white solid (0.034 g, Yield: 26.1%). ¹H NMR (DMSO-d6, 400 MHz, 353K): δ ppm 2.26-2.35 (m, 1H), 3.06 (s, 4H), 3.14-3.19 (m, 2H), 3.32-3.33 (m, 2H), 3.50-3.56 (m, 4H), 4.34-4.36 (m, 1H), 4.52 (bs, 2H), 5.00 (s, br, 3H), 6.04-6.05 (s, br, 2H), 6.51 (d, J=7.6 Hz, 1H), 6.60 (d, J=8 Hz, 1H), 7.03 (t, J=7.6 Hz, 1H), 7.29 (s, br, 5H), 9.18 (d, J=15.6 Hz, 2H). LCMS (Method A): 1.676 min, MS ES+: 509.1 (M+1). Analytical HPLC (Method A): 6.332 min. 98.7%. Preparative chiral HPLC separation of Example 147 used an Agilent 1200 series infinity-II purification system with UV detector, Chiral PakAD-H, 250*10 mm, 5 μm column and compound eluted with: Mobile phase A: Heptane, Mobile phase B: Isopropyl alcohol:Methanol (70:30) with isocratic elution of T=0 min (80% A, 20% B) to T=40 min (80% A, 20% B); Flow rate=8 ml/min; analysis time 40 min. Chiral HPLC gave 2 fractions: Fraction 1: Example 147a. LCMS (Method A): 1.685 min, MS ES+: 508.7 (M+1). Analytical HPLC (Method A): 6.292 min, 100%. Fraction 2: Example 147b. LCMS (Method A): 1.684 min, MS ES+: 508.7 (M+1). Analytical HPLC (Method A): 6.279 min. 100%.

### Example 148: (2-(Benzyloxy)-4,6-dihydroxyphenyl)(8-((4-methoxytetrahydrofuran-3-yl)amino)-3,4-dihydroisoquinolin-2(1H)-yl)methanone

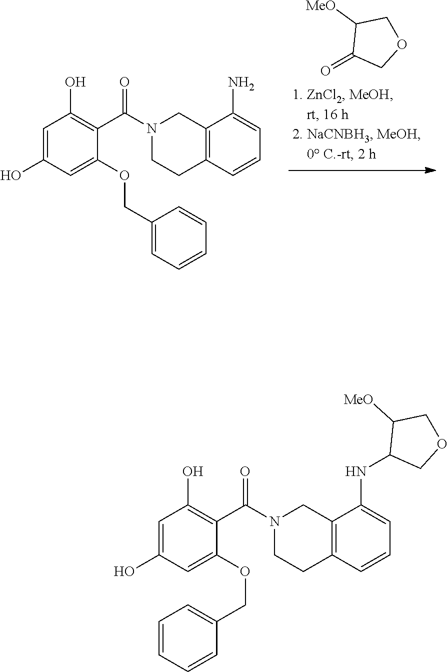 [SMILES: COC1COCC1=O ; Nc1cccc2c1CN(C(=O)c1c(O)cc(O)cc1OCc1ccccc1)CC2 ; COC1COCC1Nc1cccc2c1CN(C(=O)c1c(O)cc(O)cc1OCc1ccccc1)CC2]

A stirred solution 8-amino-3,4-dihydroisoquinolin-2(1H)-yl)(2-(benzyloxy)-4,6-dihydroxyphenyl)methanone (Example 100) (0.090 g, 0.23 mmol, 1 eq.) and 4-methoxydihydrofuran-3(2H)-one (0.053 g, 0.45 mmol, 2 eq.) (CAS: 625100-11-0) in MeOH (2 mL) at room temperature was treated with ZnCl₂ (0.094 g, 0.69 mmol, 3 eq.). The resulting reaction mixture was stirred at room temperature for 16 h, then cooled to 0° C. and treated with NaCNBH₃ (0.073 g, 1.16 mmol, 5 eq.) and stirred for 2 h at room temperature. The reaction mixture was poured into ice cold water (30 mL) and extracted using ethyl acetate (3×50 mL). The combined organic layer was dried over Na₂SO₄, filtered, and concentrated under vacuum. Crude material (0.150 g) was purified by flash chromatography followed by Prep TLC purification (5% MeOH in DCM) yielding the tile compound as an off-white solid (0.013 g, Yield: 10.6%). ¹H NMR (DMSO-d6, 400 MHz): δ ppm 1.24 (s, 2H), 1.76-1.81 (m, 4H), 1.91-1.95 (m, 4H), 1.98 (s, 2H), 2.99 (s, 1H), 3.16 (s, 1H), 3.38-3.44 (m, 3H), 3.5-3.6 (m, 3H), 3.64-3.7 (m, 2H), 3.78-3.93 (m, 4H), 3.95-4.25 (m, 6H), 4.40-4.70 (m, 2H), 4.72-4.9 (m, 2H), 5.02 (s, 2H), 5.99 (s, 3H), 6.43 (d, J=7.2 Hz, 1H), 6.50-6.53 (m, 1H), 6.62 (d, J=7.2 Hz, 1H), 7.00-7.04 (m, 2H), 7.25-7.30 (m, 7H), 9.48 (s, 2H). LCMS (Method A): 1.766 min, MS: ES+491.1 (M+1). Analytical HPLC (Method A): 6.591 min. 92.5%.

### Example 85. (2-(Benzyloxy)-4,6-dihydroxyphenyl)(7-(((tetrahydrofuran-3-yl)amino)methyl)-3,4-dihydroisoquinolin-2(1H)-yl)methanone

Prepared in an analogous manner to Example 84 using tetrahydrofuran-3-amine hydrochloride. High temperature ¹H NMR (DMSO-d6, 400 MHz): δ 1.66-1.67 (m, 1H) 1.90-1.92 (m, 1H), 2.04-2.05 (m, 1H), 2.67-2.74 (m, 2H), 3.27-3.43 (m, 2H), 3.53-3.78 (m, 6H), 4.95 (s, 1H), 6.00 (s, 2H), 7.04-7.11 (m, 3H), 7.23 (s, 5H), 9.23 (bs, 2H). LCMS (Method A): 1.191 min, MS ES+475.2 (M+1).

### Example 86. 2-(2-(Benzyloxy)-4,6-dihydroxybenzoyl)-N-methylisoindoline-5-carboxamide

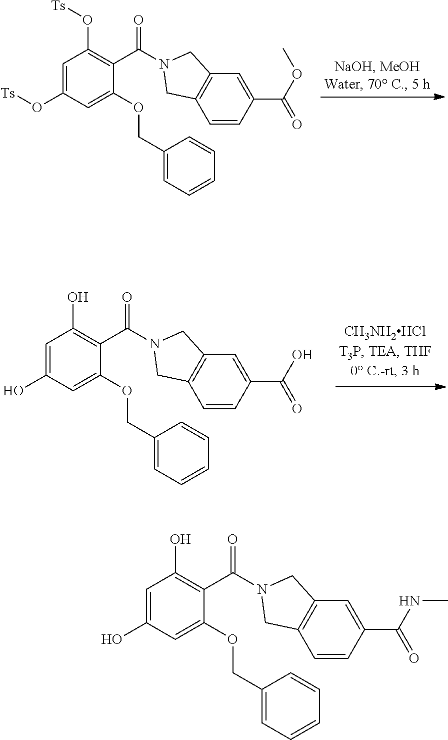 [SMILES: COC(=O)c1ccc2c(c1)CN(C(=O)c1c(OCc3ccccc3)cc(OS(=O)(=O)c3ccc(C)cc3)cc1OS(=O)(=O)c1ccc(C)cc1)C2 ; O=C(O)c1ccc2c(c1)CN(C(=O)c1c(O)cc(O)cc1OCc1ccccc1)C2 ; CNC(=O)c1ccc2c(c1)CN(C(=O)c1c(O)cc(O)cc1OCc1ccccc1)C2]

### 2-(2-(Benzyloxy)-4,6-dihydroxybenzoyl)isoindoline-5-carboxylic acid (Intermediate 62)

A stirred solution of methyl 2-(2-(benzyloxy)-4,6-bis(tosyloxy)benzoyl)isoindoline-5-carboxylate [prepared from 2-(benzyloxy)-4,6-bis(tosyloxy)benzoic acid (Intermediate 61) and methyl isoindoline-5-carboxylate (CAS 742666-57-5)](1.5 g, 2.06 mmol, 1.0 eq) in MeOH:H₂O (5 mL, 1:1) at rt was treated with NaOH (0.825 g, 20.62 mmol, 10 eq.). The resulting reaction mixture was heated to 70° C. for 5 h. The resulting reaction mixture was cooled to room temperature, poured into water (200 mL), acidified using dil. HCl (pH˜6) and extracted with ethyl acetate (3×220 mL). The combined organic layer was dried over Na₂SO₄, filtered and dried under high vacuum yielding the title compound (Intermediate 62) (0.700 g; Yield: 83.9%). LCMS (Method A): 1.493 min, MS: ES+406.27 (M+1).

### 2-(2-(Benzyloxy)-4,6-dihydroxybenzoyl)-N-methylisoindoline-5-carboxamide (Example 86)

A stirred solution of 2-(2-(benzyloxy)-4,6-dihydroxybenzoyl)isoindoline-5-carboxylic acid (Intermediate 62) (0.250 g, 0.617 mmol, 1.0 eq.) in THF (2 mL) was cooled to 0° C. and treated with T3P (50% in EtOAc) (0.294 g, 0.92 mmol, 1.5 eq) and TEA (0.18 mL, 1.22 mmol, 2 eq) and stirred for 10 min. Methylamine.hydrochloride (0.043 g, 0.63 mmol, 1.1 eq) was added to the reaction mixture at 0′C. The resulting reaction mixture was stirred at rt for 16 h. After completion of reaction, the reaction mixture was evaporated and the crude material purified by flash chromatography (product eluted in 6.2% MeOH:DCM) yielding the title compound 2-(2 (0.025 g, Yield: 9.7%). High temperature ¹H NMR (DMSO-d6, 400 MHz, 335K): δ 2.79 (s, 3H), 4.52-4.53 (m, 2H), 4.77-4.78 (m, 2H), 5.03 (s, 2H), 6.05 (s, 2H), 7.23-7.26 (m, 3H), 7.29-7.31 (m, 2H), 7.43-7.45 (m, 1H), 7.68-7.84 (m, 2H), 8.20-8.25 (m, 1H), 9.33-9.37 (m, 2H). LCMS (Method E): 1.400 min, MS ES+419.30 (M+1).

### Example 87. 2-(2-(Benzyloxy)-4,6-dihydroxybenzoyl)-N,N-dimethylisoindoline-5-carboxamide

Prepared in an analogous manner to Example 86 using dimethylamine.hydrochloride. ¹H NMR (DMSO-d6, 400 MHz), compound is a mixture of rotamers: δ 2.87-2.97 (m, 3H), 4.49 (bs, 2H), 4.75 (bs, 2H), 5.03 (s, 2H), 6.02 (s, 2H), 7.23-7.44 (m, 8H). LCMS (Method E): 1.469 min, MS ES+433.22 (M+1).

### Example 88. (2-(Benzyloxy)-4,6-dihydroxyphenyl)(4-((tetrahydrofuran-3-yl)oxy)isoindolin-2-yl)methanone

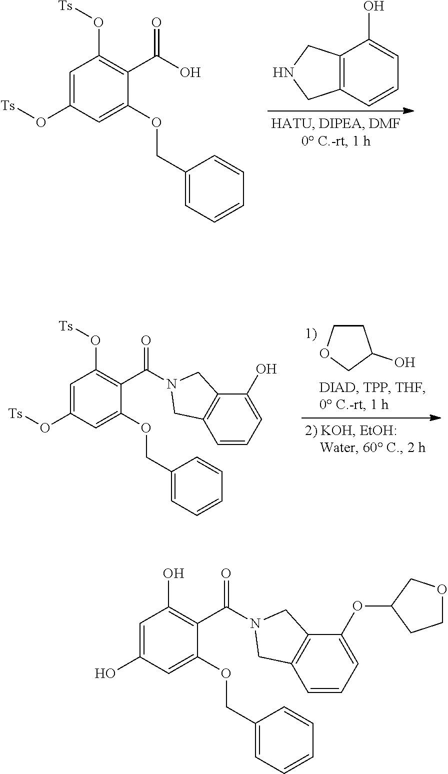 [SMILES: Cc1ccc(S(=O)(=O)Oc2cc(OCc3ccccc3)c(C(=O)O)c(OS(=O)(=O)c3ccc(C)cc3)c2)cc1 ; Oc1cccc2c1CNC2 ; Cc1ccc(S(=O)(=O)Oc2cc(OCc3ccccc3)c(C(=O)N3Cc4cccc(O)c4C3)c(OS(=O)(=O)c3ccc(C)cc3)c2)cc1 ; OC1CCOC1 ; O=C(c1c(O)cc(O)cc1OCc1ccccc1)N1Cc2cccc(OC3CCOC3)c2C1]

### 5-(Benzyloxy)-4-(4-hydroxyisoindoline-2-carbonyl)-1,3-phenylene bis(4-methylbenzenesulfonate) (Intermediate 63)

A stirred solution of 2-(benzyloxy)-4,6-bis(tosyloxy)benzoic acid (Intermediate 61) (5 g, 8.83 mmol, 1.0 eq.) in DMF (50 mL) at 0° C. under a nitrogen atmosphere was treated with HATU (5.03 g, 13.25 mmol, 1.5 eq.) and DIPEA (2.27 g, 17.66 mmol, 2.0 eq.) and stirred for 15 min. Isoindolin-4-ol hydrochloride (CAS: 72695-20-6) (1.65 g, 9.71 mmol, and 1.1 eq.) was added and the reaction mixture stirred at 0° C. for 1 h, diluted with ethyl acetate (150 mL) and washed with saturated chilled brine solution (4×150 mL). The organic layer was dried over Na₂SO₄, filtered, and concentrated under reduced pressure. The crude material (6.5 g) was purified by normal phase flash chromatography (product eluted in 3% methanol in DCM) yielding the title compound (4 g, 5.83 mmol, Yield: 67%). LCMS (Method A): 2.384 min, MS: ES+686.01 (M+1)

### 5-(Benzyloxy)-4-(4-((tetrahydrofuran-3-yl) oxy) isoindoline-2-carbonyl)-1, 3-phenylene bis (4-methylbenzenesulfonate)

5-(Benzyloxy)-4-(4-hydroxyisoindoline-2-carbonyl)-1,3-phenylene-bis(4-methylbenzenesulfonate) (Intermediate 63) (0.5 g, 0.72 mmol, 1.0 eq.), tetrahydrofuran-3-ol (CAS: 453-20-3) (0.064 g, 0.72 mmol, 1.0 eq.) and TPP (0.38 g, 1.45 mmol, 2.0 eq.) in THF (0.5 mL) (minimum solvent as a paste) was sonicated for 15 min. DIAD (98%) (0.29 g, 1.45 mmol, 2.0 eq.) was added dropwise and the resulting reaction mixture stirred under nitrogen atmosphere at 60° C. for 30 min. The resulting reaction mixture was allowed to cool to room temperature, poured into ice-water (75 mL) and extracted with ethyl acetate (3×75 mL). The combined organic layer was dried over Na₂SO₄, filtered, and concentrated under reduced pressure. Crude material (0.68 g) was purified by normal phase flash chromatography (product eluted with 0.5% MeOH in DCM) yielding the title compound (0.25 g, 0.04 mmol, Yield: 45%). LCMS (Method A): 2.614 min, 2.683 min, MS: ES+756.2 (M+1)

### (2-(Benzyloxy)-4,6-dihydroxyphenyl)(4-((tetrahydrofuran-3-yl)oxy)isoindolin-2-yl)methanone (Example 88)

A stirred solution 5-(benzyloxy)-4-(4-((tetrahydrofuran-3-yl) oxy) isoindoline-2-carbonyl)-1, 3-phenylene bis (4-methylbenzenesulfonate) (0.22 g, 0.49 mmol, 1.0 eq.) in EtOH:water (2.2 mL, 1:1) at rt was treated with aqueous KOH (0.10 g, 19.67 mmol, 40 eq. minimum water). The mixture was heated at 60° C. for 2 h. The resulting reaction mixture was allowed to cool to room temperature, poured into ice cooled water (40 mL) and neutralized with diluted HCl (pH˜7) and extracted with ethyl acetate (3×40 mL). The combined organic layer was dried over Na₂SO₄, filtered, and concentrated under reduced pressure. The crude material was purified by flash chromatography (product eluted in 8% MeOH in DCM) yielding) (0.055 g, 0.12 mmol, Yield: 42%). ¹H NMR (DMSO-d6, 400 MHz): δ 1.87-2.01 (m, 1H), 2.15-2.23 (m, 1H), 3.68-3.94 (m, 2H), 4.33-4.48 (m, 2H), 4.59-4.73 (m, 2H), 5.03-5.10 (m, 3H), 6.01 (s, 2H), 6.84-6.97 (m, 2H), 7.24-7.30 (m, 6H), 9.51-9.59 (m, 2H). LCMS (Method A): 1.697 min, MS: ES+448.2 (M+1).

### Example 89 (2-(Benzyloxy)-4,6-dihydroxyphenyl)(4-(pyrimidin-5-ylmethoxy)isoindolin-2-yl)methanone

Prepared in an analogous manner to Example 88 using pyrimidin-5-ylmethanol (CAS: 25193-95-7). ¹H NMR (DMSO-d6, 400 MHz); compound is a mixture of rotamers: δ 4.39 (s, 1H), 4.48 (s, 1H), 4.71 (d, J=20.4 Hz, 2H), 5.03 (1s, 2H), 5.24 (d, J=31.2 Hz, 2H), 5.99 (two singlets, 2H), 6.98-7.03 (m, 2H), 7.22-7.29 (m, 6H), 8.84 (s, 1H), 8.95 (s, 1H), 9.12-9.18 (m, 1H), 9.50 (s, 1H), 9.57 (s, 1H). LCMS (Method A): 1.525 min, MS ES+470.3 (M+1).

### Example 90. (4-(Azetidin-3-ylmethoxy)isoindolin-2-yl)(2-(benzyloxy)-4,6-dihydroxyphenyl) methanone

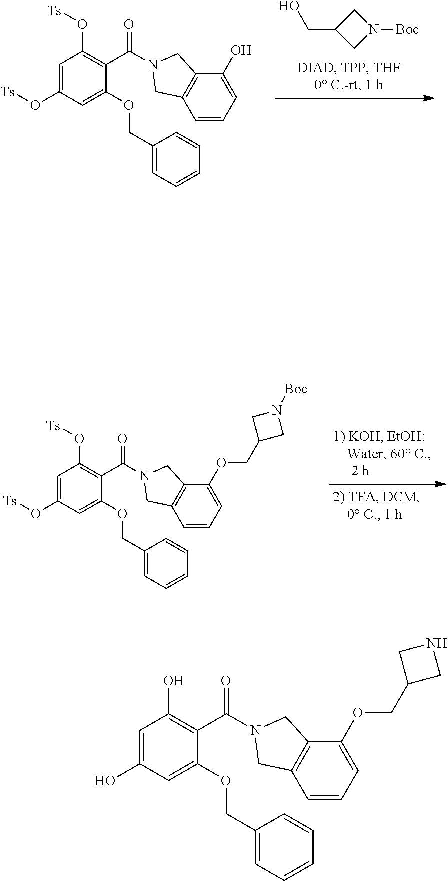 [SMILES: Cc1ccc(S(=O)(=O)Oc2cc(OCc3ccccc3)c(C(=O)N3Cc4cccc(O)c4C3)c(OS(=O)(=O)c3ccc(C)cc3)c2)cc1 ; CC(C)(C)OC(=O)N1CC(CO)C1 ; Cc1ccc(S(=O)(=O)Oc2cc(OCc3ccccc3)c(C(=O)N3Cc4cccc(OCC5CN(C(=O)OC(C)(C)C)C5)c4C3)c(OS(=O)(=O)c3ccc(C)cc3)c2)cc1 ; O=C(c1c(O)cc(O)cc1OCc1ccccc1)N1Cc2cccc(OCC3CNC3)c2C1]

### t-Butyl 3-(((2-(2-(benzyloxy)-4,6-bis(tosyloxy)benzoyl)isoindolin-4-yl)oxy)methyl) azetidine-1-carboxylate

5-(Benzyloxy)-4-(4-hydroxyisoindoline-2-carbonyl)-1,3-phenylene bis(4-methylbenzenesulfonate) (Intermediate 63) (0.7 g, 1.02 mmol, 1.0 eq.), t-butyl 3-(hydroxymethyl) azetidine-1-carboxylate (CAS: 142253-56-3) (0.19 g, 1.02 mmol, 1.0 eq.) and triphenyl phosphine (0.53 g, 2.04 mmol, 2.0 eq.) in THF (0.7 mL, minimum solvent to make a paste) were sonicated for 15 min. DIAD (98%) (0.41 g, 2.04 mmol, 2.0 eq.) was added drop wise and the resulting reaction mixture stirred under nitrogen at 60° C. for 1 h. The resulting reaction mixture was allowed to cool to room temperature, poured into ice-water (100 mL) and extracted with ethyl acetate (4×100 mL). The combined organic was dried over Na₂SO₄, filtered, and concentrated under reduced pressure. Crude material was purified by flash chromatography (0.5% methanol in DCM) to give the title compound (1.0 g. Yield: quantitative). LCMS (Method A): 2.788 min, 2.882 min, MS: ES+755.2 (M−100).

### (4-(Azetidin-3-ylmethoxy)isoindolin-2-yl)(2-(benzyloxy)-4,6-dihydroxyphenyl)methanone Example 90

A stirred solution of t-butyl-3-(((2-(2-(benzyloxy)-4,6-bis(tosyloxy)benzoyl)isoindolin-4-yl)oxy)methyl) azetidine-1-carboxylate (0.95 g, 1.11 mmol, 1.0 eq.) in EtOH (9.5 mL) at rt was treated with aqueous hydroxide (2.49 g, 44.49 mmol, 40 eq. in minimum amount of water). The reaction mixture was heated to 60° C. and stirred for 2 h. The resulting reaction mixture was allowed to cool to room temperature, poured into water (80 mL) and neutralized with diluted HCl (pH˜7) and extracted with ethyl acetate (4×100 mL). The combined organic layer was dried over sodium sulphate, filtered, and concentrated under reduced pressure. The crude material was purified by flash chromatography (product eluted in 3.8% MeOH in DCM) yielding t-butyl 3-(((2-(2-(benzyloxy)-4,6-dihydroxybenzoyl)isoindolin-4-yl)oxy)methyl)azetidine-1-carboxylate (0.21 g, 0.38 mmol, Yield: 35%). LCMS (Method A): 2.024 min, purity: 90.13%, MS: ES+447.2 (M−100).

A stirred solution of t-butyl 3-(((2-(2-(benzyloxy)-4,6-dihydroxybenzoyl)isoindolin-4-yl)oxy)methyl)azetidine-1-carboxylate (0.18 g, 0.32 mmol, 1.0 eq.) in DCM (1.8 mL, 10 v) at 0° C. was treated with TFA (0.9 ml) for 1 h. The resulting reaction mixture was poured into water (30 mL) and neutralized (pH˜8) with saturated sodium bicarbonate solution and extracted with ethyl acetate (3×30 mL). The combined organic layer was dried over sodium sulphate, filtered, and concentrated under reduced pressure. Trituration with n-pentane (50 ml) gave a solid that was dried under high vacuum to give the title compound as an off-white solid (0.055 g, Yield: 37%, 0.06 mmol). ¹H NMR (DMSO-d6, 400 MHz): δ 3.63-3.73 (m, 3H), 4.06-4.12 (m, 2H), 4.34 (bs, 2H), 4.48 (s, 1H), 4.60 (s, 1H), 4.72 (s, 2H), 5.01 (s, 2H), 5.99-6.04 (m, 2H), 6.81-6.96 (m, 2H), 7.23-7.29 (m, 6H), 9.12 (bs, 2H). LCMS (Method A): 1.289 min, MS: ES+447.2 (M+1).

### Example 91 2-(2-(Benzyloxy)-4,6-dihydroxybenzoyl)isoindoline-4-carbonitrile

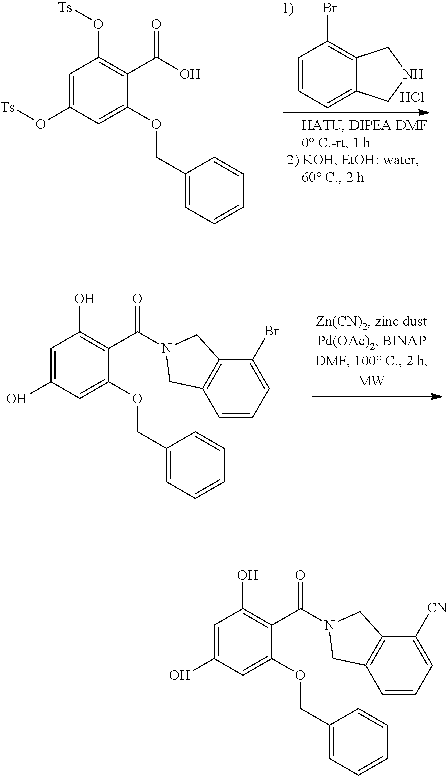 [SMILES: Cc1ccc(S(=O)(=O)Oc2cc(OCc3ccccc3)c(C(=O)O)c(OS(=O)(=O)c3ccc(C)cc3)c2)cc1 ; Brc1cccc2c1CNC2 ; Cl ; O=C(c1c(O)cc(O)cc1OCc1ccccc1)N1Cc2cccc(Br)c2C1 ; N#Cc1cccc2c1CN(C(=O)c1c(O)cc(O)cc1OCc1ccccc1)C2]

### 5-(Benzyloxy)-4-(4-bromoisoindoline-2-carbonyl)-1,3-phenylene bis(4-methylbenzenesulfonate)

A stirred solution of 2-(benzyloxy)-4,6-bis (tosyloxy) benzoic acid (Intermediate 61) (0.5 g, 0.88 mmol, 1 eq) in DMF (5 mL) was treated with HATU (0.434 g, 1.15 mmol, 1.3 eq) and DIPEA (0.171 g, 1.32 mmol, 1.5 eq). 4-bromoisoindoline hydrochloride (0.247 g, 0.97 mmol, 1.1 eq) was added to reaction mixture and stirred for 10 min at 0° C. The resulting reaction mixture was stirred at room temperature for 1 h. The reaction mixture poured into water (100 mL) and extracted using ethyl acetate (3×110 mL). The combined organic layer was washed with ice cold water (3×50 mL), dried over Na₂SO₄, filtered, and concentrated under vacuum. The crude was purified by flash column chromatography (product eluted with 20% EtOAc in hexane) yielding the title compound (0.400 g, Yield: 62.28%, 0.53 mmol). LCMS (Method A): 2.863 min, 2.878 min, MS: ES+748.2 (M+1).

### ((2-(Benzyloxy)-4,6-dihydroxyphenyl)(4-bromoisoindolin-2-yl)methanone (Intermediate

A stirred solution of 5-(benzyloxy)-4-(4-bromoisoindoline-2-carbonyl)-1,3-phenylene bis(4-methylbenzenesulfonate) (0.4 g, 0.53 mmol, 1 eq.) in EtOH:H₂O (4 mL) (1:1) at rt was treated with KOH (1.20 g, 30 mmol, 40 eq.). The reaction mixture was heated at 60° C. for 2 h, poured into water (100 mL), acidified using dil. HCl solution (pH˜ 7) and extracted with ethyl acetate (3×110 mL). The combined organic layer was dried over Na₂SO₄, filtered, and concentrated under vacuum. The crude material was purified by flash chromatography (product eluted with 3.6% MeOH in DCM) yielding the title compound as an off-white solid (0.20 g, Yield: 50.1%). ¹H NMR (DMSO-d6, 400 MHz): δ ppm 3.16 (d, J=4.8 Hz, 4H), 4.12 (d, J=4.8 Hz), 4.60-4.67 (m, 2H), 4.85 (bs, 1H), 5.04 (d, J=7.6 Hz, 1H), 6.02 (s, 2H), 7.25-7.29 (m, 6H), 7.40-7.51 (m, 2H), 9.54-9.66 (m, 2H). LCMS (Method A): 1.787 min, MS: ES+440.01, 442 (M) (M+2).

### 2-(2-(Benzyloxy)-4,6-dihydroxybenzoyl)isoindoline-4-carbonitrile (Example 91)

A stirred solution of (2-(benzyloxy)-4,6-dihydroxyphenyl)(4-bromoisoindolin-2-yl)methanone (Intermediate 64) (0.2 g, 0.45 mmol, 1 eq.) in DMF (2 mL) at rt was treated with Zn(CN)₂ (0.26 g, 0.22 mmol, 0.5 eq.), zinc dust (0.005 g, 0.076 mmol, 0.2 eq.), and BINAP (0.028 g, 0.045 mmol, 0.1 eq.). The reaction mixture was degassed using N₂ for 20 mins. Pd(OAc)2 (0.010 g, 0.04 mmol, 0.1 eq.). The reaction mixture was heated at 100° C. for 2 h. The reaction mixture was poured into water (100 mL) and extracted in ethyl acetate (3×110 mL). The combined organic layer was dried over Na₂SO₄, filtered, and concentrated under vacuum. The crude material was purified by flash chromatography (product eluted with 40% EtOAc in hexane) to give the title compound as off-white solid (0.020 g, Yield: 11.4%). ¹H NMR (DMSO-d6, 400 MHz), compound is a mixture of two rotamers: δ 4.55 (bs, 2H), 4.82-4.88 (m, 2H), 5.04 (d, J=6.8 Hz, 2H), 6.05 (2 singlets, 2H), 7.21-7.28 (m, 5H), 7.48-7.54 (m, 1H), 7.61 (d, J=5.2 Hz, 1H), 7.73-7.80 (m, 1H), 9.55-9.62 (m, 2H). LCMS (Method F): 1.677 min, MS ES+387.22 (M+1).

### Example 92 1-(2-(2-(Benzyloxy)-4,6-dihydroxybenzoyl)isoindolin-4-yl)azetidine-3-carbonitrile

A stirred solution of (2-(benzyloxy)-4,6-dihydroxyphenyl)(4-bromoisoindolin-2-yl)methanone (Intermediate 64) (0.4 g, 0.91 mmol, 1 eq), azetidine-3-carbonitrile hydrochloride (CAS 345954-83-8) (0.21 g, 1.78 mmol, 2 eq), K₃PO₄ (0.56 g, 2.64 mmol, 3 eq) and Xanthphos (0.084 g, 0.17 mmol, 0.2 eq) in 1,4-dioxane (8 mL) in a 35 mL microwave glass vial was degassed using N₂ gas for 10 min. Pd₂(dba)₃ (0.08 g, 0.087 mmol, 0.1 eq) was added and the reaction mixture heated to 100° C. for 1.5 h under microwave conditions. The reaction mixture was filtered through celite and the bed washed with ethyl acetate (100 mL). The obtained filtrate was poured into ice cold water (100 mL) and extracted in ethyl acetate (3×100 mL). The combined organic layer was dried over Na₂SO₄, filtered, and concentrated under vacuum. The crude material was purified by flash chromatography followed by prep TLC using 3% MeOH in DCM yielding the title compound as an off white solid (0.013 g, Yield: 3.2%, 0.03 mmol). ¹H NMR (DMSO-d6, 400 MHz): 5 ppm: 3.48-3.49 (m, 1H), 3.70-3.71 (m, 1H), 3.83-3.85 (m, 1H), 3.95-3.99 (m, 1H), 4.19-4.23 (m, 1H), 4.41 (m, 2H), 4.60-4.62 (m, 2H), 5.04-5.06 (m, 2H), 6.01 (s, 2H), 6.36 (d, J=8 Hz, 1H), 6.68-6.84 (m, 1H), 7.12-7.20 (m, 1H), 7.26-7.3 (m, 5H), 9.56 (d, J=27.6 Hz, 2H). LCMS (Method A): 1.718 min, MS: ES+442.2 (M+1).

### Example 93 (4-Aminoisoindolin-2-yl)(2-(benzyloxy)-4,6-dihydroxyphenyl)methanone

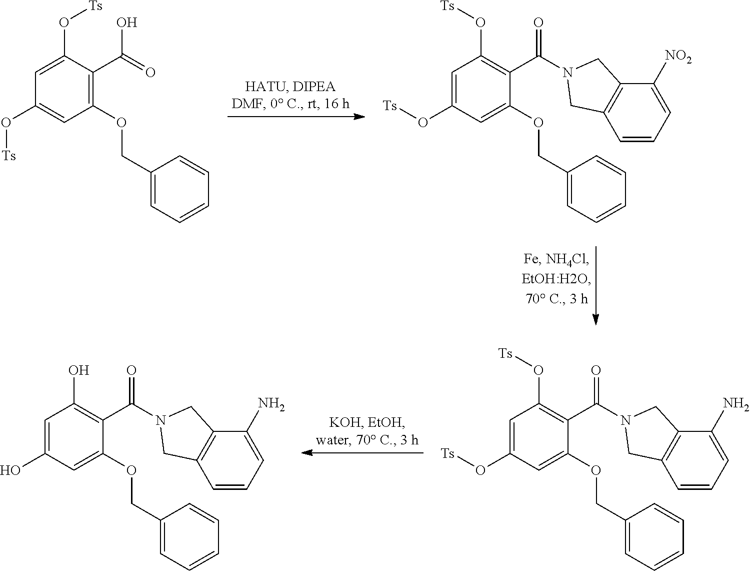 [SMILES: Cc1ccc(S(=O)(=O)Oc2cc(OCc3ccccc3)c(C(=O)O)c(OS(=O)(=O)c3ccc(C)cc3)c2)cc1 ; Cc1ccc(S(=O)(=O)Oc2cc(OCc3ccccc3)c(C(=O)N3Cc4cccc([N+](=O)[O-])c4C3)c(OS(=O)(=O)c3ccc(C)cc3)c2)cc1 ; Nc1cccc2c1CN(C(=O)c1c(O)cc(O)cc1OCc1ccccc1)C2 ; Cc1ccc(S(=O)(=O)Oc2cc(OCc3ccccc3)c(C(=O)N3Cc4cccc(N)c4C3)c(OS(=O)(=O)c3ccc(C)cc3)c2)cc1]

### 5-(Benzyloxy)-4-methyl-6-(4-nitroisoindoline-2-carbonyl)-1,3-phenylene bis(4-methylbenzenesulfonate) (Intermediate 65)

A stirred solution 2-(benzyloxy)-4,6-bis(tosyloxy)benzoic acid (Intermediate 61) (2.0 g, 3.56 mmol, 1 eq) in DMF (10 mL) at 0° C. was treated with HATU (1.73 g, 4.55 mmol, 1.3 eq) and DIPEA (0.6 ml, 4.63 mmol, 1.5 eq). The reaction mixture was stirred at 0° C. for 10 min. 4-Nitroisoindoline (CAS:748735-45-7) (0.635 g, 3.87 mmol, 1.1 eq) was added at 0° C. and the reaction mixture stirred at room temperature for 16 h. The reaction mixture was poured into ice cold water (200 mL) and extracted using ethyl acetate (3×220 mL), dried over Na₂SO₄, filtered, and concentrated under vacuum. The crude material was purified by flash column chromatography (product eluted with 23.6% ethyl acetate in hexane) to give the title compound as a yellow solid (1.2 g, Yield: 47.7%)._LCMS (Method A): 2.565 min. MS: ES+715.23 (M+1).

### 4-(4-Aminoisoindoline-2-carbonyl)-5-(benzyloxy)-1,3-phenylene bis(4-methylbenzenesulfonate) (Intermediate 66)

A stirred solution of 5-(benzyloxy)-4-methyl-6-(4-nitroisoindoline-2-carbonyl)-1,3-phenylene bis(4-methylbenzenesulfonate) (Intermediate 65) (1.2 g, 1.68 mmol, 1 eq.) in EtOH:H₂O (2:1) at rt was treated with Fe (0.56 g, 10.03 mmol, 6 eq.) and NH₄Cl (1.35 g, 25.23 mmol, 15 eq). The reaction mixture was heated at 70° C. and stirred for 3 h. The reaction mixture was cooled and passed through a celite bead using ethyl acetate (20 mL) and poured into ice cold water (20 mL) and extracted in ethyl acetate (2×20 mL). The combined organic layer was dried over Na₂SO₄, filtered, and concentrated under vacuum yielding the title compound as a brown sticky solid (0.75 g, Yield: 65.2%). LCMS (Method A): 2.431 min, 2.487 min, MS: ES+685.2 (M+1).

### (4-Aminoisoindolin-2-yl)(2-(benzyloxy)-4,6-dihydroxyphenyl)methanone (Example 93)

A stirred solution of 4-(4-aminoisoindoline-2-carbonyl)-5-(benzyloxy)-1,3-phenylene bis(4-methylbenzenesulfonate) (Intermediate 66) (0.7 g, 1.02 mmol, 1 eq.) in EtOH:H₂O (4:1) at rt was treated with KOH (2.29 g, 40.89 mmol, 40 eq.). The reaction mixture was heated at 60° C. and stirred for 2 h. The reaction mixture was acidified using saturated KHSO₄ solution (pH˜7) and extracted into ethyl acetate (3×45 mL). The combined organic layer was dried over Na₂SO₄, filtered, and concentrated under vacuum. The crude material was purified by normal phase column chromatography (product eluted with 5% MeOH in DCM) yielding the title compound as an orange solid (0.098 g, Yield: 25.5%). ¹H NMR (DMSO-d6, 400 MHz), compound is a mixture of rotamers: δ 4.33-4.39 (m, 2H), 4.52-4.63 (m, 2H), 5.03-5.08 (m, 3H), 5.17 (s, 1H), 5.99-6.01 (2 singlets, 2H), 6.37-6.52 (m, 2H), 6.91-6.98 (m, 1H), 7.22-7.30 (m, 5H), 9.45-9.54 (m, 2H)._LCMS (Method A): 1.491 min, MS ES+377.22 (M+1).

### Example 94 N-(2-(2-(benzyloxy)-4,6-dihydroxybenzoyl)isoindolin-4-yl)acetamide

A stirred solution of (4-aminoisoindolin-2-yl)(2-(benzyloxy)-4,6-dihydroxyphenyl)methanone (Example 93) (0.05 g, 0.13 mmol, 1 eq.) in AcOH (0.5 mL) at 0° C. was treated dropwise with acetic anhydride (0.1 mL). The resulting reaction mixture was stirred at room temperature for 1 h. The reaction mixture was neutralized using sat. NaHCO₃ solution and extracted in ethyl acetate (3×10 mL). The combined organic layer was washed with brine solution (10 mL) and dried over Na₂SO₄, filtered, and concentrated under vacuum. The crude material was purified by Prep TLC yielding the title compound as an off white solid (0.020 g, Yield: 36%). ¹H NMR (DMSO-d6, 400 MHz), compound is a mixture of rotamers: δ 1.985 and 2.085 (2 singlets, 3H), 4.49 (s, br, 2H), 4.69-4.73 (m, 2H), 5.03 (s, br, 2H), 6.00 (s, 2H), 6.99-7.29 (m, 7H), 7.53-7.55 (m, 1H), 9.47-9.60 (m, 3H). LCMS (Method A): 1.462 min, MS ES+419.2 (M+1).

### Example 95 N-((2-(2-(Benzyloxy)-4,6-dihydroxybenzoyl)isoindolin-4-yl)methyl)acetamide

A stirred solution of (4-(aminomethyl)isoindolin-2-yl)(2-(benzyloxy)-4,6-dihydroxyphenyl)methanone hydrochloride (Example 83) (0.050 g, 0.11 mmol, 1 eq) in DMF (1 mL) at 0° C. was treated with TEA (0.023 g, 0.23 mmol, 2 eq) for 10 min. Acetic anhydride (0.012 g, 0.12 mmol, 1 eq) was added and the reaction mixture was allowed to warm to room temperature and stirred for 15 min. The reaction mixture was poured into ice cold water (100 mL) and extracted with ethyl acetate (3×100 mL). The combined organic layer washed with ice cold water (3×50 mL), dried over Na₂SO₄, filtered, and concentrated under vacuum. The crude material was purified by prep TLC to give the title compound as an off-white solid (0.015 g, Yield: 7.4%). ¹H NMR (DMSO-d6, 400 MHz), compound is a mixture of rotamers: δ 1.77 (s, 3H), 4.09-4.25 (m, 2H), 4.51-4.54 (m, 2H), 4.67-4.78 (m, 3H), 5.04 (s, 2H), 6.04-6.05 (m, 2H), 7.16-7.20 (m, 1H), 7.22-7.33 (m, 7H), 8.06-8.12 (m, 1H), 9.26-9.29 (m, 2H). LCMS (Method A): 1.420 min, MS: ES+433.1 (M+1).

### Example 96 (2,4-dihydroxy-6-(pyridin-3-ylmethoxy)phenyl)(4-((tetrahydrofuran-3-yl)amino)isoindolin-2-yl)methanone

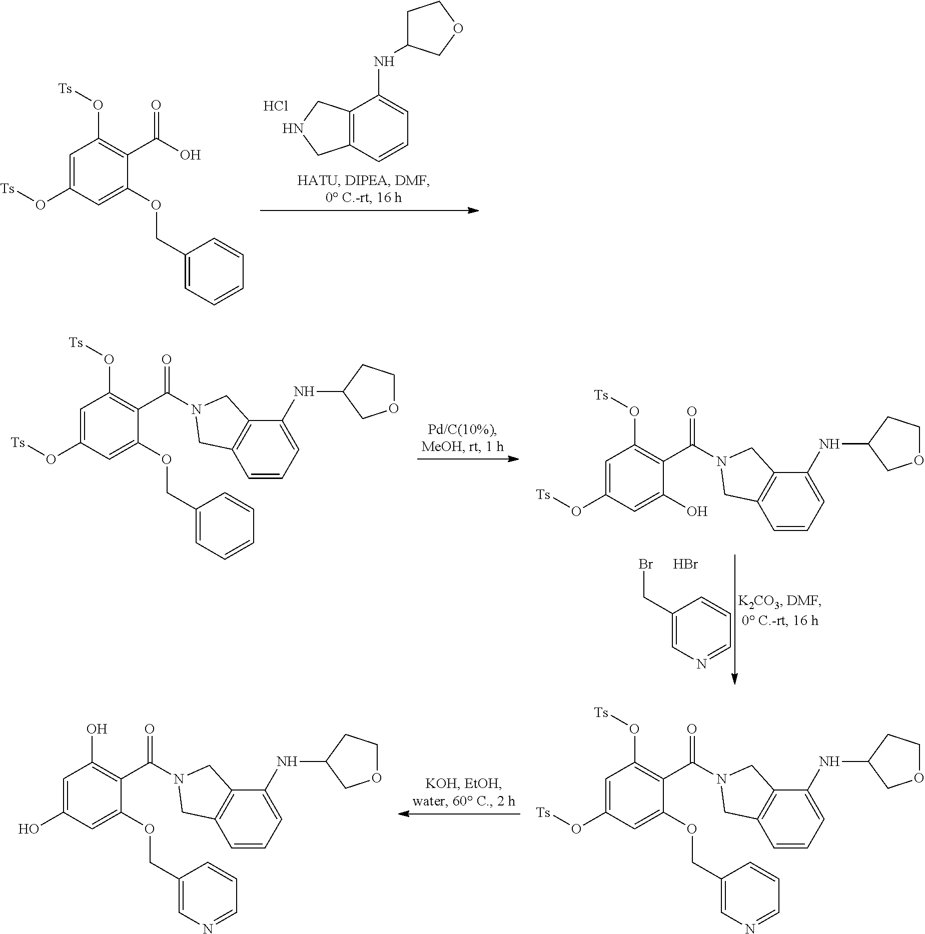

### 5-(Benzyloxy)-4-(4-((tetrahydrofuran-3-yl)amino)isoindoline-2-carbonyl)-1,3-phenylene bis(4-methylbenzenesulfonate) (Intermediate 67)

A stirred solution of 2-(benzyloxy)-4,6-bis(tosyloxy) benzoic acid (Intermediate 61) (0.5 g, 0.88 mmol, 1 eq) in DMF (5 mL) \t 0° C. was treated with HATU (0.501 g, 1.32 mmol, 1.5 eq), DIPEA (0.227 g, 1.75 mmol, 2 eq) and then N-(tetrahydrofuran-3-yl) isoindolin-4-amine hydrochloride (Intermediate 3) (0.233 g, 0.969 mmol, 1.1 eq). The resulting reaction mixture was allowed to stir at room temperature for 1 h. The reaction mixture was poured into ice cold water (100 mL) and extracted using ethyl acetate (3×100 mL). The combined organic layer washed with ice cold water (3×50 mL) and dried over Na₂SO₄, filtered and concentrated under vacuum.

The crude material was purified by flash column chromatography (product eluted in 45% EtOAc in hexane) yielding the title compound as an off white solid (0.4 g, Yield: 60.2%).

### 5-Hydroxy-4-(4-((tetrahydrofuran-3-yl)amino)isoindoline-2-carbonyl)-1,3-phenylenebis(4-methylbenzenesulfonate) (intermediate 68)

A stirred solution of 5-(benzyloxy)-4-(4-((tetrahydrofuran-3-yl)amino)isoindoline-2-carbonyl)-1,3-phenylene bis(4-methylbenzenesulfonate) (Intermediate 67) (0.7 g, 0.93 mmol, 1 eq) in MeOH (10 mL) at rt was charged with 10% Pd/C (0.35 g, 50% w/w). The resulting reaction mixture was stirred at room temperature under an H₂ atmosphere for 1 h. The reaction mixture was passed through celite bed, the bed was washed with MeOH (200 mL). The combined filtrate was concentrated under vacuum and the crude material triturated using n-pentane (3×10 mL) and diethyl ether (20 mL) yielding the title compound as an off-white solid (0.6 g, Yield: 92.6%). LCMS (Method A): 2.213 min, 2.258 min, MS: ES+665.0 (M+1).

### 5-(Pyridin-3-ylmethoxy)-4-(4-((tetrahydrofuran-3-yl)amino)isoindoline-2-carbonyl)-1,3-phenylene bis(4-methylbenzenesulfonate)

A stirred solution of 5-hydroxy-4-(4-((tetrahydrofuran-3-yl)amino)isoindoline-2-carbonyl)-1,3-phenylenebis(4-methylbenzenesulfonate) (Intermediate 68) (0.250 g, 0.37 mmol, 1.0 eq) in DMF (5 mL) at 0° C. was treated with 3-(bromomethyl)pyridine hydrobromide (0.105 g, 0.41 mmol, 1.1 eq) (CAS: 4916-55-6) and K₂CO₃ (0.156 g, 1.13 mmol, 3 eq). The reaction mixture was stirred at room temperature for 16 h. The reaction mixture was poured into ice-cold water (50 mL) and extracted in ethyl acetate (3×50 mL). The combined organic layer was further washed with water (3×50 mL). The combined organic layer was dried over Na₂SO₄, filtered, and concentrated under vacuum. The crude material was purified by flash chromatography (product eluted in 2.5% MeOH:DCM) yielding the title compound as an off-white solid (0.2 g, Yield: 70.5%). LCMS (Method A): 2.138 min, 2.220 min, MS: ES+756.2 (M+1).

### (2,4-Dihydroxy-6-(Pyridin-3-ylmethoxy)phenyl)(4-((tetrahydrofuran-3-yl)amino)isoindolin-2-yl)methanone (Example 96)

A stirred solution of 5-(pyridin-3-ylmethoxy)-4-(4-((tetrahydrofuran-3-yl)amino)isoindoline-2-carbonyl)-1,3-phenylene bis(4-methylbenzenesulfonate) (0.180 g, 0.24 mmol, 1.0 eq.) in EtOH:H₂O (1:1) (3 mL) at rt was treated with KOH (0.534 g, 9.54 mmol, 40 eq.). The reaction mixture was heated to 60° C. for 2 h. The resulting reaction mixture was allowed to cool to room temperature, neutralized using saturated KHSO₄ solution (pH 7) and extracted in EtOAc (3×100 mL). The combined organic layer was dried over Na₂SO₄, filtered, and concentrated under reduced pressure. The obtained crude material was purified by flash column chromatography yielding the title compound as an off-white solid (0.050 g, Yield: 46.9%). High temperature ¹H NMR (DMSO-d6, 400 MHz, 345 K): δ 1.79-1.86 (m, 1H), 2.17-2.28 (m, 1H), 3.53-3.58 (m, 1H), 3.7-3.79 (m, 2H), 3.86-4.06 (m, 2H), 4.41 (d, J=22.4 Hz, 2H), 4.63 (d, J=30 Hz, 2H), 5.09 (s, 2H), 5.2-5.21 (m, br, 1H), 6.06-6.07 (m, 2H), 6.46-6.61 (m, 2H), 7.07 (s, br, 1H), 7.26-7.29 (m, 1H), 7.7 (d, J=8 Hz, 1H), 8.45 (d, J=4.0 Hz, 1H), 8.56 (s, 1H), 9.32 (s, br, 2H). LCMS (Method B): 1.151 min, MS: ES+448.1 (M+1).

The compounds in Table 3 were prepared by methods analogous to Example 96.

TABLE 3

| Ex | | | |
|---|---|---|---|
| No | Name | Structure | Data |
| 97 | (2,4-Dihydroxy-6- (pyridin-4- ylmethoxy)phenyl) (4-((tetrahydrofuran- 3-yl)amino) isoindolin- 2-yl)methanone | 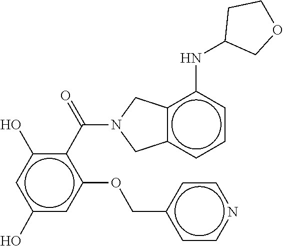 | Prepared by O-alkylation of Intermediate 68 with 4-(bromomethyl)pyridine (CAS 73870-24-3). High temperature ¹H NMR (DMSO-d6, 400 MHz, 345 K): δ 1.7 − 1.95 (m, 1H), 2.1 − 2.29 (m, 1H), 3.52 − 3.76 (m, 2H), 3.76 − 3.86 (m, 2H), 3.86 − 3.40 (m, 2H), 4.44 (d, J = 19.6 Hz, 2H), 4.67 (d, J = 30.4 Hz, 2H), 5.10 (s, 2H), 5.11 − 5.13 (m, 1H), 6.0 − 6.06 (2 singlets, 2H), 6.47 − 6.63 (m, 2H), 7.06 − 7.10 (m, 1H), 7.3 (d, J = 5.2 Hz, 2H), 8.45 (d, J = 4.8 Hz, 2H), 9.32 (m, 2H). LCMS (Method A): 1.122 min, MS: ES+ 448.1 (M + 1). |
| 98 | (2,4-Dihydroxy-6- ((tetrahydro-2H- pyran-4- yl)methoxy)phenyl) (4-((tetrahydrofuran- 3- yl)amino)isoindolin- 2-yl)methanone | 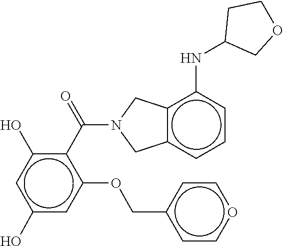 | Prepared by O-alkylation of Intermediate 68 using 4-(bromomethyl)tetrahydro-2H- pyran (CAS125552-89-8). ¹H NMR (DMSO-d6, 400 MHz): δ 1.15 − 1.18 (m, 2H), 1.46 − 1.49 (m, 2H) 1.79 − 1.82 (m, 2H), 2.09 − 2.21 (m, 1H), 3.17 − 3.18 (m, 2H), 3.34 − 3.52 (m, 1H), 3.57 − 3.58 (m, 1H), 3.59 − 3.70 (m, 5H), 3.76 − 4.03 (m, 2H), 4.3 − 4.37 (m, 2H), 4.54 − 4.62 (m, 2H), 5.33- 5.43 (m, 1H), 5.91 − 5.99 (m, 2H), 6.42 − 6.60 (m, 2H), 7.03 − 7.10 (m, 1H), 9.43 − 9.48 (m, 2H). LCMS (Method A): 1.463 min, MS ES+ 455.2 (M + 1). |
| 99 | (2,4-Dihydroxy-6- (pyridazin-3- ylmethoxy)phenyl)(4- ((tetrahydrofuran-3- yl)amino)isoindolin- 2-yl)methanone | 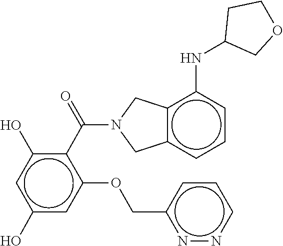 | Prepared by O-alkylation of Intermediate 68 using 3-(chloromethyl)pyridazine hydrochloride (CAS 27349-66-2). High temperature ¹H NMR (DMSO-d6, 400 MHz, 345 K) compound is a mixture of rotamers: δ 1.80 − 1.87 (m, 1H), 2.17 (bs, 1H), 3.34 − 3.60 (m, 2H), 3.7 − 3.87 (m, 1H), 3.93 − 3.99 (m, 1H), 3.99 − 4.17 (m, 1H), 4.44 (d, J = 22.8 Hz, 2H), 4.65 (d, J = 32.8, 2H), 5.17 (d, J = 37.2 Hz, 1H), 5.34 (s, 2H), 6.06 − 6.07 (m, 2H), 6.46 − 6.62 (m, 2H), 7.59 − 7.63 (m, 2H), 9.12 (s, 1H), 9.34 (m, 2H). LCMS (Method B): 1.279 min, MS: ES+ 449.1 (M + 1). |

### Example 100 8-Amino-3,4-dihydroisoquinolin-2(1H)-yl(2-(benzyloxy)-4,6-dihydroxyphenyl)methanone

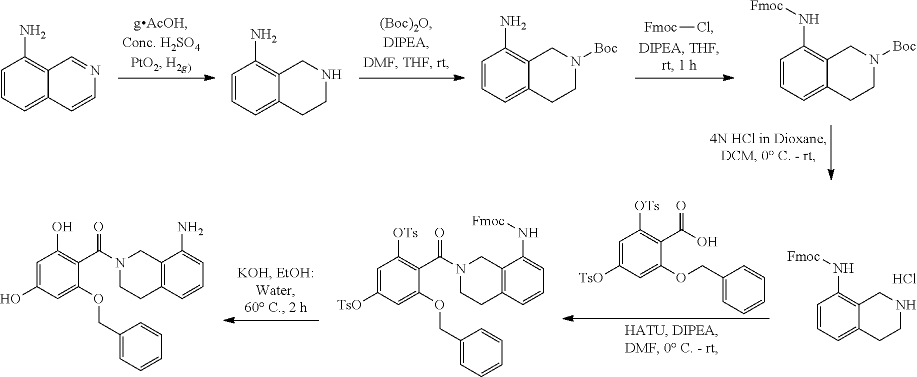

### 1,2,3,4-Tetrahydroisoquinolin-8-amine

A solution of isoquinolin-8-amine (5 g, 34.67 mmol, 1.0 eq.) (CAS: 23687-27-6) in glacial acetic acid (75 mL) was treated with cH₂SO₄ (0.5 mL) and PtO₂ (0.5 g, 10% w/w) at room temperature in an autoclave. The resulting reaction mixture was placed under an H₂ gas pressure of 55 kg/cm² at room temperature and stirred for 24 h. The reaction mixture was concentrated under reduced pressure and the residue was treated with aqueous K₂CO₃ solution (50 mL) and extracted with DCM (5×150 mL). The combined organic layer was dried over Na₂SO₄, filtered, and concentrated under reduced pressure. The crude material was triturated using diethyl ether (100 mL) and dried over high vacuum yielding the title compound (4.0 g, Yield: 77.8%). ¹H NMR (DMSO-d6, 400 MHz): δ ppm 2.63 (t, J=5.6 Hz, 2H), 2.94 (t, J=5.6 Hz, 2H), 3.63 (s, 2H), 4.70-4.75 (m, 2H), 6.29 (d, J=7.6 Hz, 1H), 6.43 (d, J=7.6 Hz, 1H), 6.81 (t, J=7.6 Hz, 1H). LCMS (Method A): 0.353 min, MS: ES+149.0 (M+1).

### t-Butyl 8-amino-3,4-dihydroisoquinoline-2(1H)-carboxylate

To a solution of 1,2,3,4-tetrahydroisoquinolin-8-amine (4.7 g, 31.75 mmol, 1 eq) in DMF:THF (470 mL, 1:1) were added DIPEA (4.09 g, 31.70 mmol, 1 eq) and Boc₂O (8.99 g, 41.28 mmol, 1.3 eq) at room temperature. The resulting reaction mixture was stirred for 1 h at rt. The reaction mixture was diluted with EtOAc (50 mL) and washed with cold brine solution (3×50 mL). The combined organic layer was dried over Na₂SO₄, filtered, and concentrated under reduced pressure. The crude material was purified by flash chromatography (product eluted in 1% MeOH in DCM) yielding the title compound (3.0 g, Yield: 38.1%). ¹H NMR (DMSO-d6, 400 MHz): δ ppm 1.43 (s, 9H), 2.64 (t, J=5.6 Hz, 2H), 3.49 (t, J=5.6 Hz, 2H), 4.12 (s, 2H), 4.86 (bs, 2H), 6.34 (d, J=7.2 Hz, 1H), 6.48 (d, J=7.6 Hz, 1H), 6.84 (t, J=7.6 Hz, 1H). LCMS (Method A): 1.762 min, MS: ES+193 (M−56).

### t-Butyl 8-((((9H-fluoren-9-yl)methoxy)carbonyl)amino)-3,4-dihydroisoquinoline-2(1H)-carboxylate

To a solution of t-butyl 8-amino-3,4-dihydroisoquinoline-2(1H)-carboxylate (3 g, 12.09 mmol, 1 eq) in THF (30 mL) at rt was added DIPEA (4.68 g, 36.29 mmol, 3 eq) and Fmoc-CI (3.12 g, 12.09 mmol, 1.3 eq). The resulting reaction mixture was stirred for 1 h at room temperature. The reaction mixture was poured into ice cold water (70 mL) and extracted with EtOAc (4×70 mL). The combined organic layer was dried over Na₂SO₄, filtered, and concentrate under reduced pressure. The crude material was purified by flash chromatography (product eluted in 7% EtOAc in Hexane) yielding (4.0 g, Yield: 70.4%). ¹H NMR (DMSO-d6, 400 MHz): δ ppm 1.40 (bs, 9H), 2.78 (t, J=4 Hz, 2H), 3.48-3.50 (m, 2H), 4.29-4.30 (m, 1H), 4.41-4.43 (m, 4H), 7.01-7.16 (m, 3H), 7.34-735 (m, 2H), 7.41-7.44 (m, 2H), 7.66-7.70 (bs, 2H), 7.90 (d, J=8 Hz, 2H), 9.12 (bs, 1H). LCMS (Method A): 2.609 min, MS: ES+493.1 (M+23).

### (9H-Fluoren-9-yl)methyl (1,2,3,4-tetrahydroisoquinolin-8-yl)carbamate hydrochloride

A stirred solution of t-butyl 8-((((9H-fluoren-9-yl)methoxy)carbonyl)amino)-3,4-dihydroisoquinoline-2(1H)-carboxylate (4 g, 8.51 mmol, 1.0 eq) in DCM (40 mL, 10 V) was cooled to 0° C. 4M HCl in dioxane (20 mL, 5 V) was added dropwise and the reaction mixture stirred at room temperature for 1 h. The reaction mixture was concentrated under vacuum. The isolated crude material was triturated using diethyl ether (100 mL) and dried under high vacuum yielding (3.3 g, Yield: 97%). ¹H NMR (DMSO-d6, 400 MHz): δ ppm 3.01-3.02 (m, 2H), 4.10 (bs, 2H), 4.29-4.33 (m, 1H), 4.46-4.48 (m, 2H), 7.05-7.25 (m, 3H), 7.35-7.46 (m, 4H), 7.72-7.74 (m, 2H), 7.92 (d, J=8 Hz, 2H), 9.24-9.36 (m, 2H).LCMS (Method A): 1.543 min, MS: ES+371.0 (M+1).

### 4-(8-((((9H-Fluoren-9-yl)methoxy)carbonyl)amino)-1,2,3,4-tetrahydroisoquinoline-2-carbonyl)-5-(benzyloxy)-1,3-phenylene bis(4-methylbenzenesulfonate)

A stirred solution of 2-(benzyloxy)-4,6-bis(tosyloxy) benzoic acid (Intermediate 61) (4.2 g, 7.39 mmol, 1.0 eq.) in DMF (42 mL) at rt was treated with HATU (4.21 g, 11.09 mmol, 1.5 eq.), DIPEA (2.86 g, 22.18 mmol, 3.0 eq.) and (9H-fluoren-9-yl) methyl (1,2,3,4-tetrahydroisoquinolin-8-yl)carbamate hydrochloride (3.30 g, 8.13 mmol, 1.1 eq.) and the mixture stirred at room temperature for 1 h. The resulting reaction mixture was diluted with ethyl acetate (100 mL) and washed with cold brine solution (4×100 mL). The organic layer was dried over Na₂SO₄, filtered, and concentrated under reduced pressure. The crude material was purified by flash chromatography (product was eluted in 8% methanol in DCM) yielding the title compound (5.50 g, Yield: 81%). LCMS (Method A): 2.933 min, 3.021 min. MS: ES+921.0 (M+1), 943 (M+23).

### 8-Amino-3,4-dihydroisoquinolin-2(1H)-yl(2-(benzyloxy)-4,6-dihydroxyphenyl)methanone Example 100

A stirred solution of 4-(8-((((9H-fluoren-9-yl)methoxy)carbonyl)amino)-1,2,3,4-tetrahydroisoquinoline-2-carbonyl)-5-(benzyloxy)-1,3-phenylene bis(4-methylbenzenesulfonate) (1 g, 1.08 mmol, 1.0 eq.) in EtOH:water (1:1) (10 mL) at rt was treated with aq KOH (2.43 g, 43.47 mmol, 40 eq.in 1 mL of water). The resulting reaction mixture was heated to 60° C. and stirred for 2 h. The resulting reaction mixture was allowed to cool to room temperature, poured into ice cold water (250 mL) and neutralized with diluted HCl (pH=˜7) and extracted with ethyl acetate (4×200 mL). The combined organic layer was dried over Na₂SO₄, filtered, and concentrated under reduced pressure. The obtained crude material was purified by flash chromatography (product eluted in 9.3% MeOH in DCM) yielding (8-amino-3,4-dihydroisoquinolin-2(1H)-yl)(2-(benzyloxy)-4,6-dihydroxyphenyl)methanone (0.22 g, Yield: 52%). High temperature ¹H NMR (DMSO-d6, 400 MHz, 345K): δ ppm 2.65-2.6 (m, 2H), 3.45-3.49 (m, 2H), 4.37-4.66 (m, 4H), 4.98 (s, 2H), 6.00 (s, 2H), 6.35 (d, J=6.8 Hz, 1H), 6.52 (d, J=7.6 Hz, 1H), 6.85 (t, J=8 Hz, 1H), 7.25-7.26 (m, 5H), 9.19-9.22 (m, 2H). LCMS (Method A): 1.532 min, MS: ES+391.0 (M+1).

### Example 101: N-(2-(2-(benzyloxy)-4,6-dihydroxybenzoyl)-1,2,3,4-tetrahydroisoquinolin-8-yl)acetamide

A stirred solution of (8-amino-3,4-dihydroisoquinolin-2(1H)-yl)(2-(benzyloxy)-4,6-dihydroxyphenyl)methanone (Example 100) (0.05 g, 0.12 mmol, 1.0 eq.) in acetic acid (0.5 mL) at 0° C. was treated dropwise with acetic anhydride (0.014 g, 0.14 mmol, 1.1 eq.). The reaction mixture was allowed to stir at room temperature for 30 min. The reaction mixture was poured into water (10 mL) and extracted with ethyl acetate (5×15 mL). The combined organic layer was dried over Na₂SO₄, filtered, and concentrated under reduced pressure. The crude material was triturated using DCM and diethyl ether yielding (0.04 g, Yield: 44%, 0.09 mmol). High temperature ¹H NMR (DMSO-d6, 400 MHz): δ ppm 1.98-2.01 (s, 3H), 2.75-2.77 (m, 2H), 3.49-3.50 (m, 2H), 4.54-4.55 (m, 2H), 4.95 (s, 2H), 6.01 (s, 2H), 6.96 (d, J=7.6 Hz, 1H), 7.11-7.24 (m, 6H), 9.17-9.20 (m, 2H). LCMS (Method A): 1.465 min, MS: ES+433.0 (M+1).

### Example 102 N-(2-(2-(Benzyloxy)-4, 6-dihydroxybenzoyl)-1, 2, 3, 4-tetrahydroisoquinolin-8-yl) tetrahydrofuran-3-carboxamide

A stirred solution of 4-(8-((((9H-fluoren-9-yl)methoxy)carbonyl)amino)-1,2,3,4-tetrahydroisoquinoline-2-carbonyl)-5-(benzyloxy)-1,3-phenylene bis(4-methylbenzenesulfonate) (2.0 g, 2.17 mmol, 1.0 eq.) in THF (20 mL) at 0° C. under nitrogen atmosphere was treated dropwise with DBU (0.66 g, 4.34 mmol, 2.0 eq). The resulting reaction mixture was stirred for 5 min. The resulting reaction mixture was poured into water (50 mL) and extracted with EtOAc (3×50 mL). The combined organic layer was dried over Na₂SO₄, filtered, and concentrated under reduced pressure. The crude material was purified by flash chromatography (product eluted in 3% methanol in DCM) yielding 4-(8-amino-1,2,3,4-tetrahydroisoquinoline-2-carbonyl)-5-(benzyloxy)-1,3-phenylene-bis(4-methylbenzenesulfonate) (1.0 g, Yield: 67%). LCMS (Method A): 2.529 min, 2.619 min, MS: ES+699.2 (M+1).

A stirred solution of tetrahydrofuran-3-carboxylic acid (CAS: 89364-31-8) (0.075 g, 0.648 mmol, 3.0 eq.) in DCM (1 mL) at 0° C. was treated with a catalytic amount of DMF. Oxalyl chloride (0.5 mL, 5.0 v) was added dropwise to the reaction mixture at 0° C. under a nitrogen atmosphere and stirred for 15 min. The resulting mixture was concentrated under high vacuum. In another vial, 4-(8-amino-1,2,3,4-tetrahydroisoquinoline-2-carbonyl)-5-(benzyloxy)-1,3-phenylenebis(4-methylbenzenesulfonate) (0.150 g, 0.143 mmol, 1.0 eq.) in THF was treated with DIPEA (0.07 mL, 0.642 mmol, 5.0 eq.) and cooled to 0° C. under nitrogen atmosphere and stirred for 15 min. The acid chloride was added dropwise and the reaction mixture stirred at 0° C. for 1 h. The resulting reaction mixture was poured into water (30 mL) and extracted using EtOAc (2×50 mL). The combined organic layer was dried over Na₂SO₄, filtered, and concentrated under reduced pressure. The crude material was purified by flash chromatography (product eluted in 3% methanol in DCM) yielding 5-(benzyloxy)-4-(8-(tetrahydrofuran-3-carboxamido)-1,2,3,4-tetrahydroisoquinoline-2-carbonyl)-1,3-phenylene bis(4-methylbenzenesulfonate) (0.270 g, Yield: 46%). LCMS (Method A): 2.491 min, 2.379 min, MS: ES+797.2 (M+1).

A stirred solution 5-(benzyloxy)-4-(8-(tetrahydrofuran-3-carboxamido)-1, 2, 3, 4-tetrahydroisoquinoline-2-carbonyl)-1, 3-phenylene bis (4-methylbenzenesulfonate) (0.270 g, 0.339 mmol, 1.0 eq.) in EtOH:water (2 mL, 1:1) at rt was treated with aq. KOH (0.800 g, 56.10 mmol, 40 eq. in minimum water) and heated to 60° C. for 2 h. The resulting reaction mixture was allowed to cool to room temperature, poured into ice cooled water (20 mL) and neutralized with dil. HCl (pH˜ 7) and extracted with ethyl acetate (4×20 mL). The combined organic layer was dried over Na₂SO₄, filtered, and concentrated under reduced pressure. The crude material was purified by prep. HPLC (see below) followed by lyophilization yielding the title compound (0.011 g, Yield: 7%). High temperature ¹H NMR (DMSO-d6, 400 MHz): δ ppm 2.01-2.05 (m, 1H), 2.30-2.31 (m, 1H), 2.77-2.78 (m, 2H), 3.51-3.88 (m, 4H), 4.51-4.52 (m, 1H), 4.91-4.93 (m, 2H), 6.00 (s, 2H), 7.00-7.23 (m, 5H), 9.26 (bs, 2H). LCMS (Method A): 1.490 min, MS: ES+489.4 (M+1). Analytical HPLC (Method C) 4.272, 98.3%. Prep HPLC conduced on a WATERS 2545 Quaternary system with WATERS 2489 UV Detector: Column X-bridge prep C18 (250×19 mm, 5 μm); compound eluted with: Mobile phase A: 0.1% Formic acid in Milli Q water, Mobile phase B: 20% Mobile phase A in Acetonitrile with a gradient of T=0 min (70% A, 30% B) to T=17 min (62% A, 38% B); T=17.01 min (2% A, 98% B) gradient to T=19 min (2% A, 98% B); T=19.01 min (70% A, 30% B) to T=22 min (70% A, 30% B);); flow rate=25 ml/min; analysis time 22 min.

### Example 103 t-Butyl (5-(4-((2-(2-(benzyloxy)-4,6-dihydroxybenzoyl)isoindolin-5-yl)methyl) piperazin-1-yl)pentyl)carbamate

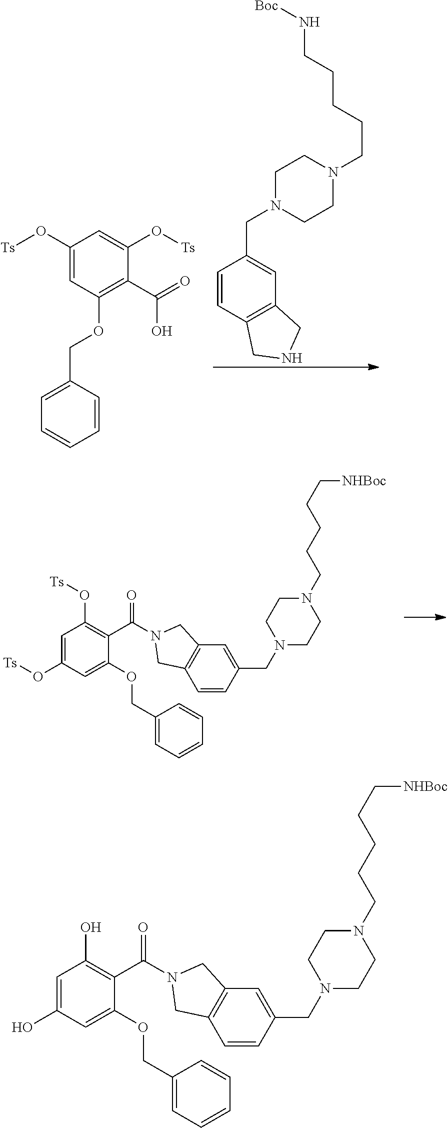

2-(Benzyloxy)-4,6-bis(tosyloxy)benzoic acid (Intermediate 61) (0.450 g, 0.792 mmol) in DMF (5 mL) at 0° C. was treated with HATU (0.451 g, 1.186 mmol), DIPEA (0.205 g, 1.586 mmol) and t-butyl (5-(4-(isoindolin-5-ylmethyl)piperazin-1-yl)pentyl)carbamate (Intermediate 69) (0.350 g, 0.869 mmol). The reaction mixture was stirred at room temperature for 1 h, poured into water (50 mL) and extracted into EtOAc (3×100 mL). The combined organic layer was washed with brine solution (3×50 mL), dried over sodium sulphate, filtered and concentrated under vacuum to give crude material (0.90 g) that was purified by column chromatography (silica gel, eluting with 3% (v/v) MeOH in DCM) yielding 5-(benzyloxy)-4-(5-((4-(5-((tert-butoxycarbonyl)amino)-pentyl)piperazin-1-yl)methyl)isoindoline-2-carbonyl)-1,3-phenylene-bis(4-methylbenzene-sulfonate) as an off-white solid (0.430 g, 57%). LCMS (Method E): 1.857 min, 1.877 min, MS: ES+953.4 (M+1); ¹H NMR (DMSO-d⁶, 400 MHz): δ 1.22-1.23 (m, 3H), 1.36 (s, 12H), 2.18 (s, 3H), 2.22-2.38 (m, 8H), 2.44 (s, 6H), 2.88 (brs, 2H), 3.46-3.48 (m, 2H), 3.70-3.74 (m, 1H), 4.28-4.34 (m, 1H), 4.54-4.66 (m, 2H), 5.05-5.13 (m, 2H), 6.64-6.65 (m, 1H), 6.79 (s, 1H), 7.03-7.12 (m, 2H), 7.19-7.32 (m, 8H), 7.52 (d, J=8.0 Hz, 2H), 7.64 (d, J=8.0 Hz, 2H), 7.77 (d, J=8.0 Hz, 2H).

A solution of 5-(benzyloxy)-4-(5-((4-(5-((tert-butoxycarbonyl)amino)pentyl) piperazin-1-yl)methyl)isoindoline-2-carbonyl)-1,3-phenylene bis(4-methyl benzene sulfonate) (0.400 g, 0.419 mmol) in EtOH:H₂O (4:1, 5 mL) was treated with KOH (0.940 g, 16.786 mmol) at room temperature. The reaction mixture was heated at 60° C. for 2 h, cooled to room temperature and acidified with saturated aqueous solution KHSO₄ and extracted with ethyl acetate (3×100 mL). The combined organic layer was dried over sodium sulphate and concentrated under vacuum to give crude material (0.175 g) that was purified by HPLC (see below). The isolated fractions were lyophilized yielding tert-butyl (5-(4-((2-(2-(benzyloxy)-4,6-dihydroxybenzoyl)isoindolin-5-yl)methyl)piperazin-1-yl)pentyl) carbamate as a white solid (0.025 g, 9%). LCMS (Method E): 1.265 min, MS: ES+645.4 (M+1); ¹H NMR (DMSO-d⁶, 400 MHz): δ 1.20-1.21 (m, 2H), 1.36 (s, 9H), 2.45-2.50 (m, 2H), 2.29-2.33 (m, 2H), 2.85-2.87 (m, 2H), 3.35-3.36 (m, 4H), 3.43-3.46 (m, 2H), 4.46 (s, 2H), 4.71 (s, 2H), 5.03 (s, 2H), 6.11 (s, 2H), 6.75-6.77 (m, 1H), 7.10-7.33 (m, 7H), 9.51 (br.s, 1H), 9.58 (br.s, 1H). Prep HPLC conditions was conducted using a Shimadzu LC20AP binary system with UV detector: Column X-Select CSH prep fluoro phenyl (250×19 mm, 5 μm); compound eluted with: Mobile phase A: 0.1% Formic acid in Milli Q water, mobile phase B: Acetonitrile with a gradient of T=0 min (95% A, 5% B) to T=15 min (76% A, 24% B); T=15.01 min (2% A, 98% B) gradient to T=17 min (2% A, 98% B); T=17.01 min (95% A, 5% B) to T=23 min (95% A, 5% B); ); flow rate=18 ml/min; analysis time 23 min.

### Example 104 (5-((4-(5-Aminopentyl)piperazin-1-yl)methyl)isoindolin-2-yl)(2-(benzyloxy)-4,6 dihydroxyphenyl)methanone hydrochloride

A solution of t-butyl (5-(4-((2-(2-(benzyloxy)-4,6-dihydroxybenzoyl)isoindolin-5-yl)methyl)piperazin-1-yl)pentyl)carbamate (Example 103) (0.020 g, 0.031 mmol) in DCM (1 mL) was treated with 4M HCl in dioxane (0.1 mL) at 0° C. The reaction mixture was stirred at room temperature for 30 mins, concentrated under vacuum and the crude material triturated with n-pentane (3×1 mL) followed by diethyl ether (3×1 mL). The obtained solid material was dried under high vacuum yielding (5-((4-(5-aminopentyl)piperazin-1-yl)methyl)isoindolin-2-yl)(2-(benzyloxy)-4,6-dihydroxyphenyl)methanone hydrochloride (Example 104) as a white solid (0.012 g, 68%). LCMS (Method E): 0.842 min, MS: ES+545.41 (M+1); ¹H NMR (DMSO-d⁶, 400 MHz): δ 1.33-1.34 (m, 2H), 1.56-1.68 (m, 4H), 2.67-2.76 (m, 2H), 3.02-3.04 (m, 2H), 3.40-3.44 (m, 8H), 3.57-3.59 (m, 2H), 4.50 (s, 2H), 4.75 (s, 2H), 5.04 (s, 2H), 6.03 (d, J=12 Hz, 2H), 7.23-7.30 (m, 5H), 7.46-7.48 (m, 2H), 7.90-7.91 (m, 3H), 9.56 (br.s, 1H), 9.65 (br.s, 1H).

### Example 105 5-((5-(4-((2-(2-(Benzyloxy)-4,6-dihydroxybenzoyl)isoindolin-5-yl)methyl)piperazin-1-yl)pentyl)carbamoyl)-2-(6-(dimethylamino)-3-(dimethyliminio)-3H-xanthen-9-yl)benzoate

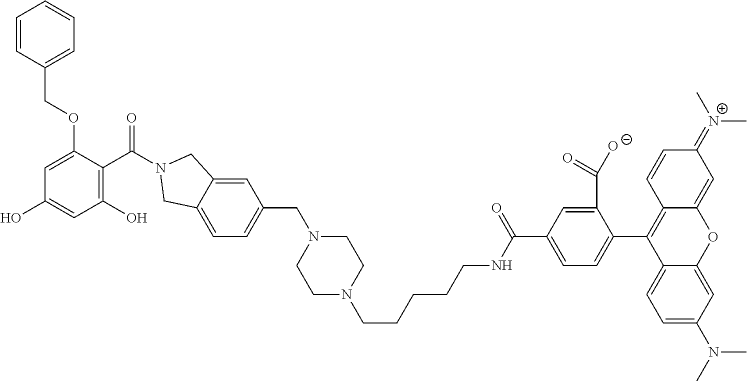 [SMILES: CN(C)c1ccc2c(-c3ccc(C(=O)NCCCCCN4CCN(Cc5ccc6c(c5)CN(C(=O)c5c(O)cc(O)cc5OCc5ccccc5)C6)CC4)cc3C(=O)[O-])c3ccc(=[N+](C)C)cc-3oc2c1]

To a solution of (5-((4-(5-aminopentyl)piperazin-1-yl)methyl)isoindolin-2-yl)(2-(benzyloxy)-4,6 dihydroxyphenyl)methanone hydrochloride (Example 104) (6.6 mg, 0.011 mmol) in N,N-dimethylformamide (1.0 mL) was added 5-TAMRA SE (5-carboxytetramethylrhodamine succinimidyl ester) (5.0 mg, 0.0095 mmol) and N,N-diisopropylethylamine (25 μL, 0.14 mmol). The reaction mixture was stirred at room temperature in the dark for 19 h. The reaction was diluted with DMSO, purified by reverse-phase HPLC (Method D) and freeze-dried to afford the title compound as a purple solid (3.0 mg, 33%). LCMS (Method I): 3.77 min, MS: ES+957.7 (M+1); ¹H NMR (DMSO-d⁶, 400 MHz): δ 1.30-1.38 (m, 2H), 1.42-1.49 (m, 2H), 1.53-1.61 (m, 2H), 2.23-2.37 (m, 1OH), 2.96 (s, 12H), 4.48 (s, 2H), 4.72 (s, 2H), 5.05 (s, 2H), 6.01-6.04 (m, 2H), 6.47-6.55 (m, 6H), 6.71 (s, 1H), 7.14-7.34 (m, 8H), 8.23 (td, J=1.7, 8.1 Hz, 1H), 8.44-8.46 (m, 1H), 8.78-8.82 (m, 1H), 9.44-9.69 (m, 2H). 4 protons obscured by H₂O peak.

### Example 106: t-Butyl (5-(2-(2-(benzyloxy)-4,6-dihydroxybenzoyl)isoindoline-5-carboxamido)pentyl)carbamate

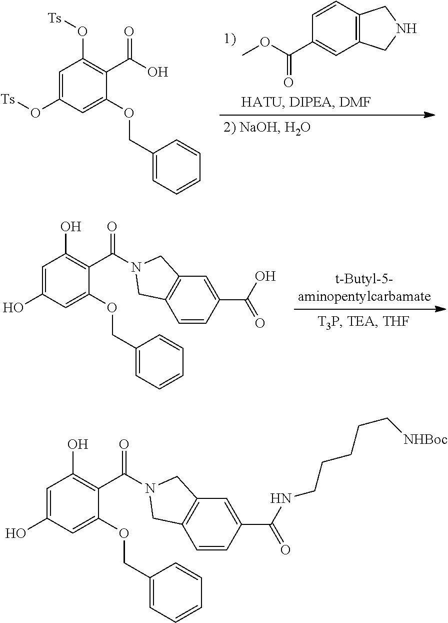 [SMILES: Cc1ccc(S(=O)(=O)Oc2cc(OCc3ccccc3)c(C(=O)O)c(OS(=O)(=O)c3ccc(C)cc3)c2)cc1 ; COC(=O)c1ccc2c(c1)CNC2 ; O=C(O)c1ccc2c(c1)CN(C(=O)c1c(O)cc(O)cc1OCc1ccccc1)C2 ; CC(C)(C)OC(=O)NCCCCCNC(=O)c1ccc2c(c1)CN(C(=O)c1c(O)cc(O)cc1OCc1ccccc1)C2]

2-(Benzyloxy)-4,6-bis(tosyloxy)benzoic acid (Intermediate 61) (2.50 g, 4.401 mmol) and methyl isoindoline-5-carboxylate hydrochloride (Intermediate 70, see preparation of Intermediate 73) (1.03 g, 4.840 mmol) in DMF (25 mL) were treated with HATU (2.5 g, 6.579 mmol) and DIPEA (1.70 g, 13.178 mmol) at 0° C. and stirred at 0° C. for 1 h. The resulting reaction mixture was diluted with EtOAc (150 mL) and washed with cold brine solution (4×150 mL). The combined organic layer was dried over sodium sulphate, filtered and concentrated under vacuum to give crude material (3.30 g) that was purified by flash chromatography (silica gel, eluting with 2.2% (v/v) MeOH in DCM) yielding methyl 2-(2-(benzyloxy)-4,6-bis(tosyloxy)benzoyl) isoindoline-5-carboxylate (2.50 g, 78%) that was progressed directly to the next step. LCMS (Method E): 2.618 min, 2.633 min, MS: ES+728.28 (M+1).

Methyl-2-(2-(benzyloxy)-4,6-bis(tosyloxy)benzoyl) isoindoline-5-carboxylate (1.0 μg, 1.375 mmol) in MeOH (5 mL) was treated with a solution of NaOH (0.270 g, 6.750 mmol) in water (5 mL) at room temperature and heated to 80° C. for 2 h. The reaction mixture was cooled to room temperature, diluted with water (150 mL) and neutralized with dilute HCl. The resulting precipitate was collected by filtration and dried under high vacuum yielding 2-(2-(benzyloxy)-4,6-dihydroxybenzoyl)isoindoline-5-carboxylic acid (Intermediate 62) as a white solid (0.51 g, 92%). LCMS (Method E): 1.478 min, MS: ES+406.17 (M+1); ¹H NMR (DMSO-d⁶, 400 MHz): δ 4.54 (s, 2H), 4.79 (s, 2H), 5.07 (s, 2H), 6.02 (s, 2H), 7.23-7.28 (m, 5H), 7.38 (d, J=8.4 Hz, 1H), 7.49 (d, J=8.0 Hz, 1H), 7.84-7.90 (m, 1H), 9.52 (s, 1H), 9.60 (d, J=2.40 Hz, 1H), 12.97 (br. s, 1H).

A mixture of 2-(2-(benzyloxy)-4,6-dihydroxybenzoyl)isoindoline-5-carboxylic acid (Intermediate 62) (0.500 g, 1.233 mmol), tert-butyl (5-aminopentyl)carbamate (CAS 51644-96-3) (0.27 g, 1.335 mmol) and TEA (0.24 g, 2.471 mmol) in THF (5 mL) was treated with propylphosphonic anhydride (T₃P) (50% solution in EtOAc) (1.176 mL, 1.849 mmol) added dropwise over 5 min at 0° C. The resulting reaction mixture was stirred at 0° C. for 1 h, diluted with EtOAc (75 mL) and washed with cold brine solution (4×80 mL). The combined organic layer was dried over sodium sulphate, filtered and concentrated under vacuum to give crude material (0.65 g) that was purified by column chromatography (silica gel, eluting with 3.5% (v/v) MeOH in DCM) yielding the title compound as a brown solid (0.110 g, 15%). LCMS (Method E): 1.844 min, MS: ES+490.27 (M−56); ¹H NMR (DMSO-d⁶, 400 MHz): δ 1.23-1.27 (m, 2H), 1.35 (d, J=4.40 Hz, 9H), 1.37-1.40 (m, 2H), 1.47-1.52 (m, 2H), 2.89 (t, J=6.0 Hz, 2H), 3.23 (d, J=6.4 Hz, 2H), 4.51 (s, 2H), 4.77 (s, 2H), 5.03 (s, 2H), 6.19 (s, 2H), 6.78 (d, J=5.2 Hz, 1H), 7.23-7.34 (m, 6H), 7.68-7.84 (m, 2H), 8.39-8.45 (m, 1H), 9.52 (d, J=2.4 Hz, 1H), 9.60 (s, 1H).

### Example 107 N-(5-Aminopentyl)-2-(2-(benzyloxy)-4,6-dihydroxybenzoyl)isoindoline-5-carboxamide hydrochloride

t-Butyl-(5-(2-(2-(benzyloxy)-4,6-dihydroxybenzoyl)isoindoline-5-carboxamido)-pentyl) carbamate (Example 106) (0.080 g, 0.136 mmol) in DCM (0.8 mL) was treated with 4M HCl in dioxane (0.8 mL) at 0° C. and stirred at room temperature for 1 h. The resulting reaction mixture was concentrated under vacuum and the crude material triturated with n-pentane (15 mL) followed by diethyl ether (15 mL) to give a solid material that was dried under high vacuum yielding the title compound as a brown solid (0.040 g, 56%). LCMS (Method E): 1.073 min, MS: ES+490.27 (M+1); ¹H NMR (DMSO-d⁶, 400 MHz): δ 1.30-1.39 (m, 2H), 1.50-1.61 (m, 4H), 2.73-2.78 (m, 2H), 3.21-3.28 (m, 2H), 4.52 (s, 2H), 4.77 (s, 2H), 5.03 (s, 2H), 6.03 (d, J=10 Hz, 2H), 7.20-7.34 (m, 5H), 7.45 (d, J=8.4 Hz, 1H), 7.71 (s, 1H), 7.77-7.85 (m, 3H), 8.45-8.51 (dt, J=5.2 Hz, 5.2 Hz, 1H), 9.57 (br. s, 1H), 9.63 (br. s, 1H).

### Example 108: 5-((5-(2-(2-(Benzyloxy)-4,6-dihydroxybenzoyl)isoindoline-5-carboxamido)pentyl) carbamoyl)-2-(6-(dimethylamino)-3-(dimethyliminio)-3H-xanthen-9-yl)benzoate

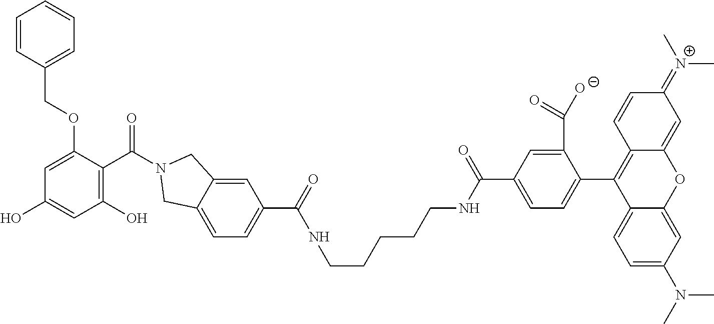 [SMILES: CN(C)c1ccc2c(-c3ccc(C(=O)NCCCCCNC(=O)c4ccc5c(c4)CN(C(=O)c4c(O)cc(O)cc4OCc4ccccc4)C5)cc3C(=O)[O-])c3ccc(=[N+](C)C)cc-3oc2c1]

To a solution of N-(5-aminopentyl)-2-(2-(benzyloxy)-4,6-dihydroxybenzoyl)isoindoline-5-carboxamide hydrochloride (Example 107) (6.0 mg, 0.011 mmol) in N,N-dimethylformamide (1.0 mL) was added 5-TAMRA SE (5-carboxytetramethylrhodamine, succinimidyl ester) (5.0 mg, 0.0095 mmol) and N,N-diisopropylethylamine (25 μL, 0.14 mmol). The reaction mixture was stirred at room temperature in the dark for 19 h. The reaction was diluted with DMSO, purified by reverse-phase HPLC (Method D) and freeze dried to afford the title compound as a purple solid (3.2 mg, 37%). LCMS (Method I): 3.59 min, MS: ES+902.5 (M+1); ¹H NMR (DMSO-d⁶, 400 MHz): δ 1.36-1.45 (m, 2H), 1.54-1.66 (m, 4H), 2.96 (s, 12H), 4.54 (d, J=7.6 Hz, 2H), 4.79 (d, J=4.4 Hz, 2H), 5.05 (s, 2H), 6.02-6.05 (m, 2H), 6.47-6.55 (m, 6H), 6.66 (s, 1H), 7.21-7.35 (m, 6H), 7.71-7.88 (m, 2H), 8.21-8.25 (m, 1H), 8.40-8.51 (m, 2H), 8.80-8.85 (m, 1H), 9.55 (s, 1H), 9.63 (s, 1H). 4 protons obscured by H₂O peak.

### Example 109: 1-(6-((5-(2-(2-(Benzyloxy)-4,6-dihydroxybenzoyl)isoindoline-5-carboxamido)pentyl)amino)-6-oxohexyl)-3,3-dimethyl-2-((1E,3E)-5-((E)-1,3,3-trimethylindolin-2-ylidene)penta-1,3-dien-1-yl)-3H-indol-1-ium formate

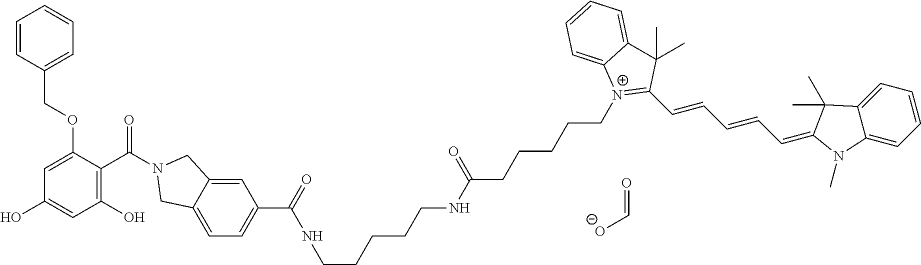

To a solution of N-(5-aminopentyl)-2-(2-(benzyloxy)-4,6-dihydroxybenzoyl)isoindoline-5-carboxamide hydrochloride (Example 107) (4.7 mg, 0.0090 mmol) in N,N-dimethylformamide (1.0 mL) was added Cyanine-5 NHS ester (CAS: 1263093-76-0, 5.0 mg, 0.0075 mmol) and N,N-diisopropylethylamine (20 μL, 0.11 mmol). The reaction mixture was stirred at room temperature in the dark for 19 h. The reaction was diluted with DMSO, purified by reverse-phase HPLC (Method D) and freeze dried to afford the title compound as a blue solid (2.6 mg, 36%). LCMS (Method J): 4.61 min, MS: ES+955.6 (M+1); ¹H NMR (DMSO-d⁶, 400 MHz): δ 1.24-1.41 (m, 7H), 1.46-1.58 (m, 5H), 1.69 (s, 12H), 2.01-2.08 (m, 2H), 2.97-3.04 (m, 2H), 3.21-3.27 (m, 2H), 3.60 (s, 3H), 4.06-4.12 (m, 2H), 4.54 (s, 2H), 4.78 (s, 2H), 5.04 (s, 2H), 6.02-6.06 (m, 2H), 6.26 (d, J=13.6 Hz, 1H), 6.32 (dd, J=3.8, 13.6 Hz, 1H), 6.54-6.61 (m, 1H), 7.05 (s, 1H), 7.22-7.46 (m, 11H), 7.62 (d, J=8.0 Hz, 2H), 7.68-7.85 (m, 3H), 8.30-8.48 (m, 3H), 9.78 (br.s, 1H), 9.93 (br.s, 1H).

### Example 110 N-(5-(4-((2-(2-(benzyloxy)-4,6-dihydroxybenzoyl)isoindolin-5-yl)methyl)piperazin-1-yl)pentyl)-3-(5,5-difluoro-7-(1H-pyrrol-2-yl)-5H-4λ⁴,5λ⁴-dipyrrolo[1,2-c:2′,1′-f][1,3,2]diazaborinin-3-yl)propenamide

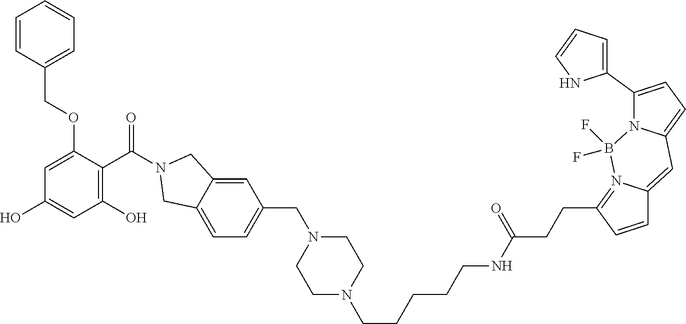

(5-((4-(5-Aminopentyl)piperazin-1-yl)methyl)isoindolin-2-yl)(2-(benzyloxy)-4,6-dihydroxyphenyl)methanone hydrochloride (Example 104) (20.0 mg, 0.034 mmol) was charged into a 100 mL RB flask and was dissolved in DMF (2 mL). N,N-diisopropylethylamine (15.0 μL, 0.086 mmol) was added and the resulting neutralized solution was stirred for 5 min. To the solution was added NanoBRET® 590-SE (17.6 mg, 0.041 mmol) and the mixture was capped and stirred for 1 hr. The mixture was diluted with 1:1:0.01 ACN, water, TFA and was subjected to reverse-phase preparative HPLC purification using Method 1 described for Examples 110 and 111 in HPLC Methods. Product containing fractions were pooled and concentrated to a dark purple solid that was dissolved in MeCN (10 mL) and concentrated to dryness in triplicate to remove TFA. The resulting residue was dried under high vacuum to give the product (25.0 mg, 84.9%) as a purple solid. MS (ESI, m/z) calcd. for C₄₈H₅₄BF₂N₇O₅ [M]⁺: 856.4, found 856.4.

### Example 111: 2-(2-(Benzyloxy)-4,6-dihydroxybenzoyl)-N-(5-(3-(5,5-difluoro-7-(1H-pyrrol-2-yl)-5H-4λ⁴,5λ⁴-dipyrrolo[1,2-c:2′,1′-f][1,3,2]diazaborinin-3-yl)propanamido)pentyl)isoindoline-5-carboxamide

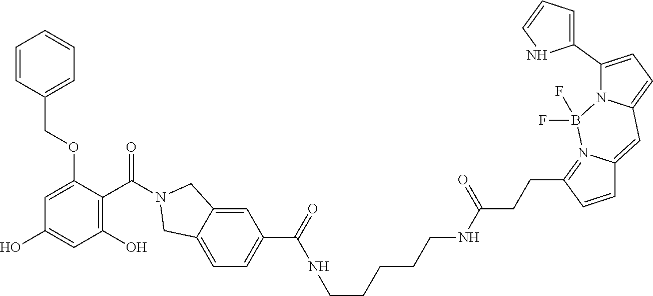

N-(5-Aminopentyl)-2-(2-(benzyloxy)-4,6-dihydroxybenzoyl)isoindoline-5-carboxamide hydrochloride (Example 107) (20.0 mg, 0.038 mmol) was charged into a 100 mL RB flask and was dissolved in DMF (2 mL). N,N-Diisopropylethylamine (16.6 μL, 0.095 mmol) was added and the resulting neutralized solution was stirred for 5 min. To the solution was added NanoBRET®590-SE (17.8 mg, 0.042 mmol) and the mixture was capped and stirred for 1 hr. The mixture was diluted with 1:1:0.01 MeCN, water, TFA and was subjected to reverse-phase preparative HPLC purification using Method 1. Product containing fractions were pooled and concentrated to a dark purple solid that was dissolved in ACN (10 mL) and concentrated to dryness in triplicate to remove TFA. The resulting residue was dried under high vacuum to give the product (25.5 mg, 83.5%) as a purple solid. MS (ESI, m/z) calcd. for C₄₄H₄₃BF₂N₆O₆ [M−F]⁺: 781.3, found 781.4.

### Example 149: 1-(3-((2-(2-(Benzyloxy)-4,6-bis(methoxymethoxy)benzoyl)-1,2,3,4-tetrahydroisoquinolin-8-yl)(methyl)amino)azetidin-1-yl)ethan-1-one

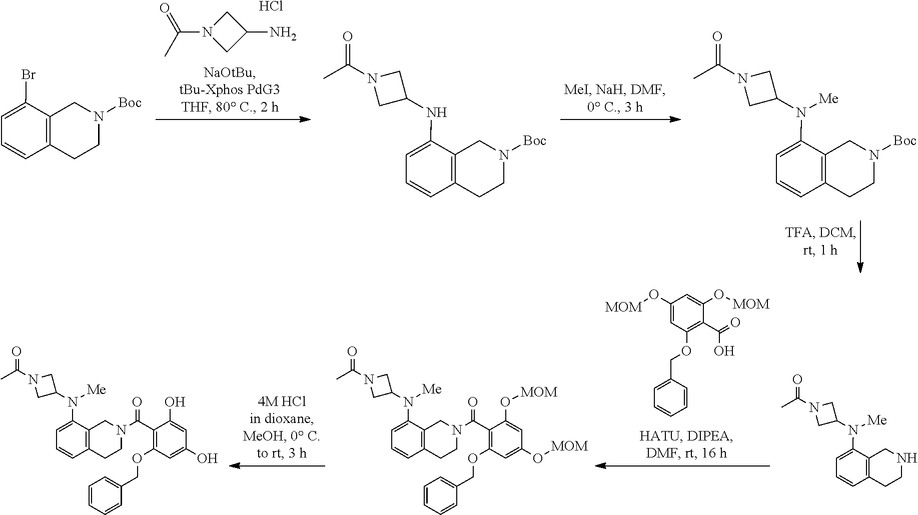

### t-Butyl 8-((1-acetylazetidin-3-yl)amino)-3,4-dihydroisoquinoline-2(1H)-carboxylate

Carried out in three parallel batches at 1.0 g scale. A stirred solution of t-butyl-8-bromo-3,4-dihydroisoquinoline-2(1H)-carboxylate (3 g, 9.61 mmol, 1 eq.) in THF (30 mL) at room temperature was treated with 1-(3-aminoazetidin-1-yl)ethan-1-one hydrochloride (2.17 g, 14.41 mmol, 1.5 eq.) (CAS: 1462921-50-1) and NaOtBu (2.77 g, 28.84 mmol, 3 eq.). The reaction mixture was degassed using N₂ gas for 30 mins. T-Buxphos PdG3 (0.40 g, 0.57 mmol, 0.06 eq.) was added and the resulting reaction mixture heated to 80° C. for 1 h, cooled, diluted with water (100 mL) and extracted with ethyl acetate (3×100 mL). The combined organic layer was dried over Na₂SO₄, filtered, and concentrated under vacuum. Crude material was purified by flash chromatography (silica 60-120) (product eluted in 5.5% MeOH in DCM) yielding the title compound (2.0 g, Yield: 60.3%). ¹H NMR (DMSO-d6, 400 MHz): δ ppm 1.44 (s, 9H), 1.77 (s, 3H), 2.67-2.70 (m, 2H), 3.50-3.53 (m, 2H), 3.71-3.76 (m, 1H), 3.91-3.94 (m, 1H), 4.16-4.19 (m, 2H), 4.31 (s, 2H), 4.43-4.47 (m, 1H), 5.52-5.54 (m, 1H), 6.23 (d, J=8 Hz, 1H), 6.51 (d, J=5.2 Hz, 1H), 6.99 (t, J=8 Hz, 1H). LCMS (Method A): 1.844 min. MS: ES+346 (M+1).

### t-Butyl 8-((1-acetylazetidin-3-yl)(methyl)amino)-3,4-dihydroisoquinoline-2(1H)-carboxylate

A stirred solution of t-butyl 8-((1-acetylazetidin-3-yl)amino)-3,4-dihydroisoquinoline-2(1H)-carboxylate (2.0 g, 5.79 mmol, 1 eq.) in DMF (20 mL) was cooled to 0° C. NaH (60% dispersion in oil) (1.3 g, 28.95 mmol, 5 eq.) was added portion-wise to the reaction mixture over 15 mins and then stirred for 1.5 h. Methyl iodide (4.0 g, 28.95 mmol, 5 eq.) (dissolved in DMF 5 mL) was added dropwise to the reaction mixture over 5 mins. The resulting reaction mixture was stirred at 0° C. for 3 h. The reaction mixture was diluted with ice cold water (100 mL) and extracted with ethyl acetate (3×100 mL). The combined organic layer was dried with Na₂SO₄, filtered, and concentrated under vacuum. Crude material was purified by trituration using n-pentane followed by high vacuum drying yielding the title compound (1.80 g, Yield: 81.7%). ¹H NMR (DMSO-d6, 400 MHz): δ ppm 1.41 (s, 9H), 1.73 (s, 3H), 2.78 (t, J=6 Hz, 2H), 3.17 (s, 3H), 3.30-3.35 (m, 2H), 3.50-3.52 (m, 1H), 3.75-3.77 (m, 1H), 3.90-3.94 (m, 1H), 3.99-4.04 (m, 1H), 4.18 (t, J=8 Hz, 1H), 4.49-4.53 (m, 2H), 6.85-6.86 (m, 1H), 6.93 (d, J=8.4 Hz, 1H), 7.15 (t, J=8 Hz, 1H). LCMS (Method A): 1.869 min, MS: ES+259.8 (M−100).

### 1-(3-(Methyl(1,2,3,4-tetrahydroisoquinolin-8-yl)amino)azetidin-1-yl)ethan-1-one.TFA

A stirred solution of t-butyl-8-((1-acetylazetidin-3-yl)(methyl)amino)-3,4-dihydroisoquinoline-2(1H)-carboxylate (1.7 g, 4.73 mmol, 1 eq.) in DCM (17 mL) was cooled to 0° C. TFA (8.5 mL) was added dropwise at 0′C and the reaction mixture stirred at room temperature for 1 h. The reaction mixture was concentrated under vacuum and crude material was triturated using diethyl ether (2×10 mL) followed by high vacuum drying yielding the title compound (3.5 g, Yield: Quantitative). ¹H NMR (DMSO-d6, 400 MHz): δ ppm 1.74 (s, 3H), 2.46 (s, 3H), 3.01 (t, J=6 Hz, 2H), 3.35-3.38 (m, 2H), 3.40-3.50 (m, 1H), 3.75-3.76 (m, 1H), 3.90 3.94 (m, 1H), 4.02-4.05 (m, 1H), 4.18-4.22 (m, 1H), 4.26 (s, 2H), 6.92 (d, J=8 Hz, 1H), 7.01 (d, J=7.6 Hz, 1H), 7.26 (t, J=7.6 Hz, 1H), 8.96 (bs, 3H). TFA salt. LCMS (Method A): 0.882 min, MS: ES+259.9 (M+1).

### 1-(3-((2-(2-(Benzyloxy)-4,6-bis(methoxymethoxy)benzoyl)-1,2,3,4-tetrahydroisoquinolin-8-yl)(methyl)amino)azetidin-1-yl)ethan-1-one

A stirred solution of 2-(benzyloxy)-4,6-bis(methoxymethoxy)benzoic acid (Intermediate 102) (1.5 g, 4.31 mmol, 1.0 eq.) in DMF (15 mL) at 0° C. was treated with HATU (2.45 g, 6.46 mmol, 1.5 eq.) and DIPEA (2.7 g, 21.55 mmol, 5.0 eq.) and stirred for 1 h. 1-(3-(methyl(1,2,3,4-tetrahydroisoquinolin-8-yl)amino)azetidin-1-yl)ethan-1-one.TFA (3.2 g, 8.62 mmol, 2.0 eq.) was added to the reaction mixture at 0° C. The resulting reaction mixture was stirred at room temperature for 16 h. The reaction mixture was diluted with ice-cold water (50 mL) and extracted with ethyl acetate (3×50 mL). The combined organic layer was washed with cold water (4×50 mL), dried over Na₂SO₄, filtered, and concentrated under vacuum to get crude material. The obtained crude material was purified by flash chromatography (silica 60-120) (product eluted in 4% MeOH in DCM) yielding the title compound (1.1 g, Yield: 32.3%). LCMS (Method A): 1.837 min, 2.057 min. MS: ES+590.09 (M+1) which was used directly in the next step.

### 1-(3-((2-(2-(Benzyloxy)-4,6-bis(methoxymethoxy)benzoyl)-1,2,3,4-tetrahydroisoquinolin-8-yl)(methyl)amino)azetidin-1-yl)ethan-1-one

A stirred solution of 1-(3-((2-(2-(benzyloxy)-4,6-bis(methoxymethoxy)benzoyl)-1,2,3,4-tetrahydroisoquinolin-8-yl)(methyl)amino)azetidin-1-yl)ethan-1-one (1.1 g, 1.69 mmol, 1 eq.) in DCM (10 ml) at 0° C. was treated with 4M HCl in dioxane (5 mL, 5.0 v) and the reaction mixture allowed to stir at room temperature for 4 h, then concentrated under vacuum. Crude material was purified by initially flash chromatography followed by Prep. HPLC purification. Pure fractions were lyophilized yielding the title compound as a white solid (0.070 g, Yield: 8.2%). ¹H NMR (DMSO-d6, 400 MHz, 348K): δ ppm 1.73 (s, 3H), 2.77-2.79 (m, 3H), 3.40-3.51 (m, 2H), 3.70-4.10 (m, 6H), 4.40-4.66 (m, 2H), 4.80-4.94 (m, 2H), 6.01 (d, J=5.2 Hz, 2H), 6.18 (s, 1H), 6.87-6.92 (m, 2H), 7.12-7.16 (m, 2H), 7.22 (s, 4H), 9.17-9.21 (m, 2H). LCMS (Method A): 1.650 min, MS: ES+501.98 (M+1). Analytical HPLC (Method B): 6.43 min. 100%. Prep. HPLC purification was conducted using SHIMADZU NEXERA prep with LH-40 autopurification system and UV detector; column Xtimate C18 (OBD 250×21.2 mm, 5 μm). Compound eluted with: Mobile phase A: 0.1% formic acid in Milli Q water, Mobile phase B: Acetonitrile; gradient of T=0 min (72% A, 28% B); to T=16.0 min (53% A, 47% B); T=16.01 min (2% A, 98% B) to T=18.0 min (2% A, 98% B); T=18.01 min (72% A, 28% B) to T=22.0 min (72% A, 28% B); flow rate=20 ml/min; analysis time 22 min.

### Example 150 (S)-1-(3-((2-(2-(Benzyloxy)-4,6-dihydroxybenzoyl)-1,2,3,4-tetrahydroisoquinolin-8-yl) (methyl)amino) pyrrolidin-1-vi)-4-methoxybutan-1-one

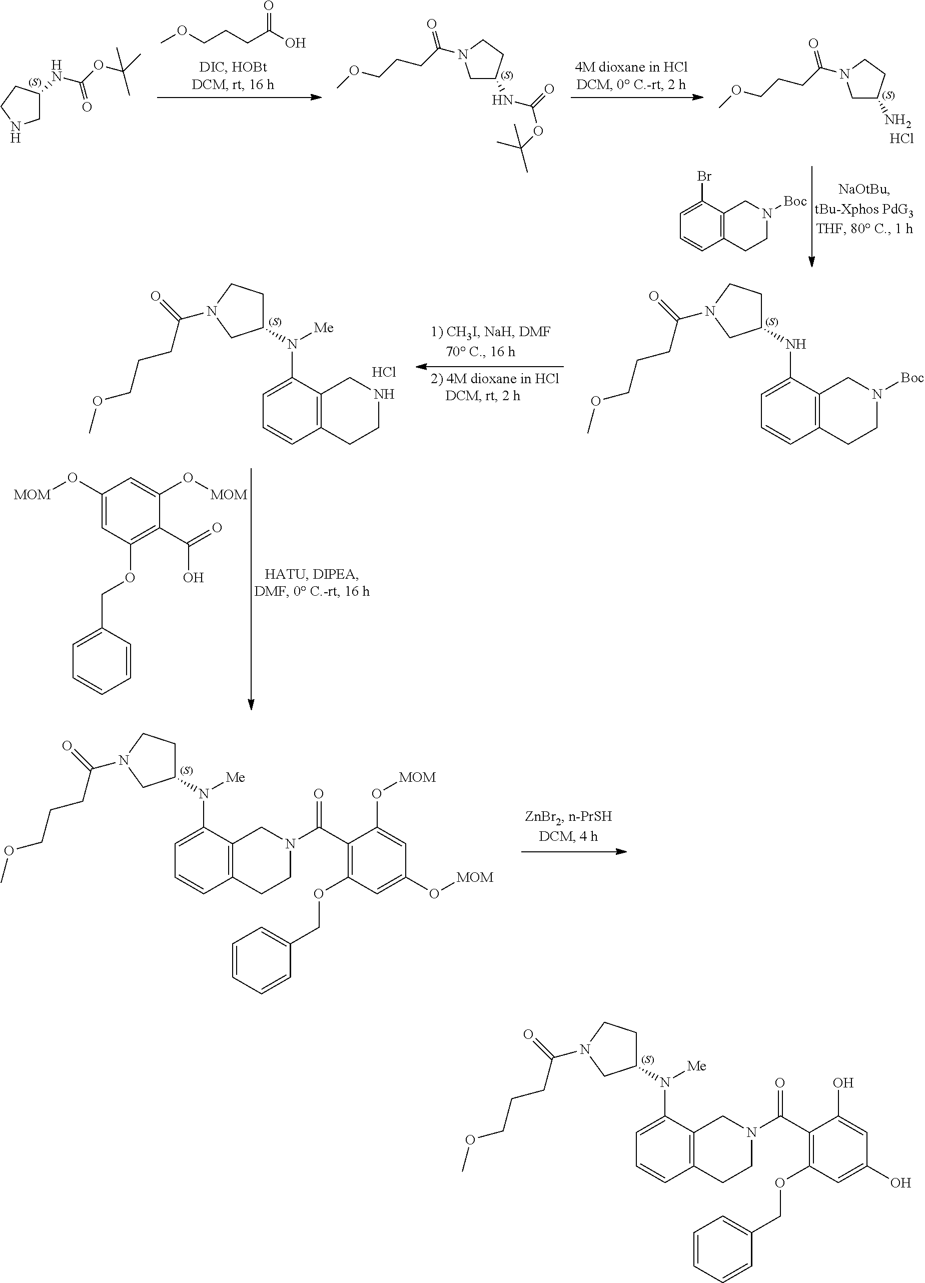

### t-Butyl (S)-(1-(4-methoxybutanoyl) pyrrolidin-3-yl) carbamate

Carried out in 2 parallel batches at 2.5 g scale. A stirred solution of 4-methoxybutanoic acid (2.5 g, 21.16 mmol, 1.0 eq.) (CAS: 29006-02-8) and t-butyl (S)-pyrrolidin-3-ylcarbamate (3.94 g, 21.16 mmol, 1.0 eq.) (CAS: 122536-76-9) in DCM (25 mL) at room temperature was treated with DIC (2.66 g, 21.16 mmol, 1.0 eq.) and HOBt (2.85 g, 21.16 mmol, 1.0 eq.). The reaction mixture was stirred at room temperature for 16 h. The reaction mixture was filtered and obtained filtrate was concentrated under vacuum. Crude material was purified by column chromatography (product was eluted at 1% MeOH in DCM solvent) yielding the title compound (4.0 g, 33%, 13.96 mmol). LCMS (Method A): 1.319 min, MS: ES+286.99 (M+1).

### (S)-1-(3-Aminopyrrolidin-1-yl)-4-methoxybutan-1-one hydrochloride

A stirred solution of t-butyl (S)-(1-(4-methoxybutanoyl) pyrrolidin-3-yl) carbamate (4.0 g, 13 mmol, 1.0 eq.) in DCM (40 mL) at 0° C. was treated with 4M HCl in dioxane (40 mL) The resulting reaction mixture was stirred at room temperature for 2 h. The reaction mixture was concentrated under vacuum and crude material purified by trituration using diethyl ether (2×25 mL) followed by high vacuum drying yielding the title compound (4.6 g, Yield: quantitative, 24.69 mmol). ¹H NMR (DMSO-d6, 400 MHz): δ ppm 1.68-1.74 (m, 2H), 1.90-2.27 (m, 4H), 3.31-3.45 (m, 2H), 3.47-3.49 (m, 2H), 3.51-3.62 (m, 3H), 3.69-3.73 (m, 2H). LCMS (Method B): 1.37 min, MS: ES+187.3 (M+1).

### t-Butyl (S)-8-((1-(4-methoxybutanoyl) pyrrolidin-3-yl) amino)-3, 4-dihydroisoquinoline-2(1H)-carboxylate

A stirred solution of t-butyl 8-bromo-3,4-dihydroisoquinoline-2(1H)-carboxylate (CAS: 893566-75-1) (4.0 g, 12.8 mmol, 1.0 eq.) and (S)-1-(3-aminopyrrolidin-1-yl)-4-methoxybutan-1-one hydrochloride (4.28 g, 19.2 mmol, 1.5 eq.) in THF at room temperature was treated with NaOtBu (3.07 g, 32 mmol, 2.5 eq.). The reaction mixture was degassed using N₂ (g) for 10 mins. T-BuxphosPdG₃ (0.61 g, 0.76 mmol, 0.06 eq.) was added and the resulting reaction mixture heated to 80° C. and stirred for 1 h. The reaction mixture was poured into water (100 mL) and extracted by ethyl acetate (2×150 mL). The combined organic layer was dried over Na₂SO₄, filtered, and concentrated under vacuum. Crude material was purified by flash chromatography (product was eluted at 5% MeOH in DCM) yielding the title compound (2.5 g, Yield: 46.7%, 5.98 mmol). ¹H NMR (DMSO-d6, D₂O Exchange, 400 MHz): δ ppm 1.41 (s, 9H), 1.68-1.74 (m, 2H), 1.85-2.16 (m, 2H), 2.19-2.28 (m, 2H), 2.66 (s, 2H), 3.18 (d, J=13.6 Hz, 3H), 3.27-3.38 (m, 3H), 3.38-3.59 (m, 3H), 3.96-4.09 (m, 1H), 4.25 (s, 2H), 6.46-6.55 (m, 2H), 7.00 (t, J=8 Hz, 1H). LCMS (Method A): 2.040 min, MS: ES+418.01 (M+1).

### t-Butyl (S)-8-((1-(4-methoxybutanoyl) pyrrolidin-3-yl) (methyl) amino)-3, 4-dihydroisoquinoline-2(1H)-carboxylate

A stirred solution of t-butyl (S)-8-((1-(4-methoxybutanoyl) pyrrolidin-3-yl) amino)-3, 4-dihydroisoquinoline-2(1H)-carboxylate (0.7 g, 1.67 mmol, 1.0 eq.) in DMF (35 mL) at 0° C. was treated with NaH (55-60% in oil) (0.64 g, 16.76 mmol, 10.0 eq.). The reaction mixture was stirred at room temperature for 3 h. Methyl iodide (3.5 mL) was added and the reaction mixture heated to 70° C. and stirred for 16 h. The reaction mixture was poured into ice-cold saturated solution of NH₄Cl (100 mL) and extracted by EtOAc (2×100 mL). The combined organic layer washed with brine solution (2×50 mL), organic layer dried by Na₂SO₄, filtered, and concentrated under vacuum. Crude material was purified by flash chromatography (product was eluted at 4% MeOH in DCM) yielding the title compound (0.4 g, Yield: 55.9%). ¹H NMR (DMSO-d6, 400 MHz): 1.41 (s, 9H), 1.66-1.72 (m, 2H), 1.72-2.04 (m, 2H), 2.05-2.23 (m, 2H), 2.50-2.57 (m, 3H), 2.77 (t, J=6 Hz, 2H), 2.95-2.96 (m, 1H), 3.14-3.20 (m, 3H), 3.25-3.29 (m, 2H), 3.40-3.62 (m, 4H), 3.62-3.73 (m, 1H), 4.49 (bs, 2H). LCMS (Method A): 2.062 min, MS ES+: 432.07 (M+1).

### (S)-4-Methoxy-1-(3-(methyl (1, 2, 3, 4-tetrahydroisoquinolin-8-yl) amino) pyrrolidin-1-vi) butan-1-one hydrochloride

A stirred solution of t-butyl-(S)-8-((1-(4-methoxybutanoyl)pyrrolidin-3-yl)(methyl)amino)-3,4-dihydroisoquinoline-2(1H)-carboxylate (0.4 g, 0.92 mmol, 1.0 eq.) in DCM (12 mL) at 0° C. was treated with 4M HCl in dioxane (4 mL). The reaction mixture was stirred at room temperature for 2 h and then concentrated under vacuum. Crude material was purified by trituration using diethyl ether (2×25 mL) followed by high vacuum drying yielding the title compound ((0.4 g, Yield: quantitative). ¹H NMR (DMSO-d6, D₂O exchange, 400 MHz): 1.65-1.69 (m, 3H), 1.70-2.10 (m, 3H), 2.16-2.23 (m, 2H), 2.97-3.00 (m, 3H), 3.14-3.18 (m, 4H), 3.25-3.31 (m, 4H), 3.39-3.45 (m, 2H), 3.54-3.84 (m, 3H), 4.18 (s, 2H), 7.02 (d, J=8.8 Hz, 1H), 7.20-7.22 (m, 1H), 7.27-7.30 (m, 1H). LCMS (Method B): 2.00 min, MS: ES+332.5 (M+1).

### (S)-1-(3-((2-(2-(Benzyloxy)-4,6-bis(methoxymethoxy)benzoyl)-1,2,3,4-tetrahydroisoquinolin-8-yl)(methyl)amino)pyrrolidin-1-yl)-4-methoxybutan-1-one

A stirred solution of 2-(benzyloxy)-4, 6-bis (methoxymethoxy) benzoic acid (Intermediate 102) (0.35 g, 1.00 mmol, 1.0 eq.) in DMF (3.5 mL) at 0° C. was treated with HATU (0.57 g, 1.50 mmol, 1.5 eq.) and DIPEA (0.38 g, 3.01 mmol, 3.0 eq.) and stirred for 10 mins. (S)-4-methoxy-1-(3-(methyl (1,2,3,4-tetrahydroisoquinolin-8-yl)amino)pyrrolidin-1-yl)butan-1-one hydrochloride (0.4 g, 1.10 mmol, 1.1 eq.) was added and the resulting reaction mixture stirred at room temperature for 16 h. The reaction mixture was poured into ice-cold water (50 mL) and extracted by ethyl acetate (2×100 mL). The combined organic layer was washed with brine solution (2×50 mL), dried with Na₂SO₄, filtered, and concentrated under vacuum. Crude material was purified by flash chromatography (product eluted at 6% MeOH in DCM) yielding the title compound (0.35 g, Yield: 52.7%). LCMS (Method A): 1.975 min, 1.992 min, 2.184 min, MS ES+: 662.25 (M+1).

### (S)-1-(3-((2-(2-(Benzyloxy)-4,6-dihydroxybenzoyl)-1,2,3,4-tetrahydroisoquinolin-8-yl) (methyl)amino) pyrrolidin-1-yl)-4-methoxybutan-1-one

A stirred solution of (S)-1-(3-((2-(2-(benzyloxy)-4,6-bis(methoxymethoxy)benzoyl)-1,2,3,4-tetrahydroisoquinolin-8-yl)(methyl)amino)pyrrolidin-1-yl)-4-methoxybutan-1-one (0.3 μg, 0.45 mmol, 1.0 eq.) in DCM (9 mL) at room temperature was treated with ZnBr₂ (0.407 g, 1.81 mmol, 4.0 eq.) followed n-PrSH (0.27 g, 3.62 mmol, 8.0 eq.). The resulting reaction mixture was stirred at room temperature for 4 h. The reaction mixture was poured into water (50 mL) and extracted by EtOAc (2×100 mL). The combined organic layer was dried by Na₂SO₄, filtered, and concentrated under vacuum. Crude material was purified by column chromatography (product eluted at 7% MeOH in DCM) yielding the title compound (0.09 g, Yield: 34.6%). ¹H NMR (DMSO-d6, 400 MHz): 1.22-1.35 (m, 4H), 1.66-1.73 (m, 3H), 2.15-2.24 (m, 2H), 2.31-2.36 (m, 2H), 2.50-2.58 (m, 2H), 2.67-2.74 (m, 2H), 3.15-3.20 (m, 3H), 3.21-3.31 (m, 3H), 3.90-4.40 (m, 2H), 4.40-5.00 (m, 4H), 5.93-5.98 (m, 2H), 6.90-6.99 (m, 2H), 7.10-7.25 (m, 6H), 9.39-9.49 (m, 2H) LCMS (Method A): 1.784 min, MS ES+: 574.04 (M+1). Analytical HPLC (Method B): 6.92 min. 96.95%.

### Example 151. (S)-1-(3-((2-(2-(Benzyloxy)-4,6-dihydroxybenzoyl)-1,2,3,4-tetrahydroisoquinolin-8-yl) amino) pyrrolidin-1-yl)-4-methoxybutan-1-one

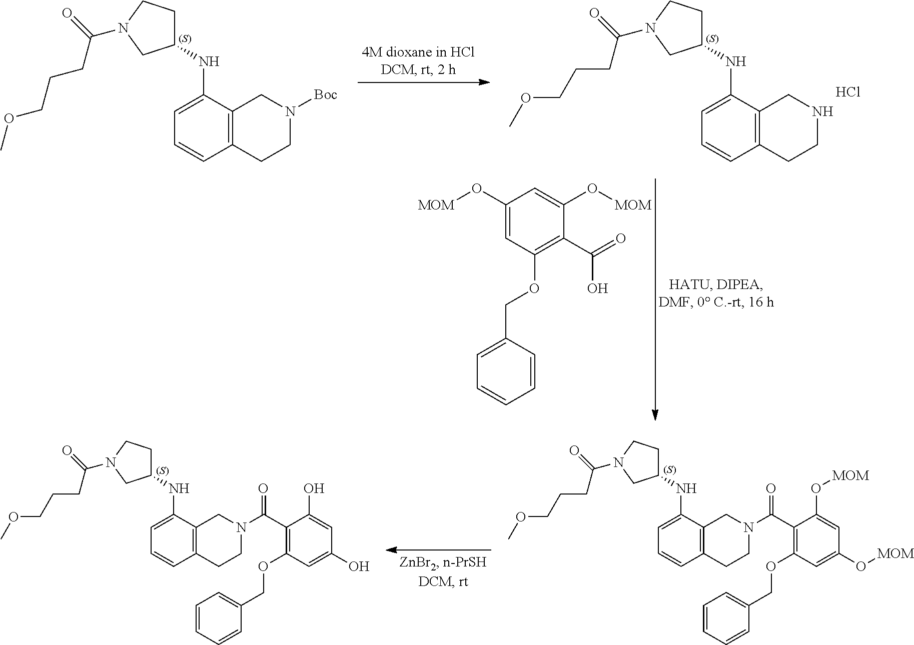

### (S)-4-Methoxy-1-(3-(1, 2, 3, 4-tetrahydroisoquinolin-8-yl) amino) pyrrolidin-1-yl) butan-1-one hydrochloride

A stirred solution of t-butyl (S)-8-((1-(4-methoxybutanoyl) pyrrolidin-3-yl) amino)-3,4-dihydroisoquinoline-2(1H)-carboxylate (0.5 g, 1.19 mmol, 1.0 eq.) in DCM (15 mL) at 0° C. was treated with 4M HCl in dioxane (5 mL). The reaction mixture was stirred at room temperature for 2 h and then concentrated under vacuum. Crude material was purified by trituration using diethyl ether (2×25 mL) followed by high vacuum drying yielding the title compound (0.45 g, Yield: quantitative). ¹H NMR (DMSO-d6, 400 MHz): δ ppm 1.69-1.74 (m, 2H), 1.80-1.90 (m, 1H), 2.00-2.10 (m, 1H), 2.20-2.30 (m, 1H), 2.28-2.30 (m, 1H), 2.93 (t, J=6 Hz, 2H), 3.22 (s, 3H), 3.25-3.26 (m, 2H), 3.30-3.38 (m, 4H), 3.45-3.50 (m, 1H), 3.58-3.99 (m, 4H), 3.99-4.00 (m, 2H), 6.55 (t, J=6.8 Hz, 1H), 6.62 (t, J=8 Hz, 1H), 7.08-7.13 (m, 1H), 9.73 (m, 2H). HCl salt. LCMS (Method A): 0.993 min, MS: ES+317.84 (M+1).

### (S)-1-(3-((2-(2-(Benzyloxy)-4, 6-bis (methoxymethoxy) benzoyl)-1, 2, 3, 4-tetrahydroisoquinolin-8-yl) amino) pyrrolidin-1-vi)-4-methoxybutan-1-one

A stirred solution of 2-(benzyloxy)-4, 6-bis (methoxymethoxy) benzoic acid (Intermediate 102) (0.35 g, 1.00 mmol, 1.0 eq.) in DMF (3.5 mL) at 0° C. was treated with HATU (0.57 g, 1.50 mmol, 1.5 eq.) and DIPEA (0.38 g, 3.01 mmol, 3.0 eq.) and stirred for 10 mins. (S)-4-methoxy-1-(3-((1,2,3,4-tetrahydroisoquinolin-8-yl) amino) pyrrolidin-1-yl) butan-1-one hydrochloride (0.42 g, 1.205 mmol, 1.2 eq.) was added to the reaction mixture at 0° C. The resulting reaction mixture was stirred at room temperature for 16 h. The reaction mixture was poured into ice-cold water (50 mL) and extracted by EtOAc (2×100 mL). The combined organic layer washed with brine solution (2×50 mL), dried with Na₂SO₄, filtered, and concentrated under vacuum. Crude material was purified by flash chromatography (product was eluted at 8% MeOH in DCM) yielding the title compound (0.38 g, Yield: 52.7%). LCMS (Method A): 1.922 min, 2.176 min, MS ES+: 648.15 (M+1).

### (S)-1-(3-((2-(2-(benzyloxy)-4,6-dihydroxybenzoyl)-1,2,3,4-tetrahydroisoquinolin-8-yl) amino) pyrrolidin-1-yl)-4-methoxybutan-1-one

A stirred solution of (S)-1-(3-((2-(2-(benzyloxy)-4,6-bis(methoxymethoxy)benzoyl)-1,2,3,4-tetrahydroisoquinolin-8-yl) amino) pyrrolidin-1-yl)-4-methoxybutan-1-one (0.35 g, 0.54 mmol, 1.0 eq.) in DCM (10.5 mL at room temperature was treated with ZnBr₂ (0.48 g, 2.16 mmol, 4.0 eq.) and n-PrSH (0.32 g, 4.32 mmol, 8.0 eq.) The reaction mixture was stirred at room temperature for 4 h. The reaction mixture was poured into water (50 mL) and extracted by EtOAc (2×100 mL). The combined organic layer was dried with Na₂SO₄, filtered, and concentrated under vacuum. Crude material was purified by column chromatography (product was eluted at 8% MeOH in DCM) yielding the title compound (0.12 g, Yield: 43%). ¹H NMR (DMSO-d6, 400 MHz): δ ppm 1.72-1.90 (m, 3H), 2.03-2.33 (m, 4H), 2.61-2.69 (m, 2H), 3.20 (d, J=8.8 Hz, 3H), 3.30-3.41 (m, 4H), 3.44-3.70 (m, 3H), 4.01 (bs, 1H), 4.11 (bs, 1H), 4.30-4.70 (m, 2H), 4.98-5.01 (m, 2H), 5.98 (s, 2H), 6.41-6.57 (m, 2H), 7.01 (t, J=7.6 Hz, 1H), 7.20-7.31 (m, 5H), 9.47-9.48 (m, 2H). LCMS (Method A): 1.748 min, MS ES+: 560.1 (M+1). Analytical HPLC (Method B): 6.46 min. 96.9%

### Preparation of Intermediates

### 2-(Benzyloxy)-4,6-bis(tosyloxy)benzoic acid (Intermediate 61)

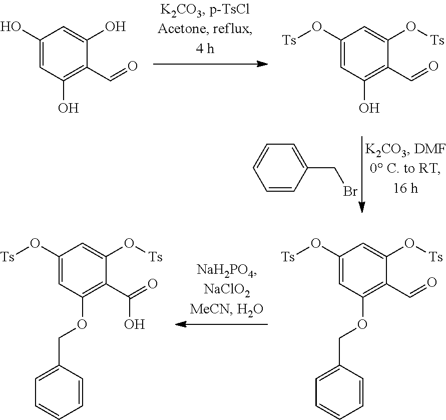 [SMILES: O=Cc1c(O)cc(O)cc1O ; Cc1ccc(S(=O)(=O)Oc2cc(O)c(C=O)c(OS(=O)(=O)c3ccc(C)cc3)c2)cc1 ; BrCc1ccccc1 ; Cc1ccc(S(=O)(=O)Oc2cc(OCc3ccccc3)c(C(=O)O)c(OS(=O)(=O)c3ccc(C)cc3)c2)cc1 ; Cc1ccc(S(=O)(=O)Oc2cc(OCc3ccccc3)c(C=O)c(OS(=O)(=O)c3ccc(C)cc3)c2)cc1]

### 5-(Benzyloxy)-4-formyl-1,3-phenylene bis(4-methylbenzenesulfonate) (Intermediate 71)

A solution of 2,4,6-trihydroxybenzaldehyde (CAS 487-70-7) (5 g, 32.2 mmol, 1.0 eq) in acetone (200 mL) at rt was treated with K₂CO₃ (22.27 g, 161 mmol, 5.0 eq) and p-toluene sulphonyl chloride (12.90, 67.7 mmol, 2.1 eq). The reaction mixture was heated at 60° C. for 4 h then allowed to cool to room temperature and concentrated under reduced pressure. The crude material was diluted with water (300 mL) and extracted in EtOAc (3×400 mL). The combined organic layer was washed with brine solution (200 mL), dried over sodium sulphate, and concentrated under reduced pressure to give crude material which was purified by column chromatography using silica gel (eluting product using 8.7% ethyl acetate in hexane) yielding 4-formyl-5-hydroxy-1,3-phenylene bis(4-methylbenzenesulfonate) as a yellow solid (5.9 g, Yield: 39.3%). ¹H NMR (DMSO-d⁶, 400 MHz): δ 2.44 (d, J=2.4 Hz, 6H), 6.38 (d, J=2.4 Hz, 1H), 6.66 (d, J=2.4 Hz, 1H), 7.47-7.54 (m, 4H), 7.69 (d, J=8.0 Hz, 2H), 7.78 (d, J=8.4 Hz, 2H), 9.89 (s, 1H), 11.52 (s, 1H). LCMS: 2.441 min, MS: ES+461.30 (M−1)

A solution of 4-formyl-5-hydroxy-1,3-phenylene bis(4-methylbenzenesulfonate) (20 g 1.0 eq) in DMF (200 mL) at 0° C. temperature was treated with K₂CO₃ (3.0 eq) and benzylbromide (6.72 g, 1.3 eq). The reaction mixture was stirred at room temperature for 16 h. The completion of reaction mixture was confirmed by TLC (20% ethyl acetate in hexane). The resulting reaction mixture was diluted with ethyl acetate (400 mL) and washed with chilled brine solution (3×500 mL). The combined organic layer was dried over Na₂SO₄, filtered and concentrated under reduced pressure. The obtained crude material (21.5 g) was purified by column chromatography using silica gel (eluting product with 16%) to give the title compound 5-(benzyloxy)-4-formyl-1,3-phenylene bis(4-methylbenzenesulfonate) (Intermediate 71) as an off-white solid (20 g, Yield: 83%). 1H NMR (DMSO-d6, 400 MHz): δ ppm 2.44 (d, J=2.4 Hz, 6H), 5.15 (s, 2H), 6.46 (d, J=2 Hz, 1H), 7.07 (d, J=2 Hz, 1H), 7.39-7.41 (m, 5H), 7.46-7.51 (m, 4H), 7.65 (d, J=8.4 Hz, 2H), 7.74 (d, J=8.4 Hz, 2H), 10.00 (s, 1H). LCMS (Method A): 2.651 min, MS: ES+574.9 (M+23).

### 2-(Benzyloxy)-4,6-bis(tosyloxy)benzoic acid (Intermediate 61)

A solution of 5-(benzyloxy)-4-formyl-1,3-phenylene bis(4-methylbenzenesulfonate) (Intermediate 71) (20 g, 1.0 eq.) in MeCN:Water (200 mL) at room temperature was treated with NaClO₂ (12.73 g, 3.7 eq.) and NaH₂PO₄ (9.12 g, 2.0 eq.). The resulting reaction mixture was stirred at room temperature for 16 h. MeCN was removed under vacuum and the resulting aqueous solution diluted with water (300 mL) and neutralized with dil. HCl (pH˜7) then extracted with ethyl acetate (2×500 mL). The combined organic layer was dried over Na₂SO₄, filtered, and concentrated under reduced pressure. Crude material was triturated using diethyl ether (2×200 mL) and n-pentane (100 mL) to give 2-(benzyloxy)-4,6-bis(tosyloxy)benzoic acid (Intermediate 61) as an off-white solid (20.0 g, Yield: 97%). ¹H NMR (DMSO-d6, 400 MHz): δ ppm 2.43 (s, 6H), 5.03 (s, 2H), 6.52 (d, J=2 Hz, 1H), 6.85 (s, 1H), 7.32-7.37 (m, 5H), 7.47-7.49 (q, J=6.4, 8 Hz, 4H), 7.70 (t, J=8.4 Hz, 4H), 13.48 (bs, 1H). LCMS (Method A): 2.344 min, MS: ES+568.91 (M+1).

The following intermediates were prepared according to the methods described above

| Intermediate | | | |
|---|---|---|---|
| No | Name | Structure | Data |
| 10 | 2-Methoxy- 4,6- bis(tosyloxy) benzoic acid | 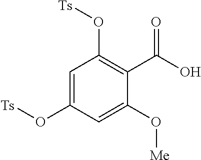 [SMILES: COc1cc(OS(=O)(=O)c2ccc(C)cc2)cc(OS(=O)(=O)c2ccc(C)cc2)c1C(=O)O] | ¹H NMR (DMSO-d6, 400 MHz): δ ppm 3.66 (s, 3H), 6.50 (s, 1H), 6.75 (s, 1H), 7.47 − 7.52 (m, 4H), 7.66 (d, J = 6.8 Hz, 2H), 7.76 (d, J = 6.4 Hz, 2H), 13.41 (bs, 1H). LCMS (Method A): 2.11 min, MS: ES+ 493.0 (M + 1). |
| 11 | 2-Ethoxy-4,6- bis(tosyloxy) benzoic acid | 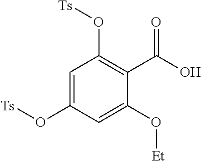 [SMILES: CCOc1cc(OS(=O)(=O)c2ccc(C)cc2)cc(OS(=O)(=O)c2ccc(C)cc2)c1C(=O)O] | ¹H NMR (DMSO-d6, 400 MHz): δ 1.15 − 1.22 (m, 3H), 2.42 − 2.50 (m, 6H), 3.86 − 3.94 (m, 2H), 6.50 − 6.53 (m, 1H), 6.70 (s, 1H), 7.45 − 7.51 (m, 4H), 7.66 − 7.76 (m, 4H), 13.33 (s, 1H). LCMS (Method A): 2.186 min, 2.217 min, MS: ES+ 507.12 (M + 1). |
| 18 | 2- (cyclopropyl methoxy)- 4,6- bis(tosyloxy) benzoic acid | 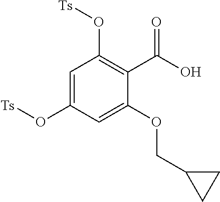 [SMILES: Cc1ccc(S(=O)(=O)Oc2cc(OCC3CC3)c(C(=O)O)c(OS(=O)(=O)c3ccc(C)cc3)c2)cc1] | ¹H NMR (DMSO-d6, 400 MHz): δ 0.21 − 0.27 (m, 2H), 0.46 − 0.54 (m, 2H), 0.98 − 1.06 (m, 1H), 2.28 − 2.43 (m, 6H), 3.72 (d, J = 6.8 Hz, 2H), 6.48 (s, 1H), 6.70 (s, 1H), 7.45 − 7.51 (m, 4H), 7.66 − 7.75 (m, 4H), 13.34 (bs, 1H). LCMS (Method A): 2.318 min, 2.350 min, MS: ES+ 555.11 (M + 23). |
| 19 | 2- (cyclohexylmethoxy)- 4,6- bis(tosyloxy) benzoic acid | 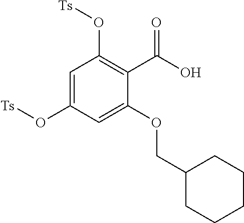 [SMILES: Cc1ccc(S(=O)(=O)Oc2cc(OCC3CCCCC3)c(C(=O)O)c(OS(=O)(=O)c3ccc(C)cc3)c2)cc1] | ¹H NMR (DMSO-d6, 400 MHz): 0.91 − 0.94 (m, 2H), 1.14 − 1.23 (m, 4H), 1.64 − 1.67 (m, 5H), 2.43 (m, 6H), 3.66 (m, 2H), 6.47 (s, 1H), 6.97 (s, 1H), 7.46 − 7.51 (m, 4H), 7.66 − 7.75 (m, 4H), 13.31 (bs, 1H). LCMS (Method A): 2.590 min, 2.784 min, MS: ES+ 575 (M + 1). |
| 27 | 2-((4- Fluorobenzyl) oxy)- bis(tosyloxy) benzoic acid | 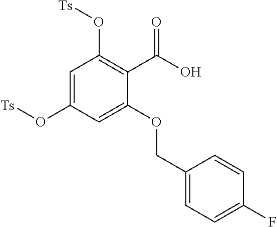 [SMILES: Cc1ccc(S(=O)(=O)Oc2cc(OCc3ccc(F)cc3)c(C(=O)O)c(OS(=O)(=O)c3ccc(C)cc3)c2)cc1] | ¹H NMR (DMSO-d6, 400 MHz): δ 2.44 (d, J = 4.0 Hz, 6H), 5.05 (s, 2H), 6.50 (d, J = 2.0 Hz, 1H), 6.93 (d, J = 2.0 Hz, 1H), 7.22 (t, J = 8.8 Hz, 2H), 7.37 − 7.40 (m, 2H), 7.48 − 7.51 (m, 4H), 7.67 (d, J = 7.6 Hz, 2H), 7.73 (d, J = 8.4 Hz, 2H), 13.42 (s, 1H). LCMS (Method A): 2.355 min, MS: ES+ 609.1 (M + 23) |
| 28 | 2-((3,4- Difluorobenzyl) oxy)-4,6- bis(tosyloxy) benzoic acid | 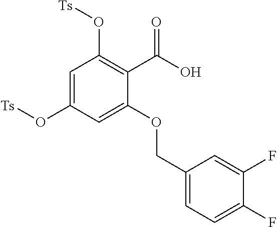 [SMILES: Cc1ccc(S(=O)(=O)Oc2cc(OCc3ccc(F)c(F)c3)c(C(=O)O)c(OS(=O)(=O)c3ccc(C)cc3)c2)cc1] | ¹H NMR (DMSO-d6, 400 MHz): 2.43 (2 singlets, 6H), 5.03 − 5.14 (m, 2H), 6.50 (d, J = 2 Hz, 1H), 6.71 (s, br, 1H), 6.92 (s, 1H), 7.19 − 7.44 (m, 1H), 7.38 (m, br, 1H), 7.41 − 7.49 (m, 4H), 7.64 − 7.75 (m, br, 4H), 13.10 − 13.45 (m, 1H). LCMS (Method A): 2.404 min, 2.440 min, MS: ES+ 627.0 (M + 23). |
| 29 | 2-((4- Methoxybenzyl) oxy)- 4,6- bis(tosyloxy) benzoic acid | 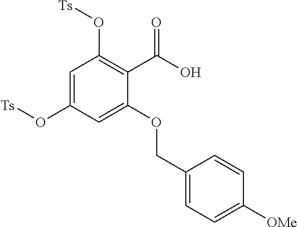 [SMILES: COc1ccc(COc2cc(OS(=O)(=O)c3ccc(C)cc3)cc(OS(=O)(=O)c3ccc(C)cc3)c2C(=O)O)cc1] | ¹H NMR (DMSO-d6, 400 MHz): δ 2.44 (d, J = 2.4 Hz, 6H), 3.76 (s, 3H), 4.95 (s, 2H), 6.50 (d, J = 2.0 Hz, 1H), 6.89 − 6.94 (m, 3H), 7.28 (t, J = 8.8 Hz, 2H), 7.41 − 7.56 (m, 24), 7.64 − 7.69 (m, 2H), 7.73 − 7.78 (m, 2H), 13.38 (s, 1H). LCMS (Method A): 2.329 min, MS: ES+ 621.1 (M+23) |
| 30 | 2-(Pyridin-2- ylmethoxy)- 4,6- bis(tosyloxy) benzoic acid | 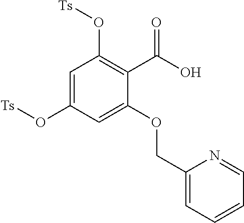 [SMILES: Cc1ccc(S(=O)(=O)Oc2cc(OCc3ccccn3)c(C(=O)O)c(OS(=O)(=O)c3ccc(C)cc3)c2)cc1] | ¹H NMR (DMSO-d6, 400 MHz): δ 2.44 (d, J = 7.2 Hz, 6H), 5.11 (s, 2H), 6.55 (d, J = 2.0 Hz, 1H), 6.90 (d, J = 2.0 Hz, 1H), 7.35 − 7.38 (m, 2H), 7.47 − 7.50 (m, 4H), 7.67 − 7.73 (m, 4H), 7.82 − 7.86 (m, 1H), 8.56 (t, J = 1.6 Hz, 1H), 13.54 (s, 1H). LCMS (Method A): 2.069 min, MS: ES+ 570.1 (M + 1) |
| 98 | 2-((3- Methylbenzyl) oxy)-4,6- bis(tosyloxy) benzoic acid | 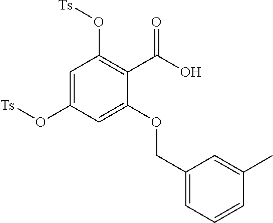 [SMILES: Cc1ccc(S(=O)(=O)Oc2cc(OCc3cccc(C)c3)c(C(=O)O)c(OS(=O)(=O)c3ccc(C)cc3)c2)cc1] | ¹H NMR (DMSO-d6, 400 MHz): δ 2.30 (s, 3H), 2.44 (d, J = 3.6 Hz, 6H), 5.00 (s, 2H), 6.50 (d, J = 1.2 Hz, 1H), 6.90 (d, J = 2.0 Hz, 1H), 7.10 − 7.15 (m, 3H), 7.25 − 7.28 (m, 1H), 7.48 − 7.51 (m, 4H), 7.67 (d, J = 8.4 Hz, 2H), 7.73 (d, J = 8.4 Hz, 2H), 13.42 (s, 1H). LCMS (Method A): 2.397 min, MS: ES+ 605.1 (M + 1) |

### (9H-Fluoren-9-yl)methyl 4-(isoindolin-5-ylmethyl)piperazine-1-carboxylate hydrochloride (Intermediate 1)

A stirred solution of t-butyl 5-(bromomethyl)isoindoline-2-carboxylate (CAS 201342-42-9) (0.5 g, 1.60 mmol, 1.0 eq) in DMF (5 mL, 10 v) at rt under a nitrogen atmosphere was treated with K₂CO₃ (0.66 g, 4.80 mmol, 3.0 eq.) and stirred for 15 min. 1-Piperazinecarboxylic acid-9H-fluoren-9-ylmethyl ester hydrochloride (CAS 215190-22-0) (0.49 g, 1.60 mmol,1.0 eq.) was added and the resulting reaction stirred at rt for 1 h. The resulting reaction mixture was poured into water (50 ml) and filtered, the residue was washed with n-pentane (30 mL) and dried under vacuum to give t-butyl 5-((4-(((9H-fluoren-9-yl)methoxy)carbonyl)piperazin-1-yl)methyl)isoindoline-2-carboxylate (Intermediate 72) (0.82 g, 1.52 mmol, Yield: 95%). LCMS (Method A): 1.661 min, MS: ES+540.3 (M+1).

A stirred solution of 5-((4-(((9H-fluoren-9-yl)methoxy)carbonyl)piperazin-1-yl)methyl)isoindoline-2-carboxylate (Intermediate 72) (0.8 g, 1.48 mmol, 1.0 eq.) in DCM (10 mL) was cooled to 0° C. 4N HCl in dioxane (5 mL) was added drop wise and the reaction mixture stirred for 16 hr at room temperature. The resulting reaction mixture was concentrated under reduced pressure and the crude material triturated using n-pentane (250 mL) yielding the title compound (0.65 g, 1.36 mmol, Yield: 65%). ¹H NMR (DMSO-d6, 400 MHz): δ 2.89-2.95 (m, 2H), 3.26-3.50 (m, 4H), 3.70-3.73 (m, 1H), 4.00 (m, br, 2H), 4.27-4.37 (m, 4H), 4.53 (s, br, 4H), 7.35 (t, J=7.2 Hz, 2H), 7.43-7.51 (m, 3H), 7.61-7.64 (m, 3H), 7.91 (d, J=7.2 Hz, 2H). 10.11 (s, br, 2H). LCMS (Method A): 1.026 min. MS: ES+440.3 (M+1)

### 3-(Isoindolin-4-yloxy)cyclobutane-1-carbonitrile (Intermediate 2)

t-Butyl-4-hydroxyisoindoline-2-carboxylate (CAS 871013-92-2) (0.5 g, 2.12 mmol, 1.0 eq.), 3-hydroxycyclobutane-1-carbonitrile (CAS 20249-17-6) (0.206 g, 2.12 mmol, 1.0 eq.) and triphenyl phosphine (1.11 g, 4.25 mmol, 2.0 eq.) in THF (0.5 mL, minimum solvent to make a paste) was sonicated for 15 min. DIAD (98%) (0.850 g, 4.25 mmol, 2.0 eq.) was added drop wise to the resulting reaction mixture under nitrogen atmosphere at 60° C. and stirred for 1 h. The resulting reaction mixture was allowed to cool to room temperature, poured into Ice-water (20 mL) and extracted with ethyl acetate (3×20 mL). The combined organic was dried over Na₂SO₄, filtered, and concentrated under reduced pressure. The crude material was purified by normal phase flash chromatography using (silica gel, 5% MeOH in DCM) yielding t-butyl 4-(3-cyanocyclobutoxy) isoindoline-2-carboxylate (0.510 g. Yield: 38.34%). ¹H NMR (DMSO-d6, 400 MHz): δ 1.22 (m, 2H), 1.46 (s, 9H), 2.79-2.82 (m, 2H), 3.46 (s, 1H), 4.47 (d, J=9.6 Hz, 2H), 4.57 (d, J=10 Hz, 2H), 5.01-5.05 (m, 1H), 6.73 (d, J=8.4 Hz, 1H), 6.92 (m, 1H), 7.24 (t, J=8.0 Hz, 1H). LCMS (Method A): 2.78 min, purity, MS: ES+215.2 (M−100)

A stirred solution of t-butyl 4-(3-cyanocyclobutoxy) isoindoline-2-carboxylate (0.5 g, 1.58 mmol, 1.0 eq.) in DCM (3 mL) at 0° C. was treated with TFA (1.80 mL, 10 eq) and stirred for 1 h.

The reaction mixture was evaporated and triturated using diethyl ether (20 mL). The solid material was dried under high vacuum to give the title compound as an off-white solid (0.3 g, yield: 88%). ¹H NMR (DMSO-d6, 400 MHz): δ 1.18-1.26 (m, 2H), 2.80-2.86 (m, 2H), 3.43-3.51 (s, 1H), 4.43 (s, 2H), 4.52 (s, 2H), 5.03-5.09 (m, 1H), 6.83 (d, J=8.4 Hz, 1H), 7.0 (d, J=7.6 Hz, 1H), 7.32 (t, J=7.6 Hz, 1H), 9.68 (s, br, 2H). LCMS (Method A): 0.841 min, MS: ES+215.14 (M+1).

### N-(Tetrahydrofuran-3-yl)isoindolin-4-amine hydrochloride (Intermediate 3)

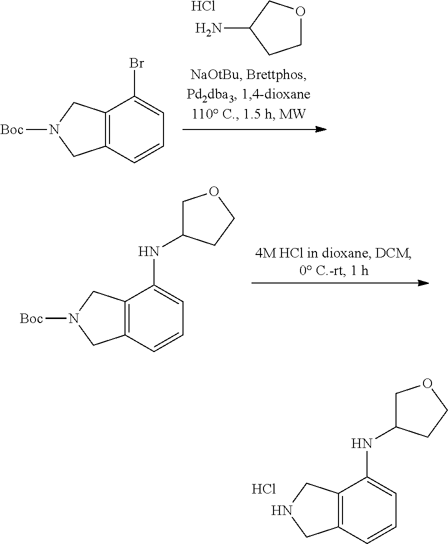 [SMILES: Cl ; NC1CCOC1 ; CC(C)(C)OC(=O)N1Cc2cccc(Br)c2C1 ; CC(C)(C)OC(=O)N1Cc2cccc(NC3CCOC3)c2C1 ; Cl ; c1cc2c(c(NC3CCOC3)c1)CNC2]

### t-Butyl 4-((tetrahydrofuran-3-yl) amino) isoindoline-2-carboxylate

t-Butyl 4-bromoisoindoline-2-carboxylate (CAS 1035235-27-8) (1.00 g, 3.36 mmol, 1 eq), tetrahydrofuran-3-amine hydrochloride (0.624 g, 5 mmol, 1.5 eq) and 1, 4-dioxane (5 mL) were placed in a 35 mL microwave glass tube and the mixture degassed at room temperature using N₂ gas for 30 min. NaOtBu (0.805 g, 8.38 mmol, 2.5 eq) and Brettphos (0.108 g, 0.20 mmol, 0.06 eq) were added to reaction mixture and again purged with N₂ for 10 min. Pd₂₍dba)₃ (0.092 g, 0.10 mmol, 0.03 eq) was added and the reaction heated to 110° C. under microwave irradiation for 1.5 h. The reaction mixture was filtered through a celite bed and the bed washed with ethyl acetate (2×50 mL). The combined filtrate was poured to ice-cold water (100 mL) and extracted in ethyl acetate (3×100 mL). The combined organic layer was dried over Na₂SO₄, filtered, and concentrated under vacuum. The crude material was purified by flash chromatography (product eluted in 20% EtOAc in hexane) to give the title compound as a yellow solid (0.6 g, Yield: 58.82%, 1.97 mmol). ¹H NMR (DMSO-d6, 400 MHz): δ ppm 1.47 (s, 9H), 1.83-1.86 (m, 1H), 2.13-2.19 (m, 1H), 3.55-3.59 (m, 1H), 3.69-3.74 (m, 1H), 3.79-3.85 (m, 1H), 3.88-3.92 (m, 1H), 4.01-4.02 (m, 1H), 4.42 (d, J=4.4 Hz, 2H), 4.49-4.52 (m, 2H), 5.34-5.39 (m, 1H, D₂O exchangeable), 6.42-6.45 (m, 1H), 6.53-6.57 (m, 1H), 7.06-7.10 (m, 1H). LCMS (Method A): 2.038 min, MS: ES+249.15 (M−56).

### N-(Tetrahydrofuran-3-yl)isoindolin-4-amine hydrochloride salt (Intermediate 3)

A stirred solution of t-butyl 4-((tetrahydrofuran-3-yl) amino) isoindoline-2-carboxylate (0.550 g, 1.81 mmol, 1.0 eq) in DCM (5 mL) at 0° C. was treated dropwise with 4M HCl in dioxane (0.5 mL). The reaction mixture was stirred for 1 h at RT, then concentrated under vacuum and crude material triturated using n-pentane (3×10 mL) followed by diethyl ether (10 mL) to give solid that was dried under high vacuum to give the title compound (0.50 g, 2.08 mmol. Yield 100%). ¹H NMR (DMSO-d6, 400 MHz): δ 1.82-1.84 (m, 1H), 2.15-2.24 (m, 1H), 3.57-3.60 (m, 2H), 3.69-3.74 (m, 1H), 3.81-3.92 (m, 2H), 4.30-4.33 (m, 2H), 4.35-4.40 (m, 2H), 6.54 (d, J=8.0 Hz, 1H), 6.64 (d, J=7.6 Hz, 1H), 7.16 (t, J=8 Hz, 15.6 Hz, 1H). LCMS (Method A): 0.702 min, MS: ES+205.18 (M+1).

The following intermediates were prepared according to the methods described above for Intermediate 3

| Intermediate | | | |
|---|---|---|---|
| No | Name | Structure | Data |
| 4 | N-(Oxetan-3- yl)isoindolin-4- amine | 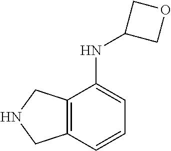 [SMILES: c1cc2c(c(NC3COC3)c1)CNC2] | LCMS (Method A): 0.735 min, MS: ES+ 191.2 (M + 1) |
| 5 | 1-(Isoindolin-4- yl)piperidin-3-ol | 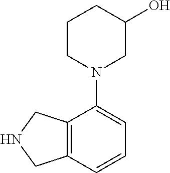 [SMILES: OC1CCCN(c2cccc3c2CNC3)C1] | ¹H NMR (DMSO-d6, 400 MHz): δ 1.24 − 1.35 (m, 1H), 1.60 − 1.63 (m, 1H), 1.80 − 1.90 (m, 2H), 2.55 − 2.60 (m, 1H), 2.68 − 2.74 (m, 1H), 3.09 − 3.12 (m, 1H), 3.18 − 3.21 (m, 1H), 3.71 (bs, 1H), 4.46 (s, br, 4H), 6.99 − 7.01 (m, 1H), 7.05 − 7.07 (m, 1H), 7.33 (t, J = 7.6 Hz, 15.6 Hz, 1H), 9.98 (s, 2H). LCMS (Method A): 0.555 min, MS ES+ 219.2 (M + 1). |
| 8 | N-(Tetrahydro- 2H-pyran-3- yl)isoindolin-4- amine | 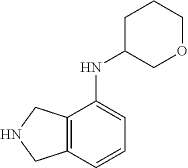 [SMILES: c1cc2c(c(NC3CCCOC3)c1)CNC2] | LCMS (Method A): 0.871 min, MS: ES+ 219.1 (M + 1). |
| 9 | N-(Tetrahydro- 2H-pyran-4- yl)isoindolin-4- amine | 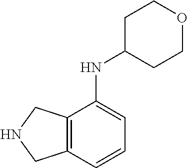 [SMILES: c1cc2c(c(NC3CCOCC3)c1)CNC2] | ¹H NMR (DMSO-d6, 400 MHz): δ ppm 1.50 − 1.57 (m, 2H), 1.84 − 1.87 (m, 2H), 3.35 − 3.41 (m, 2H), 3.55 − 3.56 (m, 3H), 3.86 − 3.89 (m, 4H), 6.75 − 6.85 (m, 2H), 7.19 − 7.23 (m, 1H), 9.95 (s, 2H). LCMS (Method A): 0.875 min, MS: ES+ 219.2 |
| 12 | 4-(Isoindolin-4- ylamino)-1- methylpyrrolidin- 2-one hydrochloride | 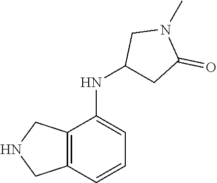 [SMILES: CN1CC(Nc2cccc3c2CNC3)CC1=O] | ¹H NMR (DMSO-d6, 400 MHz, D₂O exchange): 2.18 − 2.23 (m, 1H), 2.66 − 2.76 (m, 3H), 3.16 − 3.19 (m, 3H), 3.54 (s, 2H), 4.11 − 4.14 (m, 2H), 4.29 (s, 2H), 4.41 (s, 2H), 6.52 (d, J = 8 Hz, 1H), 6.66 (d, J = 7.2 Hz, 1H), 7.17 (t, J = 7.6 Hz, 1H). LCMS (Method A): 0.746 min, MS: ES+ 232.14 (M + 1). |
| 13 | N-(Pyrimidin-5- yl)isoindolin-4- amine hydrochloride | 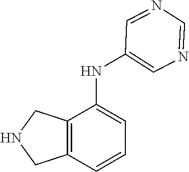 [SMILES: c1cc2c(c(Nc3cncnc3)c1)CNC2] | ¹H NMR (DMSO-d6, 400 MHz): δ ppm 4.45 − 4.52 (m, 4H), 7.03 (d, J = 7.2 Hz, 1H), 7.23 (d, J = 8 Hz, 1H), 7.31 (t, J = 7.6 Hz, 15.6 Hz, 1H), 8.55 (s, 2H), 8.60 (bs, 1H), 8.71 (s, 2H), 9.93 (bs, 2H). LCMS (Method A): 0.698 min, MS: ES+ 213.2. |
| 14 | N-(Oxazol-4- ylmethyl)isoindol in-4-amine hydrochloride | 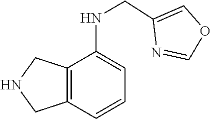 [SMILES: c1cc2c(c(NCc3cocn3)c1)CNC2] | ¹H NMR (DMSO-d6, 400 MHz): δ ppm 4.22 (s, 2H) 4.31 (t, J = 4.8, 10.4 Hz, 2H), 4.40 (t, J = 5.2 Hz, 10.8 Hz, 1H), 6.59 (t, J = 7.6 Hz, 16 Hz, 2H), 7.12 (t, J = 7.6 Hz, 16 Hz, 1H), 7.97 (s, 1H), 8.32 (s, 1H). 9.58 (s, br, 2H). LCMS (Method B): 1.75 min, MS: ES+ 216.31 (M + 1). |
| 15 | N-(1-Methyl-1H- pyrazol-4- yl)isoindolin-4- amine hydrochloride | 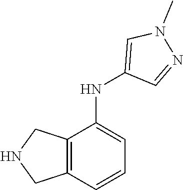 [SMILES: Cn1cc(Nc2cccc3c2CNC3)cn1] | ¹H NMR (DMSO-d6, 400 MHz): 3.81 (s, 3H), 4.33 − 4.43 (m, 4H), 6.65 − 6.69 (m, 2H), 7.11 (t, J = 7.6 Hz, 1H), 7.34 (s, 1H), 7.67 (s, 1H), 9.97 (s, br, 2H). LCMS (Method A): 0.791 min, MS: ES+ 215.2 (M + 1) |
| 16 | (S)- Tetrahydrofuran- 3-amine hydrochloride | 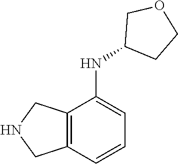 [SMILES: c1cc2c(c(N[C@H]3CCOC3)c1)CNC2] | ¹H NMR (DMSO-d6, 400 MHz): δ ppm 1.8 − 1.83 (m, 1H) 2.2 − 2.22 (m, 1H), 3.55 − 3.58 (m, 1H), 3.69 − 3.74 (m, 1H), 3.81 − 3.86 (m, 1H), 3.88 − 3.92 (m, 1H), 4.02 − 4.07 (m, 1H), 4.30 − 4.33 (m, 3H), 4.39 − 4.42 (m, 2H), 6.52 (d, J = 8 Hz, 1H), 6.62 (d, J = 7.6 Hz, 1H), 7.16 (t, J = 7.6 Hz, 2H), 9.63 (bs, 2H). LCMS (Method A): 0.822 min, MS: ES+ 205.1 (M + 1). |
| 17 | 4-(Isoindolin-4- ylamino)-5- methylpyridin- 2(1H)-one hydrochloride | 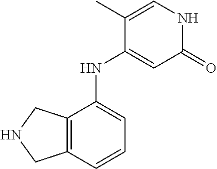 [SMILES: Cc1c[nH]c(=O)cc1Nc1cccc2c1CNC2] | ¹H NMR (DMSO-d6, 400 MHz): δ 2.18 (s, 3H), 4.45 (s, 2H), 4.57 (s, 2H), 5.93 (s, 1H), 7.32 (d, J = 8 Hz, 1H), 7.40 (d, J = 7.2 Hz, 1H), 7.50 (t, J = 8 Hz, 1H), 7.72 (s, 1H), 9.02 (s, 1H), 10.13 (s, 2H). LCMS (Method A): 0.640 min, MS: ES+ 242.1 (M + 1). |
| 22 | N-(5,5- Difluorotetrahydro- 2H-pyran-3- yl)isoindolin-4- amine hydrochloride | 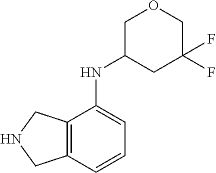 [SMILES: FC1(F)COCC(Nc2cccc3c2CNC3)C1] | ¹H NMR (DMSO-d6, 400 MHz): δ ppm 2.04 − 2.13 (m, 1H), 3.20 − 3.86 (m, 1H), 3.53 − 3.63 (m, 2H), 3.74 − 3.79 (m, 1H), 3.94 − 3.97 (m, 2H), 4.30 (s, br, 2H), 4.40 (s, br, 2H), 6.63 − 6.58 (d, J = 8 Hz, 2H), 7.15 (t, J = 8 Hz, 1H). 9.74 (bs, 2H) HCl salt. LCMS (Method A): 1.00 min, MS ES+ 255.0 (M + 1). |
| 23 | N- ((Tetrahydrofuran- 2-yl)methyl) isoindolin- 4-amine hydrochloride | 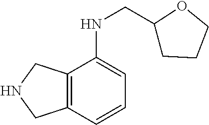 [SMILES: c1cc2c(c(NCC3CCCO3)c1)CNC2] | ¹H NMR (DMSO-d6, 400 MHz): δ ppm. 1.55 − 1.62 (m, 1H), 1.79 − 1.97 (m, 2H), 3.10 − 3.18 (m, 1H), 3,62 − 3.66 (m, 1H), 3.74 − 3.82 (m, 1H), 3.97 − 4.03 (m, 1H), 4.31- 4.33 (m, 2H), 4.36 − 4.39 (m, 2H), 6.62 (d, J = 8 Hz, 2H), 7.15 (t, J = 8 Hz, 1H). LCMS (Method A): 0.962 min, MS: ES+ 219.2 (M + 1) |
| 24 | N-(1H-pyrazol- 4-yl)isoindolin-4- amine hydrochloride) | 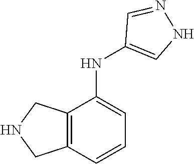 [SMILES: c1cc2c(c(Nc3cn[nH]c3)c1)CNC2] | ¹H NMR (DMSO-d6, 400 MHz): δ ppm 4.35 − 4.36 (m, 2H), 4.41 − 4.42 (m, 2H), 6.68 (t, J = 7.2 Hz, 2H), 7.12 (t, J = 7.6 Hz, 1H), 7.72 (s, 2H), 9.17 − 9.18 (m, 2H), 10.1 (s, 2H). LCMS (Method A): 0.648 min, MS: ES+ 201.1 (M + 1). Prepared using 1-(tetrahydro- 2H-pyran-2-yl)-1H-pyrazol-4-amine (CAS: 1216165-35-3) |
| 25 | N-(2-methyl-2H- 1,2,3-triazol-4- yl)isoindolin-4- amine hydrochloride | 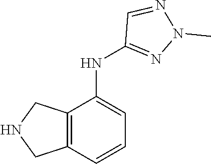 [SMILES: Cn1ncc(Nc2cccc3c2CNC3)n1] | ¹H NMR (DMSO-d6, 400 MHz): δ ppm 3.56 − 3.70 (m, 3H), 4.05 (s, 3H), 4.45 − 4.47 (m, 4H), 6.84 (d, J = 7.2 Hz, 1H), 7.25 (t, J = 8 Hz, 1H), 7.4 (s, 1H), 7.58 (d, J = 8.4 Hz, 1H), 8.52 (s, 1H, 9.93 (s, 2H). LCMS (Method A): 0.815 min, MS: ES+ 216.1 (M + 1). |
| 26 | 1-(3-(isoindolin- 4-ylamino) pyrrolidin- 1-yl)ethan-1- one hydrochloride | 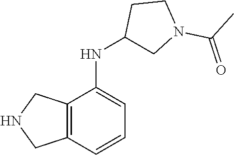 [SMILES: CC(=O)N1CCC(Nc2cccc3c2CNC3)C1] | ¹H NMR (DMSO-d6, 400 MHz): δ ppm 1.80 − 1.90 (m, 1H), 1.92 and 1.95( 2 singlets, 3H), 2.05 − 2.15 (m, 1H), 3.23 − 3.60 (m, 4H), 3.79 − 3.82 (m, 1H), 4.0 − 4.14 (m, 1H), 4.31, (m, br, 2H), 4.40 (m, br, 2H), 6.57 − 6.41 (m, 2H), 7.17 (t, J = 8 Hz, 1H), LCMS (Method A): 1.060 min, MS: ES+ 246.4 (M + 1). |
| 35 | N-(1-(Oxetan-3- yl)ethyl)isoindolin- 4-amine | 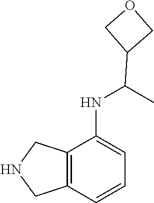 [SMILES: CC(Nc1cccc2c1CNC2)C1COC1] | ¹H NMR (DMSO-d6, 400 MHz): δ ppm: 1.02 (d, J = 6 Hz, 3H), 3.04 − 3.09 (m, 1H), 4.03 − 4.05 (m, 1H), 4.28 − 4.35 (m, br, 3H), 4.40 (s, br, 2H), 4.59 − 4.64 (m, 2H), 6.58 (d, J = 8hz, 1H), 6.64 (d, J = 8 Hz, 1H), 7.15 (t, J = 8 Hz, 1H), 9.26, s, br, 2H). LCMS (Method A): 0.860 min, MS: ES+ 219.09 (M + 1). Prepared using 1-(oxetan-3-yl)ethan-1- amine (CAS 1544892-89-8) |
| 36 | N-(1-Methyl-2- oxabicyclo[2.1.1] hexan-4- yl)isoindolin-4- amine | 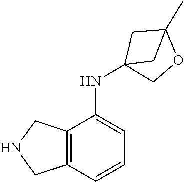 [SMILES: CC12CC(Nc3cccc4c3CNC4)(CO1)C2] | ¹H NMR (DMSO-d6, 400 MHz): δ ppm 2.38 (s, 3H), 1.83 − 1.92 (m, 4H), 3.65 (s, 2H), 4.28 − 4.30 (m, 2H), 4.40 − 4.41 (m, 2H), 6.63 (d, J = 8 Hz, 2H), 7.14 (t, J = 7.6 Hz, 1H), 9.31 (bs, 2H). LCMS (Method A): 0.868 min, MS: ES+ 231.2 (M + 1). Prepared using 1-methyl-2-oxabicyclo[2.1.1]hexan- 4-amine hydrochloride (CAS 2170372-24-2) |
| 37 | N-(2- Oxabicyclo[2.2.1] heptan-4- yl)isoindolin-4- amine | 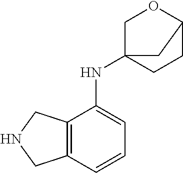 [SMILES: c1cc2c(c(NC34CCC(C3)OC4)c1)CNC2] | ¹H NMR (DMSO-d6, 400 MHz): δ ppm 1.70 − 1.90 (m, 6H), 3.58 − 3.59 (m, 1H), 3.85 − 3.86 (m, 1H), 4.22 (s, 1H), 4.32 (s, br, 2H), 4.39 (s, br, 2H), 6.53 (d, J = 8 Hz, 1H), 6.75 (d, J = 8 Hz, 1H), 7.13 (t, J = 8 Hz, 1H), 9.75 (bs, 2H). LCMS (Method A): 0.87 min, MS: ES+ 231 (M + 1). Prepared using 2- oxabicyclo[2.2.1 ]heptan-4-amine hydrochloride (0.23 g, 1.51 mmol, 1.5 eq) (CAS: 2253632-53-8) |
| 74 | N- ((Tetrahydrofuran- 3-yl) methyl)isoindolin- 4-amine hydrochloride | 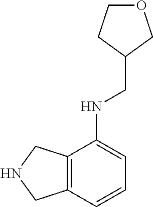 [SMILES: c1cc2c(c(NCC3CCOC3)c1)CNC2] | ¹H NMR (DMSO-d6, 400 MHz): δ ppm 1.24 (s, 2H) 1.58 − 1.64 (m, 1H), 1.94 − 2.03 (m, 1H), 3,05 − 3.07(m, 2H), 3.47 − 3.50 (m, 1H), 3.60 − 3.69 (m, 1H), 3.71 − 3.78 (m, 2H), 4.30 − 4.42 (dt, J = 10.8 Hz, 5.6 Hz, 4H), 6.56 − 6.61 (m, 2H), 7.15 (t, J = 8 Hz, 7.6 Hz, 1H), 9.84 (s, 2H, br, exchangeable). LCMS (Method A): 0.879 min, MS: ES+ 219.0 (M + 1). Prepared using (tetrahydrofuran-3- yl)methanamine. |
| 93 | (R)-4- (Isoindolin-4- ylamino)-1- methylpyrrolidin- 2-one hydrochloride | 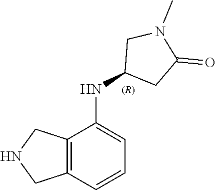 | ¹H NMR (DMSO-d6, 400 MHz): δ ppm 2.21 − 2.27 (m, 1H), 2.69 − 2.76 (m, 4H), 3.20 (dd, J = 3.6 Hz, J = 10 Hz, 1H), 3.74 (q, J = 6.8 Hz, 1H), 4.12 − 4.16 (m, 1H), 4.31 (t, J = 5.6 Hz 2H), 4.40 (t, J = 4.42 Hz, 2H), 6.52 (d, J = 8 Hz, 1H), 6.65 (t, J = 7.2 Hz, 1H), 7.11 (t, J = 8 Hz, 1H). LCMS (Method A): 0.736 min, MS: ES+ 231.9 (M + 1). Prepared from (R)- 4-amino-1-methylpyrrolidin-2-one. |
| 94 | (S)-4- (Isoindolin-4- ylamino)-1- methylpyrrolidin- 2-one hydrochloride | 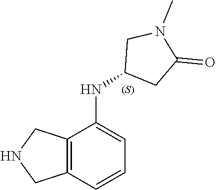 | ¹H NMR (DMSO-d6, 400 MHz): δ ppm: 2.22 − 2.27 (m, 1H), 2.69 − 2.76 (m, 1H), 2.76 (s, 3H), 3.20 (dd, J = 3.6 Hz, 10 Hz, 1H), 3.74 (q, J = 10 Hz, 1H), 4.11 − 4.17 (m, 1H), 4.31 (t, J = 5.2 Hz 2H), 4.40 (t, J = 5.6 Hz, 2H), 6.52 (d, J = 8 Hz, 1H), 6.65 (d, J = 7.2 Hz, 1H), 7.17 (t, J = 8 Hz, 1H). LCMS (Method A): 0.727 min, MS: ES+ 231.9 (M + 1). Prepared from (S)-4-amino-1- methylpyrrolidin-2-one. |

### N-(Tetrahydrofuran-3-yl)-1,2,3,4-tetrahydroisoquinolin-5-amine hydrochloride (Intermediate 20)

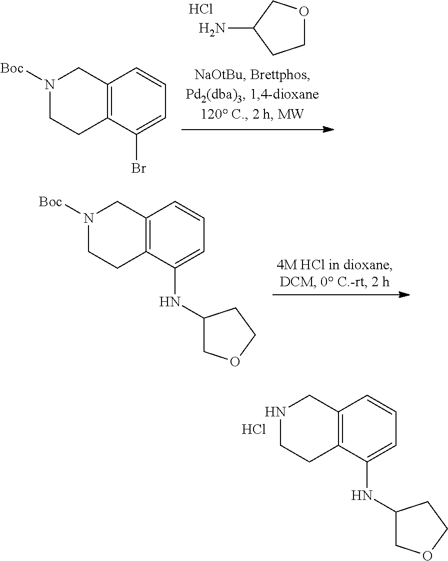

A mixture of t-butyl 5-bromo-3,4-dihydroisoquinoline-2(1H)-carboxylate (1.5 g, 4.8 mmol, 1 eq) (CAS: 215184-78-4) and tetrahydrofuran-3-amine hydrochloride (1.18 g, 9.54 mmol, 1.99 eq) (CAS: 204512-94-7), NaOtBu (1.15 g, 11.97 mmol, 2.5 eq) and Brettphos (0.153 g, 0.28 mmol, 0.06 eq) in 1, 4-dioxane (15 ml) in 35 mL microwave glass vial at rt was degassed (N₂ gas) for 20 min. Pd₂(dba)₃ (0.174 g, 0.19 mmol, 0.04 eq) was added and the reaction mixture was heated to 120° C. for 2 h under microwave irradiation. The reaction mixture was filtered through a celite bed and the bed washed using ethyl acetate (2×30 mL) and water (2×30 mL). The combined organic layer was washed with brine solution (50 mL), dried over Na₂SO₄, filtered, and concentrated under vacuum. The crude material was purified by flash chromatography (product eluted in 15% EtOAc in hexane) yielding t-butyl 5-((tetrahydrofuran-3-yl)amino)-3,4-dihydroisoquinoline-2(1H)-carboxylate as a yellow solid (2 g, Yield: quantitative). ¹H NMR (DMSO-d6, 400 MHz): δ ppm 1.41 (s, 9H), 1.81-1.85 (m, 1H), 2.15-2.20 (m, 1H), 2.45-2.47 (m, 2H), 3.54-3.57 (m, 3H), 3.68-3.73 (m, 1H), 3.79-3.85 (m, 1H), 3.89-3.98 (m, 1H), 3.99-4.00 (m, 1H), 4.41 (s, br, 2H), 4.78-4.8 (m, 1H), 6.42 (t, J=7.6 Hz, 2H), 7.00 (d, J=8 Hz, 1H). LCMS (Method A): 2.086 min, MS: ES+268.2 (M−56).

A stirred solution of t-butyl 5-((tetrahydrofuran-3-yl)amino)-3,4-dihydroisoquinoline-2(1H)-carboxylate (2 g, 6.28 mmol, 1.0 eq) in DCM (20 mL) was cooled to 0° C. 4M HCl in Dioxane (20 mL, 10 V) was added dropwise and the reaction mixture stirred at room temperature for 2 h. The reaction mixture was concentrated under vacuum and crude material was triturated using n-pentane (3×30 mL) followed by diethyl ether (30 mL) and obtained material was dried under high vacuum yielding the title compound. (1.4 g, Yield: 87.5%). ¹H NMR (DMSO-d6, 400 MHz): δ ppm 1.84-1.87 (m, 1H), 2.16-2.21 (m, 1H), 2.67-2.72 (m, 1H), 2.76-2.80 (m, 1H), 3.35 (s, br, 2H), 3.58-3.61 (m, 1H), 3.69-3.74 (m, 1H), 3.81-3.86 (m, 1H), 3.89-3.93 (m, 1H), 4.02-4.04 (m, 1H), 4.15 (m, br, 2H), 6.50-6.56 (m, 1H), 6.62-6.77 (m, 1H), 7.02-7.10 (m, 1H), 9.38 (s, 2H). LCMS (Method A): 0.813 min, MS: ES+219.2

### N-(Tetrahydrofuran-3-yl)-1,2,3,4-tetrahydroisoquinolin-8-amine (Intermediate 21)

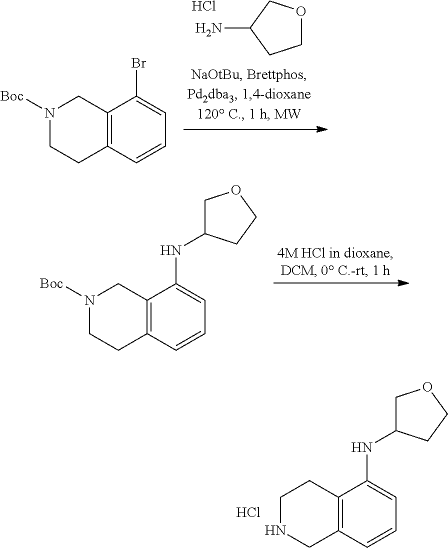 [SMILES: Cl ; NC1CCOC1 ; CC(C)(C)OC(=O)N1CCc2cccc(Br)c2C1 ; CC(C)(C)OC(=O)N1CCc2cccc(NC3CCOC3)c2C1 ; Cl ; c1cc2c(c(NC3CCOC3)c1)CCNC2]

A mixture of t-butyl 8-bromo-3,4-dihydroisoquinoline-2(1H)-carboxylate (2.5 g, 8.0 mmol, 1 eq) (CAS. 893566-75-1), tetrahydrofuran-3-amine hydrochloride (1.5 g, 12.0 mmol, 1.5 eq), NaOtBu (1.92 g, 20 mmol, 2.5 eq) and Brettphos (0.26 g, 0.48 mmol, 0.06 eq) in 1, 4-dioxane (5 mL) was prepared in 35 mL microwave glass vial. The reaction mixture was degassed (N₂ gas) for 30 min. Pd₂(dba)₃ (0.293 g, 0.32 mmol, 0.04 eq) was added to reaction mixture at rt and the reaction heated to 120° C. under microwave irradiation for 1 h. The reaction mixture was filtered through a celite bed and the bed washed using ethyl acetate (3×100 mL). The combined organic layer was dried over Na₂SO₄, filtered, and concentrated under vacuum. The crude material was purified by flash chromatography (product eluted with 22% EtOAc in hexane) yielding t-butyl 8-((tetrahydrofuran-3-yl)amino)-3,4-dihydroisoquinoline-2(1H)-carboxylate as a yellow sticky solid (1.9 g, Yield: 74.6%). ¹H NMR (DMSO-d6, 400 MHz): δ ppm 1.43 (s, 9H), 1.81-1.89 (m, 1H), 2.13-2.22 (m, 1H), 2.66-2.69 (m, 2H), 3.5 (s, br, 2H), 3.57-3.6 (m, 1H), 3.69-3.75 (m, 1H), 3.8-3.86 (m, 1H), 3.90-3.92 (m, 1H), 3.99-4.01 (m, 1H), 4.27-4.33 (m, 2H), 4.83 (bs, 1H), 6.44-6.46 (m, 1H), 6.57-6.65 (m, 1H), 6.94-7.01 (m, 1H). LCMS (Method A): 2.153 min, MS: ES+263.1 (M−56).

A stirred solution of t-butyl 8-((tetrahydrofuran-3-yl)amino)-3,4-dihydroisoquinoline-2(1H)-carboxylate (1.9 g, 5.97 mmol, 1.0 eq) in DCM (10 mL) was cooled to 0° C. 4M HCl in Dioxane (10 mL) was added dropwise to the reaction mixture which was stirred at room temperature for 1 h. The reaction mixture was concentrated under vacuum and the crude material triturated using n-pentane (3×40 mL) followed by diethyl ether (2×30 mL) and dried under high vacuum yielding the title compound (1.9 g, Yield: quantitative). ¹H NMR (DMSO-d6, 400 MHz): δ ppm 1.80-1.83 (m, 1H) 2.20-2.22 (m, 1H), 2.90-2.94 (m, 2H), 3.25-3.27 (m, 2H), 3.55-3.58 (m, 1H), 3.69-3.74 (m, 1H), 3.81-3.86 (m, 1H), 3.88-3.92 (m, 1H), 3.99-4.02 (m, 3H), 6.54-6.75 (m, 2H), 7.04-7.11 (m, 1H) 9.72 (bs, 2H)._LCMS (Method A): 0.891 min, MS: ES+219.1 (M+1).

The following intermediates were prepared according to the methods described above for Intermediate 21:

| Intermediate | | | |
|---|---|---|---|
| No | Name | Structure | Data |
| 31 | N-(Oxetan-3-yl)- 1,2,3,4- tetrahydroisoquinoline- 8-amine hydrochloride | 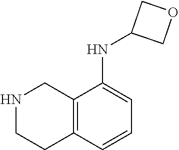 [SMILES: c1cc2c(c(NC3COC3)c1)CNCC2] | ¹H NMR (DMSO-d6, 400 MHz): δ ppm 2.88 − 3.95 (m, 2H), 3.30 − 3.40 (m, 3H), 3.50 (m, 1H), 3.90 (s, 1H), 4.06 (s, 2H), 4.40 − 4.57 (m, 5H), 4.60 − 4.80 (bs, 5H), 4.87 (t, J = 6 Hz, 12 Hz, 2H), 6.17 (d, J = 8 Hz, 1H), 6.55 − 6.67 (m, 1H), 7.06 (t, J = 7.6 Hz, 15.2 Hz, 1H), 9.07 (bs, 2H). LCMS (Method A): 0.796 min, MS: ES+ 205.1 (M + 1) |
| 32 | 1-Methyl-4- ((1,2,3,4- tetrahydroisoquinoline- 8- yl)amino)pyrrolid in-2-one hydrochloride | 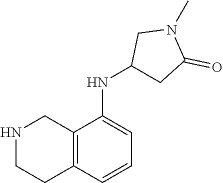 [SMILES: CN1CC(Nc2cccc3c2CNCC3)CC1=O] | ¹H NMR (DMSO-d6, 400 MHz): δ ppm 2.69 − 2.75 (m, 3H), 2.92 (m, 2H), 3.18 − 3.22 (m, 1H), 3.27 (bs, 2H), 3.56 − 3.58 (m, 2H), 3.72 − 3.76 (m, 1H), 3.96 (s, br, 2H), 4.11 − 4.13 (m, 1H), 6.49 − 6.55 (m, 2H), 7.09 (t, J = 8 Hz, 1H) 9.59 (bs, 2H). LCMS (Method A): 0.822 min, MS: ES+ 246.1 (M + 1). |
| 33 | N-methyl- 1,2,3,4- tetrahydroisoquinoline- 8-amine hydrochloride | 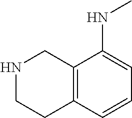 | ¹H NMR (DMSO-d6, 400 MHz, D₂O exchange) δ ppm:2.74 (s, 3H), 2.93 − 2.96 (m, 2H), 3.28 − 3.31 (m, 2H), 4.05 (s, 2H), 6.68 (d, J = 7.6 Hz, 2H), 7.18 (t, J = 8 Hz, 1H). LCMS (Method A): 0.767 min, MS: ES+ 163.17 (M + 1) |
| 34 | N-(2- methoxyethyl)- 1,2,3,4- tetrahydroisoquinoline- 8-amine hydrochloride | 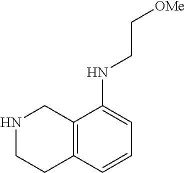 [SMILES: COCCNc1cccc2c1CNCC2] | ¹H NMR (DMSO-d6, 400 MHz, D₂O exchange) δ ppm: (2.89 − 2.92 (m, 2H), 3.23 − 3.31 (m, 4H), 3.47 − 3.50 (m, 2H), 3.95 − 4.04 (m, 2H), 6.50 − 6.57 (m, 2H), 7.087 (t, J = 8 Hz, 1H). LCMS (Method B): 2.10 min, MS: ES+ 207.31 (M + 1). |
| 38 | 6-Methoxy-N- (tetrahydrofuran- 3-yl)-1,2,3,4- tetrahydro- isoquinolin-8- amine | 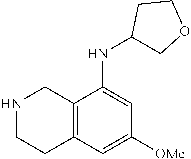 [SMILES: COc1cc2c(c(NC3CCOC3)c1)CNCC2] | ¹H NMR (DMSO-d6, 400 MHz): δ ppm 1.82 − 1.83 (m, 1H) 2.12 − 2.16 (m, 1H), 2.88 (s, 2H), 3.16 (bs, 2H), 3.45 − 3.49 (m, 1H), 3.57 (s, 3H), 3.68 − 3.85 (m, 5H), 3.90 − 4.00 (m, 1H), 6.03 (s, 1H), 6.11 (s, 1H), 9.47 (s, 2H). LCMS (Method A): 0.915 min, 99.11%, 288 nm MS: ES+ 249.1. Starting material t-butyl 8- bromo-6-methoxy-3,4-dihydroisoquinoline- 2(1H)-carboxylate was prepared from 8- bromo-6-methoxy-1,2,3,4tetrahydro- isoquinoline hydrochloride (CAS 2411640- 34-9) by treatment with Boc₂O and triethylamine in THF at rt. |
| 41 | (S)-1-Methyl-4- ((1,2,3,4- tetrahydroisoquinolin- 8-yl)amino) pyrrolidin-2-one | 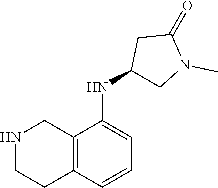 [SMILES: CN1C[C@@H](Nc2cccc3c2CNCC3)CC1=O] | ¹H NMR (DMSO-d6, 400 MHz): δ ppm 2.25 − 2.32 (m, 1H), 2.70 (s, 3H), 2.66 − 2.74 (m, 1H), 2.91 (t, J =7.2 Hz, 2H), 3.18 − 3.36 (m, 3H), 3.73 (t, J =6.8 Hz, 1H), 3.96 (s, br, 2H), 4.09 − 4.12 (m, 1H), 6.49 − 6.54 (m, 2H), 7.08 (t, J = 8 Hz, 1H), 9.64 (bs, 2H). LCMS (Method A): 0.826 min, MS: ES+ 246.0 (M + 1) |
| 42 | (R)-1-Methyl-4- ((1,2,3,4- tetrahydroisoquinolin- 8- yl)amino)pyrrolidin- 2-one | 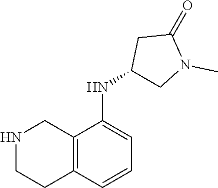 [SMILES: CN1C[C@H](Nc2cccc3c2CNCC3)CC1=O] | ¹H NMR (DMSO-d6, 400 MHz): δ ppm 2.25 − 2.30 (m, 1H), 2.70 (s, 3H), 2.66 − 2.74 (m, 1H), 2.92 (t, J = 6 Hz, 11.6 Hz, 2H), 3.18 − 3.21 (m, 1H), 3.27 (bs, 2H), 3.72 − 3.76 (m, 1H), 3.96 (s, br, 2H), 4.10 − 4.13 (m, 1H), 6.49 − 6.55 (m, 2H), 7.09 (t, J = 8 Hz, 1H), 9.55 (bs, 2H). LCMS (Method A): 0.795 min, MS: ES+ 246.1 (M + 1). |
| 43 | (R)-6-(2- Methoxyethoxy)- N- (tetrahydrofuran- 3-yl)-1,2,3,4- tetrahydro isoquinolin-8- amine | 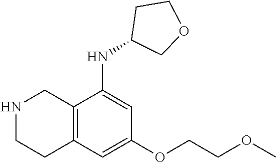 [SMILES: COCCOc1cc2c(c(N[C@@H]3CCOC3)c1)CNCC2] | ¹H NMR (DMSO-d6, 400 MHz): δ ppm 1.82 − 1.83 (m, 1H), 2.15 − 2.20 (m, 1H), 2.85 − 2.88 (m, 2H), 3.25 − 3.29 (m, 2H), 3.37 (s, 3H), 3.59 − 4.05 (m, 11H), 6.03 (s, 1H), 6.11 (s, 1H). 9.40 (bs, 2H). LCMS (Method A): 0.90 min, MS: ES+ 293 (M + 1). Starting material t- butyl-8-bromo-6-(2-methoxyethoxy)-3,4- dihydro-isoquinoline-2(1H)-carboxylate was prepared from t-butyl-8-bromo-6-hydroxy- 3,4-dihydroisoquinoline-2(1H)-carboxylate (CAS: 1579518-76-5) by treatment with 1- bromo-2-methoxyethane and K₂CO₃ in DMF at rt. |
| 45 | (S)-6-(2- methoxyethoxy)- N- (tetrahydrofuran- 3-yl)-1,2,3,4- tetrahydro isoquinolin-8- amine | 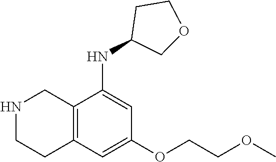 [SMILES: COCCOc1cc2c(c(N[C@H]3CCOC3)c1)CNCC2] | LCMS (Method A): 0.924 min, MS: ES+ 293.1 (M + 1). Starting material t-butyl 8- bromo-6-(2-methoxyethoxy)-3,4-dihydro- isoquinoline-2(1H)-carboxylate was prepared from t-butyl-8-bromo-6-hydroxy- 3,4-dihydroisoquinoline-2(1H)-carboxylate (CAS: 1579518-76-5) by treatment with 1- bromo-2-methoxyethane and K₂CO₃ in DMF at rt. |
| 46 | (S)-1-(3- ((1,2,3,4- tetrahydroisoquinolin- 8- yl)amino)pyrrolidin- 1-yl)ethan-1- one | 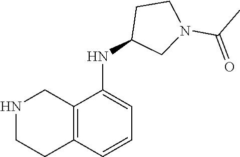 [SMILES: CC(=O)N1CC[C@H](Nc2cccc3c2CNCC3)C1] | ¹H NMR (DMSO-d6, 400 MHz): δ ppm: 1.91 and 1.94 (2 singlets, 3H), 1.60 − 2.05 (m, 1H), 2.08 − 2.22 (m, 1H), 2.91 (t, br, 2H), 3.27 − 3.32 (m, 2H), 3.42 − 3.60 (m, 3H), 3.79 3.83 (m, 1H) 3.97 − 4.00 (m, 2H), 4.01 − 4.15 (m, 1H), 6.52 − 6.61 (m, 2H), 7.09 (t, J = 8 Hz, 1H), 9.44 − 9.52 (m, 2H). LCMS (Method A): 0.859 min, MS: ES+ 260 (M + 1). Using starting material (S)-1-(3-aminopyrrolidin-1- yl)ethan-1-one HCl (CAS 1013921-14-6) |
| 47 | (R)-1-(3- ((1,2,3,4- tetrahydroisoquinolin- 8-yl)amino) pyrrolidin-1-yl) ethan-1- one | 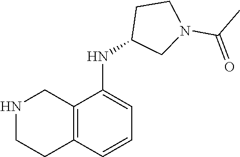 [SMILES: CC(=O)N1CC[C@@H](Nc2cccc3c2CNCC3)C1] | LCMS (Method A): 0.851 min, MS: ES+ 260.1 (M + 1). Using starting material (R)-1- (3-aminopyrrolidin-1-yl)ethan-1-one HCl (CAS 11286208-55-6) |
| 49 | (R)-1-(3- ((1,2,3,4- Tetrahydro isoquinolin-8- yl)amino)piperidin- 1-yl)ethan-1- one | 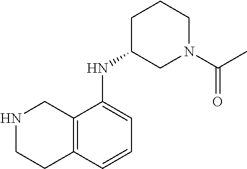 [SMILES: CC(=O)N1CCC[C@@H](Nc2cccc3c2CNCC3)C1] | ¹H NMR (DMSO-d6, 400 MHz): δ ppm 1.49 − 1.55 (m, 1H), 1.59 − 1.69 (m, 1H), 1.70 − 1.76 (m, 1H), 1.95 − 2.01 (m, 4H), 2.38 − 2.44 (m, 1H), 2.90 − 2.94 (m, 2H), 2.96 − 3.01 (m, 1H), 3.16 − 3.17 (m, 1H), 3.18 − 3.21 (m, 2H), 3.73 − 3.81 (m, 1H), 3.97 − 3.98 (m, 2H), 4.00 − 4.51 (m, 1H), 6.52 − 6.54 (m, 1H), 6.65 − 6.7 (m, 1H), 7.07 − 7.12 (m, 1H), 9.60 − 9.69 (m, 1H). LCMS (Method A): 0.942 min, MS: ES+ 274.5 (M + 1). Using starting material (R)-1- (3-aminopiperidin-1-yl)ethan-1-one (CAS 1177014-59-3) |
| 52 | N-(Pyrimidin-5- yl)-1,2,3,4- tetrahydroisoquinolin- 8-amine | 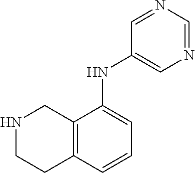 [SMILES: c1cc2c(c(Nc3cncnc3)c1)CNCC2] | ¹H NMR (DMSO-d6, 400 MHz): δ ppm 3.01 − 3.04 (m, 2H), 3.30 − 3.32 (m, 2H), 4.15 − 4.18 (m, 2H), 7.01 (d, J = 5.2 Hz, 1H), 7.19 − 7.28 (m, 2H), 8.42 − 8.47 (s, 2H), 8.69 (s, 1H), 9.73 − 9.90 (bs, 2H). HCl salt. LCMS (Method A): 0.748 min, MS: ES+ 227.0 (M + 1). Using starting material 5-pyridinamine (CAS 591- 55-9) |
| 48 | (S)-1-(3- ((1,2,3,4- Tetrahydroisoquinolin- 8- yl)amino)piperidin- 1-yl)ethan-1- one | 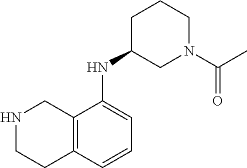 [SMILES: CC(=O)N1CCC[C@H](Nc2cccc3c2CNCC3)C1] | ¹H NMR (DMSO-d6, 400 MHz): 1.53 − 1.55 (m, 2H), 1.72 (m, br,1H), 1.98 (m, 4H), 2.36 (m, 1H), 2.97 −2.90 (m, 3H), 3.16 (s, 1H), 3.30 (s, 2H), 3.75 (m, br, 1H), 3.95 (s, br, 2H), 4.1 − 4.15 and 4.45 − 4.55 (m, 1H), 6.49 (d, J = 8 Hz, 1H), 6.67 − 6.61 (m, 1H), 7.11 − 7.05 (m, 1H), 9.34 (s, br, 2H). LCMS (Method A): 0.957 min, MS: ES+ 274.4 (M + 1). Using starting material (S)-1-(3-aminopiperidin-1- yl)ethan-1-one. 2HCl (CAS: 1207602-55-8) |
| 50 | (1-(4-((1,2,3,4- Tetrahydroisoquinolin- 8- yl)amino)piperidin- 1-yl)ethan-1- one | 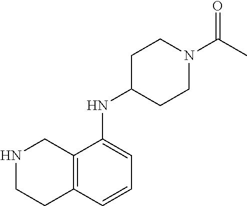 [SMILES: CC(=O)N1CCC(Nc2cccc3c2CNCC3)CC1] | ¹H NMR (DMSO-d6, 400 MHz): δ ppm 1.31 − 1.34 (m, 1H), 1.43 − 1.46 (m, 1H), 1.86 − 1.95 (m, 2H), 2.00 (s, 3H), 2.66 − 2.71 (m, 1H), 2.91 − 2.94 (m, 2H), 3.10 − 3.16 (m, 1H), 3.27 − 3.28 (m, 2H), 3.54 − 3.59 (m, 1H), 3.80 − 3.84 (m, 1H), 4.05 (s, br, 2H), 4.29 − 4.32 (m, 1H), 6.60 − 6.65 (m, 1H), 6.72 − 6.76 (m, 1H), 7.04 − 7.14 (m, 1H), 9.56 (bs, 2H). LCMS (Method A): 0.904 min, MS: ES+ 274.0 (M + 1). Using starting material 1-(4- aminopiperidin-1-yl)ethan-1-one) (CAS: 160357-94-8) |
| 51 | 1-Methyl-5- ((1,2,3,4- tetrahydroisoquinolin- 8- yl)amino)piperidin- 2-one | 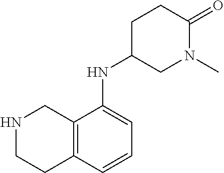 [SMILES: CN1CC(Nc2cccc3c2CNCC3)CCC1=O] | ¹H NMR (DMSO-d6, 400 MHz): δ ppm 1.79 − 1.82 (m, 1H), 1.96 − 1.98 (m, 1H), 2.32 − 2.35 (m, 2H), 2.8 (s, 3H), 2.90 − 2.93 (m, 2H), 3.21 − 3.27 (m, 3H), 3.44 − 3.48 (m, 1H), 3.85 − 3.86 (m, 1H), 3.98 (s, 2H), 6.52 (d, J = 7.6 Hz, 1H), 6.68 (d, J = 8 Hz, 1H), 7.08 (t, J = 8 Hz, 1H), 9.58 (s, 2H). LCMS (Method A): 0.846 min, MS: ES+ 260.1 (M + 1). Using starting material 5-amino-1-methylpiperidin-2-one (CAS: 90485-53-3). |
| 53 | 3-((1,2,3,4- Tetrahydroisoquinolin- 8- yl)amino)benzamide | 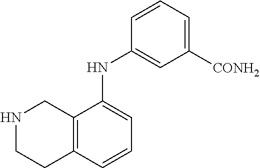 [SMILES: NC(=O)c1cccc(Nc2cccc3c2CNCC3)c1] | ¹H NMR (DMSO-d6, 400 MHz): δ ppm 1.07 − 1.10 (m, 1H), 3.06 − 3.07 (m, 2H), 3.33 − 3.37 (m, 3H), 4.13 (s, br, 3H), 7.15 − 7.31 (m, 3H), 7.44 − 7.46 (m, 1H), 7.52 − 7.59 (m, 1H), 7.76 − 7.90 (m, 2H), 9.55 (bs, 3H), 10.31 − 10.37 (m, 1H). LCMS (Method A): 0.664 min, MS: ES+ 267.9 (M + 1). Using starting material 3- aminobenzamide. |
| 54 | 4-((1,2,3,4- Tetrahydroisoquinolin- 8- yl)amino)benzamide | 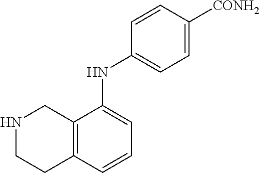 [SMILES: NC(=O)c1ccc(Nc2cccc3c2CNCC3)cc1] | ¹H NMR (DMSO-d6, 400 MHz): δ ppm 3.06 (s, br, 2H), 3.33 (s, br, 2H), 4.11 (s, 2H), 6.96 − 7.29 (m, 5H), 7.88 (d, J = 7.2 Hz, 2H). LCMS (Method B): 2.16 min, MS: ES+ 268.49 (M + 1). Using starting material 4- aminobenzamide. |
| 55 | N-Cyclopentyl- 1,2,3,4- tetrahydroisoquinolin- 8-amine | 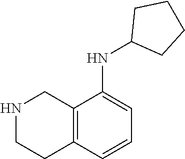 [SMILES: c1cc2c(c(NC3CCCC3)c1)CNCC2] | ¹H NMR (DMSO-d6, 400 MHz): δ ppm 1.53 − 1.57 (m, 2H), 1.64 − 1.72 (m, 3H), 1.89 − 1.92 (m, 2H), 2.96 − 2.98 (m, 2H), 3.28 − 3.29 (m, 2H), 3.77 − 3.80 (m, 1H), 4.2 (s, br, 2H), 6.79 − 6.89 (m, 2H), 7.18 − 7.22 (m, 1H), 9.72 (s, 2H). LCMS (Method A): 1.228 min, MS: ES+ 217.0 (M + 1). Using starting material cyclopentylamine. |
| 56 | N-(1- Methylpiperidin- 4-yl)-1,2,3,4- tetrahydroisoquinolin- 8-amine | 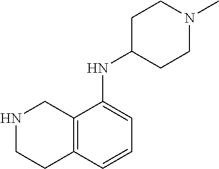 [SMILES: CN1CCC(Nc2cccc3c2CNCC3)CC1] | ¹H NMR (DMSO-d6, 400 MHz): δ ppm 1.80 − 1.89 (m, 2H) 1.97 − 2.07 (m, 2H), 2.66 − 2.75 (2 singlets, 3H), 2.91 − 2.94 (m, 2H), 3.00 − 3.09 (m, 2H), 3.20 − 3.30 (M, 2H), 3.38 − 3.42 (m, 2H), 3.48 − 3.56 (m, 1H), 3.98 (bs, 2H), 6.52 − 6.65 (m, 2H), 7.08 (t, J = 7.6 Hz, 1H), 9.75 (bs, 2H), 10.83 (bs, 1H). LCMS (Method A): 0.503 min, MS: ES+ 246 (M + 1). Using starting material 1-methylpiperidin-4-amine. |
| 57 | N-(1- Methylpyrrolidin- 3-yl)-1,2,3,4- tetrahydroisoquinolin- 8-amine | 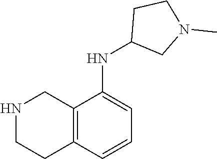 [SMILES: CN1CCC(Nc2cccc3c2CNCC3)C1] | ¹H NMR (DMSO-d6, 400 MHz): δ ppm 1.92 − 2.05 (m, 1H), 2.21 − 2.33 and 2.50 − 2.58 (m, 1H), 2.81 − 2.86 (m, 3H), 2.93 (s, br, 2H), 3.05 − 3.07 (m, 1H), 3.26 − 3.32 (m, 2H), 3.45 − 3.51 (m, 1H), 3.51 − 3.60 (m, 1H), 3.9 − 3.92 (m, 1H), 4.0 − 4.1 (m, 2H), 4.3 (s, br, 1H), 6.49 − 6.57 (m, 2H), 7.11 (t, J = 8 Hz, 1H), 9.66 (m, 2H), 11.1 (s,1H). LCMS (Method A): 0.370 min, MS: ES+ 232.5 (M + 1). Using starting material 1-methylpyrrolidin-3-amine) (CAS: 13220-27-4) |
| 75 | N,N-Dimethyl-3- ((1,2,3,4- tetrahydroisoquinolin- 8- yl)amino) pyrrolidine-1- carboxamide. hydrochloride | 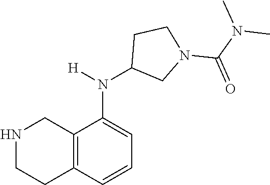 [SMILES: CN(C)C(=O)N1CCC(Nc2cccc3c2CNCC3)C1] | ¹H NMR (DMSO-d6, 400 MHz): δ ppm 1.83 − 1.88 (m, 1H) 2.09 − 2.12 (m, 1H), 2.73 (s, 4H), 2.94 (t, J = 6, 3H), 3.18 − 3.23 (m, 3H), 3.57 (s, 6H), 5.76 (s, 1H), 6.52 − 6.54 (d, J = 7.6 Hz, 1H), 6.59 − 6.61 (d, J = 20 Hz, 2H), 6.73 − 6.73 (d, J = 7.6 Hz, 1H), 7.05 − 7.11 (m, 2H). LCMS (Method A): 0.991 min, MS: ES+ 289.09 (M + 1). Prepared from 3-amino-N,N- dimethylpyrrolidine-1-carboxamide |
| 76 | 1-Methyl-4- ((1,2,3,4- tetrahydroisoquinolin- 8- yl)amino)piperidin- 2-one hydrochloride | 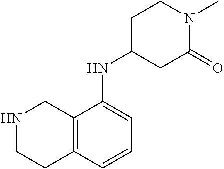 [SMILES: CN1CCC(Nc2cccc3c2CNCC3)CC1=O] | ¹H NMR (DMSO-d6, 400 MHz): δ ppm 1.73 − 1.78 (m, 1H) 2.03 (d, J = 12.8 Hz, 1H), 2.32 − 2.39 (m, 1H), 2.57 − 2.58 (m, 1H), 2.90 (t, J = 5.6 Hz, 2H), 2.92 (t, J = 4.4 Hz, 2H), 3.27 − 3.30 (m, 2H), 3.56 (s, 1H), 3.80 (t, J = 4 Hz, 1H), 4.02 (s, 2H), 6.57 (d, J = 7.2 Hz, 1H), 6.68 (d, J = 8 Hz, 1H), 7.10 (t, J = 7.6 Hz, 1H). LCMS (Method A): 0.913 min, MS: ES+ 260.0 (M + 1). Prepared from 4-amino-1- methylpiperidin-2-one. |
| 79 | N-(1-Methyl-1H- pyrazol-4-yl)- 1,2,3,4- tetrahydroisoquinolin- 8-amine hydrochloride | 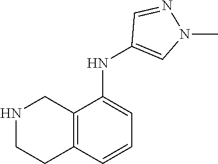 [SMILES: Cn1cc(Nc2cccc3c2CNCC3)cn1] | ¹H NMR (DMSO-d6, 400 MHz): δ ppm 2.93 − 2.96 (m, 2H), 3.32 − 3.33 (m, 2H), 3.81 (s, 3H), 4.07 (s, 2H), 6.56 (d, J = 7.6 Hz, 1H), 6.67 (d, J = 8 Hz, 1H), 7.03 (t, J = 7.6 Hz, 1H), 7.29 (s, 1H), 7.60 (s, 1H), 9.45 (s, 2H). LCMS (Method A): 0.869 min, MS: ES+ 229 (M + 1). Prepared from 1-methyl-1H-pyrazol-4-amine |
| 80 | N-(Pyridin-3-yl)- 1,2,3,4- tetrahydroisoquinolin- 8-amine hydrochloride | 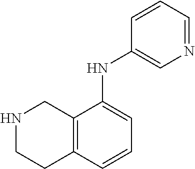 [SMILES: c1cncc(Nc2cccc3c2CNCC3)c1] | ¹H NMR (DMSO-d6, 400 MHz): δ ppm 3.06 − 3.075 (m, br, 2H), 3.35 (s, br, 2H), 4.21 (s, br, 2H), 7.13 (d, J = 7.2 Hz, 1H), 7.28 − 7.36 (m, 2H), 7.77 − 7.89 (m, 2H), 8.21 − 8.26 (m, 2H), 9.23 (s, 1H). 9.68 (s, br, 2H). LCMS (Method A): 0.505 min, MS: ES+ 226 (M + 1). Prepared from 3-amino-pyridine. |
| 81 | N-(Pyrazin-2-yl)- 1,2,3,4- tetrahydroisoquinolin- 8-amine hydrochloride | 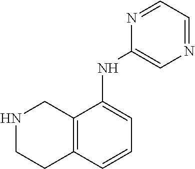 [SMILES: c1cc2c(c(Nc3cnccn3)c1)CNCC2] | ¹H NMR (DMSO-d6, 400 MHz): δ ppm 3.05 (t, J = 6 − 12.4 Hz, 2H), 3.35 (s, br, 2H), 4.17 (s, br, 2H), 7.05 (d, J = 3.2, 1H), 7.28 (t, J = 8- 15.6 Hz, 1H), 7.43 (d, J = 7.6 Hz, 1H), 7.93 (d, J = 2.4 Hz, 1H), 8.03-8.4 (m, 1H), 8.27 (d, J = 1.2 Hz, 1H), 9.00 (s, 1H), 9.50 (s, 2H). LCMS (Method A): 0.774 min, MS: ES+ 227.0. Prepared from 2-aminopyrazine. |
| 82 | N-(Pyridazin-4- yl)-1,2,3,4- tetrahydroisoquinolin- 8-amine hydrochloride | 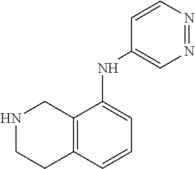 [SMILES: c1cc2c(c(Nc3ccnnc3)c1)CNCC2] | ¹H NMR (DMSO-d6, 400 MHz): δ ppm 3.09 (t, J = 5.6 Hz, 2H) 3.35 (bs, 2H), 3.57 (s, 3H), 4.29 (s, 2H), 5.77 (s, 1H), 7.31 − 7.46 (m, 3H), 8.86 (d, J = 7.2 Hz, 2H), 9.78 (s, 2H), 11.62 (s, 1H). LCMS (Method A): 0.193 min, MS: ES+ 227.0 (M + 1). Prepared from 4- aminopyridazine. |
| 84 | N-(4- Methylpyrimidin- 5-yl)-1,2,3,4- tetrahydroisoquinolin- 8-amine. hydrochloride | 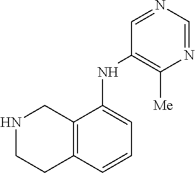 [SMILES: Cc1ncncc1Nc1cccc2c1CNCC2] | ¹H NMR (DMSO-d6, 400 MHz): δ ppm 2.42 (s, 3H), 3.01 − 3.06 (m, 3H), 4.10 − 4.17 (m, 4H), 4.28 − 4.29 (m, 2H), 6.79 (d, J = 8 Hz, 1H), 6.99 − 7.01 (m, 2H), 7.22 − 7.07 (m, 1H), 8.04 (s, 1H), 8.70 (s, 1H), 9.58 − 9.69 (m, 3H). LCMS (Method A): 0.771 min, MS: ES+ 240.96 (M + 1). Prepared from 4-methyl-5- aminopyrimidine. |
| 90 | 1-Methyl-4- ((1,2,3,4- tetrahydroisoquinolin- 8-yl)amino)pyridin- 2(1H)-one hydrochloride | 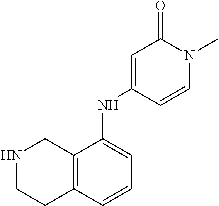 [SMILES: Cn1ccc(Nc2cccc3c2CNCC3)cc1=O] | ¹H NMR (DMSO-d6, 400 MHz): δ ppm 3.03 − 3.07 (m, 2H), 3.33 − 3.36 (m, 2H), 3.57 (s, 3H), 4.15 (s, br, 2H), 5.88 (s, 1H), 6.29 − 6.31 (m, 1H), 7.19 (d, J = 8 Hz, 2H), 7.34 − 7.38 (m, 1H), 7.27 − 7.45 (m, 1H), 9.33 − 9.56 (bs, 2H). LCMS (Method A): 0.757 min, MS: ES+ 255.7 (M + 1). Prepared from 4-amino-1- methylpyridin-2(1H)-one |

### (S)—N-(Tetrahydrofuran-3-yl)-5,6,7,8-tetrahydro-1,6-naphthyridin-4-amine (Intermediate 44)

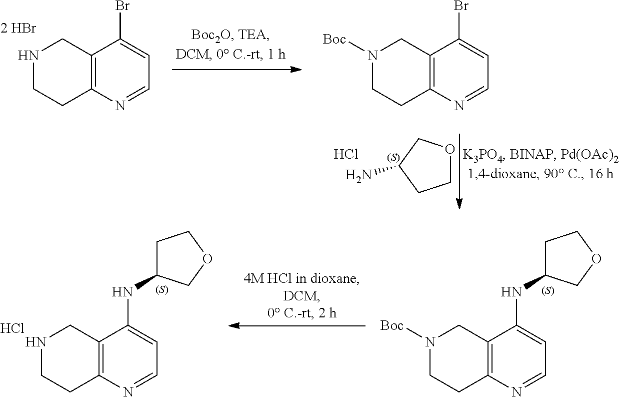

A stirred solution of 4-bromo-5,6,7,8-tetrahydro-1,6-naphthyridine dihydrobromide (0.5 g, 1.33 mmol, 1.0 eq) (CAS 1909337-65-0) in DCM (5 mL) was treated with TEA (0.403 g, 3.99 mmol, 3.0 eq) at 0° C. and stirred for 5 mins. Boc anhydride (0.32 g, 1.46 mmol, 1.1 eq) was added dropwise and the mixture was stirred at room temperature for 1 h. The reaction mixture was poured into water (50 mL) and extracted with ethyl acetate (2×80 mL). The combined organic layer was dried over Na₂SO₄, filtered, and concentrated under vacuum. The crude material was purified by flash chromatography (product eluted with 15% EtOAc in hexane) yielding t-butyl 4-bromo-7,8-dihydro-1,6-naphthyridine-6(5H)-carboxylate (0.4 g, Yield: 95.7%). ¹H NMR (DMSO-d6, 400 MHz): 1.51 (s, 9H), 3.02 (t, J=5.6 Hz, 2H), 3.75 (t, J=6 Hz, 2H), 4.58 (s, 2H), 7.40 (d, J=5.2 Hz, 1H), 8.23 (d, J=5.2 Hz, 1H). LCMS (Method A): 1.764 min, MS: ES+314.91 (M+1).

A mixture of t-butyl 4-bromo-7,8-dihydro-1,6-naphthyridine-6(5H)-carboxylate (0.18 g, 0.574 mmol, 1.0 eq) and, (S)-tetrahydrofuran-3-amine hydrochloride (0.070 g, 0.574 mmol, 1.0 eq) (CAS:204512-95-8), K₃PO₄ (0.645 g, 3.042 mmol, 5.3 eq), BINAP (0.071 g, 0.114 mmol, 0.2 eq) and Pd(OAc)₂ (0.0128, 0.057 mmol, 0.1 eq) in 1,4-dioxane (9 mL) was degassed with argon (gas) for 5 mins. The reaction mixture was heated to 90° C. and stirred for 16 h. The reaction mixture was filtered through celite bed and the celite bed washed with 10% MeOH in DCM (2×50 mL); the combined filtrate was concentrated under vacuum. The crude material was purified by flash chromatography (product eluted with 10% MeOH in DCM) yielding t-butyl (S)-4-((tetrahydrofuran-3-yl)amino)-7,8-dihydro-1,6-naphthyridine-6(5H)-carboxylate (0.34 g, Yield: 61.7%). ¹H NMR (DMSO-d6, 400 MHz): 1.42 (s, 9H), 1.86-1.93 (m, 1H), 2.15-2.23 (m, 1H), 2.69 (t, J=5.6 Hz, 2H), 3.56-3.64 (m, 3H), 3.69-3.74 (m, 1H), 3.81-3.92 (m, 2H), 4.09 (s, 1H), 4.27 (s, 2H), 5.87 (s, 1H), 6.45 (d, J=5.6 Hz, 1H), 7.98 (d, J=5.6 Hz, 1H). LCMS (Method A): 1.084 min. MS: ES+264.0 (M−56).

A stirred solution of t-butyl (S)-4-((tetrahydrofuran-3-yl)amino)-7,8-dihydro-1,6-naphthyridine-6(5H)-carboxylate (0.3 g, 0.93 mmol, 1.0 eq) in DCM (3 mL) was cooled to 0° C. 4M HCl in 1,4-dioxane (1.5 mL)was added and the reaction mixture was stirred at room temperature for 2 h. The reaction mixture was concentrated under vacuum and crude material triturated using diethyl ether (2×15 mL) yielding (S)—N-(tetrahydrofuran-3-yl)-5,6,7,8-tetrahydro-1,6-naphthyridin-4-amine hydrochloride (0.25 g, Yield: quantitative). ¹H NMR (DMSO-d6, 400 MHz): 1.98-2.02 (m, 1H), 2.25-2.32 (m, 1H), 3.15 (t, J=5.6 Hz, 2H), 3.39 (s, 2H), 3.72-3.77 (m, 2H), 3.86-3.93 (m, 2H), 4.06 (s, 2H), 6.99 (d, J=7.2 Hz, 1H), 7.86 (d, J=6 Hz, 1H), 8.25-8.27 (m, 1H), 10.34 (s, 2H). LCMS (Method B): 1.93 min. MS: ES+220.34 (M+1).

### 4-((4-Methylpiperazin-1-yl)methyl)isoindoline hydrochloride (Intermediate 6)

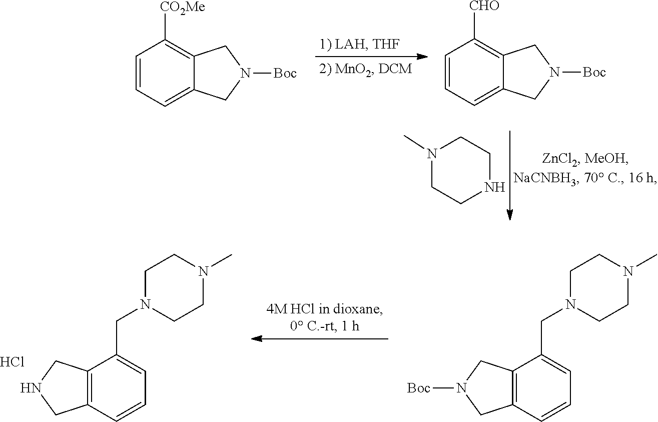

### t-Butyl-4-(hydroxymethyl)isoindoline-2-carboxylate

A stirred solution of 2-(t-butyl)-4-methyl isoindoline-2, 4-dicarboxylate (CAS 1710854-30-0) (4.0 g, 14.4 mmol, 1.0 eq) in THF (80 mL) at 0° C. was treated dropwise with LiAIH₄ solution (1.0 M in THF) (14.4 mL, 14.4 mmol, 1.0 eq). The resulting reaction mixture was stirred at 0° C. for 1 h and carefully poured onto ice cold saturated NH₄Cl in water (50 mL) and extracted with ethyl acetate (3×100 mL). The combined organic layer was dried over Na₂SO₄, filtered, and concentrated under vacuum yielding the title compound (3.5 g. Yield: 97.34%). ¹H NMR (DMSO-d6, 400 MHz): δ 1.46 (s, 9H), 4.46-4.48 (m, 2H), 4.56-4.59 (m, 4H), 5.16-5.20 (m, 1H), 7.21-7.28 (m, 3H). LCMS (Method A): 1.609 min, MS: ES+150.1 (M−100).

### t-Butyl 4-formylisoindoline-2-carboxylate (Intermediate 58)

To a stirred solution of t-butyl 4-(hydroxymethyl) isoindoline-2-carboxylate (3.5 g, 14.04 mmol, 1.0 eq) in DCM (70 mL) was added MnO₂ (35 g, 10% w/w) at room temperature. The resulting reaction mixture was stirred at room temperature for 16 h, filtered through a celite bed, washed with 10% MeOH in DCM (3×100 mL) and the combined filtrate concentrated under vacuum. The crude material was purified by flash chromatography (product eluted with 8% ethyl acetate in n-hexane) to give the title compound (2.0 g. Yield: 57.6%). ¹H NMR (DMSO-d6, 400 MHz): 1.46-1.47 (m, 9H), 4.61 (d, J=10.4 Hz, 2H), 4.83 (d, J=8 Hz, 2H), 7.58 (t, J=6.4 Hz, 1H), 7.67 (t, J=5.6 Hz, 1H), 7.90 (d, J=6.0 Hz, 1H), 10.06 (d, J=5.6 Hz, 1H). LCMS (Method A): 1.993 min. MS: ES+192.1 (M−56).

### t-Butyl-4-((4-methylpiperazin-1-yl)methyl)isoindoline-2-carboxylate

Three batches of 0.3 g scale progressed in parallel. A stirred solution of t-butyl 4-formylisoindoline-2-carboxylate (Intermediate 58) (0.3 g, 1.21 mmol, 1.0 eq) in MeOH (10 mL) at room temperature was treated with N-methyl piperazine (0.121 g, 1.21 mmol, 1.0 eq) and ZnCl₂ (0.078 g, 0.60 mmol, 0.5 eq). The reaction mixture was heated to 70° C. and stirred for 16 h. NaCNBH₃ (0.152 g, 2.42 mmol, 2.0 eq) was added portionwise to the reaction mixture at 0° C. The resulting reaction mixture was stirred at room temperature for 16 h. The reaction mixture was concentrated under vacuum, poured into water (50 mL) and extracted with ethyl acetate (3×100 mL). The combined organic layer was washed by brine solution (30 mL), dried with Na₂SO₄, filtered, and concentrated under vacuum. The crude material was purified by flash chromatography (product eluted with 4% MeOH in DCM) to give the title compound (0.6 g. Yield 49.7%). ¹H NMR (DMSO-d6, 400 MHz): δ 1.46 (s, 9H), 2.61-2.63 (m, 5H), 2.95-2.97 (m, br, 4H), 3.34-3.35 (m, br, 4H), 4.57-4.63 (m, 4H), 7.19-7.25 (m, 3H). LCMS (Method A): 1.212 min, MS: ES+332.37 (M+1).

### 4-((4-Methylpiperazin-1-yl)methyl) isoindoline hydrochloride (Intermediate 6)

To a stirred solution of t-butyl 4-((4-methylpiperazin-1-yl) methyl)isoindoline-2-carboxylate (0.3 g, 0.9 mmol, 1.0 eq) in DCM (2 mL) was added 4M HCl in dioxane (1 mL) at 0° C. The resulting reaction mixture was stirred at room temperature for 2 h. The reaction mixture was concentrated under vacuum yielding the title compound which was used directly in the next step (0.3 g, Yield: quantitative, 1.29 mmol). LCMS (Method A): 0.165 min, MS: ES+232.19 (M+1).

### 4-(Isoindolin-4-ylmethyl)morpholine hydrochloride (Intermediate 7)

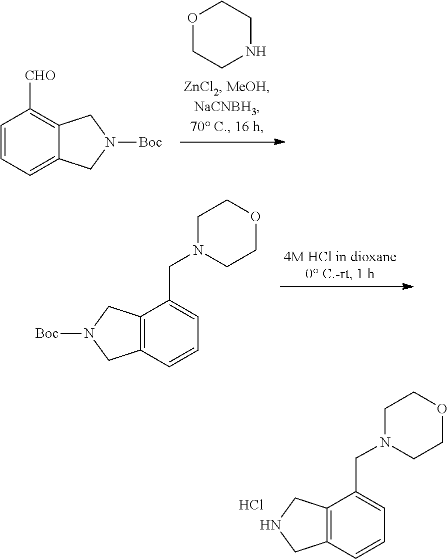 [SMILES: C1COCCN1 ; CC(C)(C)OC(=O)N1Cc2cccc(C=O)c2C1 ; CC(C)(C)OC(=O)N1Cc2cccc(CN3CCOCC3)c2C1 ; Cl ; c1cc2c(c(CN3CCOCC3)c1)CNC2]

A stirred solution of t-butyl 4-formylisoindoline-2-carboxylate (Intermediate 58) (0.4 g, 1.62 mmol, 1.0 eq) in MeOH (5 mL) at rt was treated with morpholine (0.169, 1.94 mmol, 1.2 eq) and ZnCl₂ (0.110 g, 0.810 mmol, 0.5 eq). The resulting reaction mixture was heated to 70° C. and stirred for 16 h. NaCNBH₃ (0.203 g, 3.23 mmol, 2.0 eq) was added to the reaction mixture at 0° C. The reaction mixture was stirred at room temperature for 2 h and concentrated under vacuum. The crude material was purified by flash chromatography (product eluted at 30% ethyl acetate in hexane) yielding t-butyl 4-(morpholinomethyl)isoindoline-2-carboxylate as a yellow solid (0.6 g, Yield: 58.25%, 1.89 mmol). ¹H NMR (DMSO-d6, 400 MHz): δ ppm 1.45-1.46 (m, 9H), 2.33 (m, 4H), 3.43 (s, 2H), 3.55-3.56 (m, 4H), 3.56-3.64 (m, 4H), 7.18-7.24 (m, 3H). LCMS (Method A): 1.221 min, MS: ES+319.2.

A stirred solution of t-butyl 4-(morpholinomethyl)isoindoline-2-carboxylate (0.6 g, 1.89 mmol, 1.0 eq) in DCM (5 mL) was cooled to 0° C. 4M HCl in dioxane (6 mL) was drop wise added to reaction mixture at 0° C. The reaction mixture was stirred at room temperature for 1 h. The resulting reaction mixture was concentrated under vacuum. The obtained crude material was triturated using n-pentane (3×10 mL) followed by diethyl ether (10 mL) and dried under high vacuum yielding 4-(isoindolin-4-ylmethyl)morpholine hydrochloride (Intermediate 7) as pink solid (0.6 g, Quantitative). ¹H NMR (DMSO-d6, 400 MHz): δ ppm 3.16-3.27 (m, 4H), 3.56 (s, 2H), 3.70 (s, 5H), 3.85-3.93 (m, 4H), 4.34-4.35 (m, 2H), 4.51-4.54 (m, 2H), 4.81-4.84 (m, 2H), 7.44-7.51 (m, 2H), 7.68-7.70 (m, 1H), 10.2 (s, 2H). LCMS (Method A): 1.65 min, MS: ES+219.0 (M+1).

### t-Butyl (5-(4-(isoindolin-5-ylmethyl)piperazin-1-yl)pentyl)carbamate (Intermediate 69)

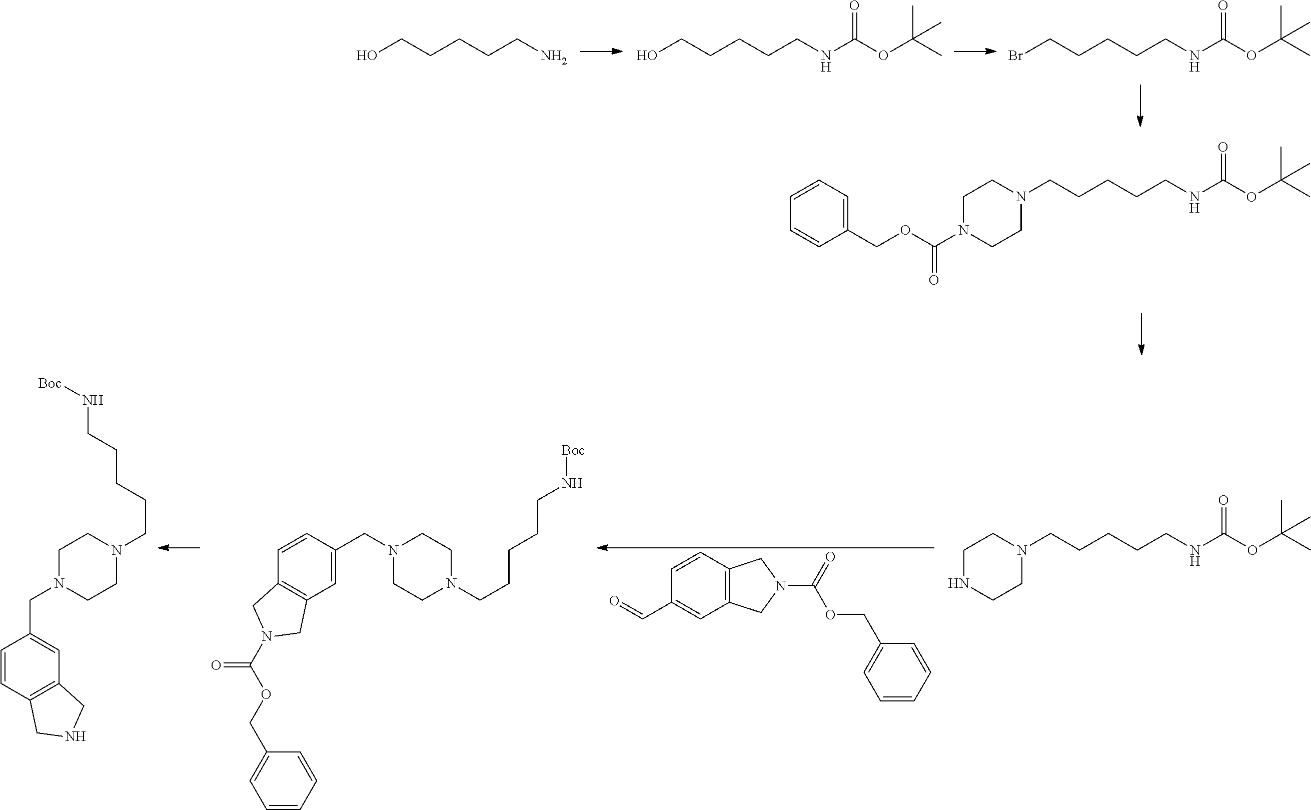

### t-Butyl (5-hydroxypentyl)carbamate

5-Aminopentan-1-ol (CAS 2508-29-4) (5.0 g, 48.54 mmol) in DCM (50 mL) was cooled to 0° C. and treated with Boc anhydride (11.16 mL, 48.46 mmol). The resulting reaction was warmed to room temperature and stirred for 16 h, poured into water (150 mL) and extracted with DCM (2×100 mL). The combined organic layer was dried over anhydrous sodium sulphate, filtered and concentrated under vacuum. The crude material was purified by flash chromatography (silica, eluting with 18% (v/v) ethyl acetate in hexane) yielding tert-butyl (5-hydroxypentyl)carbamate as a pale yellow oil (15.0 g, 76%). ¹H NMR (CDC₃, 400 MHz): δ 1.34-1.40 (m, 2H), 1.43 (s, 9H), 1.46-1.51 (m, 2H), 1.54-1.61 (m, 2H), 1.97 (br. s, 1H), 3.10-3.11 (m, 2H), 6.63 (t, J=6.40 Hz, 2H), 4.63 (bs, 1H).

### t-Butyl (5-bromopentyl) carbamate

A mixture of tert-butyl (5-hydroxypentyl)carbamate (8.0 g, 39.41 mmol) in THF (80 mL) was treated with triphenyl phosphine (15.48 g, 59.11 mmol) and CBr₄ (19.60 g, 59.11 mmol) at 0° C. The resulting reaction mixture was slowly warmed to room temperature and stirred for 16 h. The reaction mixture was poured into water (150 mL) and extracted with ethyl acetate (3×150 mL). The combined organic layer was dried over sodium sulphate, filtered, and concentrated under reduced pressure. The crude material was purified by column chromatography (silica gel, eluting with 10% (v/v) ethyl acetate in hexane) yielding t-butyl (5-bromopentyl) carbamate as a colorless oil. (9.0 g, 86%). LCMS (Method A): 2.150 min, MS: ES+210.1, 212.1 (M−56); ¹H NMR (CDCl₃, 400 MHz): δ 1.32-1.36 (m, 13H), 1.75-1.79 (m, 2H), 2.88-2.90 (m, 2H), 3.51 (t, J=6.8 Hz, 2H), 6.81 (t, J=5.6 Hz, 1H).

### Benzyl 4-(5-((t-butoxycarbonyl)amino)pentyl)piperazine-1-carboxylate

Benzyl piperazine-1-carboxylate (CAS 31166-44-6) (4.135 g, 18.79 mmol) in acetone (50 mL) was treated with K₂CO₃ (7.78 g, 56.38 mmol) and NaI (1.40 g, 9.39 mmol) at room temperature and stirred for 15 min. t-Butyl(5-bromopentyl)carbamate (5.0 g, 18.79 mmol) was added and the reaction mixture heated at 70° C. for 16 h, cooled to room temperature, diluted with water (150 mL) and extracted with ethyl acetate (3×150 mL). The combined organic layer was dried over anhydrous sodium sulphate, filtered and concentrated under vacuum to give crude material that was purified by flash chromatography (silica gel, eluting with 3% (v/v) MeOH in DCM) yielding benzyl 4-(5-((t-butoxycarbonyl) amino)pentyl)piperazine-1-carboxylate as a brown oil (6.5 g, 85%). LCMS (Method A): 1.492 min, MS: ES+406.42 (M+1); ¹H NMR (DMSO-d⁶, 400 MHz): δ 1.21-1.23 (m, 2H), 1.35 (s, 11H), 2.28-2.32 (m, 6H), 2.85-2.90 (m, 2H), 3.32-3.34 (m, 6H), 5.06 (m, 2H), 6.77 (s, 1H), 7.31-7.38 (m, 5H).

### t-Butyl (5-(piperazin-1-yl)pentyl)carbamate

Benzyl 4-(5-((tert-butoxycarbonyl)amino)pentyl)piperazine-1-carboxylate (6.4 g, 15.80 mmol) in MeOH (70 mL) was treated with 10% Pd/C (50% moisture) (0.640 g, 10% w/w) at room temperature. The reaction mixture was purged with H₂(g) for 5 h, filtered through a celite bed, washed with MeOH (2×10 mL) and the combined filtrate concentrated under reduced pressure to give t-butyl (5-(piperazin-1-yl)pentyl)carbamate as a brown oil (4.0 g, 93%). This material was carried forward to the next step without further purification. MS: ES+272.20 (M+1). ¹H NMR (CDCl₃, 400 MHz): δ 1.28-1.35 (m, 2H), 1.43 (s, 9H), 1.43-1.50 (m, 2H), 2.14 (br. s, 3H), 2.29-2.33 (m, 2H), 2.42 (bs, 4H), 2.90-2.92 (m, 3H), 3.10-3.11 (m, 2H), 3.47 (s, 1H), 4.61 (bs, 1H).

### Benzyl-5-((4-(5-((t-butoxycarbonyl)amino)pentyl)piperazin-1-yl)methyl)isoindoline-2-carboxylate

Benzyl 5-formylisoindoline-2-carboxylate (Intermediate 73) (1.6 g, 5.69 mmol) and t-butyl (5-(piperazin-1-yl)pentyl)carbamate 1.54 g, 5.69 mmol) in DCM (50 mL) were treated with acetic acid (0.2 mL) and stirred at room temperature for 2 h. The reaction mixture was cooled to 0° C. and NaBH(OAc)₃ (3.618 g, 17.078 mmol) added. The reaction mixture was slowly warmed to room temperature and stirred for 16 h, poured into water (50 mL) and extracted with DCM (2×150 mL). The combined organic layer was dried over sodium sulphate, filtered and concentrated under vacuum; the crude material was purified by flash chromatography (silica gel, eluting with 3% (v/v) MeOH in DCM) to give benzyl-5-((4-(5-((t-butoxycarbonyl)amino)pentyl)-piperazin-1-yl)methyl)isoindoline-2-carboxylate as a brown oil (2.2 g, 72%). LCMS (Method A): 1.552 min, MS: ES+537.41 (M+1); ¹H NMR (DMSO-d⁶, 400 MHz): δ 1.15-1.23 (m, 2H), 1.36 (s, 11H), 1.51 (br. s, 2H), 1.90 (s, 1H), 2.32-2.39 (m, 2H), 2.88-2.95 (m, 7H), 3.53 (br. s, 2H), 4.63-4.69 (m, 4H), 5.14 (s, 2H), 6.82 (br. s, 1H), 7.23-7.42 (m, 8H), 10.04 (br. s, 1H), 11.99 (br. s, 1H).

### t-Butyl (5-(4-(isoindolin-5-ylmethyl)piperazin-1-yl)pentyl)carbamate (Intermediate 69)

Benzyl-5-((4-(5-((t-butoxycarbonyl)amino)pentyl)piperazin-1-yl)methyl)isoindoline-2-carboxylate (2.1 g, 3.92 mmol) in EtOH (60 mL) was treated with 10% Pd/C (50% moisture) (0.420 g, 20% w/w) at room temperature and then purged with H₂(g) for 5 h. The reaction mixture was filtered through a celite bed, washed with MeOH (2×20 mL) and the combined filtrate concentrated under vacuum to yield tert-butyl (5-(4-(isoindolin-5-ylmethyl)piperazin-1-yl)pentyl)carbamate (Intermediate 69) as brown oil (1.7 g, quantitative). This material was carried forward without further purification. LCMS (Method A): 0.757 min, MS: ES+403.37 (M+1); ¹H NMR (DMSO-d⁶, 400 MHz): δ 1.19-1.22 (m, 2H), 1.36 (s, 13H), 2.19-2.21 (m, 2H), 2.23-2.27 (m, 3H), 2.28-2.32 (m, 3H), 2.86-2.87 (m, 2H), 2.96-2.97 (m, 1H), 3.40-3.43 (m, 4H), 4.32-4.33 (m, 4H), 6.77-6.79 (m, 1H), 7.15-7.29 (m, 3H).

### Benzyl 5-formylisoindoline-2-carboxylate (Intermediate 73)

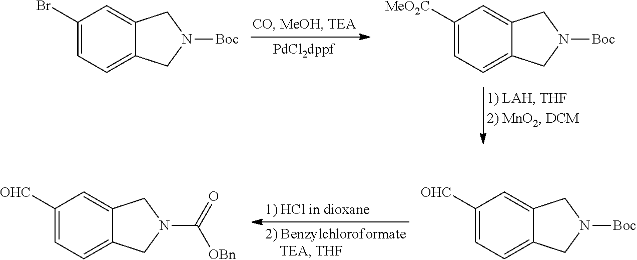 [SMILES: CC(C)(C)OC(=O)N1Cc2ccc(Br)cc2C1 ; COC(=O)c1ccc2c(c1)CN(C(=O)OC(C)(C)C)C2 ; O=Cc1ccc2c(c1)CN(C(=O)OCc1ccccc1)C2 ; CC(C)(C)OC(=O)N1Cc2ccc(C=O)cc2C1]

### t-Butyl-5-methyl isoindoline-2,5-dicarboxylate

tert-Butyl 5-bromoisoindoline-2-carboxylate (CAS 201940-08-1) (10.0 g, 33.5 mmol) in MeOH:DMF (9:1, 100 mL) at room temperature was treated with TEA (14 mL, 137.81 mmol). The reaction mixture was degassed using N₂ for 10-15 min. PdCl₂(dppf) (4.91 g, 6.71 mmol) was added and the reaction mixture subject to 30 kg/cm² of CO_{(gas)} pressure and heated to 120° C. under stirring for 16 h. The reaction mixture was cooled to room temperature and concentrated under reduced pressure. The obtained crude material was diluted with water (200 mL) and extracted in EtOAc (3×100 mL). The combined organic layer was washed with brine solution (200 mL), dried over sodium sulphate and concentrated under reduced pressure to give crude material which was purified by column chromatography (silica gel, eluting with 12% ethyl acetate in hexane) yielding tert-butyl-5-methyl isoindoline-2,5-dicarboxylate as a brown solid (10.0 g). LCMS ( ): 2.657 min, MS: ES+278.18 (M+1); ¹H NMR (DMSO-d⁶, 400 MHz): δ 1.45 (s, 9H), 3.84 (s, 3H), 4.61 (t, J=7.6 Hz, 4H), 7.43-7.47 (m, 1H), 7.86-7.91 (m, 2H).

### t-Butyl 5-(hydroxymethyl)isoindoline-2-carboxylate

LiAIH₄ solution (1.0M in THF) (36 mL, 36.10 mmol) was added dropwise to a stirred solution of 2-tert-butyl-5-methyl isoindoline-2,5-dicarboxylate (10.0 g, 36.07 mmol) in THF (200 mL) at 0° C. The reaction mixture was stirred at 0° C. for 3 h and then poured into cold saturated aqueous ammonium chloride solution (100 mL) and extracted into ethyl acetate (3×100 mL). The combined organic layer was dried over anhydrous sodium sulphate, filtered and concentrated under vacuum yielding tert-butyl 5-(hydroxymethyl)isoindoline-2-carboxylate as brown solid (5.0 g, 56%). This material was used in the next step without further purification. LCMS (Method H): 2.061 min, MS: ES+250.18 (M+1); ¹H NMR (DMSO-d⁶, 400 MHz): δ 1.45 (s, 9H), 4.48-4.55 (m, 6H), 5.21 (br. s, 1H), 7.19-7.26 (m, 3H).

### t-Butyl 5-formylisoindoline-2-carboxylate

t-Butyl 5-(hydroxymethyl)isoindoline-2-carboxylate (3.0 g, 12.04 mmol) in DCM (30 mL) at room temperature was treated with activated MnO₂ (10.36 g, 120.40 mmol) and stirred for 16 h. The reaction mixture was filtered through a celite bed, washed with 10% MeOH:DCM (3×50 mL). The combined filtrate was concentrated under vacuum yielding tert-butyl-5-formylisoindoline-2-carboxylate as a grey solid (2.6 g, 86%). This material was used in the next step without any further purification. LCMS (Method C): 2.387 min, MS: ES+248.18 (M+1); ¹H NMR (CDCl₃, 400 MHz): δ 1.54 (s, 9H), 4.73-4.76 (m, 4H), 7.40-7.45 (dd, J=8.4, 7.6 Hz, 1H), 7.77-7.82 (m, 2H), 10.20 (s, 1H).

### Isoindoline-5-carbaldehyde hydrochloride

4M HCl in dioxane (20 mL) was added dropwise to a solution of t-butyl-5-formylisoindoline-2-carboxylate (1.30 g, 5.26 mmol) in DCM (20 mL) 0° C. The reaction mixture was slowly warmed to room temperature and stirred for 6 h, concentrated under reduced pressure and the crude material purified by trituration with diethyl ether (3×20 mL). The resultant solid material was dried under high vacuum yielding isoindoline-5-carbaldehyde hydrochloride as an off-white solid (0.80 g, 83%). LCMS (Method H): 2.154 min, MS: ES+148.18 (M+1); ¹H NMR (DMSO-d⁶, 400 MHz) δ 4.57 (d, J=4.0 Hz, 4H), 7.62 (d, J=8.0 Hz, 1H), 7.92 (d, J=7.6 Hz, 2H), 10.21 (s, 1H), 10.63 (s, 1H).

### Benzyl 5-formylisoindoline-2-carboxylate (Intermediate 73)

Isoindoline-5-carbaldehyde hydrochloride (0.80 g, 4.37 mmol) in THF (10 mL) at room temperature was treated with TEA (2.30 mL, 13.02 mmol). The reaction mixture was cooled to 0° C. and benzyl chloroformate (0.745 g, 4.38 mmol) was added dropwise. The reaction mixture was stirred at 0° C. for 30 min, concentrated under vacuum, diluted with 2M HCl (10 mL) and extracted with ethyl acetate (3×30 mL). The combined organic layer was washed with water (100 mL), dried over anhydrous sodium sulphate, filtered and concentrated under vacuum. The crude material was purified by column chromatography (silica gel, eluting with 25% (v/v) ethyl acetate in hexane) yielding benzyl 5-formylisoindoline-2-carboxylate (Intermediate 73) as a yellow solid (1.0 g, 82%). ¹H NMR (DMSO-d⁶, 400 MHz) δ 4.72-4.80 (m, 4H), 5.15 (s, 2H), 7.32-7.43 (m, 5H), 7.53-7.58 (m, 1H), 7.84-7.88 (m, 2H), 9.99 (d, J=1.6 Hz, 1H).

### (S)—N-(Tetrahydrofuran-3-yl)-5,6,7,8-tetrahydropyrido[4,3-d]pyrimidin-4-amine hydrochloride (Intermediate 77)

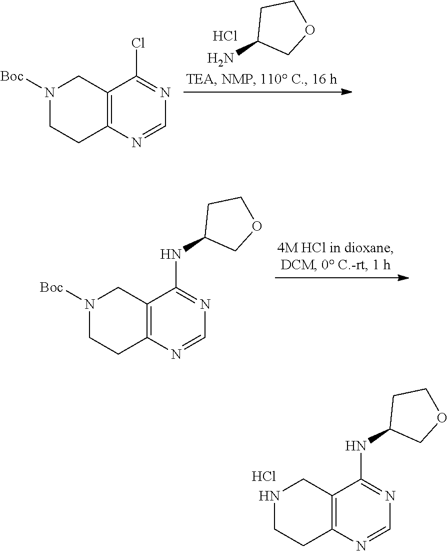 [SMILES: CC(C)(C)OC(=O)N1CCc2ncnc(Cl)c2C1 ; Cl ; N[C@H]1CCOC1 ; CC(C)(C)OC(=O)N1CCc2ncnc(N[C@H]3CCOC3)c2C1 ; Cl ; c1nc2c(c(N[C@H]3CCOC3)n1)CNCC2]

### t-Butyl (S)-4-((tetrahydrofuran-3-yl) amino)-7, 8-dihydropyrido [4,3d] pyrimidine-6(5H)-carboxylate

A mixture of t-butyl 4-chloro-7,8-dihydropyrido[4,3-d]pyrimidine-6(5H)-carboxylate (0.7 g, 2.59 mmol, 1 eq.) (CAS: 1056934-87-2), (S)-tetrahydrofuran-3-amine hydrochloride (0.47 g, 3.88 mmol, 1.5 eq.), TEA (0.7 mL, 5.18 mmol, 2.0 eq.) in NMP (7 mL) was placed in a 35 mL glass vial. The reaction mixture was heated to 110° C. and stirred for 16 h, cooled, poured into water (80 mL) and extracted using ethyl acetate (3×60 mL). The combined organic layer was washed with cold brine solution (3×80 mL) and dried over Na₂SO₄, filtered, and concentrated under vacuum. Crude material was purified by flash chromatography (product eluted in 3.2% MeOH in DCM) yielding the title compound as a yellow sticky solid (0.56 g, Yield: 68.3%, 1.75 mmol). ¹H NMR (DMSO-d6, 400 MHz): δ ppm 1.43 (s, 9H), 1.95-1.98 (m, 1H), 2.13-2.19 (m, 1H), 2.51-2.63 (m, 2H), 3.56-3.61 (m, 4H), 3.68-3.73 (m, 1H), 3.83-3.91 (m, 2H) 4.24 (s, 2H), 4.58-4.60 (m, 1H) 6.85 (bs, 1H, D₂O exchangeable), 8.29 (s, 1H). LCMS (Method A): 1.000 min, MS: ES+321.2 (M+1).

### (S)—N-(Tetrahydrofuran-3-yl)-5,6,7,8-tetrahydropyrido[4,3-d]pyrimidin-4-amine hydrochloride

A stirred solution of t-butyl (S)-4-((tetrahydrofuran-3-yl) amino)-7, 8-dihydropyrido [4,3d]pyrimidine-6(5H)-carboxylate as (0.53 g, 1.56 mmol, 1.0 eq.) in DCM (5 mL) was cooled to 0° C. 4M HCl in 1,4-dioxane (5 mL) was added dropwise to the reaction mixture at 0° C. The reaction mixture was stirred at room temperature for 1 h and then concentrated under vacuum. Crude material was triturated using n-pentane (2×5 mL) followed by diethyl ether (2×5 mL) and the obtained material was dried under high vacuum yielding the title compound (0.53 g, Yield: quantitative, 2.40 mmol). ¹H NMR (DMSO-d6, 400 MHz): δ ppm 2.04-2.09 (m, 1H), 2.19-2.26 (m, 1H), 3.09-3.10 (m, 2H), 3.30 (s, 2H), 3.69-3.77 (m, 3H), 3.88-3.94 (m, 3H), 4.06 (s, 2H), 4.76-4.77 (m, 1H), 8.84 (s, 1H). LCMS (Method A): 1.68 min, MS: ES+221 (M+1).

### 7-Fluoro-N-(tetrahydrofuran-3-yl)-1,2,3,4-tetrahydroisoquinolin-8-amine. hydrochloride (Intermediate 78)

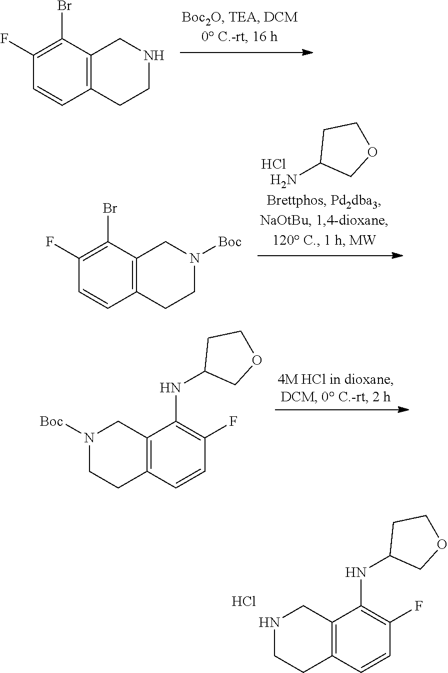

### t-Butyl-8-bromo-7-fluoro-3,4-dihydroisoquinoline-2(1H)-carboxylate

A stirred solution of 8-bromo-7-fluoro-1,2,3,4-tetrahydroisoquinoline (CAS: 178581-08-9) (0.5 g, 2.17 mmol, 1 eq.) in DCM (10 mL) was treated with TEA (0.44 g, 4.35 mmol, 2 eq.) and stirred for 5 mins. Boc anhydride (0.71 g, 3.26 mmol, 1.5 eq.) was added to the reaction mixture and stirred for 16 h. The reaction mixture was poured into water (20 mL) and extracted in ethyl acetate (2×30 mL). The combined organic layer was dried over Na₂SO₄, filtered, and concentrated under vacuum. Crude material was purified by flash chromatography (product eluted in 10% EtOAc in Hexane) yielding the title compound as an off white solid (0.45 g, Yield: 62%, 1.36 mmol). ¹H NMR (DMSO-d6, 400 MHz): δ ppm 1.48 (s, 9H), 2.79 (t, J=5.6 Hz, 2H), 3.56 (t, J=6 Hz, 2H), 4.48 (s, 2H), 7.23-7.26 (m, 2H). LCMS (Method A): 2.549 min, MS: ES+273.9 (M−56).

### t-Butyl 7-fluoro-8-((tetrahydrofuran-3-yl)amino)-3,4-dihydroisoquinoline-2(1H)-carboxylate

A mixture of t-butyl 8-bromo-7-fluoro-3,4-dihydroisoquinoline-2(1H)-carboxylate (0.45 g, 1.36 mmol, 1 eq.), tetrahydrofuran-3-amine hydrochloride (CAS: 204512-94-7) (0.25 g, 2.04 mmol, 1.5 eq.), NaOtBu (0.327 g, 3.40 mmol, 2.5 eq.) and in 1,4-dioxane (10 mL) was added to a 35 mL glass vial and degassed using argon gas for 5 mins. Brettphos (0.044 g, 0.082 mmol, 0.06 eq.) and Pd₂dba₃ (0.050 g, 0.054 mmol, 0.04 eq.) were added to the reaction mixture at room temperature. The resulting reaction mixture was heated to 120° C. under microwave irradiation for 2 h. The reaction mixture was poured into water (20 mL) and extracted with ethyl acetate (2×25 mL). The combined organic layer was dried over Na₂SO₄, filtered, and concentrated under vacuum. Crude material was purified by flash chromatography (product eluted in 20% EtOAc in n-Hexane) yielding the title compound as a brown sticky solid (0.28 g, Yield: 61%, 0.83 mmol). LCMS (Method A): 2.155 min, MS: ES+281 (M−56).

### 7-Fluoro-N-(tetrahydrofuran-3-yl)-1,2,3,4-tetrahydroisoquinolin-8-amine hydrochloride

A stirred solution of t-butyl-7-fluoro-8-((tetrahydrofuran-3-yl)amino)-3,4-dihydroisoquinoline-2(1H)-carboxylate (0.28 g, 0.832 mmol, 1.0 eq.) in DCM (5 mL) was cooled to 0° C. 4M HCl in dioxane (1 mL) was added dropwise to the reaction mixture at 0° C. The reaction mixture was stirred at room temperature for 2 h. The reaction mixture was concentrated under vacuum and crude material was triturated using diethyl ether (2×5 mL) and dried under high vacuum yielding the title compound (0.23 g, Yield: quantitative, 0.843 mmol). ¹H NMR (DMSO-d6, 400 MHz): δ ppm 1.63-1.84 (m, 4H), 2.04-2.09 (m, 2H), 2.91-2.94 (m, 2H), 3.20-3.31 (m, 2H), 4.07-4.13 (m, 3H), 6.68-6.72 (m, 1H), 7.02-7.17 (m, 1H), 9.36 (s, br, 2H). LCMS (Method A): 0.909 min, MS: ES+237 (M+1).

### (S)-6-((4-Methylpiperazin-1-yl)methyl)-N-(tetrahydrofuran-3-yl)-1,2,3,4-tetrahydroisoquinolin-8-amine hydrochloride (Intermediate 83)

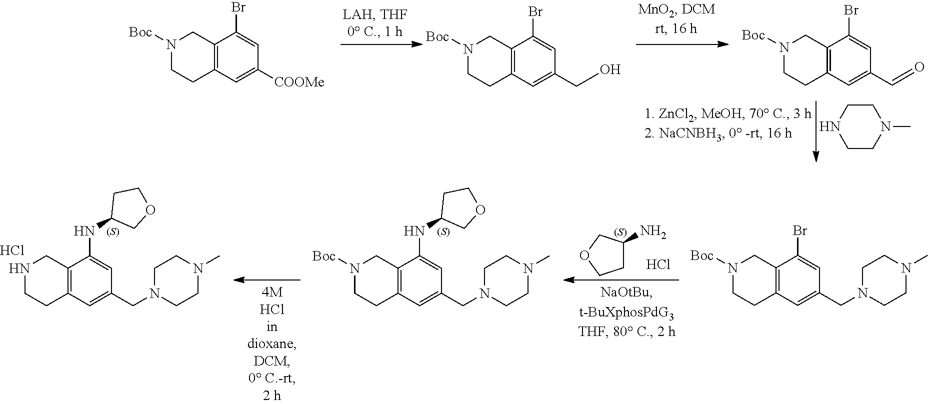

### t-Butyl 8-bromo-6-(hydroxymethyl)-3,4-dihydroisoquinoline-2(1H)-carboxylate

A stirred solution of 2-(t-butyl) 6-methyl 8-bromo-3, 4-dihydroisoquinoline-2, 6(1H)-dicarboxylate (Intermediate 99) (7.0 g, 18.9 mmol, 1.0 eq.) in THF was treated with lithium aluminium hydride solution (1.0 M in THF) (18.9 mL, 18.9 mmol, 1.0 eq.) at 0′C. The reaction mixture was stirred for 1 h at 0° C. and then slowly poured into ice cold water (100 mL) and extracted by ethyl acetate (2×150 mL). The combined organic layer was dried over Na₂SO₄, filtered, and concentrated under vacuum. Crude material was purified by flash chromatography (product eluted with 25% EtOAc in hexane) yielding the title compound (4.0 g, Yield: 62.0%,). ¹H NMR (DMSO-d6, 400 MHz): δ ppm 1.43 (s, 9H), 2.78 (t, J=6.0 Hz, 2H), 3.55 (t, J=6.0 Hz, 2H), 4.42-4.46 (m, 4H), 5.29 (t, J=5.6 Hz, 1H, D₂O exchangeable), 7.13 (s, 1H), 7.42 (s, 1H). LCMS (Method A): 2.016 min, MS: ES+241.7 (M−100).

### t-Butyl-8-bromo-6-formyl-3, 4-dihydroisoquinoline-2(1H)-carboxylate

A stirred solution of t-butyl 8-bromo-6-(hydroxymethyl)-3,4-dihydroisoquinoline-2(1H)-carboxylate (4.5 g, 13 mmol, 1.0 eq.) in MeOH (135 mL, 30 v) was treated with MnO₂ (Activated) (45.88 g, 527 mmol, 40.0 eq.) at room temperature and stirred for 16 h. The reaction mixture was filtered through celite bed and washed with 10% MeOH in DCM (3×100 mL) solution. The filtrate was concentrated under vacuum. Crude material was purified by flash chromatography (product eluted with 12% EtOAc in hexane) yielding the title compound (3.2 g, Yield: 71.3%,). ¹H NMR (DMSO-d6, 400 MHz): δ ppm 1.44 (s, 9H), 2.90 (t, J=5.6 Hz, 2H), 3.59 (t, J=6.0 Hz, 2H), 4.52 (s, 2H), 7.75 (s, 1H), 7.42 (s, 1H), 7.99 (d, J=1.2 Hz, 1H), 9.94 (s, 1H). LCMS (Method A): 2.295 min, MS: ES+283.6 (M−56).

### t-Butyl-8-bromo-6-((4-methylpiperazin-1-yl) methyl)-3, 4-dihydroisoquinoline-2(1H)-carboxylate

Carried out in two parallel batches at 0.6 g scale. A stirred solution of t-butyl 8-bromo-6-formyl-3, 4-dihydroisoquinoline-2(1H)-carboxylate (0.6 g, 1.769 mmol, 1.0 eq.) in MeOH (6 mL) at room temperature was treated with N-methyl piperazine (0.35 g, 3.53 mmol, 1.0 eq.) and ZnCl₂ (0.229 g, 1.76 mmol, 1.0 eq.). The reaction mixture was heated to 70° C. and stirred for 3 h then cooled. NaCNBH₃ (0.22 g, 3.53 mmol, 2.0 eq.) was added to the reaction mixture at 0° C. The resulting reaction mixture was stirred at room temperature for 16 h and concentrated under vacuum. Crude material was diluted with ethyl acetate (250 mL) and washed with water (2×50 mL). The organic layer was dried over Na₂SO₄, filtered, and concentrated under vacuum. Crude material was purified by flash chromatography (product was eluted with 5% MeOH in DCM) yielding the title compound (1.1 g, Yield: 73.5%). ¹H NMR (DMSO-d6, 400 MHz): δ ppm 1.43 (s, 9H), 2.16 (s, 3H), 2.34-2.46 (m, 6H), 2.76-2.79 (m, 2H), 3.40 (s, 2H), 3.53-3.56 (m, 2H), 4.41 (s, 2H), 7.11 (s, 1H), 7.39 (s, 1H). LCMS (Method A): 1.505 min. MS: ES+423.9 (M+1).

### t-Butyl-(S)-6-((4-methylpiperazin-1-yl) methyl)-8-((tetrahydrofuran-3-yl) amino)-3, 4-dihydroisoquinoline-2(1H)-carboxylate

Carried out in two parallel batches at 0.35 g scale. A stirred solution of t-butyl 8-bromo-6-((4-methylpiperazin-1-yl)methyl)-3,4-dihydroisoquinoline-2(1H)-carboxylate (0.35 μg, 0.82 mmol, 1.0 eq.) in THF (5 mL) at room temperature was treated with (S)-tetrahydrofuran-3-amine hydrochloride (CAS:204512-95-8) (0.15 g, 1.24 mmol, 1.5 eq.) and NaOtBu (0.198 g, 2.06 mmol, 2.5 eq.). The reaction mixture was degassed using N₂(_{g}) for 15 minutes. t-BuXPhosPdG3 (0.039 g, 0.049 mmol, 0.06 eq.) was added to the reaction and the mixture stirred at 80° C. for 2 h. The reaction mixture was poured into water (50 mL), extracted with ethyl acetate (2×100 mL) and the combined organic layer dried over Na₂SO₄, filtered, and concentrated under vacuum. Crude material was purified by column chromatography (product was eluted with 7% MeOH in DCM) yielding the title compound (0.27 g, 37.9%,). ¹H NMR (DMSO-d6, 400 MHz): δ ppm 1.43 (s, 9H), 1.82-1.89 (m, 1H), 2.16 (s, 4H), 2.33-2.34 (m, 6H), 2.66 (t, J=5.2 Hz, 2H), 3.49-3.53 (m, 2H), 3.57-3.60 (m, 1H), 3.72-3.76 (m, 1H), 3.80-3.90 (m, 1H), 3.91-3.94 (m, 1H), 3.99-4.00 (m, 1H), 4.25 (s, 2H), 4.82 (bs, 1H), 6.39 (s, 1H). 6.412 (s, 1H). LCMS (Method A): 1.235 min, MS: ES+431.12 (M+1).

### (S)-6-((4-Methylpiperazin-1-yl)methyl)-N-(tetrahydrofuran-3-yl)-1,2,3,4-tetrahydroisoquinolin-8-amine hydrochloride (Intermediate 83)

A stirred solution of t-butyl (S)-6-((4-methylpiperazin-1-yl) methyl)-8-((tetrahydrofuran-3-yl) amino)-3, 4-dihydroisoquinoline-2(1H)-carboxylate (0.27 g, 0.62 mmol, 1.0 eq) in DCM (2.7 mL) at 0° C. was treated with 4M HCl in dioxane (1.35 mL). The reaction mixture was stirred at room temperature for 2 h and then concentrated under vacuum. Crude material was purified by trituration using diethyl ether (2×15 mL) yielding (0.3 g, Yield: quantitative, 0.9 mmol). ¹H NMR (DMSO-d6, 400 MHz): δ ppm 1.84-1.92 (m, 1H), 2.45-2.27 (m, 1H), 2.49 (s, br, 2H), 2.95 (m, 2H), 3.27 (s, br, 2H), 3.36-3.41 (m, 2H), 3.50-3.70 (m, 8H), 3.68-3.75 (m, 1H), 3.80-3.90 (m, 1H), 3.91-3.94 (m, 3H), 4.1-4.28 (m, 1H), 4.38 (s, br, 2H), 5.76 (s, 2H), 6.75 (s, 1H). 6.97 (s, 1H). 9.73 (m, br, 2H). LCMS (Method B): 1.67 min, MS: ES+331.36 (M+1).

### (S)-6-((Dimethylamino)methyl)-N-(tetrahydrofuran-3-yl)-1,2,3,4-tetrahydroisoquinolin-8-amine hydrochloride (Intermediate 85)

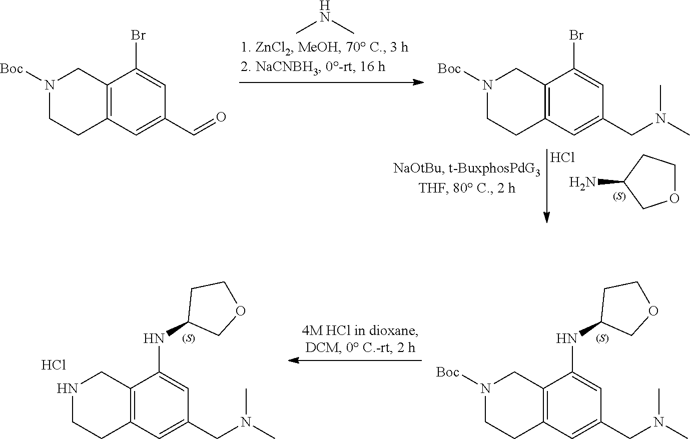

### t-Butyl-8-bromo-6-((dimethylamino)methyl)-3,4-dihydroisoquinoline-2(1H)-carboxylate

Carried out in two parallel batches at 0.7 g scale. A stirred solution of t-butyl 8-bromo-6-formyl-3,4-dihydroisoquinoline-2(1H)-carboxylate (Intermediate 100) (0.7 g, 2.064 mmol, 1.0 eq.) in MeOH (7 mL) at room temperature was treated with dimethyl amine (2.0 M in THF) (2.06 mL, 4.129 mmol, 1.0 eq.) and ZnCl₂(0.268 g, 2.064 mmol, 1.0 eq.). The resulting reaction mixture was heated to 70° C. and stirred for 3 h and then cooled. NaCNBH₃ (0.259 g, 4.12 mmol, 2.0 eq.) was added to the reaction at 0° C. and the mixture stirred at room temperature for 16 h. The reaction mixture was concentrated under vacuum. Crude material was diluted with ethyl acetate (150 mL) and washed with water (2×50 mL). The organic layer was dried over Na₂SO₄, filtered, and concentrated under vacuum. Crude material was purified by flash chromatography (product eluted with 5% MeOH in DCM) yielding the title compound (1.25 g, Yield: 82.3%,). ¹H NMR (DMSO-d6, 400 MHz): 1.51 (s, 9H), 2.29 (s, 6H), 2.84 (t, J=5.2 Hz, 2H), 3.41 (s, 2H), 3.64 (t, J=5.2 Hz, 2H), 4.53 (s, 2H), 7.11 (s, 1H), 7.40 (s, 1H). LCMS (Method A): 1.441 min, MS: ES+370.8 (M+1).

### t-Butyl-(S)-6-((dimethylamino)methyl)-8-((tetrahydrofuran-3-yl) amino)-3,4-dihydroisoquinoline-2(1H)-carboxylate

Carried out in two parallel batches at 0.3 g scale. A stirred solution of t-butyl-8-bromo-6-((dimethylamino)methyl)-3,4-dihydroisoquinoline-2(1H)-carboxylate (0.3 g, 0.81 mmol, 1.0 eq.) in THF (9 mL) at room temperature was treated with (S)-tetrahydrofuran-3-amine hydrochloride (0.15 g, 1.21 mmol, 1.5 eq.) (CAS: 204512-95-8) and NaOtBu (0.195 g, 2.03 mmol, 2.5 eq.). The reaction mixture was degassed using N₂ (g) for 10 minutes. t-BuXPhosPdG3 (0.038 g, 0.048 mmol, 0.06 eq.) was added to the reaction mixture and heated to 80° C. for 2 h. The reaction mixture was poured into water (50 mL) and extracted by ethyl acetate (2×100 mL). The combined organic layer dried over Na₂SO₄, filtered, and concentrated under vacuum. The crude material was purified by column chromatography, product was eluted at 8% MeOH in DCM yielding the title compound (0.38 g, 62.4%). LCMS (Method A): 1.282 min, MS: ES+376.21 (M+1).

### (S)-6-((Dimethylamino)methyl)-N-(tetrahydrofuran-3-yl)-1,2,3,4-tetrahydroisoquinolin-8-amine hydrochloride

A stirred solution of t-butyl (S)-6-((dimethylamino)methyl)-8-((tetrahydrofuran-3-yl) amino)-3,4-dihydroisoquinoline-2(1H)-carboxylate (0.35 g, 0.93 mmol, 1.0 eq.) in DCM (3.5 mL) was cooled to 0° C. 4M HCl in dioxane (1.75 mL) was added dropwise to the reaction mixture. The reaction mixture was stirred at room temperature for 2 h. The reaction mixture was concentrated under vacuum. The obtained crude material was purified by trituration using diethyl ether (2×20 mL) followed by high vacuum drying yielding the title compound (0.37 g, Yield: quantitative). ¹H NMR (DMSO-d6, 400 MHz): 1.86-1.89 (m, 1H), 2.23-2.33 (m, 1H), 2.66-2.27 (s, br, 6H), 2.92-2.97 (m, 3H), 3.28 (s, br, 3H), 3.73-3.76 (m, 1H), 3.82-3.84 (m, 1H), 3.98-4.07 (m, 5H), 6.26 (m, 1H), 6.86 (s, 1H), 6.89 (d, 1H). LCMS (Method B): 1.56 min, MS: ES+276.34 (M+1).

### (S)-(4-Methylpiperazin-1-yl) (8-((tetrahydrofuran-3-yl) amino)-1, 2, 3, 4-tetrahydroisoquinolin-6-yl) methanone hydrochloride (Intermediate 86)

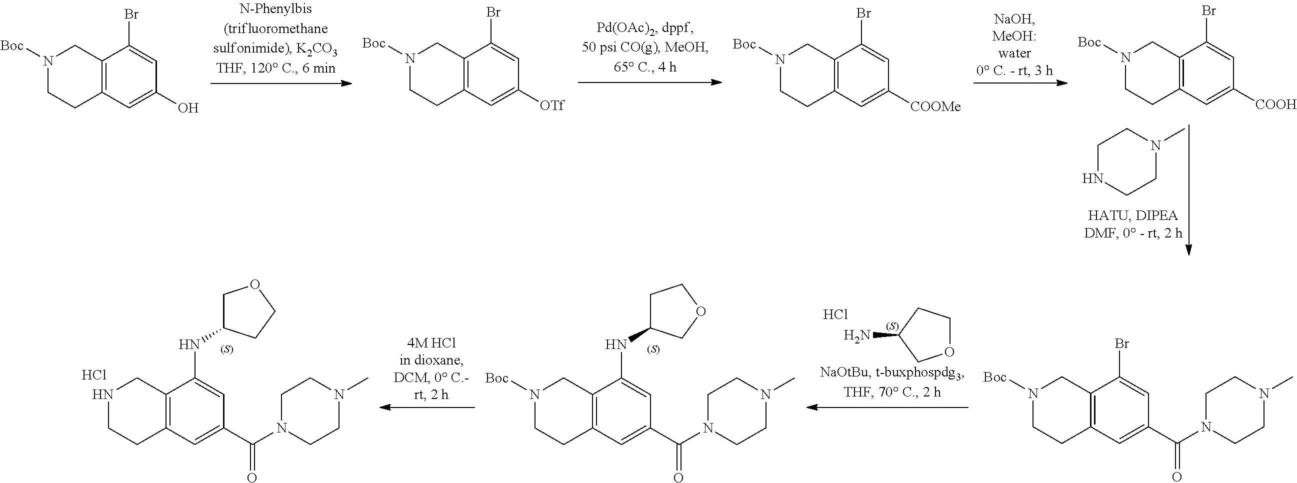

### t-Butyl 8-bromo-6-(((trifluoromethyl) sulfonyl) oxy)-3,4-dihydroisoquinoline-2(1H)-carboxylate

A stirred solution of t-butyl 8-bromo-6-hydroxy-3,4-dihydroisoquinoline-2(1H)-carboxylate (Intermediate 101) (1.0 g, 1 mmol, 1.0 eq.) in THF at room temperature was treated with K₂CO₃ (0.829 g, 6.0 mmol, 2.0 eq.) and N-Phenyl-bis(trifluoromethanesulfonimide) (CAS: 37595-74-7) (1.30 g, 3.6 mmol, 1.2 eq.). The reaction mixture was heated to 120° C. for 6 mins under microwave irradiation. The reaction mixture was cooled, poured into water (50 mL) and extracted with ethyl acetate (2×100 mL). The combined organic layer was washed with brine solution (50 mL) and dried over Na₂SO₄, filtered, and concentrated under vacuum. The crude material was purified by flash chromatography (product eluted at 10% EtOAc in hexane) yielding (2.8 g, Yield: 49.9%,). ¹H NMR (DMSO-d6, 400 MHz): δ ppm 1.44 (s, 9H), 2.87 (t, J=5.6 Hz, 2H), 3.56 (t, J=5.2 Hz, 2H), 4.47 (s, 2H), 7.45 (s, 1H), 7.79 (s, br, 1H). LCMS (Method A): 2.771 min, MS: ES+359.9 (M−100).

### 2-(t-Butyl)-6-methyl-8-bromo-3,4-dihydroisoquinoline-2,6(1H)-dicarboxylate (Intermediate 99)

A stirred solution of t-butyl 8-bromo-6-(((trifluoromethyl)sulfonyl) oxy)-3,4-dihydroisoquinoline-2(1H)-carboxylate (2.0 g, 4.34 mmol, 1.0 eq.), in MeOH (20 mL) at room temperature was treated with TEA (1.31 g, 13 mmol, 3.0 eq.). The reaction mixture was degassed using N_{2 (g)} for 10 minutes. DPPP (0.39 g, 0.95 mmol, 0.22 eq.) and Pd(OAc)₂ (0.39 g, 1.77 mmol, 0.41 eq.) were added and the reaction mixture heated to 65° C. and stirred for 4 h in autoclave under 50 psi CO_{(g)} pressure. The reaction mixture was filtered through celite bed and the bed washed with 10% MeOH in DCM (3×50 mL) solution. The filtrate was concentrated under vacuum and crude material purified by flash chromatography (product eluted at 8% EtOAc in hexane) yielding (1.64 g, Yield: 68%). ¹H NMR (DMSO-d6, 400 MHz): δ ppm 1.44 (s, 9H), 2.89 (t, J=5.2 Hz, 2H), 3.58 (t, J=5.2 Hz, 2H), 3.87 (s, 3H), 4.51 (s, br, 2H), 7.81 (s, 1H), 7.97 (s, 1H). LCMS (Method A): 2.531 min, MS: ES+313.8, 315.8 (M−56).

### 8-Bromo-2-(t-butoxycarbonyl)-1,2,3,4-tetrahydroisoquinoline-6-carboxylic acid

A stirred solution of 2-(t-butyl)-6-methyl-8-bromo-3,4-dihydroisoquinoline-2,6(1H)-dicarboxylate (Intermediate 99) (1.2 g, 4.34 mmol, 1.0 eq.), in MeOH:Water (8:2) (10 mL) at 0° C. was treated with NaOH (0.64 g, 16.2 mmol, 5.0 eq.). The reaction mixture was stirred at room temperature for 3 h. The reaction mixture was concentrated under vacuum and crude material was acidified using 1 N HCl at 0° C. The aqueous layer was extracted using ethyl acetate (2×100 mL). The combined organic layer was dried over Na₂SO₄, filtered, and concentrated under vacuum yielding the title compound (1.1 g, 95.3%). ¹H NMR (DMSO-d6, 400 MHz): δ ppm 1.41 (s, 9H), 2.87 (t, J=5.6 Hz, 2H), 3.57 (t, J=5.6 Hz, 2H), 4.49 (s, br, 2H), 7.77 (s, 1H), 7.94 (d, J=1.2 Hz, 1H), 13.24 (bs, 1H). LCMS (Method A): 2.046 min, MS: ES+299.8 (M−56).

### t-Butyl 8-bromo-6-(4-methylpiperazine-1-carbonyl)-3,4-dihydroisoquinoline-2(1H)-carboxylate

A stirred solution of 8-bromo-2-(t-butoxycarbonyl)-1,2,3,4-tetrahydroisoquinoline-6-carboxylic acid (1.1 g, 3.08 mmol, 1.0 eq.) in DMF (10 mL) at 0° C. was treated with added DIPEA (1.19 g, 9.24 mmol, 3.0 eq.) and HATU (2.34 g, 6.16 mmol, 2.0 eq.) and stirred for 10 minutes. N-methyl piperazine (0.37 g, 3.7 mmol, 1.2 eq.) was added and the resulting reaction mixture was stirred at room temperature for 2 h. poured into water (70 mL) and extracted using ethyl acetate (2×100 mL). The combined organic layer washed with brine solution (2×50 mL), dried over Na₂SO₄, filtered, and concentrated under vacuum. Crude material was purified by column chromatography, (product eluted with 3% MeOH in DCM) yielding the title compound (1.05 g, 77.6%, 2.39 mmol). ¹H NMR (DMSO-d6, 400 MHz): δ ppm 1.43 (s, 9H), 2.21 (s, 3H), 2.29-2.34 (m, 4H), 2.83 (t, J=5.6 Hz, 2H), 3.57 (t, J=5.6 Hz, 2H), 4.46 (s, 2H), 7.23 (s, 1H), 7.48 (d, J=1.6 Hz, 1H) 4 piperazine protons merged under solvent. LCMS (Method A): 1.350 min, MS: ES+348.05 (M−100).

### t-Butyl (S)-6-(4-methylpiperazine-1-carbonyl)-8-((tetrahydrofuran-3-yl) amino)-3, 4-dihydroisoquinoline-2(1H)-carboxylate

A stirred solution of t-butyl 8-bromo-6-(4-methylpiperazine-1-carbonyl)-3, 4-dihydroisoquinoline-2(1H)-carboxylate (0.35 g, 0.79 mmol, 1.0 eq.) in THF (3.5 mL) at room temperature was treated with (S)-tetrahydrofuran-3-amine hydrochloride (CAS:204512-95-8) (0.148 g, 1.19 mmol, 1.5 eq.) and NaOtBu (0.19 g, 1.99 mmol, 2.5 eq.). The reaction mixture was degassed using N₂(_{g}) for 10 minutes. t-BuXPhosPdG3 (0.063 g, 0.079 mmol, 0.1 eq.) was added and the resulting reaction mixture was heated to 70° C. and stirred for 2 h. The reaction mixture was poured into water (50 mL) and extracted by ethyl acetate (2×150 mL). The combined organic layer dried over Na₂SO₄, filtered, and concentrated under vacuum. The crude material was purified by column chromatography (product was eluted at 2.5% MeOH in DCM) yielding the title compound (0.4 g, 56.3%). ¹H NMR (DMSO-d6, 400 MHz): δ ppm 1.44 (s, 9H), 1.86-1.87 (m, 1H), 2.13-2.33 (m, 9H), 2.70 (t, J=5.2 Hz, 3H), 3.56-3.64 (m, 4H), 3.70-3.74 (m, 1H), 3.85-3.93 (m, 2H), 4.02 (s, 1H), 4.30 (s, 2H), 5.07 (bs, 1H), 6.38 (s, 1H), 6.45 (s, 1H). LCMS (Method A): 1.265 min, MS: ES+445.17 (M+1).

### (S)-(4-Methylpiperazin-1-yl) (8-((tetrahydrofuran-3-yl) amino)-1, 2, 3, 4-tetrahydro-isoquinolin-6-yl) methanone hydrochloride (Intermediate 86)

A stirred solution of t-butyl (S)-6-(4-methylpiperazine-1-carbonyl)-8-((tetrahydrofuran-3-yl) amino)-3, 4-dihydroisoquinoline-2(1H)-carboxylate (0.3 g, 0.67 mmol, 1.0 eq.) in DCM (3 mL) was cooled to 0° C. 4M HCl in dioxane (1.5 mL) was added dropwise to the reaction mixture. The resulting reaction mixture was stirred at room temperature for 2 h. The reaction mixture was concentrated under vacuum and crude material was triturated using diethyl ether (3×15 mL) followed by high vacuum drying yielding the title compound (0.31 g, Yield: quantitative). ¹H NMR (DMSO-d6, 400 MHz): δ ppm 1.97-1.89 (m, 1H), 2.18-2.25 (m, 1H), 2.73-2.76 (m, 2H), 2.94-3.06 (m, 4H), 3.29-3.40 (m, 4H), 3.57 (s, 3H), 3.60-3.63 (m, 1H), 3.88-3.91 (m, 1H), 3.93-3.94 (m, 2H), 3.95-4.06 (m, br, 1H), 4.50 (s, br, 1H), 6.51 (s, 1H), 6.58 (s, 1H), 9.73 (s, br, 2H), 11.31 (s, br, 1H).LCMS (Method B): 1.48 min, MS: ES+345.29 (M+1).

### 6-(2-Methoxyethoxy)-N-methyl-1,2,3,4-tetrahydroisoquinolin-8-amine hydrochloride (Intermediate 87)

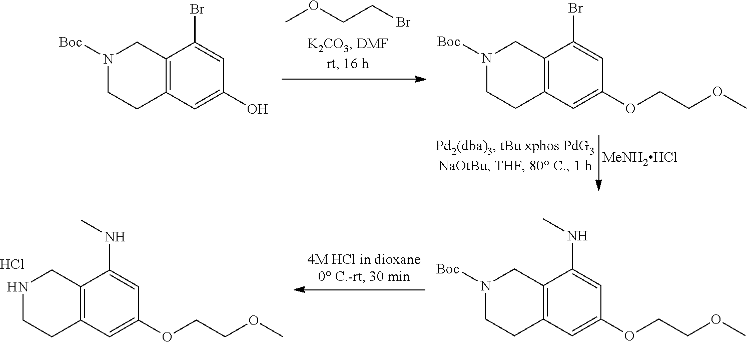

### t-Butyl 8-bromo-6-(2-methoxyethoxy)-3,4-dihydroisoquinoline-2(1H)-carboxylate

A mixture of t-butyl 8-bromo-6-hydroxy-3,4-dihydroisoquinoline-2(1H)-carboxylate (Intermediate 101) (1.0 g, 3.04 mmol, 1 eq.) in DMF at room temperature was treated with 1-bromo-2-methoxyethane (CAS: 6482-24-2) (0.63 g, 4.57 mmol, 1.5 eq.) and K₂CO₃ (0.83 g, 6.09 mmol, 2.0 eq.). The resulting reaction mixture was stirred at room temperature for 16 h, poured into ice cold water (80 mL) and extracted using ethyl acetate (3×80 mL). The combined organic layer washed with ice cold water (3×100 mL) and dried over Na₂SO₄, filtered, and concentrated under vacuum. Crude material was purified by flash chromatography (product eluted in 7% EtOAc in Hexane) (0.8 g, Yield: 67.9%). ¹H NMR (DMSO-d6, 400 MHz): δ ppm 1.43 (s, 9H), 2.75 (d, J=5.6 Hz, 2H), 3.29 (s, 3H), 3.52 (t, J=6 Hz, 2H), 3.62-3.64 (m, 2H) 4.08-4.10 (m, 2H), 4.37 (s, 2H), 6.82 (d, J=2.4 Hz, 1H), 7.10 (d, J=2.4 Hz, 1H). LCMS (Method A): 2.374 min, MS: ES+287 (M−100).

### t-Butyl 6-(2-methoxyethoxy)-8-(methylamino)-3,4-dihydroisoquinoline-2(1H)-carboxylate

A mixture of t-butyl 8-bromo-6-(2-methoxyethoxy)-3,4-dihydroisoquinoline-2(1H)-carboxylate (0.4 g, 1.03 mmol, 1 eq.), methanamine hydrochloride (0.10 g, 1.55 mmol, 1.5 eq.), NaOtBu (0.19 g, 2.07 mmol, 2 eq.) in THF (4 mL) was placed in a 35 mL glass vial at room temperature. The reaction mixture was degassed using N₂ gas for 30 min. t-BuXphosPdG3 (0.035 g, 0.06 mmol, 0.06 eq.) was added and the reaction mixture heated to 80° C. and stirred for 2 h. The reaction mixture was poured into ice cold water (60 mL) and extracted using ethyl acetate (3×60 mL) dried over Na₂SO₄, filtered, and concentrated under vacuum. Crude material was purified by flash chromatography (product eluted in 7% EtOAc in Hexane) yielding the title compound as a yellow solid (0.14 g, Yield: 20.10%, 0.41 mmol). ¹H NMR (DMSO-d6, 400 MHz): δ ppm 1.43 (s, 9H), 2.62 (t, J=5.6 Hz, 2H), 2.68 (d, J=4.8 Hz, 2H), 3.29 (d, J=6.4 Hz, 3H), 3.48 (t, J=5.6 Hz, 2H), 3.60-3.63 (m, 2H), 3.99-4.02 (m, 2H), 4.16 (s, 2H), 5.12 (s, br, 1H), 5.92 (s, 1H), 5.99 (s, 1H). LCMS (Method A): 1.982 min. MS: ES+337 (M+1).

### 6-(2-Methoxyethoxy)-N-methyl-1,2,3,4-tetrahydroisoquinolin-8-amine hydrochloride

A stirred solution of t-butyl 6-(2-methoxyethoxy)-8-(methylamino)-3, 4-dihydroisoquinoline-2(1H)-carboxylate (0.14 g, 0.41 mmol, 1.0 eq.) in DCM (2 mL) was cooled to 0° C. 4M HCl in dioxane (2 mL) was added dropwise and the reaction mixture stirred at room temperature for 1 h. The reaction mixture was concentrated under vacuum. Crude material was triturated using diethyl ether (2×5 mL) and dried under high vacuum yielding the title compound as a yellow solid (0.14 g, Yield: quantitative). ¹H NMR (DMSO-d6, 400 MHz): δ ppm 2.70 (s, 3H), 2.88 (t, J=6 Hz, 2H), 3.25-3.30 (m, 2H), 3.57 (s, 3H), 3.62-3.65 (m, 2H), 3.91 (s, 2H), 4.03-4.07 (m, 2H), 5.09 (bs, 2H), 6.09 (s, 1H), 6.15 (s, 1H), 9.54 (s, 2H). LCMS (Method A): 0.857 min, MS: ES+237 (M+1).

### (R)—N,N-Dimethyl-8-((tetrahydrofuran-3-yl)amino)-1,2,3,4-tetrahydroisoquinoline-6-carboxamide hydrochloride (Intermediate 88)

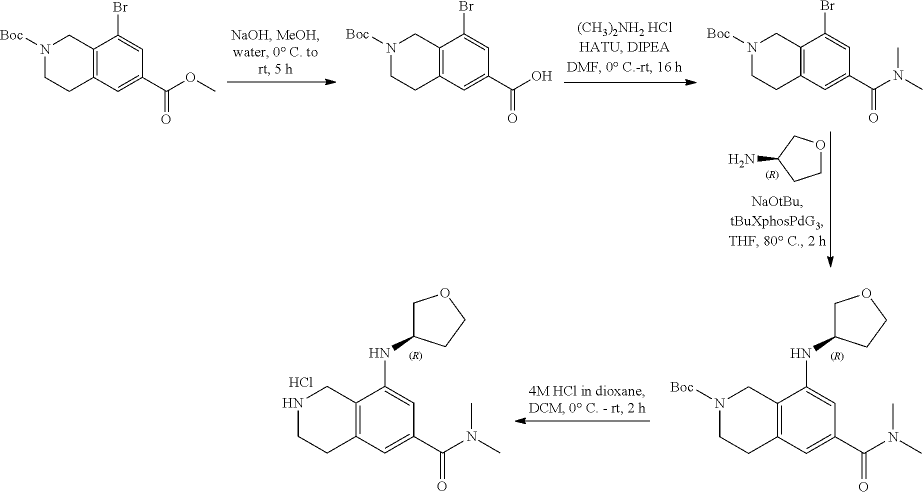

### 8-Bromo-2-(tert-butoxycarbonyl)-1,2,3,4-tetrahydroisoquinoline-6-carboxylic acid

A stirred solution of 2-(t-butyl)-6-methyl-8-bromo-3,4-dihydroisoquinoline-2,6(1H)-dicarboxylate (Intermediate 99) (2.5 g, 6.75 mmol, 1 eq.) in MeOH (20 mL) at room temperature was treated with NaOH (1.35 g, 33.78 mmol, 5.0 eq.) in water (5 mL). The resulting reaction mixture was stirred at room temperature for 5 h, then concentrated under reduced pressure. Crude material was acidified using dil. HCl and extracted into ethyl acetate (2×50 mL). The combined organic layer was dried over Na₂SO₄, filtered, and concentrated under vacuum yielding the title compound (2.2 g, Yield: 91%). ¹H NMR (DMSO-d6, 400 MHz): δ ppm 1.43 (s, 9H), 2.87 (t, J=5.6 Hz, 2H), 3.57 (t, J=5.6 Hz, 2H), 4.49 (s, 2H), 7.76 (s, 1H), 7.94 (s, 1H), 12.92 (bs, 1H). LCMS (Method A): 2.075 min, MS: ES+299.4, 301.4 (M+1, M+2).

### t-Butyl 8-bromo-6-(dimethylcarbamoyl)-3,4-dihydroisoquinoline-2(1H)-carboxylate

A stirred solution of 8-bromo-2-(t-butoxycarbonyl)-1,2,3,4-tetrahydroisoquinoline-6-carboxylic acid (2.1 g, 5.89 mmol, 1 eq.) in DMF (20 mL) at 0° C. under N₂ atmosphere was treated with HATU (4.48 g, 11.79 mmol, 2.0 eq.), DIPEA (3.1 mL, 17.68 mmol, 3.0 eq.) and dimethyl amine hydrochloride (0.72 g, 8.84 mmol, 1.5 eq.). The resulting reaction mixture was stirred at room temperature for 2 h. The reaction mixture was poured into water (30 mL) and extracted using ethyl acetate (2×30 mL). The combined organic layer was dried over Na₂SO₄, filtered, and concentrated under vacuum. Crude material was purified by flash chromatography (product eluted in 5% MeOH in DCM) yielding the title compound (1.8 g, Yield: 80%). ¹H NMR (DMSO-d6, 400 MHz): δ ppm 1.43 (s, 9H), 2.82 (t, J=6 Hz, 2H), 2.91 (s, 3H), 2.96 (s, 3H), 3.57 (t, J=5.6 Hz, 2H), 4.46 (s, br, 2H), 7.25 (s, 1H), 7.50 (s, 1H). LCMS (Method A): 2.020 min, MS: ES+326.7, 328.6 (M−56).

### t-Butyl (R)-6-(dimethylcarbamoyl)-8-((tetrahydrofuran-3-yl)amino)-3,4-dihydroisoquinoline-2(1H)-carboxylate

A stirred solution of t-butyl 8-bromo-6-(dimethylcarbamoyl)-3,4-dihydroisoquinoline-2(1H)-carboxylate (0.5 g, 1.30 mmol, 1.0 eq.), (R)-tetrahydrofuran-3-amine (0.24 g, 1.95 mmol, 1.5 eq.) and NaOtBu (0.31 g, 3.26 mmol, 2.5 eq.) in THF (10 mL) at room temperature was de-gassed with N₂(g) for 10 mins, then t-BuXPhosPdG3 (0.062 g, 0.07 mmol, 0.06 eq.) was added and the reaction heated to 80° C. for 2 h. The reaction mixture was cooled, poured into water (30 mL) and extracted with ethyl acetate (2×50 mL). The combined organic layer was dried over Na₂SO₄, filtered, and concentrated under vacuum. Crude material (0.85 g) was purified by flash chromatography (product eluted in 5% MeOH in DCM) yielding the title compound (0.36 g, Yield: 72%). ¹H NMR (DMSO-d6, 400 MHz): δ ppm 1.43 (s, 9H), 1.86 (bs, 1H), 2.15-2.20 (m, 1H), 2.69-2.71 (m, 2H), 2.91-2.94 (m, 6H), 3.52 (s, br, 2H), 3.61-3.62 (dd, J=4 Hz, 8.8 Hz, 1H), 3.71-3.75 (m, 1H), 3.81-3.85 (m, 1H), 3.89-3.93 (m, 1H), 4.03-4.04 (m, 1H), 4.30 (s, br, 2H), 4.85-5.10 (bs, 1H), 6.41 (s, 1H), 6.47 (s, 1H). LCMS (Method A): 1.721 min, MS: ES+334.0, 390.1 (M−56, M+1).

### (R)—N,N-Dimethyl-8-((tetrahydrofuran-3-yl)amino)-1,2,3,4-tetrahydroisoquinoline-6-carboxamide hydrochloride

A stirred solution of t-butyl-(R)-6-(dimethylcarbamoyl)-8-((tetrahydrofuran-3-yl)amino)-3,4-dihydro isoquinoline-2(1H)-carboxylate (0.35 g, 0.89 mmol, 1.0 eq) in DCM (5 mL) at 0° C. was treated dropwise with 4M HCl in dioxane (2 mL). The resulting reaction mixture was stirred at room temperature for 2 h. The reaction mixture was concentrated under vacuum and the crude material was triturated using diethyl ether (10 mL) yielding the title compound (0.4 g, Quantitative). ¹H NMR (DMSO-d6, 400 MHz): δ ppm 1.85-1.86 (m, 1H), 2.16-2.21 (m, 1H), 2.93-2.94 (m, 8H), 3.27-3.29 (m, 2H), 3.60-3.62 (dd, J=4 Hz, 8.8 Hz, 1H), 3.69-3.75 (m, 1H), 3.82-3.85 (q, J=7.6 Hz, 15.6 Hz, 1H), 3.89-3.93 (m, 1H), 4.00-4.06 (m, 3H), 6.46 (s, 1H), 6.53 (s, 1H) LCMS (Method A): 0.752 min, MS: ES+290.1 (M+1).

### (R)-6-(2-(Dimethylamino)ethoxy)-N-(tetrahydrofuran-3-yl)-1,2,3,4-tetrahydroisoquinolin-8-amine hydrochloride (Intermediate 89)

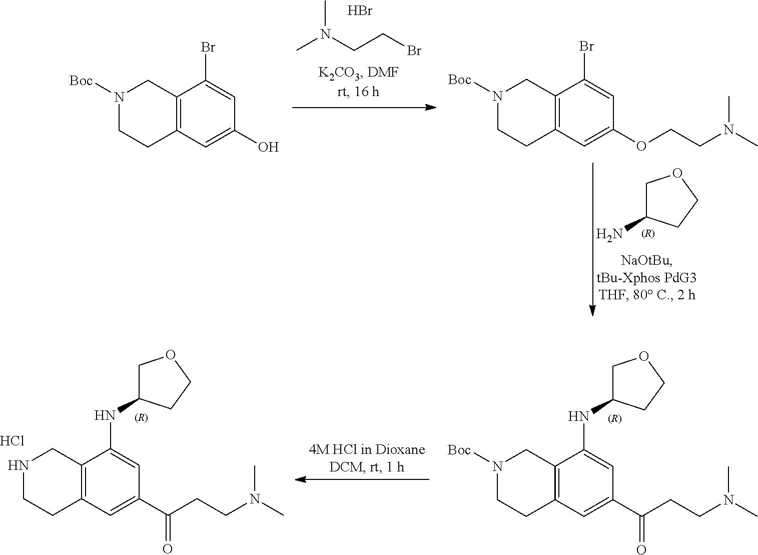

### t-Butyl-8-bromo-6-(2-(dimethylamino)ethoxy)-3,4-dihydroisoquinoline-2(1H)-carboxylate

A stirred solution of t-butyl-8-bromo-6-hydroxy-3,4-dihydroisoquinoline-2(1H)-carboxylate (Intermediate 101) (5.0 g, 15.38 mmol, 1.0 eq.) in water:THF (1:5) (60 mL) at room temperature was treated with K₂CO₃ (10.52 g, 76.23 mmol, 5.0 eq.) and stirred for 15 mins, then 2-bromo-N,N-dimethylethan-1-amine hydrobromide (7.16 g, 30.73 mmol, 2.0 eq.) (CAS: 2862-39-7) and KI (0.253 g, 1.52 mmol, 0.1 eq.) were added and the resulting reaction mixture heated to 80° C. and stirred for 16 h. The reaction mixture was poured into water (200 mL) and extracted in ethyl acetate (3×150 mL). The combined organic layer was dried over Na₂SO₄, filtered, and concentrated under vacuum. Crude material was purified by flash chromatography (product eluted in 3.7% MeOH in DCM) yielding the title compound as a white solid (1.7 g, Yield: 28%) and recovered starting material t-butyl-8-bromo-6-hydroxy-3,4-dihydroisoquinoline-2(1H)-carboxylate (Intermediate 101) (2.7 g). ¹H NMR (DMSO-d6, 400 MHz): δ ppm 1.43 (s, 9H), 2.19 (s, 6H), 2.57-2.60 (m, 2H), 2.74-2.77 (m, 2H), 3.50-3.53 (m, 2H), 4.03 (t, J=4 Hz, 2H), 4.36 (s, 2H), 6.82 (d, J=2.4 Hz, 1H), 7.09 (d, J=1.2 Hz, 1H). LCMS (Method A): 1.447 min, MS: ES+400.86 (M+1).

### t-Butyl-(R)-6-(2-(dimethylamino)ethoxy)-8-((tetrahydrofuran-3-yl)amino)-3,4-dihydroisoquinoline-2(1H)-carboxylate

A solution of t-butyl-8-bromo-6-(2-(dimethylamino)ethoxy)-3,4-dihydroisoquinoline-2(1H)-carboxylate (0.700 g, 1.75 mmol, 1 eq.), (R)-tetrahydrofuran-3-amine (0.228 g, 2.62 mmol, 1.5 eq.) and NaOtBu (0.421 g, 4.38 mmol, 2.5 eq.) in THF (10 mL) at room temperature was placed in a 35 mL glass vial. The reaction mixture was degassed using N₂ gas for 15 min. t-BuXPhosPdG3 (0.083 g, 0.104 mmol, 0.06 eq.) was added and the reaction heated to 80° C. and stirred for 2 h. The reaction mixture was concentrated under vacuum. Crude material was purified by flash chromatography (product eluted with 3% MeOH in DCM) yielding the title compound as sticky liquid (0.6 g, Yield: 84.3%). ¹H NMR (DMSO-d6, 400 MHz): δ ppm 1.41 (s, 9H), 1.83-1.84 (m, 1H), 2.14-2.17 (m, 1H), 2.20 (s, 6H), 2.57-2.64 (m, 4H), 3.47-3.49 (m, 2H), 3.56-3.59 (m, 2H), 3.68-3.74 (m, 1H), 3.79-3.83 (m, 1H), 3.87-3.91 (m, 1H), 3.95-3.99 (m, 3H), 4.18 (s, 2H), 5.98 (s, 1H), 6.05 (s, 1H). LCMS (Method A): 1.300 min, MS: ES+406.36 (M+1).

### (R)-6-(2-(Dimethylamino)ethoxy)-N-(tetrahydrofuran-3-yl)-1,2,3,4-tetrahydroisoquinolin-8-amine hydrochloride

A stirred solution of t-butyl (R)-6-(2-(dimethylamino)ethoxy)-8-((tetrahydrofuran-3-yl)amino)-3,4-dihydroisoquinoline-2(1H)-carboxylate (0.6 g, 1.48 mmol, 1.0 eq.) in DCM (5 mL) at 0° C. was treated with 4M HCl in dioxane (5 mL). The reaction mixture was stirred at room temperature for 1 h then concentrated under vacuum. Crude material was triturated using diethyl ether (3×3 mL) followed by n-pentane (3×3 mL) and dried under high vacuum yielding the title compound as a brown solid (0.480 g, Quantitative). ¹H NMR (DMSO-d6, 400 MHz): δ ppm 1.85-1.88 (m, 1H), 2.17-2.22 (m, 1H), 2.82 (d, J=4.8 Hz, 6H), 2.90 (t, J=6 Hz, 12 Hz, 2H), 3.25 (s, br, 2H), 3.44-3.48 (m, 2H), 3.58-3.62 (m, 1H), 3.72-3.74 (m, 1H), 3.82-3.85 (m, 1H), 3.91-3.95 (m, 3H), 4.00 (bs, 1H), 4.26-4.32 (m, 3H), 6.11 (s, 1H), 6.19 (s, 1H), 9.62 (bs, 1H). LCMS (Method A): 0.333 min, MS: ES+305.9 (M+1).

### 5-((1,2,3,4-Tetrahydroisoquinolin-8-yl)amino)pyridin-2(1H)-one hydrochloride (Intermediate 91)

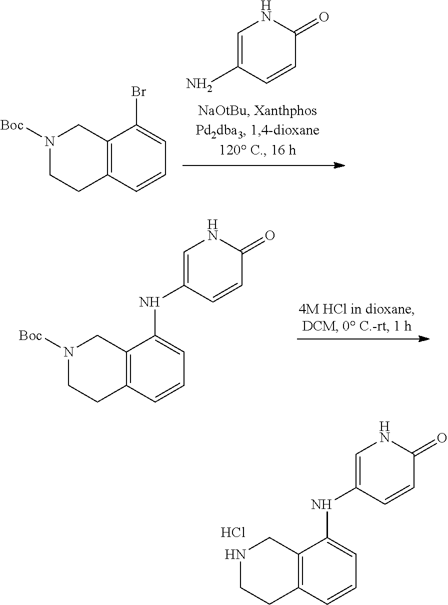 [SMILES: Nc1ccc(=O)[nH]c1 ; CC(C)(C)OC(=O)N1CCc2cccc(Br)c2C1 ; CC(C)(C)OC(=O)N1CCc2cccc(Nc3ccc(=O)[nH]c3)c2C1 ; Cl ; O=c1ccc(Nc2cccc3c2CNCC3)c[nH]1]

### t-Butyl 8-((6-oxo-1,6-dihydropyridin-3-yl)amino)-3,4-dihydroisoquinoline-2(1H)-carboxylate

A stirred solution of t-butyl 8-bromo-3,4-dihydroisoquinoline-2(1H)-carboxylate (0.5 g, 1.60 mmol, 1.0 eq.) (CAS: 893566-75-1), 5-aminopyridin-2(1H)-one (0.26 g, 2.41 mmol, 1.5 eq.) and NaOtBu (0.38 g, 4.01 mmol, 2.5 eq.) in 1,4-dioxane at room temperature was de-gassed with N₂ gas for 30 mins and treated with Xanthphos (0.055 g, 0.09 mmol, 0.06 eq.) and Pd₂(dba)₃ (0.058 g, 0.06 mmol, 0.04 eq.). The resulting reaction mixture was heated to 120° C. under microwave irradiation for 1 h. The resulting reaction mixture was cooled, filtered through a celite bed and concentrated under reduced pressure. Crude material (0.9 g) was triturated using diethyl ether (2×5 mL) followed by high vacuum drying yielding the title compound (0.6 g, Yield: 94%). LCMS (Method A): 1.673 min, MS: ES+285.7 (M−56), 341.7 (M+1).

### 5-((1,2,3,4-Tetrahydroisoquinolin-8-yl)amino)pyridin-2(1H)-one hydrochloride

A stirred solution of t-butyl-8-((6-oxo-1,6-dihydropyridin-3-yl)amino)-3,4-dihydroisoquinoline-2(1H)-carboxylate (0.6 g, 1.75 mmol, 1.0 eq.) in DCM (6 mL) at 0° C. was treated with 4M HCl in dioxane (3 mL). The resulting reaction mixture was stirred at room temperature for 1 h, concentrated under reduced pressure and crude material triturated using diethyl ether (4×30 mL) followed by high vacuum drying yielding the title compound (0.5 g, Yield: 100%). ¹H NMR (DMSO-d6, D2O-exchange 400 MHz): ppm 2.99 (t, J=6 Hz, 2H), 3.33 (t, J=6.4 Hz, 2H), 4.11 (s, 2H), 6.66-6.69 (m, 2H), 6.76 (d, J=7.6 Hz, 1H), 7.13 (t, J=8 Hz, 1H), 7.26-7.27 (m, 1H), 7.51-7.54 (m, 1H). LCMS (Method A): 0.746 min, MS: ES+241.8 (M+1).

### 1-Methyl-5-((1,2,3,4-tetrahydroisoquinolin-8-yl)amino) pyridin-2(1H)-one hydrochloride (Intermediate 92)

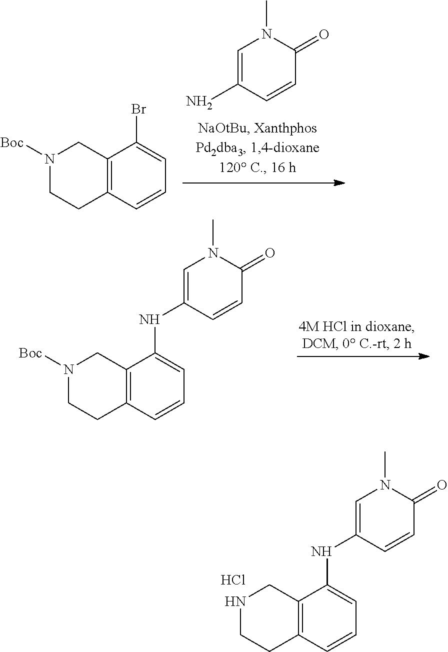

### t-Butyl 8-((1-methyl-6-oxo-1,6-dihydropyridin-3-yl) amino)-3,4-dihydroisoquinoline-2(1H)-carboxylate

A mixture of t-butyl-8-bromo-3,4-dihydroisoquinoline-2(1H)-carboxylate (0.6 g, 1.92 mmol, 1 eq.) (CAS: 893566-75-1), 5-amino-1-methylpyridin-2(1H)-one (CAS:33630-96-5) (0.358 g, 2.88 mmol, 1.5 eq.) in 1,4-dioxane (5 mL) at room temperature was treated with NaOtBu (0.461 g, 4.8 mmol, 2.5 eq.). The reaction mixture was degassed using N₂ gas for 10 mins then Xanthphos (0.066 g, 0.115 mmol, 0.06 eq.) and Pd₂ (dba)₃ (0.070 g, 0.076 mmol, 0.04 eq.) were added and the reaction mixture heated to 120° C. and stirred for 16 h. The reaction mixture was cooled, poured into water (50 mL) and extracted using ethyl acetate (2×100 mL). The combined organic layer was dried over Na₂SO₄, filtered, and concentrated under vacuum. Crude material was purified by flash chromatography (product eluted in 10% MeOH in DCM) yielding the title compound (0.5 g, Yield: 73.2%). ¹H NMR (DMSO-d6, 400 MHz): δ ppm 1.44 (s, 9H), 2.73 (t, J=5.6 Hz, 2H), 3.41 (s, 3H), 3.54 (t, J=5.6 Hz, 2H), 4.39 (s, 2H), 6.41 (d, J=9.6 Hz, 1H), 6.48 (d, J=8 Hz, 1H), 6.59 (d, J=7.6 Hz, 1H), 6.78 (bs, 1H), 6.97 (t, J=8 Hz, 1H), 7.28-7.31 (dd, J=2.8 Hz, 9.6 Hz, 1H), 7.42 (d, J=2.8 Hz, 1H). LCMS (Method A): 1.767 min, MS: ES+356 (M+1).

### 1-Methyl-5-((1,2,3,4-tetrahydroisoquinolin-8-yl) amino) pyridin-2(1H)-one hydrochloride

A stirred solution of t-butyl 8-((1-methyl-6-oxo-1,6-dihydropyridin-3-yl) amino)-3,4-dihydroisoquinoline-2(1H)-carboxylate (0.5 g, 1.406 mmol, 1.0 eq.) in DCM (5 mL) was cooled to 0° C. 4M HCl in dioxane (2.5 mL) was added dropwise. The resulting reaction mixture was stirred at room temperature for 2 h and then concentrated under vacuum. Crude material was triturated using diethyl ether (2×15 mL) and dried under high vacuum yielding the title compound (0.5 g, Yield: quantitative). ¹H NMR (DMSO-d6, 400 MHz): δ ppm 2.98 (t, J=6.0 Hz, 2H), 3.30-3.32 (m, 2H), 3.49 (s, 3H), 4.00-4.10 (m, 2H), 6.61 (t, J=6.4 Hz, 2H), 6.69 (d, J=7.6 Hz, 1H), 7.08 (t, J=7.6 Hz, 1H), 7.39-7.42 (m, 1H), 7.52 (d, J=2.8 Hz, 1H), 9.78 (bs, 1H). HCl salt. LCMS (Method B): 1.59 min, MS: ES+256.24 (M+1).

### 1-Methyl-5-((1,2,3,4-tetrahydroisoquinolin-8-yl)amino) pyridin-2(1H)-one hydrochloride (Intermediate 95)

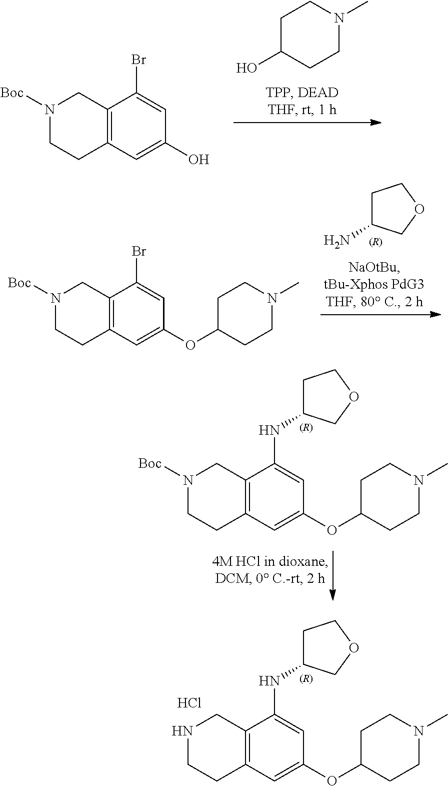

### t-Butyl-8-bromo-6-((1-methylpiperidin-4-yl)oxy)-3,4-dihydroisoquinoline-2(1H)-carboxylate

A stirred solution t-butyl 8-bromo-6-hydroxy-3,4-dihydroisoquinoline-2(1H)-carboxylate (Intermediate 101) (1.0 g, 3.0 mmol, 1 eq.) in THF (10 mL) at room temperature was treated with 1-methylpiperidin-4-ol (1.053 g, 9.1 mmol, 3.0 eq.) (CAS:106-52-51) and DEAD (40% in toluene) (4.13 mL, 9.1 mmol, 3.0 eq. ˜2.2 M in toluene). TPP (2.38 g, 9.1 mmol, 3.0 eq.) was added portion-wise and the reaction mixture stirred at room temperature for 1 h. The reaction mixture was diluted with ethyl acetate (100 mL) and organic layer washed with brine solution (2×50 mL). The organic layer was dried over Na₂SO₄, filtered, and concentrated under vacuum. Crude material was purified using flash chromatography (product was eluted at 7% MeOH in DCM) yielding the title compound (1.1 g, 85.2%). ¹H NMR (DMSO-d6, 400 MHz): δ ppm 1.42 (s, 9H), 1.57-1.64 (m, 2H), 1.87-1.91 (m, 2H), 2.13-2.20 (m, 5H), 2.49-2.76 (m, 4H), 3.51 (t, J=5.6 Hz, 2H), 4.03-4.06 (m, 2H), 4.35-4.40 (m, 3H), 6.83 (d, J=2 Hz, 1H), 7.10 (d, J=2 Hz, 1H). LCMS (Method A): 1.557 min, MS: ES+424.9 (M+1).

### t-Butyl (R)-6-((1-methylpiperidin-4-yl)oxy)-8-((tetrahydrofuran-3-yl) amino)-3,4-dihydro-isoquinoline-2(1H)-carboxylate

A mixture of t-butyl-8-bromo-6-((1-methylpiperidin-4-yl)oxy)-3,4-dihydroisoquinoline-2(1H)-carboxylate (0.5 g, 1.17 mmol, 1 eq.), (R)-tetrahydrofuran-3-amine (CAS: 111769-26-7) (0.154 g, 1.76 mmol, 1.5 eq.) in THF (5 mL) at room temperature was treated with NaOtBu (0.283 g, 2.94 mmol, 2.5 eq.). The reaction mixture was degassed using N₂ gas for 10 min. t-BuXPhosPdG3 (0.056 g, 0.070 mmol, 0.06 eq.) was added and the resulting reaction mixture was heated to 80° C. and stirred for 2 h. The reaction mixture was poured into water (100 mL) and extracted using ethyl acetate (2×100 mL). The combined organic layer was dried over Na₂SO₄, filtered, and concentrated under vacuum. Crude material was purified by flash chromatography (product eluted in 8% MeOH in DCM) yielding the title compound (0.38 g, Yield: 74.8%). ¹H NMR (DMSO-d6, 400 MHz): δ ppm 1.15-1.20 (m, 1H), 1.42 (s, 9H), 1.56-1.62 (m, 2H), 1.82-1.87 (m, 2H), 2.14-2.16 (m, 2H), 2.18 (s, 3H), 2.66-2.67 (m, 2H), 2.75-2.82 (m, 1H), 3.47-3.57 (m, 2H), 3.58-3.60 (m, 1H), 3.68-3.74 (m, 1H), 3.79-3.91 (m, 2H), 3.99-4.06 (m, 2H), 4.19 (s, 2H), 4.24-4.28 (m, 1H), 4.86 (bs, 1H), 5.98 (bs, 1H), 6.06 (bs, 1H). LCMS (Method A): 1.361 min, MS: ES+432.17 (M+1).

### 1-Methyl-5-((1,2,3,4-tetrahydroisoquinolin-8-yl) amino) pyridin-2(1H)-one hydrochloride

A stirred solution of t-butyl 8-((1-methyl-6-oxo-1,6-dihydropyridin-3-yl) amino)-3,4-dihydroisoquinoline-2(1H)-carboxylate (0.38 g, 0.88 mmol, 1.0 eq.) in DCM (4 mL) at 0° C. was treated with 4M HCl in dioxane (1.9 mL) and the reaction mixture was stirred at room temperature for 2 h. The reaction mixture was concentrated under vacuum and crude material was triturated using diethyl ether (2×15 mL), followed by drying under high vacuum yielding the title compound (0.38 g, Yield: quantitative). ¹H NMR (DMSO-d6, 400 MHz): δ ppm 1.86-2.02 (m, 6H), 2.65-2.75 (m, 3H), 2.89-2.90 (m, 2H), 3.00-3.25 (m, 2H), 3.25-3.30 (m, 2H), 3.35-3.45 (m, 1H), 3.57-3.60 (m, 1H), 3.68-3.74 (m, 1H), 3.80-4.10 (m, 5H), 4.50-4.70 (m, 1H), 6.06-6.23 (m, 2H). LCMS (Method A): 0.586 min, MS: ES+331.9 (M+1).

### (S)-6-((1-Methylpiperidin-4-yl)oxy)-N-(tetrahydrofuran-3-yl)-1,2,3,4-tetrahydroisoquinolin-8-amine (Intermediate 96)

Prepared from t-Butyl-8-bromo-6-((1-methylpiperidin-4-yl)oxy)-3,4-dihydroisoquinoline-2(1H)-carboxylate using (S)-tetrahydrofuran-3-amine and methods described above for Intermediate 95.

### N-Methyl-N-(tetrahydrofuran-3-yl)-1,2,3,4-tetrahydroisoquinolin-8-amine hydrochloride (Intermediate 97)

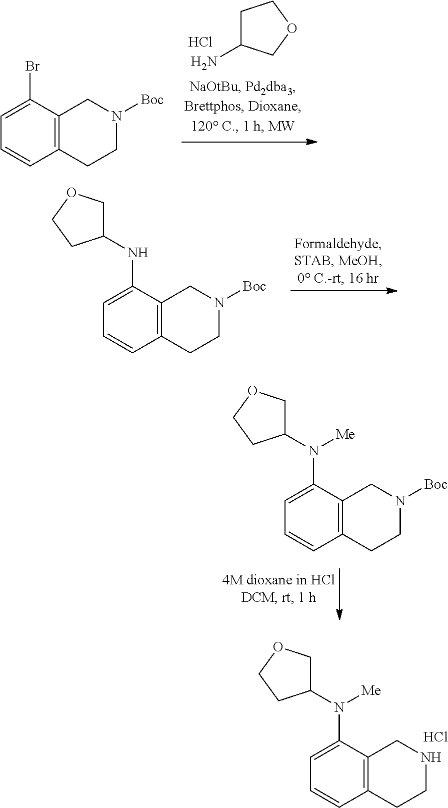

### t-Butyl 8-((tetrahydrofuran-3-yl)amino)-3,4-dihydroisoquinoline-2(1H)-carboxylate

t-Butyl 8-bromo-3,4-dihydroisoquinoline-2(1H)-carboxylate (1.0 g, 3.20 mmol, 1 eq.), tetrahydrofuran-3-amine hydrochloride (0.59 g, 4.77 mmol, 1.5 eq.), NaOtBu (0.77 g, 8.01 mmol, 2.5 eq.) and Brettphos (0.103 g, 0.18 mmol, 0.06 eq.) in 1,4-dioxane (5 mL) at room temperature were added to a 35 mL microwave glass vial. The reaction mixture was degassed using N₂ gas for 30 min. and Pd₂(dba)₃ (0.12 g, 0.131 mmol, 0.04 eq.) was added. The reaction mixture was heated to 120° C. under microwave irradiation for 1 h. The reaction mixture was cooled, filtered through a celite bed, the bed was washed with 10% MeOH:DCM (500 mL). The combined organic layer was dried over Na₂SO₄, filtered, and concentrated under vacuum. Crude material was purified by flash chromatography (product eluted with 12% EtOAc in hexane) yielding the title compound as a light yellow solid (0.62 g, Yield: 60.8%). ¹H NMR (DMSO-d6, 400 MHz): δ ppm 1.43 (s, 9H), 1.84-1.86 (m, 1H), 2.15-2.20 (m, 1H), 2.68-2.69 (m, 2H), 3.35-3.51 (m, 2H), 3.57-3.60 (m, 1H), 3.69-3.75 (m, 1H), 3.80-3.82 (m, 1H), 3.84-3.90 (m, 1H), 3.92-4.00 (m, 1H), 4.27 (s, 2H), 4.85 (bs, 1H), 6.45 (d, J=7.6 Hz, 1H), 6.57-6.65 (m, 1H), 6.95-7.01 (m, 1H). LCMS (Method A): 2.170 min, MS: ES+263 (M−56).

### t-Butyl 8-(methyl(tetrahydrofuran-3-yl)amino)-3,4-dihydroisoquinoline-2(1H)-carboxylate

A stirred solution of t-butyl 8-((tetrahydrofuran-3-yl)amino)-3,4-dihydroisoquinoline-2(1H)-carboxylate (0.44 g, 1.38 mmol, 1.0 eq.) in MeOH (5 mL) was cooled to 0° C. Formaldehyde solution (37% in water) (0.125 g, 4.17 mmol, 3.0 eq.) and STAB (0.88 g, 4.15 mmol, 3.0 eq.) were added to reaction mixture at 0° C. The reaction mixture was stirred at room temperature for 20 min. Additional STAB (0.88 g, 4.15 mmol, 3.0 eq.) was added and the resulting reaction mixture stirred at room temperature for 16 h. The reaction mixture was concentrated under vacuum and crude material purified by flash chromatography (product eluted with 9% EtOAc in hexane) yielding the title compound as a light-yellow sticky solid (0.3 g, Yield: 63.1%). ¹H NMR (DMSO-d6, 400 MHz): δ ppm 1.43 (m, 9H), 1.73 (m, 1H), 1.94 (m, 1H), 2.77 (t, J=12 Hz, 2H), 3.44 (m, 3H), 3.69 (m, 3H), 3.79 (m, 1H), 4.34 (s, br, 2H), 6.93 (d, J=24.4 Hz, 1H), 7.09 (d, J=7.6 Hz, 1H), 7.16 (t, J=16.4 Hz, 1H). LCMS (Method A): 2.307 min, MS: ES+333 (M+1).

### N-Methyl-N-(tetrahydrofuran-3-yl)-1,2,3,4-tetrahydroisoquinolin-8-amine hydrochloride

A stirred solution of t-butyl 8-(methyl (tetrahydrofuran-3-yl)amino)-3,4-dihydroisoquinoline-2(1H)-carboxylate (0.33 g, 0.90 mmol, 1.0 eq.) in DCM (3 mL) was cooled to 0° C. 4M HCl in dioxane (2 mL, 5 v) was added dropwise and the reaction mixture was stirred at room temperature for 1 h and then concentrated under vacuum. Crude material was triturated using diethyl ether (3×10 mL) and further dried under high vacuum yielding the title compound (0.25 g, Yield: Quantitative). ¹H NMR (DMSO-d6, 400 MHz): δ ppm 1.75-1.76 (m, 1H), 1.95-1.99 (m, 1H), 3.00 (t, J=6.4 Hz, 2H), 3.29-3.33 (m, 2H), 3.46-3.49 (m, 1H), 3.57 (s, 3H), 3.63-3.71 (m, 2H), 3.77-3.83 (m, 2H), 4.01-4.19 (m, 2H), 7.01 (d, J=5.2 Hz, 1H), 7.19 (d, J=7.6 Hz, 1H), 7.28 (d, J=8 Hz, 1H), 9.58 (s, 2H). HCl salt. LCMS (Method A): 0.972 min, MS: ES+233.03 (M+1).

### 2-(Benzyloxy)-4,6-bis(methoxymethoxy)benzoic acid (Intermediate 102)

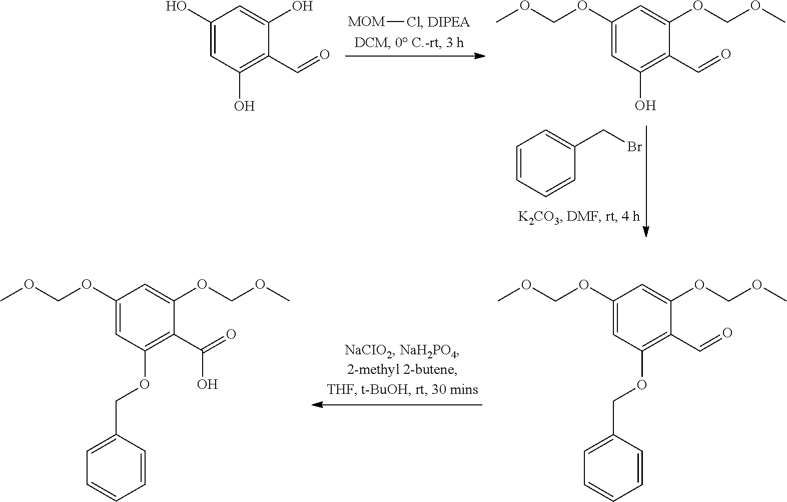

### 2-Hydroxy-4,6-bis(methoxymethoxy)benzaldehye

A stirred solution of 2,4,6-trihydroxybenzaldehyde (CAS:487-70-7) (10 g, 64.93 mmol, 1 eq.) in DCM (100 mL) at room temperature was treated with DIPEA (41.88 g, 324.67 mmol, 5 eq.) and MOM-CI (15.68 g, 194.80 mmol, 3 eq.) and the resulting reaction mixture stirred room temperature for 3 h. The resulting reaction mixture was diluted with water (100 mL) and extracted using EtOAc (3×100 mL). The combined organic layer was dried over Na₂SO₄ and concentrated under vacuum. Crude material was purified by flash chromatography (product eluted at 7% EtOAc in Hexane) yielding the title compound (9.2 g, Yield: 58%). ¹H NMR (DMSO-d6, 400 MHz): δ ppm 3.39 (s, 3H), 3.44 (s, 3H), 5.27 (s, 2H), 5.32 (s, 2H), 6.29 (s, 1H), 6.30 (s, 1H), 10.11 (s, 1H), 12.19 (s, 1H). LCMS (Method A): 1.773 min, MS: ES+242.9 (M+1).

### 2-(Benzyloxy)-4,6-bis(methoxymethoxy)benzaldehyde

A stirred solution of 2-hydroxy-4,6-bis(methoxymethoxy)benzaldehyde (9.2 g, 38.01 mmol, 1 eq.) in DMF (100 mL) at room temperature was treated with K₂CO₃ (15.7 g, 114.05 mmol, 3 eq.) followed by benzyl bromide (7.91 g, 49.42 mmol, 1.3 eq.). The resulting reaction mixture was stirred at room temperature for 4 h, then diluted with water (100 mL) and extracted using ethyl acetate (3×200 mL). The combined organic layer was washed with brine (3×300 mL), dried over Na₂SO₄, filtered, and concentrated under vacuum. Crude material was purified by flash chromatography (product eluted with 10% EtOAc in Hexane) yielding the title compound (9.2 g, Yield: 84%). ¹H NMR (DMSO-d6, 400 MHz): δ ppm 3.39 (s, 3H), 3.40 (s, 3H), 5.20 (s, 2H), 5.27 (s, 2H), 5.29 (s, 2H), 6.44 (d, J=1.6 Hz, 1H), 6.53 (d, J=1.6 Hz, 1H), 7.31-7.35 (m, 1H), 7.39-7.42 (m, 2H), 7.50 (d, J=5.2 Hz, 2H), 10.31 (s, 1H). LCMS (Method A): 1.99 min, MS: ES+332.9 (M+1).

### 2-(Benzyloxy)-4,6-bis(methoxymethoxy)benzoic acid

A stirred solution of 2-(benzyloxy)-4,6-bis(methoxymethoxy)benzaldehyde (2.0 g, 6.02 mmol, 1 eq.) in THF:t-Butanol (4:1) (25 mL) at room temperature was treated with a solution of NaH₂PO4 (4.33 g, 36.08 mmol, 6 eq.) in minimum water, NaClO₂ (1.63 g, 18.03 mmol, 3 eq.) in minimum water and 2-methyl-2-butene (8.43 g, 119.77 mmol, 20 eq.). The resulting reaction mixture was allowed to stir at room temperature for 30 mins. The resulting reaction mixture was diluted with ice cold water (100 mL) and the aqueous layer extracted with diethyl ether (4×100 mL). The combined organic layer was dried over Na₂SO₄, filtered, and concentrated under vacuum to give the title compound (2 g, Yield: 97%) which directly used for the next step without further purification. ¹H NMR (DMSO-d6, 400 MHz): δ ppm 3.39 (s, 3H), 3.40 (s, 3H), 5.11 (s, 2H), 5.17 (s, 2H), 5.19 (s, 2H), 6.44 (d, J=2 Hz, 1H), 6.49 (d, J=2 Hz, 1H), 7.32-7.34 (m, 1H), 7.37-7.43 (m, 4H), 12.69 (bs, 1H). LCMS (Method A): 1.796 min, MS: ES+348.80 (M+1), 370.8 (M+23).

### Assay Protocols

### Fluorescence Polarisation Assay for MLH1 Using Example 105

Test compounds, as 10 mM DMSO stocks, were dispensed into a 384-well Low Flange Black Flat Bottom Polystyrene Non-binding Surface plate (Corning®, item number 3575) using a Labcyte Echo acoustic liquid handler. Test compounds were added to wells in columns 1-22 whilst DMSO was added to wells in columns 23 and 24 in order to normalise the plate. 20 μL of a 2×solution (200 nM) of recombinant N-terminal MLH1 (residues 15-340) in assay buffer (25 mM HEPES, pH 7.5, 250 mM NaCl, 10 mM MgCl₂, 0.01% Triton X-100, 5 mM Dithiothreitol) was added to all wells in columns 1-23 and 20 μL assay buffer was added to all wells in column 24 using an E1-ClipTip pipette (ThermoFisher). Plates were centrifuged for 1 minute at 250×g and were incubated at room temperature for 30 minutes prior to the addition of 20 μL of 2× (10 nM) Example 105 in assay buffer (prepared from a 1 mM DMSO stock) with an E1-ClipTip pipette (ThermoFisher). The final concentration of N-terminal MLH1 was 100 nM and the final concentration of Example 105 was 5 nM. Compound plates were centrifuged for 1 minute at 250×g for 1 minute and immediately read on a PheraStar FSX (fitted with 384-well aperture spoon and 590 675 675 FP optic module). The gain and focus were adjusted before each plate was read so that the polarisation of a no enzyme control (column 24) was equal to 35 mP. Data were normalised against the no inhibitor controls (column 23) and no enzyme controls (column 24).

### Fluorescence Polarisation Assay for MLH1

Test compounds, as 10 mM DMSO stocks, were dispensed into a 384-well Low Flange Black Flat Bottom Polystyrene Non-binding Surface plate (Corning®, item number 3575) using a Labcyte Echo acoustic liquid handler. Test compounds were added to wells in columns 1-22 whilst DMSO was added to wells in columns 23 and 24 in order to normalise the plate. 20 μL of a 2× solution (200 nM) of recombinant N-terminal MLH1 (residues 15-340) in assay buffer (25 mM HEPES, pH 7.5, 250 mM NaCl, 10 mM MgCl₂, 0.01% Triton X-100, 5 mM dithiothreitol) was added to all wells in columns 1-23 and 20 μL assay buffer was added to all wells in column 24 using an E1-ClipTip pipette (ThermoFisher). Plates were centrifuged for 1 minute at 250×g and were incubated at room temperature for 30 minutes prior to the addition of 20 μL of 2× (10 nM) 5-((5-(4-((2-(2,4-dihydroxy-5-isopropylbenzoyl)isoindolin-5-yl)methyl)piperazin-1-yl)pentyl)carbamoyl)-2-(6-(dimethylamino)-3-(dimethyliminio)-3H-xanthen-9-yl)benzoate in assay buffer (prepared from a 1 mM DMSO stock) with an E1-ClipTip pipette (ThermoFisher). The final concentration of N-terminal MLH1 was 100 nM and the final concentration of the probe compound was 5 nM. Compound plates were centrifuged for 1 minute at 250×g for 1 minute and immediately read on a PheraStar FSX (fitted with 384-well aperture spoon and 590 675 675 FP optic module). The gain and focus were adjusted before each plate was read so that the polarisation of a no enzyme control (column 24) was equal to 35 mP. Data were normalised against the no inhibitor controls (column 23) and no enzyme controls (column 24).

Data obtained in this assay is shown in Table 4 below.

### Fluorescence Polarisation Assay for PMS2

Test compounds, as 10 mM DMSO stocks, were dispensed into a Black Fluotrac 200 384 well medium binding plate (Greiner Bio-One, item number 781076) using a Labcyte Echo acoustic liquid handler. For single point screening, test compounds were added to wells in columns 1-22 whilst DMSO was added to wells in columns 23 and 24 in order to normalise the plate. For potency determination, serial dilutions of test compounds were added to wells in columns 3-22 and DMSO volume was normalised across the plate.

20 μL of a 2× solution (20 nM) of recombinant N-terminal PMS2 (residues 1-365) in assay buffer (25 mM HEPES, pH 7.5, 250 mM NaCl, 10 mM MgCl2, 0.01% Triton X-100, 5 mM Dithiothreitol) was added to all wells in columns 2-23 for potency determination or columns 1-23 for single point screening. 20 μL assay buffer was added to all wells in columns 1 and 24 (column 24 only for single point screening) using a MultiDrop Combi (ThermoFisher). Plates were centrifuged for 1 minute at 250×g and were incubated at room temperature for 30 minutes prior to the addition of 20 μL of 2× (20 nM) of 5-((5-(4-((2-(2,4-dihydroxy-5-isopropylbenzoyl)isoindolin-5-yl)methyl)piperazin-1-yl)pentyl)carbamoyl)-2-(6-(dimethylamino)-3-(dimethyliminio)-3H-xanthen-9-yl)benzoate (referred to hereinafter as “probe compound”) in assay buffer (prepared from a 100 μM DMSO stock) with a MultiDrop Combi (ThermoFisher). The final concentration of N-terminal PMS2 was 10 nM and the final concentration of probe compound was 5 nM.

Compound plates were centrifuged for 1 minute at 250×g for 1 minute and were incubated at room temperature for 1 hour before being read on a PheraStar FSX (fitted with 384-well aperture spoon and 540 590 590 FP optic module). The gain and focus were adjusted before each plate was read so that the polarisation of a no enzyme control (column 24) was equal to 35 mP. Data were normalised against the no inhibitor controls (column 23) and no enzyme controls (column 24).

Data obtained in this assay is shown in Table 4 below.
*>10 μM
** 1-10 μM
*** less than 1 μM

| Example | Name | MLH1 IC₅₀ | PMS2 IC₅₀ |
|---|---|---|---|
| 1 | (R)-(2-(Benzyloxy)-4,6-dihydroxyphenyl)(8- | *** | * |
| | ((tetrahydrofuran-3-yl)amino)-3,4-dihydro- | | |
| | isoquinolin-2(1H)-yl)methanone | | |
| 2 | (S)-(2-(Benzyloxy)-4,6-dihydroxyphenyl)(8- | *** | ** |
| | ((tetrahydrofuran-3-yl)amino)-3,4-dihydro- | | |
| | isoquinolin-2(1H)-yl)methanone | | |
| 3 | (2-(Benzyloxy)-4,6-dihydroxyphenyl)(5- | *** | ** |
| | (piperazin-1-ylmethyl)isoindolin-2- | | |
| | yl)methanone | | |
| 4 | (2-(Benzyloxy)-4,6-dihydroxyphenyl)(5- | *** | *** |
| | ((methylamino)methyl)isoindolin-2- | | |
| | yl)methanone | | |
| 5 | 3-((2-(2-(Benzyloxy)-4,6- | ** | ** |
| | dihydroxybenzoyl)isoindolin-4- | | |
| | yl)oxy)cyclobutane-1-carbonitrile | | |
| 6 | (2-(Benzyloxy)-4,6-dihydroxyphenyl)(4- | *** | ** |
| | ((tetrahydrofuran-3-yl)amino)isoindolin-2- | | |
| | yl)methanone | | |
| 7 | (2-(Benzyloxy)-4,6-dihydroxyphenyl)(4- | *** | ** |
| | (oxetan-3-ylamino)isoindolin-2- | | |
| | yl)methanone | | |
| 8 | (2-(Benzyloxy)-4,6-dihydroxyphenyl)(4-(3- | ** | ** |
| | hydroxypiperidin-1-yl)isoindolin-2- | | |
| | yl)methanone | | |
| 9 | (2-(Benzyloxy)-4,6-dihydroxyphenyl)(4- | ** | ** |
| | ((4-methylpiperazin-1- | | |
| | yl)methyl)isoindolin-2-yl)methanone | | |
| 10 | (2-(Benzyloxy)-4,6-dihydroxyphenyl)(4- | ** | ** |
| | (morpholinomethyl)isoindolin-2- | | |
| | yl)methanone | | |
| 11 | (2-(Benzyloxy)-4,6-dihydroxyphenyl)(5- | ** | ** |
| | ((dimethylamino)methyl)isoindolin-2- | | |
| | yl)methanone | | |
| 12 | (2-(Benzyloxy)-4,6-dihydroxyphenyl)(5- | *** | *** |
| | (morpholinomethyl)isoindolin-2- | | |
| | yl)methanone | | |
| 13 | (2-(Benzyloxy)-4,6-dihydroxyphenyl)(1- | *** | ** |
| | (hydroxymethyl)isoindolin-2- | | |
| | yl)methanone | | |
| 14 | (2-(Benzyloxy)-4,6-dihydroxyphenyl)(4- | *** | ** |
| | ((tetrahydro-2H-pyran-3- | | |
| | yl)amino)isoindolin-2-yl)methanone | | |
| 15 | (2-(Benzyloxy)-4,6-dihydroxyphenyl)(4- | *** | ** |
| | ((tetrahydro-2H-pyran-4- | | |
| | yl)amino)isoindolin-2-yl)methanone | | |
| 16 | (2,4-Dihydroxy-6-methoxyphenyl)(4- | ** | * |
| | ((tetrahydrofuran-3-yl)amino)isoindolin-2- | | |
| | yl)methanone | | |
| 17 | (2-Ethoxy-4,6-dihydroxyphenyl)(4- | *** | * |
| | ((tetrahydrofuran-3-yl)amino)isoindolin-2- | | |
| | yl)methanone | | |
| 18 | 4-((2-(2-(Benzyloxy)-4,6- | *** | ** |
| | dihydroxybenzoyl)isoindolin-4-yl)amino)- | | |
| | 1-methylpyrrolidin-2-one | | |
| 19 | (2-(Benzyloxy)-4,6-dihydroxyphenyl)(4- | *** | *** |
| | (pyrimidin-5-ylamino)isoindolin-2- | | |
| | yl)methanone | | |
| 20 | (2-(Benzyloxy)-4,6-dihydroxyphenyl)(4- | *** | ** |
| | ((oxazol-4-ylmethyl)amino)isoindolin-2- | | |
| | yl)methanone | | |
| 21 | (2-(Benzyloxy)-4,6-dihydroxyphenyl)(4- | ** | ** |
| | ((1-methyl-1H-pyrazol-4- | | |
| | yl)amino)isoindolin-2-yl)methanone | | |
| 22 | (S)-(2-(Benzyloxy)-4,6- | *** | *** |
| | dihydroxyphenyl)(4-((tetrahydrofuran-3- | | |
| | yl)amino)isoindolin-2-yl)methanone | | |
| 23 | (R)-(2-(Benzyloxy)-4,6- | *** | ** |
| | dihydroxyphenyl)(4-((tetrahydrofuran-3- | | |
| | yl)amino)isoindolin-2-yl)methanone | | |
| 24 | 4-((2-(2-(benzyloxy)-4,6- | ** | ** |
| | dihydroxybenzoyl)isoindolin-4-yl)amino)- | | |
| | 5-methylpyridin-2(1H)-one | | |
| 25 | (2-(Cyclopropylmethoxy)-4,6- | *** | ** |
| | dihydroxyphenyl)(4-((tetrahydrofuran-3- | | |
| | yl)amino)isoindolin-2-yl)methanone | | |
| 26 | (2-(cyclohexylmethoxy)-4,6- | ** | ** |
| | dihydroxyphenyl)(4-((tetrahydrofuran-3- | | |
| | yl)amino)isoindolin-2-yl)methanone | | |
| 27 | (2-(Benzyloxy)-4,6-dihydroxyphenyl)(5- | ** | ** |
| | ((tetrahydrofuran-3-yl)amino)-3,4- | | |
| | dihydroisoquinolin-2(1H)-yl)methanone | | |
| 28 | (2-(Cyclohexylmethoxy)-4,6- | ** | ** |
| | dihydroxyphenyl)(8-((tetrahydrofuran-3- | | |
| | yl)amino)-3,4-dihydroisoquinolin-2(1H)- | | |
| | yl)methanone | | |
| 29 | (2-(Benzyloxy)-4,6-dihydroxyphenyl)(4- | ** | ** |
| | (methylamino)isoindolin-2-yl)methanone | | |
| 30 | (2-(Benzyloxy)-4,6-dihydroxyphenyl)(4- | *** | ** |
| | ((tetrahydro-2H-pyran-3- | | |
| | yl)amino)isoindolin-2-yl)methanone | | |
| 31 | (2-(Benzyloxy)-4,6-dihydroxyphenyl)(4- | ** | * |
| | ((tetrahydro-2H-pyran-3- | | |
| | yl)amino)isoindolin-2-yl)methanone | | |
| 32 | (2-(Benzyloxy)-4,6-dihydroxyphenyl)(4- | ** | ** |
| | ((5,5-difluorotetrahydro-2H-pyran-3- | | |
| | yl)amino)isoindolin-2-yl)methanone | | |
| 33 | (2-(Benzyloxy)-4,6-dihydroxyphenyl)(4- | *** | ** |
| | (((tetrahydrofuran-2- | | |
| | yl)methyl)amino)isoindolin-2- | | |
| | yl)methanone | | |
| 34 | (4-((1H-Pyrazol-4-yl)amino)isoindolin-2- | *** | ** |
| | yl)(2-(benzyloxy)-4,6- | | |
| | dihydroxyphenyl)methanone | | |
| 35 | (2-(Benzyloxy)-4,6-dihydroxyphenyl)(4- | *** | ** |
| | ((2-methyl-2H-1,2,3-triazol-4- | | |
| | yl)amino)isoindolin-2-yl)methanone | | |
| 36 | 1-(3-((2-(2-(Benzyloxy)-4,6- | *** | ** |
| | dihydroxybenzoyl)isoindolin-4- | | |
| | yl)amino)pyrrolidin-1-yl)ethan-1-one | | |
| 37 | (2-((4-Fluorobenzyl)oxy)-4,6- | *** | ** |
| | dihydroxyphenyl)(4-((tetrahydrofuran-3- | | |
| | yl)amino)isoindolin-2-yl)methanone | | |
| 38 | (2-((3,4-Difluorobenzyl)oxy)-4,6- | *** | ** |
| | dihydroxyphenyl)(4-((tetrahydrofuran-3- | | |
| | yl)amino)isoindolin-2-yl)methanone | | |
| 39 | (2,4-Dihydroxy-6-((4- | *** | ** |
| | methoxybenzyl)oxy)phenyl)(4- | | |
| | ((tetrahydrofuran-3-yl)amino)isoindolin-2- | | |
| | yl)methanone | | |
| 40 | (2,4-Dihydroxy-6-(pyridin-2- | *** | * |
| | ylmethoxy)phenyl)(4-((tetrahydrofuran-3- | | |
| | yl)amino)isoindolin-2-yl)methanone | | |
| 41 | (2-(Benzyloxy)-4,6-dihydroxyphenyl)(8- | *** | ** |
| | (oxetan-3-ylamino)-3,4- | | |
| | dihydroisoquinolin-2(1H)-yl)methanone | | |
| 42 | 4-((2-(2-(Benzyloxy)-4,6- | *** | *** |
| | dihydroxybenzoyl)-1,2,3,4- | | |
| | tetrahydroisoquinolin-8-yl)amino)-1- | | |
| | methylpyrrolidin-2-one | | |
| 43 | (2-(Benzyloxy)-4,6-dihydroxyphenyl)(8- | *** | ** |
| | (methylamino)-3,4-dihydroisoquinolin- | | |
| | 2(1H)-yl)methanone | | |
| 44 | (2-(Benzyloxy)-4,6-dihydroxyphenyl)(8- | *** | ** |
| | ((2-methoxyethyl)amino)-3,4- | | |
| | dihydroisoquinolin-2(1H)-yl)methanone | | |
| 45 | (2-(Benzyloxy)-4,6-dihydroxyphenyl)(4- | ** | ** |
| | ((1-(oxetan-3-yl)ethyl)amino)isoindolin-2- | | |
| | yl)methanone | | |
| 46 | (2-Benzyloxy-4,6-dihydroxy-phenyl)-[6- | ** | ** |
| | [(dimethylamino)methyl]-3,4-dihydro-1H- | | |
| | isoquinolin-2-yl]methanone | | |
| 47 | (2-Benzyloxy-4,6-dihydroxy-phenyl)-[7- | ** | ** |
| | [(dimethylamino)methyl]-3,4-dihydro-1H- | | |
| | isoquinolin-2-yl]methanone | | |
| 48 | (2-Benzyloxy-4,6-dihydroxy-phenyl)-(6- | ** | ** |
| | methoxy-3,4-dihydro-1 H-isoquinolin-2- | | |
| | yl)methanone | | |
| 49 | (2-Benzyloxy-4,6-dihydroxy-phenyl)-(7- | ** | ** |
| | methoxy-3,4-dihydro-1H-isoquinolin-2- | | |
| | yl)methanone | | |
| 50 | (2-Benzyloxy-4,6-dihydroxy-phenyl)-(4- | ** | * |
| | hydroxy-3,4-dihydro-1H-isoquinolin-2- | | |
| | yl)methanone | | |
| 51 | (2-Benzyloxy-4,6-dihydroxy-phenyl)-(4- | ** | * |
| | hydroxy-3,4-dihydro-1H-isoquinolin-2- | | |
| | yl)methanone | | |
| 52 | (2-(Benzyloxy)-4,6-dihydroxyphenyl)(4- | *** | ** |
| | ((1-methyl-2-oxabicyclo[2.1.1 ]hexan-4- | | |
| | yl)amino)isoindolin-2-yl)methanone | | |
| 53 | (4-((2-Oxabicyclo[2.2.1 ]heptan-4- | *** | ** |
| | yl)amino)isoindolin-2-yl)(2-(benzyloxy)- | | |
| | 4,6-dihydroxyphenyl)methanone | | |
| 54 | (2-(Benzyloxy)-4,6-dihydroxyphenyl)(6- | *** | ** |
| | methoxy-8-((tetrahydrofuran-3-yl)amino)- | | |
| | 3,4-dihydroisoquinolin-2(1H)- | | |
| | yl)methanone | | |
| 55 | (S)-(2,4-Dihydroxy-6-(pyridin-2- | *** | ** |
| | ylmethoxy)phenyl)(8-((tetrahydrofuran-3- | | |
| | yl)amino)-3,4-dihydroisoquinolin-2(1H)- | | |
| | yl)methanone | | |
| 56 | (R)-(2,4-dihydroxy-6-(pyridin-2- | *** | * |
| | ylmethoxy)phenyl)(8-((tetrahydrofuran-3- | | |
| | yl)amino)-3,4-dihydroisoquinolin-2(1H)- | | |
| | yl)methanone | | |
| 57 | (2,4-Dihydroxy-6-(pyridin-2- | *** | * |
| | ylmethoxy)phenyl)(8-(oxetan-3-ylamino)- | | |
| | 3,4-dihydroisoquinolin-2(1H)- | | |
| | yl)methanone | | |
| 58 | (S)-4-((2-(2,4-Dihydroxy-6-(pyridin-2- | *** | * |
| | ylmethoxy)benzoyl)-1,2,3,4- | | |
| | tetrahydroisoquinolin-8-yl)amino)-1- | | |
| | methylpyrrolidin-2-one | | |
| 59 | (R)-4-((2-(2,4-dihydroxy-6-(pyridin-2- | *** | ** |
| | ylmethoxy)benzoyl)-1,2,3,4- | | |
| | tetrahydroisoquinolin-8-yl)amino)-1- | | |
| | methylpyrrolidin-2-one | | |
| 60 | (R)-(2-(Benzyloxy)-4,6- | *** | * |
| | dihydroxyphenyl)(6-(2-methoxyethoxy)-8- | | |
| | ((tetrahydrofuran-3-yl)amino)-3,4- | | |
| | dihydroisoquinolin-2(1H)-yl)methanone | | |
| 61 | 4-((2-(2-(Benzyloxy)-4,6- | *** | * |
| | dihydroxybenzoyl)-1,2,3,4- | | |
| | tetrahydroisoquinolin-8-yl)amino)-1- | | |
| | methylpyrrolidin-2-one | | |
| 62 | 4-((2-(2-(Benzyloxy)-4,6- | *** | *** |
| | dihydroxybenzoyl)-1,2,3,4- | | |
| | tetrahydroisoquinolin-8-yl)amino)-1- | | |
| | methylpyrrolidin-2-one | | |
| 63 | (S)-(2-(Benzyloxy)-4,6- | *** | ** |
| | dihydroxyphenyl)(4-((tetrahydrofuran-3- | | |
| | yl)amino)-7,8-dihydro-1,6-naphthyridin- | | |
| | 6(5H)-yl)methanone | | |
| 64 | (S)-(2-(Benzyloxy)-4,6- | *** | ** |
| | dihydroxyphenyl)(6-(2-methoxyethoxy)-8- | | |
| | ((tetrahydrofuran-3-yl)amino)-3,4- | | |
| | dihydroisoquinolin-2(1H)-yl)methanone | | |
| 65 | (S)-1-(3-((2-(2-(Benzyloxy)-4,6- | *** | * |
| | dihydroxybenzoyl)-1,2,3,4- | | |
| | tetrahydroisoquinolin-8- | | |
| | yl)amino)pyrrolidin-1-yl)ethan-1-one | | |
| 66 | (R)-1-(3-((2-(2-(Benzyloxy)-4,6- | *** | * |
| | dihydroxybenzoyl)-1,2,3,4- | | |
| | tetrahydroisoquinolin-8- | | |
| | yl)amino)pyrrolidin-1-yl)ethan-1-one | | |
| 67 | (S)-1-(3-((2-(2-(Benzyloxy)-4,6- | ** | * |
| | dihydroxybenzoyl)-1,2,3,4- | | |
| | tetrahydroisoquinolin-8- | | |
| | yl)amino)piperidin-1-yl)ethan-1-one | | |
| 68 | (R)-1-(3-((2-(2-(benzyloxy)-4,6- | *** | * |
| | dihydroxybenzoyl)-1,2,3,4- | | |
| | tetrahydroisoquinolin-8- | | |
| | yl)amino)piperidin-1-yl)ethan-1-one | | |
| 69 | 1-(4-((2-(2-(Benzyloxy)-4,6- | ** | * |
| | dihydroxybenzoyl)-1,2,3,4- | | |
| | tetrahydroisoquinolin-8- | | |
| | yl)amino)piperidin-1-yl)ethan-1-one | | |
| 70 | 5-((2-(2-(Benzyloxy)-4,6- | *** | * |
| | dihydroxybenzoyl)-1,2,3,4- | | |
| | tetrahydroisoquinolin-8-yl)amino)-1- | | |
| | methylpiperidin-2-one | | |
| 71 | (2-(Benzyloxy)-4,6-dihydroxyphenyl)(8- | *** | ** |
| | (pyrimidin-5-ylamino)-3,4- | | |
| | dihydroisoquinolin-2(1H)-yl)methanone | | |
| 72 | (2-(Benzyloxy)-4,6-dihydroxyphenyl)(6- | *** | ** |
| | methoxy-8-((tetrahydrofuran-3-yl)amino)- | | |
| | 3,4-dihydroisoquinolin-2(1H)- | | |
| | yl)methanone | | |
| 73 | (2-(Benzyloxy)-4,6-dihydroxyphenyl)(6- | ** | * |
| | methoxy-8-((tetrahydrofuran-3-yl)amino)- | | |
| | 3,4-dihydroisoquinolin-2(1H)- | | |
| | yl)methanone | | |
| 74 | 3-((2-(2-(Benzyloxy)-4,6- | * | * |
| | dihydroxybenzoyl)-1,2,3,4- | | |
| | tetrahydroisoquinolin-8- | | |
| | yl)amino)benzamide | | |
| 75 | 4-((2-(2-(Benzyloxy)-4,6- | * | * |
| | dihydroxybenzoyl)-1,2,3,4- | | |
| | tetrahydroisoquinolin-8- | | |
| | yl)amino)benzamide | | |
| 76 | (2-(Benzyloxy)-4,6-dihydroxyphenyl)(8- | ** | * |
| | (cyclopentylamino)-3,4- | | |
| | dihydroisoquinolin-2(1H)-yl)methanone | | |
| 77 | (2-(Benzyloxy)-4,6-dihydroxyphenyl)(8- | * | * |
| | ((1-methylpiperidin-4-yl)amino)-3,4- | | |
| | dihydroisoquinolin-2(1H)-yl)methanone | | |
| 78 | (2-(Benzyloxy)-4,6-dihydroxyphenyl)(8- | *** | * |
| | ((1-methylpyrrolidin-3-yl)amino)-3,4- | | |
| | dihydroisoquinolin-2(1H)-yl)methanone | | |
| 79 | (2-(Benzyloxy)-4,6-dihydroxyphenyl) (4- | ** | ** |
| | ((methylamino)methyl) isoindolin-2-yl) | | |
| | methanone | | |
| 80 | (2-(Benzyloxy)-4,6-dihydroxyphenyl)(4- | ** | ** |
| | (((tetrahydrofuran-3- | | |
| | yl)amino)methyl)isoindolin-2- | | |
| | yl)methanone | | |
| 81 | (2-(Benzyloxy)-4,6-dihydroxyphenyl)(4- | ** | ** |
| | ((oxetan-3-ylamino)methyl)isoindolin-2- | | |
| | yl)methanone | | |
| 82 | (2-(Benzyloxy)-4,6-dihydroxyphenyl)(4- | *** | ** |
| | (((3-methyloxetan-3- | | |
| | yl)amino)methyl)isoindolin-2- | | |
| | yl)methanone | | |
| 83 | (4-(Aminomethyl)isoindolin-2-yl)(2- | *** | ** |
| | (benzyloxy)-4,6-dihydroxyphenyl)methanone | | |
| | hydrochloride | | |
| 84 | (2-(Benzyloxy)-4,6-dihydroxyphenyl)(7- | ** | ** |
| | ((oxetan-3-ylamino)methyl)-3,4- | | |
| | dihydroisoquinolin-2(1H)-yl)methanone | | |
| 85 | (2-(Benzyloxy)-4,6-dihydroxyphenyl)(7- | ** | ** |
| | (((tetrahydrofuran-3-yl)amino)methyl)-3,4- | | |
| | dihydroisoquinolin-2(1H)-yl)methanone | | |
| 86 | 2-(2-(Benzyloxy)-4,6-dihydroxybenzoyl)-N- | *** | *** |
| | methylisoindoline-5-carboxamide | | |
| 87 | 2-(2-(Benzyloxy)-4,6-dihydroxybenzoyl)-N,N- | *** | *** |
| | dimethylisoindoline-5-carboxamide | | |
| 88 | (2-(Benzyloxy)-4,6-dihydroxyphenyl)(4- | ** | ** |
| | ((tetrahydrofuran-3-yl)oxy)isoindolin-2- | | |
| | yl)methanone | | |
| 89 | (2-(Benzyloxy)-4,6-dihydroxyphenyl)(4- | *** | *** |
| | (pyrimidin-5-ylmethoxy)isoindolin-2- | | |
| | yl)methanone | | |
| 90 | (4-(Azetidin-3-ylmethoxy)isoindolin-2-yl)(2- | ** | ** |
| | (benzyloxy)-4,6-dihydroxyphenyl) methanone | | |
| 91 | 2-(2-(Benzyloxy)-4,6- | ** | *** |
| | dihydroxybenzoyl)isoindoline-4-carbonitrile | | |
| 92 | 1-(2-(2-(Benzyloxy)-4,6- | ** | ** |
| | dihydroxybenzoyl)isoindolin-4-yl)azetidine-3- | | |
| | carbonitrile | | |
| 93 | (4-Aminoisoindolin-2-yl)(2-(benzyloxy)-4,6- | *** | ** |
| | dihydroxyphenyl)methanone | | |
| 94 | N-(2-(2-(benzyloxy)-4,6- | ** | ** |
| | dihydroxybenzoyl)isoindolin-4-yl)acetamide | | |
| 95 | N-((2-(2-(Benzyloxy)-4,6- | ** | ** |
| | dihydroxybenzoyl)isoindolin-4- | | |
| | yl)methyl)acetamide | | |
| 96 | (2,4-Dihydroxy-6-(pyridin-3- | *** | * |
| | ylmethoxy)phenyl)(4-((tetrahydrofuran-3- | | |
| | yl)amino)isoindolin-2-yl)methanone | | |
| 97 | (2,4-Dihydroxy-6-(pyridin-4- | *** | ** |
| | ylmethoxy)phenyl)(4-((tetrahydrofuran-3- | | |
| | yl)amino)isoindolin-2-yl)methanone | | |
| 98 | (2,4-Dihydroxy-6-((tetrahydro-2H-pyran- | ** | * |
| | 4-yl)methoxy)phenyl)(4-((tetrahydrofuran- | | |
| | 3-yl)amino)isoindolin-2-yl)methanone | | |
| 99 | (2,4-Dihydroxy-6-(pyridazin-3- | ** | * |
| | ylmethoxy)phenyl)(4-((tetrahydrofuran-3- | | |
| | yl)amino)isoindolin-2-yl)methanone | | |
| 100 | 8-Amino-3,4-dihydroisoquinolin-2(1H)-yl(2- | ** | ** |
| | (benzyloxy)-4,6-dihydroxyphenyl)methanone | | |
| 101 | N-(2-(2-(benzyloxy)-4,6-dihydroxybenzoyl)- | ** | ** |
| | 1,2,3,4-tetrahydroisoquinolin-8-yl)acetamide | | |
| 102 | N-(2-(2-(Benzyloxy)-4, 6-dihydroxybenzoyl)-1, | * | * |
| | 2, 3, 4-tetrahydroisoquinolin-8-yl) | | |
| | tetrahydrofuran-3-carboxamide | | |
| 103 | t-Butyl (5-(4-((2-(2-(benzyloxy)-4,6- | *** | *** |
| | dihydroxybenzoyl)isoindolin-5-yl)methyl) | | |
| | piperazin-1-yl)pentyl)carbamate | | |
| 104 | (5-((4-(5-Aminopentyl)piperazin-1- | *** | *** |
| | yl)methyl)isoindolin-2-yl)(2-(benzyloxy)-4,6 | | |
| | dihydroxyphenyl)methanone hydrochloride | | |
| 112 | (2-(Benzyloxy)-4,6-dihydroxyphenyl)(4- | *** | ** |
| | (((tetrahydrofuran-3- | | |
| | yl)methyl)amino)isoindolin-2-yl)methanone | | |
| 113 | 3-((2-(2-(Benzyloxy)-4,6-dihydroxybenzoyl)- | *** | * |
| | 1,2,3,4-tetrahydroisoquinolin-8-yl)amino)-N,N- | | |
| | dimethylpyrrolidine-1-carboxamide | | |
| 114 | 4-((2-(2-(Benzyloxy)-4,6-dihydroxybenzoyl)- | ** | * |
| | 1,2,3,4-tetrahydroisoquinolin-8-yl)amino)-1- | | |
| | methylpiperidin-2-one | | |
| 115 | (S)-(2-(Benzyloxy)-4,6-dihydroxyphenyl)(4- | ** | ** |
| | ((tetrahydrofuran-3-yl)amino)-7,8- | | |
| | dihydropyrido[4,3-d]pyrimidin-6(5H)- | | |
| | yl)methanone | | |
| 116 | (2-(Benzyloxy)-4,6-dihydroxyphenyl)(7-fluoro- | ** | ** |
| | 8-((tetrahydrofuran-3-yl)amino)-3,4- | | |
| | dihydroisoquinolin-2(1H)-yl)methanone | | |
| 117 | (2-(Benzyloxy)-4,6-dihydroxyphenyl)(8-((1- | *** | ** |
| | methyl-1H-pyrazol-4-yl)amino)-3,4- | | |
| | dihydroisoquinolin-2(1H)-yl)methanone | | |
| 118 | (2-(Benzyloxy)-4,6-dihydroxyphenyl)(8- | ** | ** |
| | (pyridin-3-ylamino)-3,4-dihydroisoquinolin- | | |
| | 2(1H)-yl)methanone | | |
| 119 | (2-(Benzyloxy)-4,6-dihydroxyphenyl)(8- | ** | ** |
| | (pyrazin-2-ylamino)-3,4-dihydroisoquinolin- | | |
| | 2(1H)-yl)methanone | | |
| 120 | (2-(Benzyloxy)-4,6-dihydroxyphenyl)(8- | *** | ** |
| | (pyridazin-4-ylamino)-3,4-dihydroisoquinolin- | | |
| | 2(1H)-yl)methanone | | |
| 121 | (S)-(2-(Benzyloxy)-4,6-dihydroxyphenyl)(6- | *** | *** |
| | ((4-methylpiperazin-1-yl)methyl)-8- | | |
| | ((tetrahydrofuran-3-yl)amino)-3,4- | | |
| | dihydroisoquinolin-2(1H)-yl)methanone | | |
| 122 | (R)-(2-(Benzyloxy)-4,6-dihydroxyphenyl)(6- | *** | ** |
| | ((4-methylpiperazin-1-yl)methyl)-8- | | |
| | ((tetrahydrofuran-3-yl)amino)-3,4- | | |
| | dihydroisoquinolin-2(1H)-yl)methanone | | |
| 123 | (2-(Benzyloxy)-4,6-dihydroxyphenyl) (8-((4- | ** | * |
| | methylpyrimidin-5-yl)amino)-3,4- | | |
| | dihydroisoquinolin-2(1H)-yl)methanone | | |
| 124 | (S)-(2,4-Dihydroxy-6-((3- | *** | *** |
| | methylbenzyl)oxy)phenyl)(8-((tetrahydrofuran- | | |
| | 3-yl)amino)-3,4-dihydroisoquinolin-2(1H)- | | |
| | yl) methanone | | |
| 125 | (S)-(2-(Benzyloxy)-4,6-dihydroxyphenyl)(6- | *** | *** |
| | ((dimethylamino)methyl)-8-((tetrahydrofuran- | | |
| | 3-yl)amino)-3,4-dihydroisoquinolin-2(1H)- | | |
| | yl)methanone | | |
| 126 | (R)-(2-(Benzyloxy)-4,6-dihydroxyphenyl)(6- | *** | ** |
| | ((dimethylamino)methyl)-8-((tetrahydrofuran- | | |
| | 3-yl)amino)-3,4-dihydroisoquinolin-2(1H)- | | |
| | yl)methanone | | |
| 127 | (S)-(2-(2-(Benzyloxy)-4,6-dihydroxybenzoyl)- | *** | *** |
| | 8-((tetrahydrofuran-3-yl)amino)-1,2,3,4- | | |
| | tetrahydroisoquinolin-6-yl)(4-methylpiperazin- | | |
| | 1-yl)methanone | | |
| 128 | (2-(Benzyloxy)-4,6-dihydroxyphenyl)(6-(2- | ** | ** |
| | methoxyethoxy)-8-(methylamino)-3,4- | | |
| | dihydroisoquinolin-2(1H)-yl)methanone | | |
| 129 | (R)-2-(2-(Benzyloxy)-4,6-dihydroxybenzoyl)- | *** | ** |
| | N,N-dimethyl-8-((tetrahydrofuran-3-yl)amino)- | | |
| | 1,2,3,4-tetrahydroisoquinoline-6-carboxamide | | |
| 130 | (S)-2-(2-(Benzyloxy)-4,6-dihydroxybenzoyl)- | *** | *** |
| | N,N-dimethyl-8-((tetrahydrofuran-3-yl)amino)- | | |
| | 1,2,3,4-tetrahydroisoquinoline-6-carboxamide | | |
| 131 | (R)-(2-(Benzyloxy)-4,6-dihydroxyphenyl)(6-(2- | *** | ** |
| | (dimethylamino)ethoxy)-8-((tetrahydrofuran-3- | | |
| | yl)amino)-3,4-dihydroisoquinolin-2(1H)- | | |
| | yl)methanone | | |
| 132 | (S)-(2-(Benzyloxy)-4,6-dihydroxyphenyl)(6-(2- | *** | *** |
| | (dimethylamino)ethoxy)-8-((tetrahydrofuran-3- | | |
| | yl)amino)-3,4-dihydroisoquinolin-2(1H)- | | |
| | yl)methanone | | |
| 133 | 4-((2-(2-(Benzyloxy)-4,6-dihydroxybenzoyl)- | ** | ** |
| | 1,2,3,4-tetrahydro-isoquinolin-8-yl)amino)-1- | | |
| | methylpyridin-2(1H)-one | | |
| 134 | 5-((2-(2-(Benzyloxy)-4,6-dihydroxybenzoyl)- | *** | * |
| | 1,2,3,4-tetrahydroisoquinolin-8- | | |
| | yl)amino)pyridin-2(1H)-one | | |
| 135 | 5-((2-(2-(Benzyloxy)-4,6-dihydroxybenzoyl)- | *** | ** |
| | 1,2,3,4-tetrahydroisoquinolin-8-yl)amino)-1- | | |
| | methylpyridin-2(1H)-one | | |
| 136 | (R)-4-((2-(2-(Benzyloxy)-4,6- | *** | *** |
| | dihydroxybenzoyl)isoindolin-4-yl)amino)-1- | | |
| | methylpyrrolidin-2-one | | |
| 137 | (S)-4-((2-(2-(Benzyloxy)-4,6- | *** | *** |
| | dihydroxybenzoyl)isoindolin-4-yl)amino)-1- | | |
| | methylpyrrolidin-2-one | | |
| 138 | (R)-(2-(Benzyloxy)-4,6-dihydroxyphenyl)(6- | *** | ** |
| | ((1-methylpiperidin-4-yl)oxy)-8- | | |
| | ((tetrahydrofuran-3-yl)amino)-3,4- | | |
| | dihydroisoquinolin-2(1H)-yl)methanone | | |
| 139 | (S)-(2-(Benzyloxy)-4,6-dihydroxyphenyl)(6- | *** | *** |
| | ((1-methylpiperidin-4-yl)oxy)-8- | | |
| | ((tetrahydrofuran-3-yl)amino)-3,4- | | |
| | dihydroisoquinolin-2(1H)-yl)methanone | | |
| 140 | (S)-(2-(Cyclohexylmethoxy)-4,6- | ** | *** |
| | dihydroxyphenyl)(8-((tetrahydrofuran-3- | | |
| | yl)amino)-3,4-dihydroisoquinolin-2(1H)- | | |
| | yl)methanone | | |
| 141 | (R)-(2-(Cyclopropylmethoxy)-4,6- | ** | * |
| | dihydroxyphenyl)(6-(2-methoxyethoxy)-8- | | |
| | ((tetrahydrofuran-3-yl)amino)-3,4- | | |
| | dihydroisoquinolin-2(1H)-yl)methanone | | |
| 142 | (R)-(2,4-Dihydroxy-6-(pyridin-2- | *** | * |
| | ylmethoxy)phenyl)(6-(2-methoxyethoxy)-8- | | |
| | ((tetrahydrofuran-3-yl)amino)-3,4- | | |
| | dihydroisoquinolin-2(1H)-yl)methanone | | |
| 143 | (2-(Benzyloxy)-4,6-dihydroxyphenyl)(8- | * | * |
| | (methyl(tetrahydrofuran-3-yl)amino)-3,4- | | |
| | dihydroisoquinolin-2(1H)-yl)methanone | | |
| 144 | 2-(Benzyloxy)-4,6-dihydroxyphenyl-(5- | *** | *** |
| | ((oxetan-3-ylamino)methyl)isoindolin-2- | | |
| | yl)methanone | | |
| 145 | (2-(Benzyloxy)-4,6-dihydroxyphenyl)(5- | *** | *** |
| | (((tetrahydrofuran-3- | | |
| | yl)amino)methyl)isoindolin-2-yl)methanone | | |
| 146 | 1-(3-((2-(2-(Benzyloxy)-4,6- | *** | ** |
| | dihydroxybenzoyl)-1,2,3,4- | | |
| | tetrahydroisoquinolin-8-yl)amino)azetidin-1- | | |
| | yl)ethan-1-one | | |
| 147 | (2-(Benzyloxy)-4,6-dihydroxyphenyl)(8-((1,1- | *** | ** |
| | dioxidotetrahydrothiophen-3-yl)amino)-3,4- | | |
| | dihydroisoquinolin-2(1H)-yl)methanone | | |
| 147a | (2-(Benzyloxy)-4,6-dihydroxyphenyl)(8-((1,1- | *** | * |
| | dioxidotetrahydrothiophen-3-yl)amino)-3,4- | | |
| | dihydroisoquinolin-2(1H)-yl)methanone | | |
| 147b | (2-(Benzyloxy)-4,6-dihydroxyphenyl)(8-((1,1- | *** | ** |
| | dioxidotetrahydrothiophen-3-yl)amino)-3,4- | | |
| | dihydroisoquinolin-2(1H)-yl)methanone | | |
| 148 | (2-(Benzyloxy)-4,6-dihydroxyphenyl)(8-((4- | ** | * |
| | methoxytetrahydrofuran-3-yl)amino)-3,4- | | |
| | dihydroisoquinolin-2(1H)-yl)methanone | | |
| 149 | 1-(3-((2-(2-(Benzyloxy)-4,6- | ** | * |
| | bis(methoxymethoxy)benzoyl)-1,2,3,4- | | |
| | tetrahydroisoquinolin-8- | | |
| | yl)(methyl)amino)azetidin-1-yl)ethan-1-one | | |
| 150 | (S)-1-(3-((2-(2-(Benzyloxy)-4,6- | ** | * |
| | dihydroxybenzoyl)-1,2,3,4- | | |
| | tetrahydroisoquinolin-8-yl) (methyl)amino) | | |
| | pyrrolidin-1-yl)-4-methoxybutan-1-one | | |
| 151 | (S)-1-(3-((2-(2-(Benzyloxy)-4,6- | *** | * |
| | dihydroxybenzoyl)-1,2,3,4- | | |
| | tetrahydroisoquinolin-8-yl) amino) pyrrolidin- | | |
| | 1-yl)-4-methoxybutan-1-one | | |

1. A compound, or a pharmaceutically acceptable salt, hydrate or solvate thereof, having the structural Formula (I), shown below: 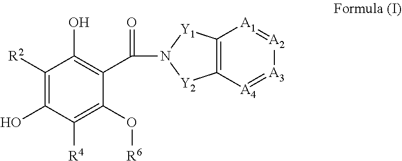
wherein
R² is hydrogen or fluoro;
R⁴ is hydrogen or fluoro;
R⁶ is (1-6C)alkyl, (3-8C)cycloalkyl, or a 4- to 7-membered heterocyclyl ring comprising one heteroatom selected from N, O or S,
or a group having a structure according to formula (A) shown below: 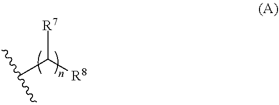
wherein
R⁷ is hydrogen or (1-3C)alkyl;
n is 1 or 2;
R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is —CH₂— or —C(═O)—;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl;
A₁ is selected from N, CH or CR¹¹;
A₂ is selected from N, CH or CR¹²;
A₃ is selected from N, CH or CR¹³;
A₄ is selected from N, CH or CR¹⁴;
with the proviso that only one or two of A₁, A₂, A₃ or A₄ can be N;
R¹¹ is cyano, halo, (1-2C)alkyl, (1-2C)alkoxy, wherein any (1-2C)alkyl or (1-2C)alkoxy is optionally substituted by one or more halo or (1-2C)alkoxy;
or —(CHR_{z})ₐ—Z¹¹,
wherein R_{z} is hydrogen or methyl;
wherein a is 0 or 1; and
Z¹¹ is —OR¹⁵, —NR¹⁶R¹⁷, —C(O)NR¹⁶R¹⁷ or —NR¹⁸C(O)R¹⁹;
wherein R¹⁵ is (1-4C)alkyl, (3-7C)cycloalkyl, phenyl, a carbon-linked 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-7C)cycloalkyl, phenyl, a carbon-linked 4 to 8-membered heterocyclyl, a 5 or 6 membered heteroaryl, —(CHR_{w})_{c}-(3-7C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-8 membered heterocyclic ring;
R¹⁸ is hydrogen or (1-2C)alkyl;
R¹⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₓ)_{d}-(3-7C)cycloalkyl, —(CHRₓ)_{d}-phenyl, —(CHRₓ)_{d}-[4 to 8-membered heterocyclyl] or —(CHRₓ)_{d}-[5 or 6 membered heteroaryl];
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
R¹² is selected from one of the following options:
(i) when R¹¹ is a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹²,
wherein Rᵥ is hydrogen or methyl;
wherein d is 0 or 1; and
Z¹² is —OR²⁰, —NR²¹R²², —C(O)NR²¹R²² or —NR²³C(O)R²⁴;
wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen or methyl and e is 0 or 1;
R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[⁴ to 8-membered heterocyclyl] or —(CHR_{q})_{f}-[5 or 6 membered heteroaryl], wherein R_{q} is hydrogen or methyl and f is 0 or 1;
or R²¹ and R²² are linked, such that, together with the nitrogen atom to which they are attached, they form a 4-8 membered heterocyclic ring;
R²³ is hydrogen or (1-2C)alkyl;
R²⁴ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
wherein each of R²⁰, R²¹, R²², R²³ or R²⁴, or any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein Rₒ is hydrogen or methyl;
wherein h is 0 or 1; and
Z¹² is —OR²⁵, —NR²⁶R²⁷, —C(O)NR²⁶R²⁷ or —NR²⁸C(O)R²⁹;
wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
or R²⁶ and R²⁷ are linked, such that, together with the nitrogen atom to which they are attached, they form a 4-8 membered heterocyclic ring;
R²⁸ is hydrogen or (1-2C)alkyl;
R²⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
wherein each of R²⁵, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen or methyl;
wherein m is 0 or 1; and
Z¹² is —OR³⁰, —NR³¹R³², —C(O)NR³¹R³² or —NR³³C(O)R³⁴;
wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein Rⱼ is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
or R³¹ and R³² are linked, such that, together with the nitrogen atom to which they are attached, they form a 4-8 membered heterocyclic ring;
R³³ is hydrogen or (1-2C)alkyl;
R³⁴ is (1-6C)alkyl, (2-6C)alkynyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[4 to 8-membered heterocyclyl] or —(CHRₕ)_{q}-[5 or 6 membered heteroaryl], wherein Rₕ is hydrogen or methyl and q is 0 or 1;
wherein R³⁰, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group
-L¹-X¹-Q
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —N(R¹⁴)—C(O)—NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-4C)alkyl, (2-4C)alkenyl, (2-4C)alkynyl, or (3-6C)cycloalkyl;
wherein when Q¹ is (1-4C)alkyl, (2-4C)alkenyl, (2-4C)alkynyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy; and wherein one of R¹² or R¹³ is optionally a group:
-L-Q
or
-Lₓ-X
wherein L is a linker;
Lₓ is a linker;
X is a functional group; and
Q is a detection moiety.

2. A compound according to claim 1, wherein the compound is a compound of the formula I-I, I-II or I-III shown below, or pharmaceutically acceptable salt, hydrate or solvate thereof: 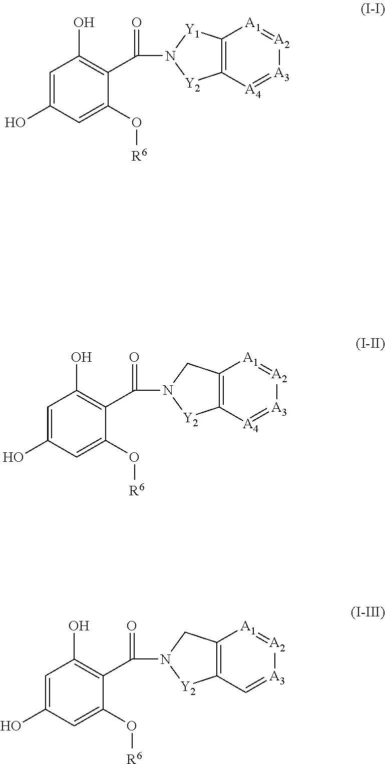
wherein R⁶, Y₁, Y₂, A₁, A₂, A₃, A₄, R¹¹, R¹², R¹³ and R¹⁴ are each as defined in claim 1.

3. A compound according to claim 1 or claim 2, or pharmaceutically acceptable salt, hydrate or solvate thereof, wherein R⁶ is (1-6C)alkyl, (3-6C)cycloalkyl, or a 4- to 6-membered heterocyclyl ring comprising one heteroatom selected from N, O or S,
or a group having a structure according to formula (A) shown below: 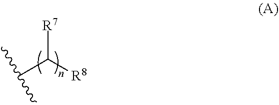
wherein
R⁷ is hydrogen or (1-3C)alkyl;
n is 1 or 2;
R⁸ is (3-8C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy.

4. A compound according to any one of claims 1 to 3, or pharmaceutically acceptable salt, hydrate or solvate thereof, wherein R⁶ is (1-4C)alkyl or a group having a structure according to formula (A) shown below: 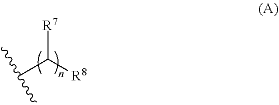
wherein
R⁷ is hydrogen or (1-2C)alkyl;
n is 1 or 2;
R⁸ is (3-6C)cycloalkyl, phenyl, 4- to 6-membered heterocyclyl, or 5- or 6-membered heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy.

5. A compound according to any one of claims 1 to 4, or pharmaceutically acceptable salt, hydrate or solvate thereof, wherein R⁶ is (1-3C)alkyl or a group having a structure according to formula (A) shown below: 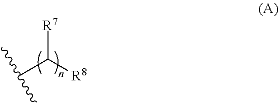
wherein
R⁷ is hydrogen or methyl;
n is 1;
R⁸ is cyclohexyl, phenyl, 6-membered heterocyclyl, 6-membered heteroaryl, each of which is optionally substituted with one or more R⁹; where each R⁹ is independently selected from the group consisting of halogen, methyl, methoxy, trifluoromethyl, or trifluoromethoxy.

6. A compound according to any one of claims 1 to 5, or pharmaceutically acceptable salt, hydrate or solvate thereof, wherein R⁶ is a group having a structure according to formula (A) shown below: 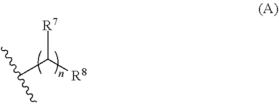
wherein
R⁷ is hydrogen or methyl;
n is 1;
R⁸ is cyclohexyl, phenyl, 6-membered heterocyclyl, pyridyl, each of which is optionally substituted with one or more R⁹; where each R⁹ is independently selected from the group consisting of halogen, methyl, methoxy, trifluoromethyl, or trifluoromethoxy.

7. A compound according to any one of claims 1 to 6, or pharmaceutically acceptable salt, hydrate or solvate thereof, wherein Y₁ is —CH₂—.

8. A compound according to any one of claims 1 to 7, or pharmaceutically acceptable salt, hydrate or solvate thereof, wherein Y₂ is —CH₂—, —CHR_{y2a}—, —CH₂—CH₂—, —CH₂—CHR_{y2b}—, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl.

9. A compound according to any one of claims 1 to 8, or pharmaceutically acceptable salt, hydrate or solvate thereof, wherein Y₂ is —CH₂— or —CH₂—CH₂—.

10. A compound according to any one of claims 1 to 9, or pharmaceutically acceptable salt, hydrate or solvate thereof, wherein A₁, A₂, A₃ or A₄ are selected from one of the following options:
(i) A₁ is selected from N, CH or CR¹¹;
A₂ is selected from N, CH or CR¹²;
A₃ is selected from N, CH or CR¹³;
A₄ is selected from N, CH or CR¹⁴;
with the proviso that only one of A₁, A₂, A₃ or A₄ can be N.
(ii) A₁ is selected from CH or CR¹;
A₂ is selected from N, CH or CR¹²;
A₃ is selected from N, CH or CR¹³;
A₄ is selected from N, CH or CR¹⁴;
with the proviso that only one of A₂, A₃ or A₄ can be N;
(iii) A₁ is CR¹¹;
A₂ is selected from N, CH or CR¹²;
A₃ is selected from N, CH or CR¹³;
A₄ is selected from N, CH or CR¹⁴;
with the proviso that only one of A₂, A₃ or A₄ can be N;
(iv) A₁ is CR¹¹;
A₂ is selected from N or CH;
A₃ is selected from N, CH or CR¹³;
A₄ is selected from N or CH;
with the proviso that only one of A₂, A₃ or A₄ can be N;
(v) A₁ is CR¹¹;
A₂ is CH;
A₃ is CH or CR¹³;
A₄ is CH;
(vi) A₁ is CR¹¹;
A₂ is CH;
A₃ is CH;
A₄ is CH;
(vii) A₁ is CH;
A₂ is CR¹²;
A₃ is CH;
A₄ is CH;
(viii) A₁ is CH;
A₂ is CH;
A₃ is CR¹³;
A₄ is CH;

11. A compound according to any one of claims 1 to 10, or pharmaceutically acceptable salt, hydrate or solvate thereof, wherein R¹¹ is cyano, halo or (1-2C)alkyl optionally substituted by one or more halo or (1-2C)alkoxy;
or —(CHR_{z})ₐ—Z¹¹,
wherein R_{z} is hydrogen or methyl;
wherein a is 0 or 1; and
Z¹¹ is —OR¹⁵, —NR¹⁶R¹⁷, —C(O)NR¹⁶R¹⁷ or —NR¹⁸C(O)R¹⁹;
wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-6 membered heterocyclic ring;
R¹⁸ is hydrogen or (1-2C)alkyl;
R¹⁹ is (1-6C)alkyl, (3-6C)cycloalkyl, phenyl, a carbon-linked 4 to 6-membered heterocyclyl, a 5 or 6 membered heteroaryl, —(CHRₓ)_{d}-(3-6C)cycloalkyl, —(CHRₓ)_{d}-phenyl, —(CHRₓ)_{d}-[4 to 6-membered heterocyclyl] or —(CHRₓ)_{d}-[5 or 6 membered heteroaryl];
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
wherein R^{a} is as defined in claim 1.

12. A compound according to any one of claims 1 to 11, or pharmaceutically acceptable salt, hydrate or solvate thereof, wherein R¹¹ is cyano or —(CHR_{z})ₐ—Z¹¹,
wherein R_{z} is hydrogen or methyl;
wherein a is 0 or 1; and
Z¹¹ is —OR¹⁵, —NR¹⁶R¹⁷ or —NR¹⁸C(O)R¹⁹;
wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-6 membered heterocyclic ring;
R¹⁸ is hydrogen or (1-2C)alkyl;
R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
wherein R^{a} is as defined in claim 1.

13. A compound according to any one of claims 1 to 12, or pharmaceutically acceptable salt, hydrate or solvate thereof, wherein R¹¹ is —(CHR_{z})ₐ—Z¹¹,
wherein R_{z} is hydrogen;
wherein a is 0 or 1; and
Z¹¹ is —NHR¹⁷;
wherein R¹⁷ is selected from;
(i) a group of the formula (A), (B), (C) or (D): 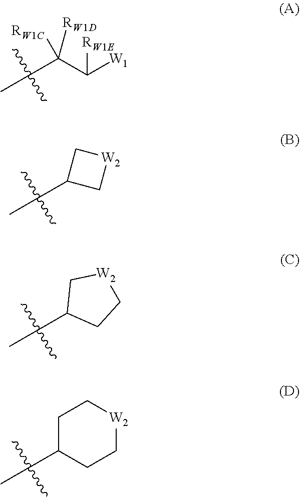
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen or methyl, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2}, S or S(O)₂ where R_{w2} is selected from hydrogen, (1-4C)alkyl or (2-4C)alkanoyl;
and each of formulae (B), (C) and (D) is optionally substituted by one or more R^{a};
wherein R^{a} is as defined in claim 1;
(ii) a bridged version of formulae (C) and (D) above, optionally selected from one of formulae (E) to (J) below: 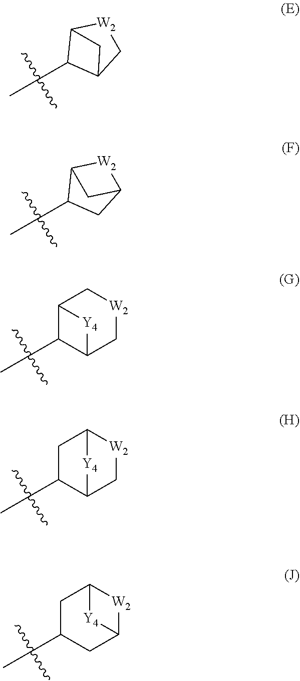
wherein Y₄ is selected from —CH₂—, —CH₂CH₂—,
and each of formulae (E), (F), (G), (H) and (J) is optionally substituted by one or more R^{a};
wherein R^{a} is as defined in claim 1;
(iii) a group of the formula (K), (L) or (M) below: 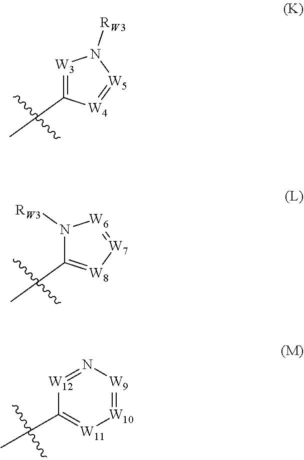
wherein:
R_{W3} is selected from hydrogen or (1-2C)alkyl;
W₃, W₄ and W₅ are selected from N or CR_{w4} where R_{w4} is hydrogen, halo, (1-2C)alkyl or (1-2C)alkoxy;
one of W₆ and W₇ is N and the other is CR_{w5} and W₈ is N or CR_{w5}, where R_{w5} is hydrogen, halo, (1-2C)alkyl or (1-2C)alkoxy;
W₉, W₁₀, W₁₁ and W₁₂ are selected from N or CR_{w6} where R_{w6} is hydrogen, halo, (1-2C)alkyl or (1-2C)alkoxy; with the proviso that only one or two of W₉, W₁₀, W₁₁, and W₁₂ are N.

14. A compound according to any one of claims 1 to 13, or pharmaceutically acceptable salt, hydrate or solvate thereof, wherein R¹¹ is —(CHR_{z})ₐ—Z¹¹,
wherein R_{z} is hydrogen;
wherein a is 0 or 1; and
Z¹¹ is —NHR¹⁷;
wherein R¹⁷ is selected from;
(i) a group of the formula (A), (B), (C) or (D): 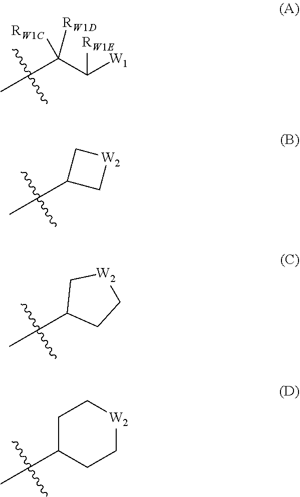
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined in claim 1;
(ii) a bridged version of formulae (C) and (D) above, optionally selected from one of formulae (E) to (J) below: 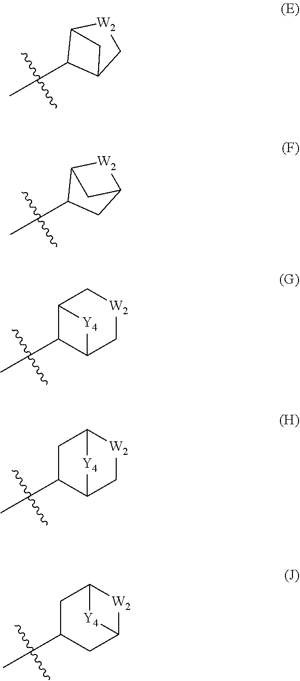
wherein Y₄ is selected from —CH₂—, —CH₂CH₂—,
and each of formulae (E), (F), (G), (H) and (J) is optionally substituted by one or more R^{a};
wherein R^{a} is as defined in claim 1;
(iii) a group of the formula (K), (L) or (M) below: 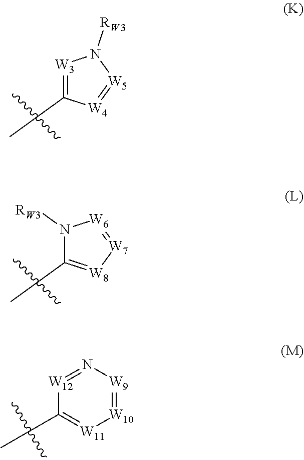
wherein:
R_{W3} is selected from hydrogen or (1-2C)alkyl;
W₃, W₄ and W₅ are selected from N or CH;
one of W₆ and W₇ is N and the other is CH and W₈ is N or CH;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N.

15. A compound according to any one of claims 1 to 14, or pharmaceutically acceptable salt, hydrate or solvate thereof, wherein R¹¹ is —(CHR_{z})ₐ—Z¹¹,
wherein R_{z} is hydrogen;
wherein a is 0 or 1; and
Z¹¹ is —NHR¹⁷;
wherein R¹⁷ is a group of the formula (A), (B), (C) or (D): 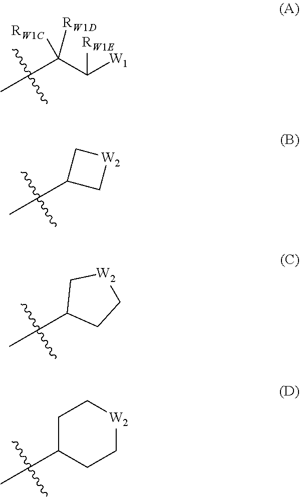
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a}:
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined in claim 1.

16. A compound according to any one of claims 1 to 15, or pharmaceutically acceptable salt, hydrate or solvate thereof, wherein R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹²
wherein Rᵥ is hydrogen or methyl;
wherein d is 0 or 1; and
Z¹² is —OR²⁰, —NR²¹R²², —C(O)NR²¹R²² or —NR²³C(O)R²⁴;
wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen and e is 0 or 1;
R²¹ and R²² are each independently selected from hydrogen, (1-6C)alkyl, (3-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
or R²¹ and R²² are linked, such that, together with the nitrogen atom to which they are attached, they form a 4-8 membered heterocyclic ring;
R²³ is hydrogen or methyl;
R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
wherein each of R²⁰, R²¹, R²², R²³ or R²⁴, or any ring formed when R²¹ and R²² are linked, is optionally substituted with one or more R^{a};
wherein R^{a} is as defined in claim 1.

17. A compound according to any one of claims 1 to 16, or pharmaceutically acceptable salt, hydrate or solvate thereof, wherein R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹² wherein Rᵥ is hydrogen;
wherein d is 0 or 1; and
Z¹² is —OR²⁰, —NR²¹R²² or —C(O)NR²¹R²²;
wherein R²⁰ is (1-4C)alkyl;
R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
or R²¹ and R²² are linked, such that, together with the nitrogen atom to which they are attached, they form a 4-8 membered heterocyclic ring;
wherein each of R²⁰, R²¹ or R²², or any ring formed when R²¹ and R²² are linked, is optionally substituted with one or more R^{a};
wherein R^{a} is as defined in claim 1.

18. A compound according to any one of claims 1 to 17, or pharmaceutically acceptable salt, hydrate or solvate thereof, wherein R¹² is selected from one of the following options R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy.

19. A compound according to any one of claims 1 to 18, or pharmaceutically acceptable salt, hydrate or solvate thereof, wherein R¹³ is selected from cyano, halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein Rₒ is hydrogen or methyl;
wherein h is 0 or 1; and
Z¹² is —OR²⁵, —NR²⁶R²⁷, —C(O)NR²⁶R²⁷ or —NR²⁸C(O)R²⁹;
wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
or R²⁶ and R²⁷ are linked, such that, together with the nitrogen atom to which they are attached, they form a 4-8 membered heterocyclic ring;
R²⁸ is hydrogen or methyl;
R²⁹ is (1-6C)alkyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
wherein each of R²⁵, R²⁶, R²⁷ or R²⁹ or any ring formed when R²⁶ and R²⁷ are linked, is optionally substituted with one or more R^{a};
wherein R^{a} is as defined in claim 1;

20. A compound according to any one of claims 1 to 19, or pharmaceutically acceptable salt, hydrate or solvate thereof, wherein R¹³ is selected from halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein Rₒ is hydrogen or methyl;
wherein h is 0 or 1; and
Z¹² is —OR²⁵, —NR²⁶R²⁷, or —C(O)NR²⁶R²⁷;
wherein R²⁵ is (1-4C)alkyl, a carbon-linked 4 to 8-membered heterocyclyl, or —(CHRₙ)ᵢ-[4 to 8-membered heterocyclyl], wherein Rₙ is hydrogen or methyl and i is 0 or 1;
R²⁶ and R²⁷ are each independently selected from hydrogen, (1-6C)alkyl, a carbon-linked 4 to 8-membered heterocyclyl, or —(CHRₘ)ⱼ-[4 to 8-membered heterocyclyl], wherein Rₘ is hydrogen or methyl and j is 0 or 1;
or R²⁶ and R²⁷ are linked, such that, together with the nitrogen atom to which they are attached, they form a 4-8 membered heterocyclic ring;
wherein each of R²⁵, R²⁶, R²⁷ or any ring formed when R²⁶ and R²⁷ are linked, is optionally substituted with one or more R^{a};
wherein R^{a} is as defined in claim 1.

21. A compound according to any one of claims 1 to 20, or pharmaceutically acceptable salt, hydrate or solvate thereof, wherein:
(i) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen;
wherein m is 0 or 1; and
Z¹² is —OR³⁰, —NR³¹R³², —C(O)NR³¹R³² or —NR³³C(O)R³⁴;
wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[4 to 8-membered heterocyclyl], wherein Rⱼ is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1 or R³¹ and R³² are linked, such that, together with the nitrogen atom to which they are attached, they form a 4-8 membered heterocyclic ring;
R³³ is hydrogen or methyl;
R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[4 to 8-membered heterocyclyl], wherein Rₕ is hydrogen or methyl and q is 0 or 1;
wherein R³⁰, R³¹, R³² or R³⁴, or any ring formed when R³¹ and R³² are linked, is optionally substituted with one or more R^{a};
wherein R^{a} is as defined in claim 1; or
(ii) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy.

22. A compound according to any one of claims 1 to 21, or pharmaceutically acceptable salt, hydrate or solvate thereof, wherein:
(i) each R^{a} is independently selected from the group consisting of oxo, halogen, cyano, hydroxy, (1-4C)alkyl, or a group
-L¹-X¹-
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-4C)alkyl, or (3-6C)cycloalkyl;
wherein when Q¹ is (1-4C)alkyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy;
(ii) each R^{a} is independently selected from the group consisting of oxo, halogen, cyano, hydroxy, (1-2C)alkyl, or a group
-L¹-X¹-Q
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-2C)alkyl, or (3-6C)cycloalkyl;
wherein when Q¹ is (1-2C)alkyl, or (3-6C)cycloalkyl, Q¹ is optionally substituted with one or more Q^{a}, where each Q^{a} is independently selected from the group consisting of hydroxy, halogen, (1-2C)alkyl, (1-2C)alkoxy, (1-2C)haloalkyl, or (1-2C)haloalkoxy;
(iii) each R^{a} is independently selected from the group consisting of oxo, halogen, cyano, hydroxy, (1-2C)alkyl, or a group
-L¹-X¹-Q¹
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
Q¹ is selected from the group consisting of hydrogen or (1-2C)alkyl; or
wherein when Q¹ is (1-2C)alkyl, Q¹ is optionally substituted with one or more Q^{a}, where each Q^{a} is independently selected from the group consisting of hydroxy, halogen, or (1-2C)alkoxy;
(iv) each R^{a} is independently selected from the group consisting of oxo, halogen, cyano, hydroxy, (1-2C)alkyl, or a group
-L¹-X¹-Q
wherein:
L¹ is absent;
X¹ is absent or is selected from the group consisting of —O—, —C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
Q¹ is selected from the group consisting of hydrogen or (1-2C)alkyl;
wherein when Q¹ is (1-2C)alkyl, Q¹ is optionally substituted with one or more Q^{a}, where each Q^{a} is methoxy.

23. The compound, or pharmaceutically acceptable salts, hydrates or solvates thereof, according to any preceding claim, wherein the compound is selected from any one of the following:
(R)-(2-(Benzyloxy)-4,6-dihydroxyphenyl)(8-((tetrahydrofuran-3-yl)amino)-3,4-dihydro-isoquinolin-2(1H)-yl)methanone;
(S)-(2-(Benzyloxy)-4,6-dihydroxyphenyl)(8-((tetrahydrofuran-3-yl)amino)-3,4-dihydro-isoquinolin-2(1H)-yl)methanone;
(2-(Benzyloxy)-4,6-dihydroxyphenyl)(5-(piperazin-1-ylmethyl)isoindolin-2-yl)methanone;
(2-(Benzyloxy)-4,6-dihydroxyphenyl)(5-((methylamino)methyl)isoindolin-2-yl)methanone;
3-((2-(2-(Benzyloxy)-4,6-dihydroxybenzoyl)isoindolin-4-yl)oxy)cyclobutane-1-carbonitrile;
(2-(Benzyloxy)-4,6-dihydroxyphenyl)(4-((tetrahydrofuran-3-yl)amino)isoindolin-2-yl)methanone;
(2-(Benzyloxy)-4,6-dihydroxyphenyl)(4-(oxetan-3-ylamino)isoindolin-2-yl)methanone;
(2-(Benzyloxy)-4,6-dihydroxyphenyl)(4-(3-hydroxypiperidin-1-yl)isoindolin-2-yl)methanone;
(2-(Benzyloxy)-4,6-dihydroxyphenyl)(4-((4-methylpiperazin-1-yl)methyl)isoindolin-2-yl)methanone;
(2-(Benzyloxy)-4,6-dihydroxyphenyl)(4-(morpholinomethyl)isoindolin-2-yl)methanone;
(2-(Benzyloxy)-4,6-dihydroxyphenyl)(5-((dimethylamino)methyl)isoindolin-2-yl)methanone;
(2-(Benzyloxy)-4,6-dihydroxyphenyl)(5-(morpholinomethyl)isoindolin-2-yl)methanone;
(2-(Benzyloxy)-4,6-dihydroxyphenyl)(1-(hydroxymethyl)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((tetrahydro-2H-pyran-4-yl)amino)isoindolin-2-yl)methanone;
(2,4-Dihydroxy-6-methoxyphenyl)(4-((tetrahydrofuran-3-yl)amino)isoindolin-2-yl)methanone;
(2-Ethoxy-4,6-dihydroxyphenyl)(4-((tetrahydrofuran-3-yl)amino)isoindolin-2-yl)methanone;
4-((2-(2-(Benzyloxy)-4,6-dihydroxybenzoyl)isoindolin-4-yl)amino)-1-methylpyrrolidin-2-one;
(2-(Benzyloxy)-4,6-dihydroxyphenyl)(4-(pyrimidin-5-ylamino)isoindolin-2-yl)methanone;
(2-(Benzyloxy)-4,6-dihydroxyphenyl)(4-((oxazol-4-ylmethyl)amino)isoindolin-2-yl)methanone;
(2-(Benzyloxy)-4,6-dihydroxyphenyl)(4-((1-methyl-1H-pyrazol-4-yl)amino)isoindolin-2-yl)methanone;
(S)-(2-(Benzyloxy)-4,6-dihydroxyphenyl)(4-((tetrahydrofuran-3-yl)amino)isoindolin-2-yl)methanone;
(R)-(2-(Benzyloxy)-4,6-dihydroxyphenyl)(4-((tetrahydrofuran-3-yl)amino)isoindolin-2-yl)methanone;
4-((2-(2-(benzyloxy)-4,6-dihydroxybenzoyl)isoindolin-4-yl)amino)-5-methylpyridin-2(1H)-one;
(2-(Cyclopropylmethoxy)-4,6-dihydroxyphenyl)(4-((tetrahydrofuran-3-yl)amino)isoindolin-2-yl)methanone;
(2-(cyclohexylmethoxy)-4,6-dihydroxyphenyl)(4-((tetrahydrofuran-3-yl)amino)isoindolin-2-yl)methanone;
(2-(Benzyloxy)-4,6-dihydroxyphenyl)(5-((tetrahydrofuran-3-yl)amino)-3,4-dihydroisoquinolin-2(1H)-yl)methanone;
(2-(Cyclohexylmethoxy)-4,6-dihydroxyphenyl)(8-((tetrahydrofuran-3-yl)amino)-3,4-dihydroisoquinolin-2(1H)-yl)methanone;
(2-(Benzyloxy)-4,6-dihydroxyphenyl)(4-(methylamino)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((tetrahydro-2H-pyran-3-yl)amino)isoindolin-2-yl)methanone;
(2-(Benzyloxy)-4,6-dihydroxyphenyl)(4-((5,5-difluorotetrahydro-2H-pyran-3-yl)amino)isoindolin-2-yl)methanone;
(2-(Benzyloxy)-4,6-dihydroxyphenyl)(4-(((tetrahydrofuran-2-yl)methyl)amino)isoindolin-2-yl)methanone;
(4-((1H-Pyrazol-4-yl)amino)isoindolin-2-yl)(2-(benzyloxy)-4,6-dihydroxyphenyl)methanone;
(2-(Benzyloxy)-4,6-dihydroxyphenyl)(4-((2-methyl-2H-1,2,3-triazol-4-yl)amino)isoindolin-2-yl)methanone;
1-(3-((2-(2-(Benzyloxy)-4,6-dihydroxybenzoyl)isoindolin-4-yl)amino)pyrrolidin-1-yl)ethan-1-one;
(2-((4-Fluorobenzyl)oxy)-4,6-dihydroxyphenyl)(4-((tetrahydrofuran-3-yl)amino)isoindolin-2-yl)methanone;
(2-((3,4-Difluorobenzyl)oxy)-4,6-dihydroxyphenyl)(4-((tetrahydrofuran-3-yl)amino)isoindolin-2-yl)methanone;
(2,4-Dihydroxy-6-((4-methoxybenzyl)oxy)phenyl)(4-((tetrahydrofuran-3-yl)amino)isoindolin-2-yl)methanone;
(2,4-Dihydroxy-6-(pyridin-2-ylmethoxy)phenyl)(4-((tetrahydrofuran-3-yl)amino)isoindolin-2-yl)methanone;
(2-(Benzyloxy)-4,6-dihydroxyphenyl)(8-(oxetan-3-ylamino)-3,4-dihydroisoquinolin-2(1H)-yl)methanone;
4-((2-(2-(Benzyloxy)-4,6-dihydroxybenzoyl)-1,2,3,4-tetrahydroisoquinolin-8-yl)amino)-1-methylpyrrolidin-2-one;
(2-(Benzyloxy)-4,6-dihydroxyphenyl)(8-(methylamino)-3,4-dihydroisoquinolin-2(1H)-yl)methanone;
(2-(Benzyloxy)-4,6-dihydroxyphenyl)(8-((2-methoxyethyl)amino)-3,4-dihydroisoquinolin-2(1H)-yl)methanone;
(2-(Benzyloxy)-4,6-dihydroxyphenyl)(4-((1-(oxetan-3-yl)ethyl)amino)isoindolin-2-yl)methanone;
(2-Benzyloxy-4,6-dihydroxy-phenyl)-[6-[(dimethylamino)methyl]-3,4-dihydro-1H-isoquinolin-2-yl]methanone;
(2-Benzyloxy-4,6-dihydroxy-phenyl)-[7-[(dimethylamino)methyl]-3,4-dihydro-1H-isoquinolin-2-yl]methanone;
(2-Benzyloxy-4,6-dihydroxy-phenyl)-(6-methoxy-3,4-dihydro-1H-isoquinolin-2-yl)methanone;
(2-Benzyloxy-4,6-dihydroxy-phenyl)-(7-methoxy-3,4-dihydro-1H-isoquinolin-2-yl)methanone;
(2-Benzyloxy-4,6-dihydroxy-phenyl)-(4-hydroxy-3,4-dihydro-1H-isoquinolin-2-yl)methanone;
(2-Benzyloxy-4,6-dihydroxy-phenyl)-(4-hydroxy-3,4-dihydro-1H-isoquinolin-2-yl)methanone;
(2-(Benzyloxy)-4,6-dihydroxyphenyl)(4-((1-methyl-2-oxabicyclo[2.1.1]hexan-4-yl)amino)isoindolin-2-yl)methanone;
(4-((2-Oxabicyclo[2.2.1]heptan-4-yl)amino)isoindolin-2-yl)(2-(benzyloxy)-4,6-dihydroxyphenyl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
(S)-(2,4-Dihydroxy-6-(pyridin-2-ylmethoxy)phenyl)(8-((tetrahydrofuran-3-yl)amino)-3,4-dihydroisoquinolin-2(1H)-yl)methanone;
(R)-(2,4-dihydroxy-6-(pyridin-2-ylmethoxy)phenyl)(8-((tetrahydrofuran-3-yl)amino)-3,4-dihydroisoquinolin-2(1H)-yl)methanone;
(2,4-Dihydroxy-6-(pyridin-2-ylmethoxy)phenyl)(8-(oxetan-3-ylamino)-3,4-dihydroisoquinolin-2(1H)-yl)methanone;
(S)-4-((2-(2,4-Dihydroxy-6-(pyridin-2-ylmethoxy)benzoyl)-1,2,3,4-tetrahydroisoquinolin-8-yl)amino)-1-methylpyrrolidin-2-one;
(R)-4-((2-(2,4-dihydroxy-6-(pyridin-2-ylmethoxy)benzoyl)-1,2,3,4-tetrahydroisoquinolin-8-yl)amino)-1-methylpyrrolidin-2-one;
(R)-(2-(Benzyloxy)-4,6-dihydroxyphenyl)(6-(2-methoxyethoxy)-8-((tetrahydrofuran-3-yl)amino)-3,4-dihydroisoquinolin-2(1H)-yl)methanone;
4-((2-(2-(Benzyloxy)-4,6-dihydroxybenzoyl)-1,2,3,4-tetrahydroisoquinolin-8-yl)amino)-1-methylpyrrolidin-2-one;
4-((2-(2-(Benzyloxy)-4,6-dihydroxybenzoyl)-1,2,3,4-tetrahydroisoquinolin-8-yl)amino)-1-methylpyrrolidin-2-one;
(S)-(2-(Benzyloxy)-4,6-dihydroxyphenyl)(4-((tetrahydrofuran-3-yl)amino)-7,8-dihydro-1,6-naphthyridin-6(5H)-yl)methanone;
(S)-(2-(Benzyloxy)-4,6-dihydroxyphenyl)(6-(2-methoxyethoxy)-8-((tetrahydrofuran-3-yl)amino)-3,4-dihydroisoquinolin-2(1H)-yl)methanone;
(S)-1-(3-((2-(2-(Benzyloxy)-4,6-dihydroxybenzoyl)-1,2,3,4-tetrahydroisoquinolin-8-yl)amino)pyrrolidin-1-yl)ethan-1-one;
(R)-1-(3-((2-(2-(Benzyloxy)-4,6-dihydroxybenzoyl)-1,2,3,4-tetrahydroisoquinolin-8-yl)amino)pyrrolidin-1-yl)ethan-1-one;
(S)-1-(3-((2-(2-(Benzyloxy)-4,6-dihydroxybenzoyl)-1,2,3,4-tetrahydroisoquinolin-8-yl)amino)piperidin-1-yl)ethan-1-one;
(R)-1-(3-((2-(2-(benzyloxy)-4,6-dihydroxybenzoyl)-1,2,3,4-tetrahydroisoquinolin-8-yl)amino)piperidin-1-yl)ethan-1-one;
1-(4-((2-(2-(Benzyloxy)-4,6-dihydroxybenzoyl)-1,2,3,4-tetrahydroisoquinolin-8-yl)amino)piperidin-1-yl)ethan-1-one;
5-((2-(2-(Benzyloxy)-4,6-dihydroxybenzoyl)-1,2,3,4-tetrahydroisoquinolin-8-yl)amino)-1-methylpiperidin-2-one;
(2-(Benzyloxy)-4,6-dihydroxyphenyl)(8-(pyrimidin-5-ylamino)-3,4-dihydroisoquinolin-2(1H)-yl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
(2-(Benzyloxy)-4,6-dihydroxyphenyl)(6-methoxy-8-((tetrahydrofuran-3-yl)amino)-3,4-dihydroisoquinolin-2(1H)-yl)methanone;
3-((2-(2-(Benzyloxy)-4,6-dihydroxybenzoyl)-1,2,3,4-tetrahydroisoquinolin-8-yl)amino)benzamide;
4-((2-(2-(Benzyloxy)-4,6-dihydroxybenzoyl)-1,2,3,4-tetrahydroisoquinolin-8-yl)amino)benzamide;
(2-(Benzyloxy)-4,6-dihydroxyphenyl)(8-(cyclopentylamino)-3,4-dihydroisoquinolin-2(1H)-yl)methanone;
(2-(Benzyloxy)-4,6-dihydroxyphenyl)(8-((1-methylpiperidin-4-yl)amino)-3,4-dihydroisoquinolin-2(1H)-yl)methanone;
(2-(Benzyloxy)-4,6-dihydroxyphenyl)(8-((1-methylpyrrolidin-3-yl)amino)-3,4-dihydroisoquinolin-2(1H)-yl)methanone;
(2-(Benzyloxy)-4,6-dihydroxyphenyl) (4-((methylamino)methyl) isoindolin-2-yl)methanone;
(2-(Benzyloxy)-4,6-dihydroxyphenyl)(4-(((tetrahydrofuran-3-yl)amino)methyl)isoindolin-2-yl)methanone;
(2-(Benzyloxy)-4,6-dihydroxyphenyl)(4-((oxetan-3-ylamino)methyl)isoindolin-2-yl)methanone;
(2-(Benzyloxy)-4,6-dihydroxyphenyl)(4-(((3-methyloxetan-3-yl)amino)methyl)isoindolin-2-yl)methanone;
(4-(Aminomethyl)isoindolin-2-yl)(2-(benzyloxy)-4,6-dihydroxyphenyl)methanone hydrochloride;
(2-(Benzyloxy)-4,6-dihydroxyphenyl)(7-((oxetan-3-ylamino)methyl)-3,4-dihydroisoquinolin-2(1H)-yl)methanone;
(2-(Benzyloxy)-4,6-dihydroxyphenyl)(7-(((tetrahydrofuran-3-yl)amino)methyl)-3,4-dihydroisoquinolin-2(1H)-yl)methanone;
2-(2-(Benzyloxy)-4,6-dihydroxybenzoyl)-N-methylisoindoline-5-carboxamide;
2-(2-(Benzyloxy)-4,6-dihydroxybenzoyl)-N,N-dimethylisoindoline-5-carboxamide;
(2-(Benzyloxy)-4,6-dihydroxyphenyl)(4-((tetrahydrofuran-3-yl)oxy)isoindolin-2-yl)methanone;
(2-(Benzyloxy)-4,6-dihydroxyphenyl)(4-(pyrimidin-5-ylmethoxy)isoindolin-2-yl)methanone;
(4-(Azetidin-3-ylmethoxy)isoindolin-2-yl)(2-(benzyloxy)-4,6-dihydroxyphenyl) methanone;
2-(2-(Benzyloxy)-4,6-dihydroxybenzoyl)isoindoline-4-carbonitrile;
1-(2-(2-(Benzyloxy)-4,6-dihydroxybenzoyl)isoindolin-4-yl)azetidine-3-carbonitrile;
(4-Aminoisoindolin-2-yl)(2-(benzyloxy)-4,6-dihydroxyphenyl)methanone;
N-(2-(2-(benzyloxy)-4,6-dihydroxybenzoyl)isoindolin-4-yl)acetamide;
N-((2-(2-(Benzyloxy)-4,6-dihydroxybenzoyl)isoindolin-4-yl)methyl)acetamide;
(2,4-Dihydroxy-6-(pyridin-3-ylmethoxy)phenyl)(4-((tetrahydrofuran-3-yl)amino)isoindolin-2-yl)methanone;
(2,4-Dihydroxy-6-(pyridin-4-ylmethoxy)phenyl)(4-((tetrahydrofuran-3-yl)amino)isoindolin-2-yl)methanone;
(2,4-Dihydroxy-6-((tetrahydro-2H-pyran-4-yl)methoxy)phenyl)(4-((tetrahydrofuran-3-yl)amino)isoindolin-2-yl)methanone;
(2,4-Dihydroxy-6-(pyridazin-3-ylmethoxy)phenyl)(4-((tetrahydrofuran-3-yl)amino)isoindolin-2-yl)methanone;
8-Amino-3,4-dihydroisoquinolin-2(1H)-yl(2-(benzyloxy)-4,6-dihydroxyphenyl)methanone;
N-(2-(2-(benzyloxy)-4,6-dihydroxybenzoyl)-1,2,3,4-tetrahydroisoquinolin-8-yl)acetamide;
N-(2-(2-(Benzyloxy)-4, 6-dihydroxybenzoyl)-1, 2, 3, 4-tetrahydroisoquinolin-8-yl) tetrahydrofuran-3-carboxamide;
t-Butyl (5-(4-((2-(2-(benzyloxy)-4,6-dihydroxybenzoyl)isoindolin-5-yl)methyl) piperazin-1-yl)pentyl)carbamate;
(5-((4-(5-Aminopentyl)piperazin-1-yl)methyl)isoindolin-2-yl)(2-(benzyloxy)-4,6 dihydroxyphenyl)methanone hydrochloride;
(2-(Benzyloxy)-4,6-dihydroxyphenyl)(4-(((tetrahydrofuran-3-yl)methyl)amino)isoindolin-2-yl)methanone;
3-((2-(2-(Benzyloxy)-4,6-dihydroxybenzoyl)-1,2,3,4-tetrahydroisoquinolin-8-yl)amino)-N,N-dimethylpyrrolidine-1-carboxamide;
4-((2-(2-(Benzyloxy)-4,6-dihydroxybenzoyl)-1,2,3,4-tetrahydroisoquinolin-8-yl)amino)-1-methylpiperidin-2-one;
(S)-(2-(Benzyloxy)-4,6-dihydroxyphenyl)(4-((tetrahydrofuran-3-yl)amino)-7,8-dihydropyrido[4,3-d]pyrimidin-6(5H)-yl)methanone;
(2-(Benzyloxy)-4,6-dihydroxyphenyl)(7-fluoro-8-((tetrahydrofuran-3-yl)amino)-3,4-dihydroisoquinolin-2(1H)-yl)methanone;
(2-(Benzyloxy)-4,6-dihydroxyphenyl)(8-((1-methyl-1H-pyrazol-4-yl)amino)-3,4-dihydroisoquinolin-2(1H)-yl)methanone;
(2-(Benzyloxy)-4,6-dihydroxyphenyl)(8-(pyridin-3-ylamino)-3,4-dihydroisoquinolin-2(1H)-yl)methanone;
(2-(Benzyloxy)-4,6-dihydroxyphenyl)(8-(pyrazin-2-ylamino)-3,4-dihydroisoquinolin-2(1H)-yl)methanone;
(2-(Benzyloxy)-4,6-dihydroxyphenyl)(8-(pyridazin-4-ylamino)-3,4-dihydroisoquinolin-2(1H)-yl)methanone;
(S)-(2-(Benzyloxy)-4,6-dihydroxyphenyl)(6-((4-methylpiperazin-1-yl)methyl)-8-((tetrahydrofuran-3-yl)amino)-3,4-dihydroisoquinolin-2(1H)-yl)methanone;
(R)-(2-(Benzyloxy)-4,6-dihydroxyphenyl)(6-((4-methylpiperazin-1-yl)methyl)-8-((tetrahydrofuran-3-yl)amino)-3,4-dihydroisoquinolin-2(1H)-yl)methanone;
(2-(Benzyloxy)-4,6-dihydroxyphenyl)(8-((4-methylpyrimidin-5-yl)amino)-3,4-dihydroisoquinolin-2(1H)-yl)methanone;
(S)-(2,4-Dihydroxy-6-((3-methylbenzyl)oxy)phenyl)(8-((tetrahydrofuran-3-yl)amino)-3,4-dihydroisoquinolin-2(1H)-yl)methanone;
(S)-(2-(Benzyloxy)-4,6-dihydroxyphenyl)(6-((dimethylamino)methyl)-8-((tetrahydrofuran-3-yl)amino)-3,4-dihydroisoquinolin-2(1H)-yl)methanone;
(R)-(2-(Benzyloxy)-4,6-dihydroxyphenyl)(6-((dimethylamino)methyl)-8-((tetrahydrofuran-3-yl)amino)-3,4-dihydroisoquinolin-2(1H)-yl)methanone;
(S)-(2-(2-(Benzyloxy)-4,6-dihydroxybenzoyl)-8-((tetrahydrofuran-3-yl)amino)-1,2,3,4-tetrahydroisoquinolin-6-yl)(4-methylpiperazin-1-yl)methanone;
(2-(Benzyloxy)-4,6-dihydroxyphenyl)(6-(2-methoxyethoxy)-8-(methylamino)-3,4-dihydroisoquinolin-2(1H)-yl)methanone;
(R)-2-(2-(Benzyloxy)-4,6-dihydroxybenzoyl)-N,N-dimethyl-8-((tetrahydrofuran-3-yl)amino)-1,2,3,4-tetrahydroisoquinoline-6-carboxamide;
(S)-2-(2-(Benzyloxy)-4,6-dihydroxybenzoyl)-N,N-dimethyl-8-((tetrahydrofuran-3-yl)amino)-1,2,3,4-tetrahydroisoquinoline-6-carboxamide;
(R)-(2-(Benzyloxy)-4,6-dihydroxyphenyl)(6-(2-(dimethylamino)ethoxy)-8-((tetrahydrofuran-3-yl)amino)-3,4-dihydroisoquinolin-2(1H)-yl)methanone;
(S)-(2-(Benzyloxy)-4,6-dihydroxyphenyl)(6-(2-(dimethylamino)ethoxy)-8-((tetrahydrofuran-3-yl)amino)-3,4-dihydroisoquinolin-2(1H)-yl)methanone;
4-((2-(2-(Benzyloxy)-4,6-dihydroxybenzoyl)-1,2,3,4-tetrahydro-isoquinolin-8-yl)amino)-1-methylpyridin-2(1H)-one;
5-((2-(2-(Benzyloxy)-4,6-dihydroxybenzoyl)-1,2,3,4-tetrahydroisoquinolin-8-yl)amino)pyridin-2(1H)-one;
5-((2-(2-(Benzyloxy)-4,6-dihydroxybenzoyl)-1,2,3,4-tetrahydroisoquinolin-8-yl)amino)-1-methylpyridin-2(1H)-one;
(R)-4-((2-(2-(Benzyloxy)-4,6-dihydroxybenzoyl)isoindolin-4-yl)amino)-1-methylpyrrolidin-2-one;
(S)-4-((2-(2-(Benzyloxy)-4,6-dihydroxybenzoyl)isoindolin-4-yl)amino)-1-methylpyrrolidin-2-one;
(R)-(2-(Benzyloxy)-4,6-dihydroxyphenyl)(6-((1-methylpiperidin-4-yl)oxy)-8-((tetrahydrofuran-3-yl)amino)-3,4-dihydroisoquinolin-2(1H)-yl)methanone;
(S)-(2-(Benzyloxy)-4,6-dihydroxyphenyl)(6-((1-methylpiperidin-4-yl)oxy)-8-((tetrahydrofuran-3-yl)amino)-3,4-dihydroisoquinolin-2(1H)-yl)methanone;
(S)-(2-(Cyclohexylmethoxy)-4,6-dihydroxyphenyl)(8-((tetrahydrofuran-3-yl)amino)-3,4-dihydroisoquinolin-2(1H)-yl)methanone;
(R)-(2-(Cyclopropylmethoxy)-4,6-dihydroxyphenyl)(6-(2-methoxyethoxy)-8-((tetrahydrofuran-3-yl)amino)-3,4-dihydroisoquinolin-2(1H)-yl)methanone;
(R)-(2,4-Dihydroxy-6-(pyridin-2-ylmethoxy)phenyl)(6-(2-methoxyethoxy)-8-((tetrahydrofuran-3-yl)amino)-3,4-dihydroisoquinolin-2(1H)-yl)methanone;
(2-(Benzyloxy)-4,6-dihydroxyphenyl)(8-(methyl(tetrahydrofuran-3-yl)amino)-3,4-dihydroisoquinolin-2(1H)-yl)methanone;
2-(Benzyloxy)-4,6-dihydroxyphenyl-(5-((oxetan-3-ylamino)methyl)isoindolin-2-yl)methanone;
(2-(Benzyloxy)-4,6-dihydroxyphenyl)(5-(((tetrahydrofuran-3-yl)amino)methyl)isoindolin-2-yl)methanone;
1-(3-((2-(2-(Benzyloxy)-4,6-dihydroxybenzoyl)-1,2,3,4-tetrahydroisoquinolin-8-yl)amino)azetidin-1-yl)ethan-1-one;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((1,1-dioxidotetrahydrothiophen-3-yl)amino)-3,4-dihydroisoquinolin-2(1H)-yl)methanone;
(2-(Benzyloxy)-4,6-dihydroxyphenyl)(8-((4-methoxytetrahydrofuran-3-yl)amino)-3,4-dihydroisoquinolin-2(1H)-yl)methanone;
1-(3-((2-(2-(Benzyloxy)-4,6-bis(methoxymethoxy)benzoyl)-1,2,3,4-tetrahydroisoquinolin-8-yl)(methyl)amino)azetidin-1-yl)ethan-1-one;
(S)-1-(3-((2-(2-(Benzyloxy)-4,6-dihydroxybenzoyl)-1,2,3,4-tetrahydroisoquinolin-8-yl) (methyl)amino) pyrrolidin-1-yl)-4-methoxybutan-1-one;
(S)-1-(3-((2-(2-(Benzyloxy)-4,6-dihydroxybenzoyl)-1,2,3,4-tetrahydroisoquinolin-8-yl) amino) pyrrolidin-1-yl)-4-methoxybutan-1-one;
5-((5-(4-((2-(2-(Benzyloxy)-4,6-dihydroxybenzoyl)isoindolin-5-yl)methyl)piperazin-1-yl)pentyl)carbamoyl)-2-(6-(dimethylamino)-3-(dimethyliminio)-3H-xanthen-9-yl)benzoate;
t-Butyl(5-(2-(2-(benzyloxy)-4,6-dihydroxybenzoyl)isoindoline-5-carboxamido)pentyl)carbamate;
N-(5-Aminopentyl)-2-(2-(benzyloxy)-4,6-dihydroxybenzoyl)isoindoline-5-carboxamide hydrochloride;
5-((5-(2-(2-(Benzyloxy)-4,6-dihydroxybenzoyl)isoindoline-5-carboxamido)pentyl) carbamoyl)-2-(6-(dimethylamino)-3-(dimethyliminio)-3H-xanthen-9-yl)benzoate;
1-(6-((5-(2-(2-(Benzyloxy)-4,6-dihydroxybenzoyl)isoindoline-5-carboxamido)pentyl)amino)-6-oxohexyl)-3,3-dimethyl-2-((1E,3E)-5-((E)-1,3,3-trimethylindolin-2-ylidene)penta-1,3-dien-1-yl)-3H-indol-1-ium formate;
N-(5-(4-((2-(2-(benzyloxy)-4,6-dihydroxybenzoyl)isoindolin-5-yl)methyl)piperazin-1-yl)pentyl)-3-(5,5-difluoro-7-(1H-pyrrol-2-yl)-5H-4λ⁴,5λ⁴-dipyrrolo[1,2-c:2′,1′-f][1,3,2]diazaborinin-3-yl)propenamide; and
2-(2-(Benzyloxy)-4,6-dihydroxybenzoyl)-N-(5-(3-(5,5-difluoro-7-(1H-pyrrol-2-yl)-5H-4λ⁴,5λ⁴-dipyrrolo[1,2-c:2′,1′-f][1,3,2]diazaborinin-3-yl)propanamido)pentyl)isoindoline-5-carboxamide.

24. A pharmaceutical composition comprising a compound as defined in any preceding claim, or a pharmaceutically acceptable salt, hydrate or solvate thereof, in admixture with a pharmaceutically acceptable diluent or carrier.

25. A compound, or a pharmaceutically acceptable salt, hydrate or solvate thereof, as defined in any one of claims 1-23, or a pharmaceutical composition as defined in claim 24:
(i) for use in therapy;
(ii) the treatment of cancer;
(iii) the treatment of cancer, wherein the compound or pharmaceutical composition is administered in combination with another anticancer agent (e.g. a chemotherapeutic agent, an immune checkpoint inhibitor, an immune stimulator or DNA damage repair modulator);
(iv) the treatment of a triplet repeat disorder.
